SEQUENCE LISTING (80 sequences)

SEQ ID NO: 1 - DNA, 21 bp, Artificial Sequence
  Description of Artificial Sequence Synthetic primer
caaggaccgcttcaaccactt

SEQ ID NO: 2 - DNA, 21 bp, Artificial Sequence
  Description of Artificial Sequence Synthetic primer
ccaggatgggtgtgtttgacc

SEQ ID NO: 3 - DNA, 20 bp, Artificial Sequence
  Description of Artificial Sequence Synthetic primer
gtaacccgttgaaccccatt

SEQ ID NO: 4 - DNA, 20 bp, Artificial Sequence
  Description of Artificial Sequence Synthetic primer
ccatccaatcggtagtagcg

SEQ ID NO: 5 - DNA, 20 bp, Artificial Sequence
  Description of Artificial Sequence Synthetic primer
cccacatgaagcgacttccc

SEQ ID NO: 6 - DNA, 21 bp, Artificial Sequence
  Description of Artificial Sequence Synthetic primer
caggtccaggagatcgttgaa

SEQ ID NO: 7 - DNA, 21 bp, Artificial Sequence
  Description of Artificial Sequence Synthetic primer
tttgtgggcctgaagaaaact

SEQ ID NO: 8 - DNA, 21 bp, Artificial Sequence
  Description of Artificial Sequence Synthetic primer
agggctgtcctgaataagcag

SEQ ID NO: 9 - DNA, 22 bp, Artificial Sequence
  Description of Artificial Sequence Synthetic primer
gtgttcagccaaaagaccatct

SEQ ID NO: 10 - DNA, 19 bp, Artificial Sequence
  Description of Artificial Sequence Synthetic primer
ggcctgcatgagggtttct

SEQ ID NO: 11 - DNA, 21 bp, Artificial Sequence
  Description of Artificial Sequence Synthetic primer
cgacaccccaatctcgatatg

SEQ ID NO: 12 - DNA, 21 bp, Artificial Sequence
  Description of Artificial Sequence Synthetic primer
gttgcacagatagtgacccgt

SEQ ID NO: 13 - DNA, 19 bp, Artificial Sequence
  Description of Artificial Sequence Synthetic primer
gtggaccgcacggaatttg

SEQ ID NO: 14 - DNA, 20 bp, Artificial Sequence
  Description of Artificial Sequence Synthetic primer
ggagattcacaccggagtca

SEQ ID NO: 15 - DNA, 21 bp, Artificial Sequence
  Description of Artificial Sequence Synthetic primer
agctcggcggtgtttttatca

SEQ ID NO: 16 - DNA, 21 bp, Artificial Sequence
  Description of Artificial Sequence Synthetic primer
ctgcacaggatcttggagacg

SEQ ID NO: 17 - DNA, 23 bp, Artificial Sequence
  Description of Artificial Sequence Synthetic primer
tatgagcctcgaatccacatagt

SEQ ID NO: 18 - DNA, 22 bp, Artificial Sequence
  Description of Artificial Sequence Synthetic primer
cctcgttctgataagcagtcac

SEQ ID NO: 19 - DNA, 22 bp, Artificial Sequence
  Description of Artificial Sequence Synthetic primer
cgaactggacacacatacagtg

SEQ ID NO: 20 - DNA, 21 bp, Artificial Sequence
  Description of Artificial Sequence Synthetic primer
ctgaggatctctggttgtggt

SEQ ID NO: 21 - DNA, 21 bp, Artificial Sequence
  Description of Artificial Sequence Synthetic primer
ccacctggacgtattccactg

SEQ ID NO: 22 - DNA, 23 bp, Artificial Sequence
  Description of Artificial Sequence Synthetic primer
tcgaatcaaatgactaggagggg

SEQ ID NO: 23 - DNA, 21 bp, Artificial Sequence
  Description of Artificial Sequence Synthetic primer
ctgctacccttgagacacctg

SEQ ID NO: 24 - DNA, 22 bp, Artificial Sequence
  Description of Artificial Sequence Synthetic primer
gggctctgatctctgcatctac

SEQ ID NO: 25 - DNA, 23 bp, Artificial Sequence
  Description of Artificial Sequence Synthetic primer
caaagtgagacctgccaaaaaga

SEQ ID NO: 26 - DNA, 21 bp, Artificial Sequence
  Description of Artificial Sequence Synthetic primer
tggacaagggatctgacagtg

SEQ ID NO: 27 - DNA, 20 bp, Artificial Sequence
  Description of Artificial Sequence Synthetic primer
tccaagtgggtccgactcaa

SEQ ID NO: 28 - DNA, 23 bp, Artificial Sequence
  Description of Artificial Sequence Synthetic primer
ggtctctgtcgattaaataggcg

SEQ ID NO: 29 - DNA, 22 bp, Artificial Sequence
  Description of Artificial Sequence Synthetic primer
aacatacgcaccttcctgaaag

SEQ ID NO: 30 - DNA, 21 bp, Artificial Sequence
  Description of Artificial Sequence Synthetic primer
gtcaaaggggtcaaacagctc

SEQ ID NO: 31 - DNA, 19 bp, Artificial Sequence
  Description of Artificial Sequence Synthetic primer
tcttcacggtgtaccccag

SEQ ID NO: 32 - DNA, 21 bp, Artificial Sequence
  Description of Artificial Sequence Synthetic primer
attagcagcggaaagttggct

SEQ ID NO: 33 - DNA, 20 bp, Artificial Sequence
  Description of Artificial Sequence Synthetic primer
ctgccccatctgtcagggta

SEQ ID NO: 34 - DNA, 22 bp, Artificial Sequence
  Description of Artificial Sequence Synthetic primer
ggtatctcacagtagcgggtaa

SEQ ID NO: 35 - DNA, 19 bp, Artificial Sequence
  Description of Artificial Sequence Synthetic primer
tcgctctgaccaccgtgat

SEQ ID NO: 36 - DNA, 22 bp, Artificial Sequence
  Description of Artificial Sequence Synthetic primer
agggaccgtaaaactggtacaa

SEQ ID NO: 37 - DNA, 23 bp, Artificial Sequence
  Description of Artificial Sequence Synthetic primer
gaacagttcgattttgacagtgg

SEQ ID NO: 38 - DNA, 21 bp, Artificial Sequence
  Description of Artificial Sequence Synthetic primer
caccagcttagtaggctctgc

SEQ ID NO: 39 - DNA, 22 bp, Artificial Sequence
  Description of Artificial Sequence Synthetic primer
aactaccatgttcctcgaacga

SEQ ID NO: 40 - DNA, 21 bp, Artificial Sequence
  Description of Artificial Sequence Synthetic primer
ctccatcaaatcccacaccag

SEQ ID NO: 41 - DNA, 22 bp, Artificial Sequence
  Description of Artificial Sequence Synthetic primer
tggagcagattaaacacgagtg

SEQ ID NO: 42 - DNA, 19 bp, Artificial Sequence
  Description of Artificial Sequence Synthetic primer
ccgcccggaactttctgac

SEQ ID NO: 43 - DNA, 19 bp, Artificial Sequence
  Description of Artificial Sequence Synthetic primer
tggtggaccctctgatccg

SEQ ID NO: 44 - DNA, 21 bp, Artificial Sequence
  Description of Artificial Sequence Synthetic primer
gagcgagcatcctcattctcc

SEQ ID NO: 45 - DNA, 19 bp, Artificial Sequence
  Description of Artificial Sequence Synthetic primer
gatccccgccacccttatc

SEQ ID NO: 46 - DNA, 21 bp, Artificial Sequence
  Description of Artificial Sequence Synthetic primer
gtctccagacggaggtcacat

SEQ ID NO: 47 - DNA, 21 bp, Artificial Sequence
  Description of Artificial Sequence Synthetic primer
ccaagtgtggctcattaggca

SEQ ID NO: 48 - DNA, 22 bp, Artificial Sequence
  Description of Artificial Sequence Synthetic primer
ccaatcttcgactggactctgt

SEQ ID NO: 49 - DNA, 21 bp, Artificial Sequence
  Description of Artificial Sequence Synthetic primer
ggatcacgatggactcgtctt

SEQ ID NO: 50 - DNA, 22 bp, Artificial Sequence
  Description of Artificial Sequence Synthetic primer
ggttgccttgtggaatgtttga

SEQ ID NO: 51 - DNA, 22 bp, Artificial Sequence
  Description of Artificial Sequence Synthetic primer
ccctacaccgtggtctatttcc

SEQ ID NO: 52 - DNA, 19 bp, Artificial Sequence
  Description of Artificial Sequence Synthetic primer
caggaggctttgagtgagc

SEQ ID NO: 53 - DNA, 23 bp, Artificial Sequence
  Description of Artificial Sequence Synthetic primer
tcaccctgacacaaataaggatg

SEQ ID NO: 54 - DNA, 22 bp, Artificial Sequence
  Description of Artificial Sequence Synthetic primer
aggatgagaactcgccaaagat

SEQ ID NO: 55 - DNA, 20 bp, Artificial Sequence
  Description of Artificial Sequence Synthetic primer
gccaacgaggatcttcgagc

SEQ ID NO: 56 - DNA, 20 bp, Artificial Sequence
  Description of Artificial Sequence Synthetic primer
cttctcgcgttccactttgc

SEQ ID NO: 57 - DNA, 21 bp, Artificial Sequence
  Description of Artificial Sequence Synthetic primer
ctctgggagtacctagaaccc

SEQ ID NO: 58 - DNA, 22 bp, Artificial Sequence
  Description of Artificial Sequence Synthetic primer
agcctggtaatcaaacaaagcc

SEQ ID NO: 59 - DNA, 21 bp, Artificial Sequence
  Description of Artificial Sequence Synthetic primer
gaagcagctcctacgaagttc

SEQ ID NO: 60 - DNA, 21 bp, Artificial Sequence
  Description of Artificial Sequence Synthetic primer
aggcaagctcctagtctgacc

SEQ ID NO: 61 - DNA, 21 bp, Artificial Sequence
  Description of Artificial Sequence Synthetic primer
cttggaggcccgttatggttc

SEQ ID NO: 62 - DNA, 21 bp, Artificial Sequence
  Description of Artificial Sequence Synthetic primer
ctgggtagtgagtcttctgga

SEQ ID NO: 63 - DNA, 21 bp, Artificial Sequence
  Description of Artificial Sequence Synthetic primer
agttcaagcggacgtgtactt

SEQ ID NO: 64 - DNA, 20 bp, Artificial Sequence
  Description of Artificial Sequence Synthetic primer
ggttgaggtctacgatgccc

SEQ ID NO: 65 - DNA, 19 bp, Artificial Sequence
  Description of Artificial Sequence Synthetic primer
tccaggagccgcacttcta

SEQ ID NO: 66 - DNA, 20 bp, Artificial Sequence
  Description of Artificial Sequence Synthetic primer
ctccgggatgtggatcttca

SEQ ID NO: 67 - DNA, 20 bp, Artificial Sequence
  Description of Artificial Sequence Synthetic primer
aatgcggcctactcaggcta

SEQ ID NO: 68 - DNA, 19 bp, Artificial Sequence
  Description of Artificial Sequence Synthetic primer
tagtcggcatagggctcgg

SEQ ID NO: 69 - DNA, 20 bp, Artificial Sequence
  Description of Artificial Sequence Synthetic primer
tccggctcaagtgtgtcaag

SEQ ID NO: 70 - DNA, 22 bp, Artificial Sequence
  Description of Artificial Sequence Synthetic primer
aggttgtcacgaatgatttgct

SEQ ID NO: 71 - DNA, 20 bp, Artificial Sequence
  Description of Artificial Sequence Synthetic primer
ggtgctgtctctcgaacaag

SEQ ID NO: 72 - DNA, 21 bp, Artificial Sequence
  Description of Artificial Sequence Synthetic primer
catctgtcgaaagccaccaaa

SEQ ID NO: 73 - DNA, 20 bp, Artificial Sequence
  Description of Artificial Sequence Synthetic primer
tgccgcgctgtttacatctt

SEQ ID NO: 74 - DNA, 23 bp, Artificial Sequence
  Description of Artificial Sequence Synthetic primer
gtgctgacctttaatcagatcca

SEQ ID NO: 75 - DNA, 21 bp, Artificial Sequence
  Description of Artificial Sequence Synthetic primer
ttcgaccagtcctaccgtaat

SEQ ID NO: 76 - DNA, 20 bp, Artificial Sequence
  Description of Artificial Sequence Synthetic primer
gaagatgcctgggtggaagc

SEQ ID NO: 77 - DNA, 21 bp, Artificial Sequence
  Description of Artificial Sequence Synthetic primer
gaagcatgtgcctagagcact

SEQ ID NO: 78 - DNA, 21 bp, Artificial Sequence
  Description of Artificial Sequence Synthetic primer
agcatcctcctttccgctaag

SEQ ID NO: 79 - DNA, 20 bp, Artificial Sequence
  Description of Artificial Sequence Synthetic primer
ccaagctgtctacggactgc

SEQ ID NO: 80 - DNA, 22 bp, Artificial Sequence
  Description of Artificial Sequence Synthetic primer
tcaagaatctcgtcaagcatgg

(19) United States
(12) Patent Application Publication
(10) Pub. No.: US 2019/0382731 A1
(43) Pub. Date: Dec. 19, 2019

(54) Artificial Brain Tissue

(71) Applicant: Trustees of Tufts College, Medford, MA (US)

(72) Inventors: David L. Kaplan, Concord, MA (US); William Cantley, Medford, MA (US); Volha Liaudanskaya, Medford, MA (US); Disha Sood, Medford, MA (US); Yu-Ting Dingle, Cambridge, MA (US); William Collins, Sharon, MA (US); Florenzo G. Omenetto, Lexington, MA (US)

(21) Appl. No.: 16/317,454

(22) PCT Filed: Jul. 11, 2017
(86) PCT No.: PCT/US2017/041599

§ 371 Date: Jan. 11, 2019

Related U.S. Application Data
(60) Provisional application No. 62/360,953, filed on Jul. 11, 2016.

(51) Int. Cl. C12N 5/071; C12N 5/079; A61L 27/58; A61L 27/56; A61L 27/54; A61L 27/44; A61L 27/36; A61L 27/22; G01N 33/50; C12N 5/00

(52) U.S. Cl. CPC C12N 5/0697; C12N 2513/00; C12N 5/0622; C12N 5/069; A61L 27/58; A61L 27/56; A61L 27/54; A61L 27/44; A61L 27/3675; A61L 27/3604; A61L 27/227; G01N 33/5088; C12N 5/0062; A61L 2430/32; C12N 2535/00; C12N 2533/90; C12N 2533/50; C12N 5/0618

(57) ABSTRACT

The present invention provides, among other things, powerful new tools for therapeutic and research uses in the central and/or peripheral nervous system. The present invention provides, inter alia, compositions including a plurality of human nerve cells, a plurality of glial cells, and silk fibroin, as well as methods for making and using such compositions.

### BACKGROUND

The human brain is one of the most complex organs in the body. It is estimated that the average human brain contains approximately 100 billion neurons with an enormous number of connections between them. Furthermore, while the brain comprises a fraction of the body's mass it far outweighs the energy consumption of other organs. The result of the complexity of the human brain is an organ capable of truly stunning processing capacity and is commonly thought of as the basis for consciousness and cognition. Accordingly, there exists a need in the art for a truly relevant model of the brain, both for research and potentially therapeutic purposes.

### SUMMARY

The present invention provides powerful new tools for therapeutic and research uses in the central and/or peripheral nervous system. In some embodiments, provided compositions allow for the creation and use of previously unattainable models of human brain tissue with true physiological relevance. Humanized animal models have been used extensively but evidence suggests these models lack translational relevance. While various aspects of the present invention provide a wide array of advantages, exemplary advantages include: the use of human cells, including human primary cells, the ability to compartmentalize portions of provided compositions to allow for comparative studies and treatments not previously possible, long term cultivation and development of provided compositions (weeks, months, even years), ability to design and implement machine-tissue interfaces including electrical and optical devices, and the ability to combine provided brain tissue-like compositions with a newly developed analog of the blood brain barrier, herein described.

According to various aspects, the present invention provides, inter alia, compositions including a plurality of mammalian (e.g. human) nerve cells, a plurality of glial cells, and silk fibroin, as well as methods for making and using such compositions. The use of silk fibroin in provided compositions allows for a surprising degree of customizability of the mechanical and resulting functional properties thereof, as well as a surprisingly positive effect on the function of both neuronal and glial cells associated therewith. In some embodiments, provided compositions may comprise one or more ECM agents. In some embodiments, provided compositions comprise at least two ECM agents.

In some embodiments, the present invention provides methods of making compositions including the step of providing a plurality of human nerve cells, providing a plurality of glial cells, and associating the plurality of human nerve cells and plurality of glial cells with a silk fibroin matrix or scaffold.

In some embodiments, the present invention provides methods including the steps of providing a composition according to any of the compositions described herein, associating one or more active agents with the composition, and characterizing the effect of the one or more active agents on the composition. In some embodiments, an effect may be or comprise: alteration of the expression of one or more gene or proteins, alteration of the production of one or more meurotransmitters, alteration of cell viability, and/or oxygen and/or glucose metabolism, relative to a control composition not associated with the active agent. In some embodiments, the present invention provides methods including the steps of providing a composition according to any of the compositions described herein, associating one or more active agents with the composition, and determining if the one or more active agents modulates the production of at least one neurotransmitter.

In some aspects, the silk fibroin in provided methods and compositions may include one or more silk fibroin structures. In some embodiments including multiple silk fibroin structures, at least two of the structures are different from one another. In some embodiments, the silk fibroin comprises a three dimensional matrix. In some embodiments, the three dimensional matrix is or comprises a scaffold. In some embodiments, a matrix or scaffold may be or comprise a tube, film, fiber, foam, gel, particle (e.g., microsphere, nanosphere, etc), ring shaped structure, concentric rings, and combinations thereof. In some embodiments, provided compositions comprise a plurality of layers. In some embodiments, compositions comprising a plurality of layers do not include an adhesive added to the composition to hold the layers together.

In accordance with various embodiments, any variety of silk fibroin may be used. For example, in some embodiments, the silk fibroin is selected from the group consisting of silkworm silk fibroin, spider silk fibroin, silk tropoelastin blends, one or more genetically engineered variants of silk fibroin, and combinations thereof.

In some embodiments, the matrix or scaffold comprises a plurality of pores. In some embodiments, at least some of the pores are interconnected. In some embodiments, substantially all of the pores are interconnected. In some embodiments, the arrangement of the plurality of pores is substantially random. In some embodiments, at least some of the plurality of pores are substantially aligned. In some embodiments, the pores are about 50 um to about 1000 um in size. In some embodiments, pore sizes of 500-600 uM may be particularly advantageous for encouraging interconnectivity and cell adhesion. In some embodiments, pore sizes of 200-300 uM may be particularly advantageous for encouraging interconnectivity and cell adhesion.

In accordance with various embodiments, the plurality of nervous system cells may be or comprise any of a variety of nervous system cells. For example, in some embodiments, the plurality of human nerve cells is selected from the group consisting of neurons, neural stem cells, neural progenitor cells, induced pluripotent stem cells, and combinations thereof. In some embodiments, the plurality of neurons comprise at least one of motor neurons and sensory neurons.

In accordance with various embodiments, any of a variety of glial cells may be used. In some embodiments, the plurality of glial cells are human glial cells. In some embodiments, the plurality of glial cells is selected from the group consisting of glial progenitor cells, ependymal cells, NG2 cells, astrocytes, microglia, oligodendrocytes, Schwann cells, tanycytes, and combinations thereof. In some embodiments, at least some of the plurality of glial cells myelinate at least one neuron in a provided composition. In some embodiments, provided compositions comprise neuronal and glial cells present in a ratio such that both cell types receive appropriate nourishment and do not substantially inhibit the function and/or proliferation of each other. In some embodiments, the presence of glial cells as described herein may supplement the exchange of neurotransmitters and contribute to chemical/enzymatic modification of said neurotransmitters.

In some embodiments, provided methods and compositions further include a plurality of endothelial cells. In some embodiments, at least some of the plurality of endothelial cells are human endothelial cells. In some embodiments, substantially all of the plurality of endothelial cells are human endothelial cells. In some embodiments, the endothelial cells comprise a monolayer. In some embodiments, the monolayer is characterized in that it is able to impede the flow of at least one fluid to or from an area substantially devoid of the plurality of human nerve cells and the plurality of glial cells into or from an area comprising the substantial majority of the plurality of human nerve cells and the plurality of glial cells. In some embodiments, the at least one fluid is a liquid or a gas. Additionally or alternatively, in some embodiments, provided compositions may include a plurality of brain microvascular endothelial cells. In some embodiments, this endothelial cell layer also serves a function for selective transport of molecules from one side of the monolayer to the other, regulating what gets into different compartments of provided compositions such as drugs, signaling factors, inflammatory compounds and others.

In accordance with some aspects of the present invention, certain provided compositions may be capable of performing certain functions and/or having certain structures classically associated with a blood brain barrier. By way of non-limiting example, in some embodiments, the present invention also provides compositions including a porous, three dimensional silk fibroin matrix comprising a plurality of pores, a conduit within the matrix sufficient to support flow of at least one fluid, a plurality of brain microvascular endothelial cells, and at least one extracellular matrix (ECM) agent. In some embodiments, the composition comprises at least two ECM agents.

In some embodiments, the at least one ECM agent may include one or more of fibronectin, laminin, fibrin, poly-lysine, poly-ornithine, polysaccharide, collagen, elastin, serum, serum albumin, proteoglycan, melanin and cell adhesion molecule. In some embodiments, the cell adhesion molecule is selected from the group consisting of cadherin, immunoglobulin, selectin, mucin, integrin and combinations thereof. In some embodiments, the at least one ECM agent comprises collagen I, collagen IV and fibronectin. In some embodiments, the at least one ECM agent facilitates and/or promotes growth and/or attachment of cells on or within the matrix.

In accordance with various embodiments, any of a variety of concentrations of ECM agent(s) are contemplated. For example, in some embodiments, the at least one ECM agent is present at a concentration of at least 1 ng/ml (e.g., at least 10 ng/ml, 100 ng/ml, 1 μg/ml, 10 μg/ml, 100 μg/ml, 1 mg/ml, 10 mg/ml, 100 mg/ml or more). In some embodiments, the at least one ECM agent is present at a concentration between 1 ng/ml and 100 mg/ml. In some embodiments, the at least one ECM agent is present substantially uniformly throughout the silk fibroin matrix.

In accordance with various embodiments, provided methods and/or compositions may include at least one active agent. In some embodiments, the at least one active agent comprises one or more of proteins, peptides, antigens, immunogens, vaccines, antibodies or portions thereof, antibody-like molecules, enzymes, nucleic acids, siRNA, shRNA, aptamers, viruses, bacteria, small molecules, cells, hormones, antibiotics, therapeutic agents, diagnostic agents, and any combinations thereof. In some embodiments the at least one active agent comprises an anti-inflammatory agent.

One of the benefits of various embodiments is the capability to include at least one electrical device, optical device, and/optical tool in a provided composition. In some embodiments, provided compositions may include at least one electronic device. In some embodiments, the at least one electronic device comprises at least one electrode. In some embodiments, provided methods and/or compositions further include at least one of an optical device and an optical tool.

As used in this application, the terms “about” and “approximately” are used as equivalents. Any citations to publications, patents, or patent applications herein are incorporated by reference in their entirety. Any numerals used in this application with or without about/approximately are meant to cover any normal fluctuations appreciated by one of ordinary skill in the relevant art.

Other features, objects, and advantages of the present invention are apparent in the detailed description that follows. It should be understood, however, that the detailed description, while indicating embodiments of the present invention, is given by way of illustration only, not limitation. Various changes and modifications within the scope of the invention will become apparent to those skilled in the art from the detailed description.

### BRIEF DESCRIPTION OF THE DRAWING

The foregoing and other objects, aspects, features, and advantages of the present disclosure will become more apparent and better understood by referring to the following description taken in conjunction with the accompanying figures in which:

FIG. 1, panels A-F show isolation and characterization of microglia and astrocytes from rat/mouse primary cultures. Microglia (panels A-C) and astrocytes (panels D-F) were immunostained with specific markers: panel A) ibal/DAPI; panel B) iNOS/DAPI; panel C) GFAP/DAPI; panel D) GFAP/DAPI; panel E) Tuj1/DAPI; and panel F) ibal/04/DAPI. O4—oligodendrocytes; iba1—microglia, iNOS—activated microglia; GFAP—astrocytes; Tuj1—neuronal markers and DAPI (blue) for nuclei counterstaining. Microglial activation was observed through treatment with lipopolysaccharide (LPS), followed by fixation and immunostaining after 3 days. Panel G: Microglia are stained with ibal (green) or markers of activated microglia, panel H: iNOS (green); or panel G: IL-1beta (green) and nuclei are counterstained with DAPI (blue). Microglial inhibition of astrocytes was examined using immunocytochemistry and cell activity in presence of inhibitors. Cells were cultures and fixed after 3 and 7 days, then immunostained with specific markers: GFAP for astrocytes (red, panels J-M), ibal for microglia (green, panels J-M), and DAPI (blue) as a nuclear counterstain. Number of microglia was determined in cells treated with CSF1R inhibitor or control (panel N), and shows that fewer microglia were present in the CSF1R groups at both 3 and 7 days as compared to the Sham control. Cultures were further evaluated via WST-1 assay (panel 0). Scale bars; 100 um.

FIG. 2, panels A-F show evaluation of media effect on neural/microglia growth and proliferation. NM—neural media, MM—glial media. Cells were immunostained with specific markers: GFAP—astrocytic; Tuj1—neuronal markers and DAPI for nuclei counterstaining. WST1-colorimetric proliferation assay. (NM) to glial media (MM), Panel A: 100:0 NM to MM; Panel B: 75:25 NM to MM; Panel C: 50:50 NM to MM; Panel D: 25:75 NM to MM; Panel E: 0:100 NM to MM and phenotypic markers (GFAP—astrocytes; tuj1—neurons; DAPI—nuclear counterstain, Panels A-E) and a WST1 proliferation assay to evaluate numbers of neurons over the course of one week, which showed that the overall number of neurons appeared to increase across all conditions after 7 days in culture (panel F).

FIG. 3, panels A-K show 2D co-culture of neurons+microglia at different ratios. Cells were fixed and immunostained at day 5 with specific markers: Tuj1 (Panels A-E)—neuronal markers; ibal (Panels G-K)—microglia; and DAPI for nuclei counterstaining. Cell ratios were 100:0 neurons to microglia (panels A and G); 95:5 neurons to microglia (panels B and H); 90:10 neurons to microglia (panels C and I); 85:15 neurons to microglia (panels D and J) and 80:20 neurons to microglia (panels E and K).

FIG. 4, panels A-J show 2D co-culture of neurons+astrocytes at different ratios. Cells were cultured in 75:25 neural:glial media for 5 days, then fixed and immunostained with specific markers: ibal (panels F-J)—microglial; GFAP—astrocytic; Tuj1 (panels A-E)—early neuronal; MAP2—late neuronal markers and DAPI for nuclei counterstaining.

FIG. 5, panels A-H show 3D co-culture of neurons with microglia and astrocytes. Cells were fixed and immunostained at 1 week (panels C-H) or 2 weeks (panels A-B) with specific markers: ibal (Panel A and F-H)—microglial; GFAP (panels B-E)—astrocytic; Tuj1 (Panels B-H)—early neuronal; MAP2 (Panel A)—late neuronal markers and DAPI (blue) for nuclei counterstaining. 3D co-culture of neurons with microglia and astrocytes in 100% neural media for 7 days.

FIG. 6, panels A and B show Dissociated Neurospheres seeded into combination hydrogel at the volume:volume ratios of silk:fibronectin:laminin, as depicted (Panel A, 40:10:50; Panel B, 50:10:40). Panels A and B show Beta-III-Tubulin-positive cells (B3T; red, neuronal marker). Panel B shows Synapsin (synaptic marker, green). DAPI nuclear counterstaining is shown in blue. Scale bars: 25 um.

FIG. 7, panels A and B show confocal z-stack sum projections. Panel A: Beta-III-tubulin+ cells within the scaffold at 11 days showing B3T (green) and DAPI (blue). Panel B: Phalloidin (green) stained scaffold at 11 days that showed no B3T+ cells.

FIG. 8, panels A and B show electrically active hiPSC derived 3D neural culture. Panel A: Confocal z-stack max projection showing the presence of neurons (beta-III-tubulin (B3T)—green) and astrocytes (glial fibrillary acidic protein (GFAP)—red) in a 12 week culture; DAPI nuclear counter stain is in blue. Panel B: Local field potential recordings of imaged scaffold. Top Panel: 5 minute control recording in extra cellular recording solution. Bottom Panel: 10 minute recording of same scaffold after addition of CNQX (AMPA/kainate receptor antagonist) and AP-5 (NMDA receptor antagonist)

FIG. 9, panels A and B show examples of Neural Network formation in 3D scaffold. Panel A: Confocal z-stack max projection of hiPSCs in scaffold after 12 weeks in culture showing synaptic density (neurons—B3T: red, synapses—synaptophysin: green). (Scale: 100 um). Panel B: Confocal z-stack max projection of hiPSCs in scaffold after 12 weeks in culture showing neurite extension into central donut pore. (neurons—B3T: red, astrocytes—GFAP: green). (Scale: 100 um).

FIG. 10, panels A-G show characterization and comparison of decellularized fetal and adult-brain derived ECM. Panel A: sGAG and collagen content of the fetal and adult porcine brain ECM over multiple extractions. Consistently higher ratio of collagen to sGAGs found in fetal ECM, while higher ratio of sGAG to collagen found in adult ECM. Panel B: DNA content in the decellularized brain tissues versus adult and fetal whole brains quantified by Pico green assay and seen to be at par with commercially available matrigel. Panel C: a protein gel showing A) Pepsin; B) Molecular Weight Marker; C) Fetal; and D) Adult ECM. Panels D and E show compositional differences between fetal (panel D) and adult (panel E) porcine brain-derived ECM. Relative percentages of the major ECM proteins found in urea-digested brain ECM as quantified by LC/MS. (Panels F and G) An exemplary comprehensive list of the ECM proteins detected via LC/MS in decellularized fetal and adult brain ECM.

FIG. 11, panels A-F show characterization of silk-HA combination scaffolds. SEM images show the pore size differences in panel A) 4% silk—1% HA freeze-dried scaffold at a ratio of 4:1; panel B) 4% silk—1% HA freeze-dried scaffold at a ratio of 3:1; panel C) 4% silk freeze-dried scaffold; and panel D) 6% silk salt-leached scaffold. Panel E shows an LDH assay (toxicity comparison) on 2 week primary rat neuronal cultures grown on freeze-dried silk alone and silk-HA scaffolds alongside the salt leached scaffolds. Panel F shows dynamic mechanical analysis (DMA) on silk-HA scaffolds to determine the modulus via unconfined compression testing.

FIG. 12, panels A-G show primary rat cortical neurons (embryonic day 18) in a 3D silk scaffold infused with collagen I gel (panels C and F) or collagen supplemented with 1000 ug/mL fetal brain ECM (panels A and D) or 1000 ug/mL adult brain ECM (panels B and E) either with (panels D-F) or without (panels A-C) astrocyte-released matricellular proteins (SPARC, Hevin, TSP2). Panel G shows average neurite density per 2D plane of corresponding 3D Z-stack as quantified by using a custom image analysis code; ***p=0.001, **p<0.005, *p<0.007; n=5; collagen I as control

FIG. 13, panels a-e show primary rat neurons in silk-collagen 3D bioengineered brain model in the presence of fetal or adult brain-derived ECM or in collagen gel alone (panel a) Growth of primary cortical rat neurons (embryonic day 18) in 3D silk scaffold infused with collagen I gel or collagen I supplemented with fetal or adult brain ECM at a concentration of 1000 μg/mL. Neurites stained by β-III Tubulin at a 2 week time point (panel b). Growth of primary cortical rat neurons in 3D silk scaffold infused with collagen I gel or collagen I supplemented with fetal or adult brain ECM at 1000 μg/mL of gel (panel c)). Relative viability of cells in fetal or adult ECM at ECM concentrations of 50 ug/mL, 100 ug/mL, 200 ug/mL, 400 ug/mL and 1000 ug/mL (panel d). Relative glutamate release in fetal or adult ECM at ECM concentrations of 50 ug/mL, 100 ug/mL, 200 ug/mL, 400 ug/mL and 1000 ug/mL (panel e).

FIG. 14, panels A-E show rheological characterization of collagen core hydrogel infused with brain ECM (Panel A); Panels B-E show primary embryonic rat neurons encapsulated in collagen I and Hystem C with (panels B and D) or without (panels C and E) brain ECM.

FIG. 15, panels A-O show human neural stem cells derived by direct reprogramming of skin fibroblasts in 3D silk scaffold supplemented with fetal (panel A) and adult (panel B) brain ECM or single component collagen I (panel C) hydrogels, imaged in the hydrogel window of the donut-shaped scaffold (panel D) and stained for neuronal maker, beta-III tubulin (red, panels A-C). Highest axonal network density observed in fetal ECM condition (panel B). The human neural stem cells in 3D silk scaffold supplemented with fetal (panel E) and adult (panel F) brain ECM or single component collagen I (panel G) hydrogels, imaged within the silk scaffold (panel H) and stained for neuronal maker, beta-III tubulin (red, panels E-G). Highest axonal network density observed in fetal ECM condition (panel E). Panels I-K show differentiation of ReNCell neural stem cells in 3D silk scaffold supplemented with fetal (panel I) and adult (panel J) brain ECM or single component collagen I (panel K) hydrogels imaged at 6 week time point within the scaffold (GFAP—astrocytes, green; β-III tubulin, red—neurons). Panels L-O show viability (panels L and M) and toxicity (panels N and O) assays at 1 month (panels L and N) and 3 month (panels M and O) time points on skin fibroblast-derived human neural stem cell 3D cultures. No statistical difference in comparison to collagen I was seen in viability or toxicity when the cells were grown in the presence of decellularized ECM; p>0.305. No increase in cell toxicity due to the presence of either fetal or adult brain-derived ECM.

FIG. 16 shows differentiation quantification of skin fibroblast-derived human neural stem cells in 6 week long 3D cultures. Neuronal markers: TUBB3(beta III tubulin), NEFL (neurofilament light chain); astrocyte makers: GFAP (glial fibrillary acidic protein), S100B; oligodendrocyte markers: MOG (myelin oligodendrocyte glycoprotein), Olig1; Excitatory synapse markers: PSD95, vGlut1; Inhibitory synapse markers: vGAT1, GPHN; stem cell markers: SOX2, NES (nestin). Greater differentiation into neurons and astrocytes was observed in brain ECM containing conditions, as opposed to collagen I condition and there was a significant increase in neuronal and astrocytic markers in the presence of native brain ECM; p<0.001.

FIG. 17, panels A and B show exemplary flowcharts of provided methods of fabricating provided BBB-type scaffolds with a silk tube and salt-leached sponge.

FIG. 18, panels a-d. shows photographs of exemplary compositions including a silk fibroin tube, specifically, a silk tube in salt-leached sponge, bioreactor units and BMEC cells stained for tight junction protein. Panel a: A silk tube embedded in a salt-leached sponge; panel b: bioreactor with screw closure; panel c: bioreactor with magnetic closure; panel d: exemplary micrograph of BMECs expressing the tight junction protein ZO-1 when cultured on an ECM gel comprising collagen I (1 mg/mL), collagen IV (400 μg/mL), and fibronectin (100 μg/mL). Scale bar: 50 um.

FIG. 19, panels A-B. show two exemplary embodiments of provided sponges for use as a 3D in vitro BBB model. Panel A: The salt-leached sponge has larger pores (500-600 μm). A silk tube is embedded in the salt-leached sponge to provide a smooth surface for EC adhesion. Panel B: The lyophilized sponge has smaller pores (20-90 μm). The filling of ECM gel provides a smooth surface for EC adhesion. In both sponges, neural cells are seeded in the bulk space of the sponge.

FIG. 20 shows an exemplary micrograph of a provided composition with HUVECs formed into a monolayer in a silk tube. GFP-HUVECs were seeded in the silk tube. Fluorescence images showed the HUVECs form a monolayer on the luminal wall of the silk tube.

FIG. 21, panels a and b. show exemplary micrographs of BMECs adhered to a provided silk tube. BMECs were injected into the silk tube and the bioreactors were placed on a roller for 2 hrs (panel a) or placed upside down for 2 hrs (panel b). BMECs were cultured for 2 days and the nuclei were stained with Hoechst.

FIG. 22, panels a-c show micrographs of exemplary BMEC morphology on an ECM gel. BMECs formed web-like morphology on Matrigel (panel a, patchy monolayer on collagen I gel (panel b), and monolayer on collagen I+collagen IV+fibronectin gel (panel c).

FIG. 23, panels A-C show exemplary graphs depicting cytotoxicity analysis of certain provided embodiments by LDH assay at varying time points and concentrations (in mM) of methylprednisolone (MP). Panel A shows relative LDH activity at up to 7 days and at doses of up to 8.7 mM MP. Panel B shows relative LDH activity at up to 8 days and up to 8.7 mM MP. Panel C shows relative LDH activity at up to 8 days and at up to 8.7 mM MP.

FIG. 24, panels A-D show representative Live/Dead cell staining at day 15, after exposure to up to 8.7 mM methylprednisolone. Panel A shows control cells, panel B shows results after exposure to 1.5 mM methylprednisolone, panel C shows results after exposure to 3 mM methylprednisolone, and panel D shows results after exposure to 8.7 mM methylprednisolone. Red signal—dead cells, Green signal—live cells.

FIG. 25, panels A-H show representative HPLC chromatograms for media collected from low- (1.5 mM) and high (8.7 mM) concentration methylprednisolone constructs after up to 8 days of exposure. Panel A, 1.5 mM MP, 1 hour of exposure; Panel B, 1.5 mM MP, 24 hours exposure; Panel C, 1.5 mM MP, 3 days exposure; Panel D, 1.5 mM MP, 8 days exposure; Panel E, 8.7 mM MP, 1 hour of exposure; Panel F, 8.7 mM MP, 24 hours exposure; Panel G, 8.7 mM MP, 3 days exposure; Panel H, 8.7 mM MP, 8 days exposure.

FIG. 26 shows an exemplary graph of cumulative methylprednisolone release from certain provided silk/hydrogel constructs as measured by HPLC at doses of 1.5, 3, 6, and 8.7 ug of methyprednisolone over a course of up to 16 days.

FIG. 27, panels A-D show: PanelA) Side view of computer aided design (CAD) models of 6 well bioreactor within which (panel B) electrostimulation inserts (white) may be placed containing tissue constructs. This bioreactor may then be connected with (panel C) an independent four channel electrostimulation devices or (panel D) alternatively to a frequency generator.

FIG. 28 shows an exemplary schematic representation of a study to evaluate post—TBI changes in vivo and in vitro using certain provided compositions and methods.

FIG. 29 shows two flow diagrams of Akt/mTOR signaling and related pathways that can be affected post TBI.

FIG. 30, panels A-K show A-F) results from an exemplary Live/Dead assay 3 hours post-Controlled Cortical Impact (CCI) on either one week (panels A-C) or two month (panels D-F) old cultures. Red signal—dead cells, Green signal—live cells. Panels G and H show Caspase 3 activity (G, 1 week; H; 2 month old cultures) and panels I and J show Caspase 9 activity (I, 1 week; J, 2 month old cultures) post-CCI at two time points: 3 and 6 hours. Panel E shows an exemplary LDH activity 1 hour post-CCI in 1 week and 2 month old cultures at depths of 1.2 mm and 0.6 mm injury depths versus sham controls.

FIG. 31, panels A-J show post-CCI evaluation of the viability and toxicity of 2 week old mouse cultures (first experimental time point). Panel A) Live and Dead assay showing injured (panel A) and sham (panel A′) conditions (live cells—green; dead cells—red). Panel B) LDH activity up to 24 hours post-injury in injured and sham control conditions. Panel C) Caspase 3 activity up to 24 hours post injury in injured and sham control conditions and panel D) Caspase 9 activity up to 24 hours post injury in sham and control conditions. Panel E) exemplary micrographs of cell viability (green—live cells; red—dead cells) in CCI-treated and sham control on mouse samples of 2, 4, and 6 weeks old. Panel F) Glutamate release after up to 24 hours in 2 week old cultures; G) Glutatmate release up to 24 hours post injury in 4 week old cultures; H) Glutatmate release up to 24 hours post injury in 6 week old cultures. I) Caspase 3 production up to 24 hours post injury in 2, 4 and 6 week old cultures; and J) Caspase 9 production up to 24 hours post-injury in 2, 4, and 6 week old cultures.

FIG. 32, panels A-D show an exemplary schematic of neuromelanin-silk sponge preparation (panel A) an image of NM-silk sponge after 4 weeks of differentiation (panel B); and exemplary micrographs of LUHMES dopaminergic neurons stained with beta-III tubulin after two weeks of differentiation), and beta-III tubulin positive neural projections (panel D, which is an inset of C). Scale bars=200 μm.

FIG. 33, panels A-E show 3D (panels A and B) cultures of LUHMES cells stained for dopaminergic markers DRD2 (not shown) and tyrosine hydroxylase (red). DAPI nuclear counterstain is shown in blue. SEM images of Neuromelanin (NM) treated-silk sponge—a high surface area provided a scaffolding on which cells adhered. Synthetic lewy bodies were prepared by combining NM solution with 0.01M FeCl3 solution and collecting the precipitate (panels C and D). Scale bars: 75 um (panels A and B); 50 um (panels C and D). Panel E shows exemplary results from a metabolic utilization assay (e.g. WST-1 assay) of LUHMES and rat cortical cells after up to 4 weeks in culture. Panel F shows levels of several developmental markers (including PAX6, VIM, SOX2, NES, NCAM, SNAP, BDNF, ASYN) validated by RT-PCR relative to the housekeeping gene GAPDH for both day 0 and week 4 cortical neurons.

FIG. 34, panels A-F show exemplary Patch Clamp Analysis and Local Field Potentials of LUHMES cells. Patch clamp analysis of undifferentiated LUHMES (panel A) differentiated LUHMES (panel B) and cortical neurons (panel C). Cortical neurons exhibited a higher density of voltage gated currents under voltage clamp. An abundance of inward currents is seen. Local field potentials show spontaneous action potential firing and electrically evoked spiking were observed with LUHMES (panel D) and cortical neurons (panel E).

FIG. 35, panels A and B show concentration of exemplary neurotransmitters in neuromelanin and cortical silk sponges (panel A) and expression level analysis of neurotransmitters in cortical, cortical NM and LUHMES NM cultures. Exemplary neurotransmitter processing enzymes evaluated include (1) TH—Tyrosine hydroxylase (2) AADC—Amino acid decarboxylase (3) PNMT—Phenylethanolamine-N-methyl transferase (4) DBH—Dopamine beta hydroxylase (5) MAO—Monoamine oxidase and COMT—Catachol-O-Methyl Transferase.

FIG. 36, panels A-I show immunocytochemistry of LUHMES cells and staining for vesicle dye FM-1-43 in (red, panel A), and the excitatory post synaptic density marker (PSD-95m red, panels B and C), synaptophysin (SYN) (red, panels H and I), alpha-synuclein (ASYN; red, panels D and E), tyrosine hydroxylase (TH, red, panels F and G), phalloidin (green, panels C, E, G, and I) and DAPI nuclear countersatin (blue, panels A-H). were observed. Scale bars: 200 μm.

FIG. 37 shows results from an RT-PCR evaluating exemplary neurotransmitter processing enzymes including Catachol-O-Methyl Transferase (COMT), Monamine oxidase (MAO), Amino acid decarboxylase (AADC), tyrosine hydroxylase (TH), Dopamine beta hydroxylase (DBH), and Phenylethanolamine-N-methyl transferase (PNMT) in Cortical and LUHMES cells. Each of the evaluated enzymes are implicated in the modification of neurotransmitters dopamine, norepinephrine, epinephrine, serotonin and their breakdown product homovanilic acid.

FIG. 38, panels A-C show a schematic describing the methods of interfacing regions of the rhombencephalon and mesencephalon to observe characteristic markers of developmental stages (panel A) and cerebellar neurons in vitro after two weeks in cultures, stained with beta-3-tubulin (green) and DAPI (blue). Scale bars: 200 μm (panel B) and 50 μM (panel C).

FIG. 39, panels A-D show methylprednisolone (“MP”) release kinetics from fibrinogen and collagen gels. MP release was measured using HPLC. Panels A and B show exemplary graphs of cumulative release (in μg) of MP (at each of four concentrations: 1.5 mM (blue circle); 3 mM (red square); 6 mM (green triangle); and 8.7 mM (purple inverted triangle) from fibrinogen (panel A) and collagen (panel B) over the course of 12 days; panels C and D show the change in concentration (in μg/mL) of MP per day, over the course of 12 days from fibrinogen (panel C) and collagen (panel D) gels. Data represent the mean±SD (n=3).

FIG. 40, panels A-G show that methylprednisolone has no negative effect on neuronal viability within 2 weeks of treatment. Panels Ai-Ev show representative images of live (calcein, green) cells in a Live/Dead assay to assess neuronal viability. Panels are shown at five different concentrations (0, 1.5, 3, 6, and 8.7 mM MP) at 1, 3, 5, 7 and 14; propidium iodide (“PI”) positive cells are not shown). Scale bar: 100 μm. Panels Fi-Fv show statistical analyses of PI positive cells at 1, 3, 5, 7, and 14 days at 0, 1, 3, 6, and 8.7 mM MP. Bars represent the average number of dead cells per culture condition. Error bars represent mean±SD (n=6). Statistical significance is indicated above each graph, as appropriate (*p<0.05 and ** p<0.01); Panels Gi-Gv show DNA quantification analysis with PicoGreen assay in 0, 1.5, 3, 6, and 8.7 mM MP at 1, 3, 5, 7, and 14 days. Statistical significance is indicated above each graph as appropriate (*p<0.05, ** p<0.01, *** p<0.001, and **** p<0.0001).

FIG. 41, panels A and B show the effects of MP on neuronal viability through a representative lactate dehydrogenase (LDH) (Panel A) assay and ROS/RNS (Panel B) release. Panel A shows LDH activity (in mU/mL) and Panel B shows ROS/RNS release (in RFUs) at 1, 3, 5, 7, and 14 days for 0 (blue), 1.5 (red), 3 (green), 6 (purple), and 8.7 (orange) mM MP. Error bars represent mean±SD (n=3). Significant differences are indicated above the graphs with asterisks, as appropriate (*p<0.05, **p<0.01, and ***p<0.001).

FIG. 42, panels A-J show representative results of effects of methylprednisolone on microglial morphology and cytokine production. Panels A and B show proliferation ability of M0, M1, and M2 polarized microglia (MG; panel A) and macrophages (RM; panel B) treated/non-treated with methylprednisolone (MP) with DNA quantification assay. *p<0.05. Panel C shows a panel of cytokines in M0, M, 1 and M2 polarized microglia and macrophages treated/non-treated with methylprednisolone. Data were normalized to a positive control, and 8.7 MP samples were compared to OMP samples. Panels Di-Gxii shows results from immunocytochemical and morphological analysis of cells after exposure to methylprednisolone. Immunocytochemistry against the pan: ibal, CD11b; and polarization ill-beta and mannose receptor (MR) markers of microglial (panels Di-Fxii) and macrophages (panel Gi-xii) at day 3 (panels Di-Dxii and Gi-Gxii) after administration of activation molecules, and Day 7 post-treatment with 0 (panels Ei-Exii) and 8.7 (panels Fi-Fxii) mM methylprednisolone. Panels H-J show RT-qPCR data, which confirms polarization of microglia in M1 and M2 stages via expression of CD11b pan-microglial/macrophage marker (panel H); M1-specific marker, CD68 (panel I); and M2-specific marker, CD163 (panel J). Gene expression was normalized to RSP18 gene, and the expression of the genes of interest was compared to the M0 non-activated control group.

FIG. 43, panels Ai-Cxii show exemplary images of M1 activated, M2 activated, or non-activated cells that were fixed for immunological analysis on days 3 (panels Ai-xii), 5 (panels Bi-Bxii), and 7 (panels Ci-Cxii) at 0, 1.5, 3, or 8.7 mM methylprednisolone (MP), stained for the microglial marker ibal (green), and analyzed using a fluorescent microscope at days 3, 5, and 7.

FIG. 44, panels A-D show measurement of secretion of a representative pro-inflammatory marker (TNF alpha) by microglia at days 1, 3, and 7 in 0 (panel A), 1.5 (panel B), 3 (panel C) or 8.7 mM (panel D) methylprednisolone (MP). Significance is indicated above each graph as appropriate (*p<0.05)

FIG. 45, panels A-D show measurement of secretion of an exemplary anti-inflammatory marker (IL-10) by microglia at days 1, 3, and 7 in 0 (panel A), 1.5 (panel B), 3 (panel C) or 8.7 mM (panel D) methylprednisolone (MP). Significance is indicated above each graph as appropriate (*p<0.05)

FIG. 46, panels A and B show representative schematics (as in Silver, Daniel J., et al. Journal of Neuroscience 33.39 (2013): 15603-15617) identifying the presence (panel A, non-invasive tumor) or absence (panel B, invasive tumor) of chondroitin sulfate proteoglycans in the tumor microenvironment.

FIG. 47, panels A-C′ show representative images from patient-derived brain tumor samples in 3D silk constructs infused with collagen I and supplemented with native porcine brain-derived fetal ECM (FECM; panels A and A′), adult ECM (AECM; panels B and B′) or pure collagen I (Col I) (panels C and C′). Dissociated medulloblastoma (grade IV) cells at 2 months were stained with synaptophysin (green, panels A-C), INI-1 (red, panels A-C), β-III tubulin (red, panels A′-C′), and/or glial fibrillary acidic protein (GFAP, green, panels A′-C′); nuclei are marked with DAPI (blue). Scale bars: 100 μm; max projections of z-stacks imaged using confocal within the scaffold ring. Panels D and E show exemplary graphs showing lactate dehydrogenase (LDH) (panel D) and Wst-1 (panel E) assays, respectively, at 1 and 2 month time points of medulloblastoma cultures; *p=0.04, ***: p<0.001, each determined using ANOVA on log transformed data.

FIG. 48 shows the amount of endogenous chondroitin sulfate proteoglycans (CSPGs) released into media at 1 and 2 months by medulloblastoma cells and human induced neural stem cells (hiNSCs) in either fetal, adult, or collagen I supplemented ECM, when cultured in silk-collagen 3D constructs supplemented with native porcine brain-derived ECM. Significance is indicated above the graph with asterisks, which correspond to the legend on the right-hand side of the figure.

FIG. 49 shows quantification of concentrations (in pg/mL) of representative matrix metalloproteinases (“MMPs”; MMP-1, MMP-2, MMP9, and MMP-10) and tissue inhibitors of metalloproteinases (“TIMPs”, TIMP-1, TIMP-2, TIMP-4) released in media after two months of culture by medulloblastoma (hatched bars) and human induced neural stem cells (hiNSCs; solid bars), in either fetal, adult, or collagen I-supplemented ECM, when cultured in 3D silk-collagen constructs supplemented with native porcine brain-derived ECM.

FIG. 50, panels A and B show characterization of decellularized fetal and adult brain ECM. Panel A shows measurements via fluorescence assisted carbohydrate electrophoresis (“FACE”) of fetal and adult ECM, and fetal and adult whole brain; Chondroitin sulfate (CS) and hyaluronan (HA) bands are visible and identified by arrows; ΔDiHA: hyaluronan, ΔDi0S: non-sulfated CS, ΔDi6S: 6-sulfated CS, ΔDi4S: 4-sulfated CS. Panel B shows quantification of CS and HA in adult and fetal brain-derived ECM; significant differences are indicated by horizontal bars above the graph, *p=0.0331;****p<0.0001.

FIG. 51 shows an exemplary schematic of a representative silk tube and salt-leached sponge. The silk tube provides a smooth 3D surface on which brain endothelial cells (bECs) can adhere, mimicking native brain microvasculature. The sponge provides support for 3D culture of astrocytes, mimicking brain tissue. The pores in the tube allow soluble factors and small cell extensions to pass through the scaffold.

FIG. 52 shows an exemplary schematic of a representative lyophilized channel sponge. The luminal surface of the channel, when filled in with ECM gels, provides a smooth 3D surface for bECs to adhere, mimicking the brain microvasculature. The sponge provides support for 3D culture of astrocytes, mimicking brain tissue. The pores in this design allow direct cell-to-cell contact.

FIG. 53 shows a schematic of an exemplary fabrication method for a silk tube and salt-leached sponge.

FIG. 54 shows a schematic of an exemplary fabrication method for a silk lyophilized channel sponge.

FIG. 55, panels a-f show that hCMEC/D3 cells adhered to and formed a monolayer on the luminal wall of the silk tubes and channel. Panel a shows a light microscopic view of the morphology of the immortalized human cerebral microvascular endothelial cell line hCMEC/D3 in 2D. hCMEC/D3 were seeded in the luminal wall of the tube and the channel. The scaffolds were cut open and cells were stained with phalloidin (panels b-f, green). Panel b shows hCMEC/D3 cells adhered to the silk tube. Panels c and d show confocal images of phalloidin-stained hCMEC/D3 cells in the tube of salt-leached sponges. Panels e and f show confocal images of phalloidin-stained hCMEC/D3 cells in the channel of lyophilized sponges.

FIG. 56 demonstrates that hCMEC/D3 survived in co-culture media. The graph shows viability, via WST1 assay, of hCMEC/D3 in—100% hCMEC/D3 media, 100% hCMEC/D3 media without bFGF, 50%: 50% mix of astrocyte and hCMEC/D3 media, and 100% astrocyte media. Dots represent readings from each sample, and lines represent averages.

FIG. 57, panels A and B show a light micrograph of primary human astrocytes as used in the study (panel A), and exemplary staining showing expression of GFAP (panel B, green; nuceli are marked via Hoescht stain in blue). Scale bar in panel B: 100 μm.

FIG. 58, panels A and B show exemplary photographs of astrocytes adhered to and spread in the bulk space of salt-leached sponges. Panel A shows a cross-section of the tube surrounded by phalloidin (green)-positive astrocytes. Panel B is an enlargement of the area inside the dashed-square of panel A. Scale bars: A, 500 μm; B, 200 μm.

FIG. 59 demonstrates that primary human astrocytes survived in co-culture media. The graph shows viability, via WST1 assay, of primary human astrocytes in—100% astrocyte media, 50%:50% mix of astrocyte and hCMEC/D3 media, and 100% hCMEC/D3 media. Dots represent readings from each sample, and lines represent averages.

FIG. 60 shows a representative bioreactor setup used to introduced physiologically-relevant shear stress to the bECs in the scaffold. A scaffold is contained within a PDMS chamber and magnet-secured lid. bECs are injected via the needle into the tube/channel within the sponge. The needles are then connected to a peristaltic pump (not shown), which circulates culture media at a user-defined rate.

FIG. 61, panels A-D show graphs of (HOX) Genes associated with the Rat Cortex and Cerebellum over a timecourse of 0 (panel A), 4 (panel B), and 8 (panel C) weeks. Panel D shows PCA reduction to 3D of control (blue dots) and experimental (red dots) conditions.

FIG. 62 shows exemplary spontaneous neural network depolarization of cerebellar neurons via local field potential (in my/ms).

FIG. 63, panels A-N show hematoxylin and eosin (“H&E”) staining (panels A-C) and immunostaining (panels D-J) of the cerebellum and cell cultures (panels K-N). The cerebellum contains stratified layers and the morphology of the embryonic cerebellum (panel A) becomes increasingly complex as development to adulthood (panel B) continues. A closer observation of the layer stratification can be observed under higher resolution (panel C). A gross anatomical comparison of the fetal (panels C-F) and adult (panels G-H) cerebellum reveals this stratification is also observed in components of the ECM as well. Panels C-N are stained for beta-III tubulin (green), with nuclei marked via DAPI stain (blue), and are also stained for chondroitin sulfate (panels D, G, and K; red), parvalbumin (panels D, H, and L), calbindin (panels E, I, and M), and calretinin (panels F, J, and N).

FIG. 64, panels A-D show representative scanning electron micrograph (“SEM”) of directionally frozen silk and H&E staining of brain cortices. Panels A and C are SEM micrographs of an exemplary traditional silk sponge (panel A; scale bar—100 μm) and silk after unidirectional freezing (panel C; scale bar—200 μm). Panels B and D show exemplary tissue slices from adult (panel B) and embryonic (panel D) brain cortex stained with H&E.

FIG. 65 shows representative metabolic utilization by cerebellar neurons in long-term (8 weeks) culture as measured by Wst-1 assay (panel A) and LDH assay (panel B) to demonstrate active cellular metabolism, showing that cerebellar neuron scaffolds remained active for the 8 week culture period.

FIG. 66, panels A-L show representative data from proteomic analyses of LUHMES cultures. Panel A shows a standard ladder, panel B shows a whole cell lysate, panel C shows results from a ubiquitin pulldown assay and panels D-I show subcellular fractions from 3 month LUHMES cell cultures. Subcellular fractions include cytoplasmic (panel D), nuclear (panel E), chromosomal (panel F), cytoskeletal (panel G), and mitochondrial (panel H) fractions as well as a collagen gel control (panel I). Panel J shows a graph of the results of a representative ELISA comparing ubiquitin concentrations in cytoplasmic, cell membrane, nuclear, and chromosomal fractions from Day-0 and 3 month timepoints for LUHMES and cortical neurons. Panel K shows subcellular fraction distribution in 1 month LUHMES cells and panel L shows a representative ELISA of H2AX in LUHMES and cortical neurons at day 0, and 1 and 4-month timepoints.

FIG. 67, panels A-E show representative analysis by RT-PCR of expression of epidemiological genes in Day 0 and 3 month cultures of cortical (panel A) and LUHMES (panel C) cultured neurons. Panels A and C compare the relative expression change of certain commonly methylated genes, relative to the expression of relative expression of Ct values (1-(ΔCT/GAPDH-CT), and panels B and D show fold changes by comparing gene expression levels in LUHMES cells to those in cortical neurons. Panel E shows fold changes in brain and blood for several genes known to be increased or decreased in Parkinson's Disease.

FIG. 68, panels A-I show representative methylation analyses of exemplary genes of interest in Parkinson's Disease. Panels A-H show annealing curves of eight genes of interest, as compared to the housekeeping control gene, GAPDH (Panel A: GAPDH; Panel B: Vesicular monoamine transporter (VMAT); Panel C: Tyrosine Hydroxylase (TH); Panel D: Dopamine transporter (DAT); Panel E: Dopamine Receptor (DRD2); Panel F: Alpha synuclein (ASYN); Panel G: Synaptophysin (SYN); Panel H: Monoamine Oxidase (MAO)). Black indicates a methyl specific primer with genomic DNA, red indicates a non-methyl specific primer with genomic DNA, green indicates a methyl specific primer with Bisulfite DNA; and blue indicates a non-methyl specific primer with Bisulfite DNA. Evaluation of the CT curves and Tm Product seen in panel I provide conformation of relative abundance and primer efficiency of the genes in panels A-H (above). CT values have been heat mapped to indicate the lowest (red) and highest (green) abundance/efficacy. Panel I shows a representative TM product column comparing the annealing temperature values shown in panels A-H. Red circles indicate higher TM/higher “GC” content and green circles indicate lower TM/higher “AT” content.

FIG. 69, panels A-C show DNA quantification of cells that did not attach to silk sponges. DNA was quantified from cells that did not attach to sponges of porosities ranging from 100 μm-700 μm in cortical (panel A), striatal (panel B), and both cortical and striatal (panel C) neurons.

FIG. 70, panels A-G show matrix structure and mechanical properties of silk sponges. Panels A-E show representative SEM micrographs of silk sponges with different pore sizes/porosity. Panel F shows an exemplary graph of a quantification of pore sizes within the sponges Panel G shows a graph of Young modulus of silk sponges with different porosity. Scale bar: 200 um;*p<0.05, **p<0.01, and ***p<0.001.

FIG. 71, panels A-E′ show optimization of a 3D brain model and the effect of different pore sizes (100, 300, 500, 600, and 700 μm) on neuronal behavior in monocultures. Panels A and B show neuronal viability using a Live/Dead dead assay and calcein staining (green) of striatal (panels A-E) and cortical (panels A′-E′) neurons. Panel F shows quantification of neuronal viability through measurement of PI-positive cells normalized against total DNA in striatal neurons.

FIG. 72, panels A-G show effect of different pore sizes on astrocytes. Panels A and B show exemplary astrocytes (green) after 7 (panels A-E) and 14 (panel A′-E′) days of culture; scale bar—100 μm. Panel F shows representative astrocyte viability at 7 and 14 days using results a Live (green cells)/Dead (red cells; not shown). Panel G shows cell proliferation ability as measured via PicoGreen assay.

FIG. 73, panels Ai-Dv show immunocytochemical analyses for markers of different cortical layers. Panels Ai-Av show neurons stained for Tbr1, which identifies layer V/VI neurons. Panels Bi-Bv show neurons stained for Satb2, which identifies layers II/IV neurons. Panels Ci-Cv show neurons stained for PSD95, a marker of excitatory synapses. Panels Di-Dv show cells stained for beta-III tubulin (clone tuj1), which is a pan-neuronal marker. Scale bar: 100 um.

FIG. 74, panels Ai-Diii show immunocytochemical analyses for different subtypes of striatal neurons. Panels Ai-Av show cells stained for the pan-neuronal marker, beta-III tubulin (clone tuj1, green). Panels Bi-Biii show neurons stained for the neuronal dendritic marker, MAP2 (red). Panels Ci-Ciii show neurons stained for an inhibitory marker, Gephyrin (red). Panels Di-Diii show neurons stained for the excitatory synapse marker, PSD95 (green).

FIG. 75, panels A-E show exemplary graphs of calcium fluctuations in cortical neurons after seven days in culture in sponges with different porosities.

FIG. 76, panels A-E show exemplary graphs of calcium fluctuations in striatal neurons after seven days in culture in sponges with different porosities.

FIG. 77 shows a schematic of a representative design flow that uses a scaffold with porosity of 300 μm and striatal neurons, glial cells, and a hemorrhage challenge, followed by a series of assays to characterize viability, metabolism, and cytotoxicity.

FIG. 78, panels A and B show a schematic of an intracranial hemorrhage (panel A) depicting possible locations and types of hemorrhage (epidural hemorrhage occurs between the dura membrane and the skull, subdural hemorrhage occurs underneath the dura membrane, and intracerebral hemorrhage occurs below the cortex), and a chart depicting characteristics/features of intracerebral hemorrhage (“ICH”) and corresponding ICH scores (panel B).

FIG. 79, panels A-G show representative SEM micrographs of silk fibroin salt-leached sponges with different porosities (panel A, 100 μm; panel B, 300 μm; panel C, 500 μm; panel D, 600 μm; panel E, 700 μm; and panel F, 600-100 μm) and a graph sowing quantification of their porosity (panel G).

FIG. 80, panels A-F show cell phenotype using immunocytochemical analyses of striatal neurons (panels A-C) and astrocytes (panels D-F) at 3 (panels A and D), 7 (panels B and E), and 14 (panels C and F) days of culture. Striatal neurons were stained for beta-III tubulin (tuj1, green (panel C) or red (panel A)), dopaminergic neuron-specific marker DARPP-32 (panels A-C; green) and astrocyte-specific antibodiesto GFAP (green), S-100 (red), and DARPP-32 (red (panels B and C) or green (panel A). DAPI (blue) was used as a nuclear marker.

FIG. 81, panels A and B show cell proliferation in 3D brain scaffolds of different porosities. Panel A shows proliferation of striatal neurons at days 7 and 14 in scaffolds with 100, 300, 500, 600, and 700 μm pores. At day 7, striatal neurons showed a significantly (p<0.05) higher cell count in scaffolds with 300 μm pores as compared to other porosities and at 14 days, cell count was higher in scaffolds with 100 μm pores as compared to other porosities. Viability measurements showed a much higher dead cell count in the 600 μm scaffold, with the number of dead cells decreasing with decreasing porosity size. Panel B shows astrocyte proliferation over 14 days.

FIG. 82, panels A-M show representative images from live (calcein; green)/dead (PI; red) assays of neurons (panels A-E) and astrocytes (panels G-K) from scaffolds with 100 (panels A and G), 300 (panels B and H), 500 (panels C and I), 600 (panels D and J), and 700 (panels E and K)μm pores. Panels F and L show quantification of percentage of dead cells in each porosity; no significant differences were observed between porosities for either neurons or astrocytes. Panel M shows results from a representative Wst-1 assay, used to assess proliferation of astrocytes at days 1, 3, 7, and 14 in scaffolds with porosities of 100, 300, 500, 600, and 700 μm. No significant differences between groups were observed.

FIG. 83, panels A-F show exemplary results of characterization of the Tri-Culture model response after hemorrhage challenge. In all panels, i) negative control, ii) sham, iii) sham+nec-1, iv) ICH, v) ICH+nec-1 groups were evaluated; 4 hour time points are blue and 24 hour time points are red. Panel A shows DNA quantification as measured by total cell number after blood injections. In the 24 hour group, significantly more cells were counted in nec-1 conditions. Panel B shows cell viability rate as determined via Live/Dead assay. Viability was lower in ICH groups after 4 hours, and all groups except the negative control showed lower cell viability after 24 hours. Panel C shows results from MTS assays higher metabolic activity in ICH groups after 4 hours and stabilization after 24 hours. Panel D shows measurement of ATP activity, which was higher in ICH groups after 4 hours and stabilized by 24 hours. Panel E shows LDH assays (normalized for cell number/DNA quantity), with higher levels of LDH activity observed in ICH groups after 4 and 24 hours, and a slightly lower level in ICH+nec-1 after 24 hours. Panel F shows ROS assays (normalized for cell number/DNA quantity), with significantly lower ROS levels in nec-1 groups as compared to negative control sham groups; nec-1-treated groups also had significantly lower ROS levels than their respective untreated samples.

FIG. 84, panels A-C show a schematic of an exemplary three-step tissue model workflow. In step 1 (panel A) fibroin sponges were coated with poly-ornithine and then laminin, then seeded with clusters of hiPSCs. The cells were cultured and expanded for 5 days after which the scaffold was filled with collagen-I hydrogel (step 2; panel B). Scaffolds were then maintained long-term in a neuronal and glial supporting media (step 3, panel C).

FIG. 85, panels A-C show representative assays used in determining optimal seeding density for hiPSC growth/differentiation. Panel A shows results from a PicoGreen assay, performed on scaffolds seeded with indicated cell densities, over time (n=3, error bars=standard deviation) ***P<0.001, *P<0.05. Panel B shows a representative image of media usage after 3 weeks of growth: the first columns contain scaffolds seeded with 1×10⁶ cells and the second three columns are seeded with 5×10⁵ cells. Panel C shows a representative brightfield image of projections growing into the central window of a scaffold seeded with 5×10⁵ cells at 8 weeks post-collagen fill; scale bar: 500 μm.

FIG. 86, panels A-G show a timeline of beta-3-tubulin within the 3D tissue model. Panels A-F show representative images (max projections of confocal z-stacks) of scaffolds taken at two (panel A), four (panel B), six (panel C), eight (panel D), ten (panel E), and twelve weeks (panel F); beta-III tubulin, green; scale bar: 100 μm. Panel G shows a box and whisker plot of binary max projections to determine percent of the imaged area covered by beta-III tubulin-positive staining at two, four, six, eight, ten, and twelve weeks; (n=5-12 imaged fields per time point)

FIG. 87 shows qPCR results expressed as relative gene expression of several representative genes of interest in cells from healthy scaffolds grown and differentiated for 3 and 5 months as compared to those cells maintained as hiPSCs. Several representative cell markers for neurons and subtypes, synaptic markers, and glial cells are shown. (NANOG—pluripotent marker; FGFS—ectodermal germ layer; GATA4—endodermal germ layer; T—mesodermal germ layer; TUBB3, ENO2, NES, SNAI2, EGR4, NEUROD2, NEUROD6—early neuronal markers; MAP2—neural maturation marker; GRIN1 and GRIA1—glutamatergic neurons; TPH1—serotonergic neurons; TH—dopaminergic; ChAT—cholinergic neurons; GABBR1—GABAergic neurons; DLG4, SYN3, and SYP—synaptic markers; FOS, TSNARE, YKT6—activity markers; GFAP—astrocyte marker; SCL2A1—glucose transporter; CNP—oligodendrocyte marker GFAP—astrocytes). Error bar: standard deviation, n=4 samples per timepoint, timepoints derived from independent preparations)

FIG. 88, panels A-D″ show electrophysiological activity in scaffolds. Panels A-D show representative local field potential recordings from spontaneously active scaffolds, taken for 5 minutes in artificial cerebrospinal fluid (ACSF); spikes were counted as peaks above threshold. Panels A′-D′ show representative brightfield images of the scaffolds from which the recordings were taken; scale bar—200 μm. Panels A″-D″ show 20× images of the same scaffolds as in A′-D′ but magnified on the central window of the model.

FIG. 89, panels A-B′ show calcium activity in 8 and 12 week samples. Panels A and B show calcium activity in scaffolds as measured by relative fluorescent intensity of Fluo-4 over a three-minute period at eight (panel A) and twelve (panel B) weeks. Individual traces demonstrate the relative fluorescence of single cells found in the recordings over time. Panels A′ and B′ are screenshots of representative calcium imaging recordings taken from 8 and 12 week old samples respectively (20× images).

FIG. 90, panels A-D′ show growth of neurons and astrocytes differentiated from hiPSCs for 8 months. Panels A, B, C, and D show representative confocal images of 3D scaffolds seeded with Alzheimer's diagnosed patient-derived hiPSCs (panels A and C) or control hiPSCS derived from a healthy patient (panels B and D) grown for 8 months and stained with GFAP and B3T. Panels A′, B′, C′, and D′ show the max projection of the confocal image in panels A, B, C, and D, respectively. B3T—beta-III-tubulin (green—neurons), GFAP—Glial fibrillary acidic protein (red—astrocytes), (scale bar: 100 μm).

FIG. 91, panels A-C show a toxicity assay and LDH assays on Anaplastic Ependymoma cells cultured in Endothelial cell media (EGM 2MV) and Complete Neurobasal media (NBM). Panel A shows a toxicity assay on ependymoma cells after one week in culture, comparing differences between fetal ECM (FECM), adult ECM (AECM) and Collagen (Col) in 3D cultures, as well as 2D culture conditions. Panels B and C show LDH assays after 2 weeks (panel B) and 1 month (panel C) in fetal ECM (FECM, green circles), adult ECM (AECM, blue squares) and Collagen (Col, red triangles), in 3D cultures. Signficant differences were observed between groups, according to the asterisks above the graphs and the accompanying figure legends.

FIG. 92, panels A-D show metabolic measurements after three months in culture via LDH assay (panel A), ROS/NOS assay (panel B) and WST-1 assay (panel D). Panel C is a representative image of cells in culture. Panels A and B show cells cultured in FECM, AECM or Collagen, either with or without combinations of chondroitinase, CSPG, Gelatin, Cisplatin, EGM-2MV+NBM, and or FBS. No significant differences were seen in LDH levels (panel A), between or within conditions. In the ROS/NOS assay (panel B), significant differences were observed within groups of several conditions, as noted by the asterisks above the bars on the graph. A WST-1 assay (panel D) showed significant differences both within and between several groups, as shown by the asterisks, lines above the bar graph and accompanying figure legends.

FIG. 93, panels A-D show representative images from metabolic imaging used to measure redox ratios and analysis of results. Cells and silk were separated (segmented), and marked by green or red, respectively, and cells were then color-coded using measured redox ratio values according to the scale shown below panel B. Panel A shows segmentation with cells in green and silk in red. Panel B shows re-coloring to create a redox ratio map of the cells according to the colormetric scale below the image. Panels C and D show results of LDH assays (panel C) and redox ratios (panel D) following chondroitinase treatment.

### DEFINITIONS

In order for the present disclosure to be more readily understood, certain terms are first defined below. Additional definitions for the following terms and other terms are set forth throughout the specification.

In this application, unless otherwise clear from context, the term “a” may be understood to mean “at least one.” As used in this application, the term “or” may be understood to mean “and/or.” In this application, the terms “comprising” and “including” may be understood to encompass itemized components or steps whether presented by themselves or together with one or more additional components or steps. Where ranges are provided herein, the endpoints are included. As used in this application, the term “comprise” and variations of the term, such as “comprising” and “comprises,” are not intended to exclude other additives, components, integers or steps.

“About”: As used in this application, the terms “about” and “approximately” are used as equivalents. Any numerals used in this application with or without about/approximately are meant to cover any normal fluctuations appreciated by one of ordinary skill in the relevant art. In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

“Administration”: As used herein, the term “administration” refers to the administration of a composition to a subject. Administration may be by any appropriate route. For example, in some embodiments, administration may be enteral, interdermal, intramedullary, intramuscular, intrathecal, intravenous, intraventricular, mucosal, nasal, subcutaneous, topical, transdermal, vaginal and vitreal.

“Amino acid”: As used herein, the term “amino acid,” in its broadest sense, refers to any compound and/or substance that can be incorporated into a polypeptide chain, e.g., through formation of one or more peptide bonds. In some embodiments, an amino acid has the general structure H2N—C(H)(R)—COOH. In some embodiments, an amino acid is a naturally-occurring amino acid. In some embodiments, an amino acid is a synthetic amino acid; in some embodiments, an amino acid is a D-amino acid; in some embodiments, an amino acid is an L-amino acid. “Standard amino acid” refers to any of the twenty standard L-amino acids commonly found in naturally occurring peptides. “Nonstandard amino acid” refers to any amino acid, other than the standard amino acids, regardless of whether it is prepared synthetically or obtained from a natural source. In some embodiments, an amino acid, including a carboxy- and/or amino-terminal amino acid in a polypeptide, can contain a structural modification as compared with the general structure above. For example, in some embodiments, an amino acid may be modified by methylation, amidation, acetylation, and/or substitution as compared with the general structure. In some embodiments, such modification may, for example, alter the circulating half-life of a polypeptide containing the modified amino acid as compared with one containing an otherwise identical unmodified amino acid. In some embodiments, such modification does not significantly alter a relevant activity of a polypeptide containing the modified amino acid, as compared with one containing an otherwise identical unmodified amino acid. As will be clear from context, in some embodiments, the term “amino acid” is used to refer to a free amino acid; in some embodiments it is used to refer to an amino acid residue of a polypeptide.

“Associated”: As used herein, the term “associated” typically refers to two or more entities in physical proximity with one another, either directly or indirectly (e.g., via one or more additional entities that serve as a linking agent), to form a structure that is sufficiently stable so that the entities remain in physical proximity under relevant conditions, e.g., physiological conditions. In some embodiments, associated entities are covalently linked to one another. In some embodiments, associated entities are non-covalently linked. In some embodiments, associated entities are linked to one another by specific non-covalent interactions (i.e., by interactions between interacting ligands that discriminate between their interaction partner and other entities present in the context of use, such as, for example streptavidin/avidin interactions, antibody/antigen interactions, etc.). Alternatively or additionally, a sufficient number of weaker non-covalent interactions can provide sufficient stability for moieties to remain associated. Exemplary non-covalent interactions include, but are not limited to, affinity interactions, metal coordination, physical adsorption, host-guest interactions, hydrophobic interactions, pi stacking interactions, hydrogen bonding interactions, van der Waals interactions, magnetic interactions, electrostatic interactions, dipole-dipole interactions, etc.

“Biocompatible”: The term “biocompatible”, as used herein, refers to materials that do not cause significant harm to living tissue when placed in contact with such tissue, e.g., in vivo. In certain embodiments, materials are “biocompatible” if they are not toxic to cells. In certain embodiments, materials are “biocompatible” if their addition to cells in vitro results in less than or equal to 20% cell death, and/or their administration in vivo does not induce significant inflammation or other such adverse effects.

“Biodegradable”: As used herein, the term “biodegradable” refers to materials that, when introduced into cells, are broken down (e.g., by cellular machinery, such as by enzymatic degradation, by hydrolysis, and/or by combinations thereof) into components that cells can either reuse or dispose of without significant toxic effects on the cells. In certain embodiments, components generated by breakdown of a biodegradable material are biocompatible and therefore do not induce significant inflammation and/or other adverse effects in vivo. In some embodiments, biodegradable polymer materials break down into their component monomers. In some embodiments, breakdown of biodegradable materials (including, for example, biodegradable polymer materials) involves hydrolysis of ester bonds. Alternatively or additionally, in some embodiments, breakdown of biodegradable materials (including, for example, biodegradable polymer materials) involves cleavage of urethane linkages. Exemplary biodegradable polymers include, for example, polymers of hydroxy acids such as lactic acid and glycolic acid, including but not limited to poly(hydroxyl acids), poly(lactic acid)(PLA), poly(glycolic acid)(PGA), poly(lactic-co-glycolic acid)(PLGA), and copolymers with PEG, polyanhydrides, poly(ortho)esters, polyesters, polyurethanes, poly(butyric acid), poly(valeric acid), poly(caprolactone), poly(hydroxyalkanoates, poly(lactide-co-caprolactone), blends and copolymers thereof. Many naturally occurring polymers are also biodegradable, including, for example, proteins such as albumin, collagen, gelatin and prolamines, for example, zein, and polysaccharides such as alginate, cellulose derivatives and polyhydroxyalkanoates, for example, polyhydroxybutyrate blends and copolymers thereof. Those of ordinary skill in the art will appreciate or be able to determine when such polymers are biocompatible and/or biodegradable derivatives thereof (e.g., related to a parent polymer by substantially identical structure that differs only in substitution or addition of particular chemical groups as is known in the art).

“Comparable”: The term “comparable”, as used herein, refers to two or more agents, entities, situations, sets of conditions, etc. that may not be identical to one another but that are sufficiently similar to permit comparison there between so that conclusions may reasonably be drawn based on differences or similarities observed. Those of ordinary skill in the art will understand, in context, what degree of identity is required in any given circumstance for two or more such agents, entities, situations, sets of conditions, etc. to be considered comparable.

“Conjugated”: As used herein, the terms “conjugated,” “linked,” and “attached,” when used with respect to two or more moieties, means that the moieties are physically connected with one another, either directly or via one or more additional moieties that serves as a linking agent, to form a structure that is sufficiently stable so that the moieties remain physically connected under the conditions in which structure is used, e.g., physiological conditions. Typically the moieties are attached either by one or more covalent bonds or by a mechanism that involves specific binding. Alternately, a sufficient number of weaker interactions can provide sufficient stability for moieties to remain physically connected.

“Corresponding to”: As used herein, the term “corresponding to” is often used to designate the position/identity of a residue in a polymer, such as an amino acid residue in a polypeptide or a nucleotide residue in a nucleic acid. Those of ordinary skill will appreciate that, for purposes of simplicity, residues in such a polymer are often designated using a canonical numbering system based on a reference related polymer, so that a residue in a first polymer “corresponding to” a residue at position 190 in the reference polymer, for example, need not actually be the 190th residue in the first polymer but rather corresponds to the residue found at the 190th position in the reference polymer; those of ordinary skill in the art readily appreciate how to identify “corresponding” amino acids, including through use of one or more commercially-available algorithms specifically designed for polymer sequence comparisons.

“Encapsulated”: The term “encapsulated” is used herein to refer to substances that are substantially completely surrounded by another material.

“Functional”: As used herein, a “functional” biological molecule is a biological molecule in a form in which it exhibits a property and/or activity by which it is characterized. A biological molecule may have two functions (i.e., bi-functional) or many functions (i.e., multifunctional).

“High Molecular Weight Polymer”: As used herein, the term “high molecular weight polymer” refers to polymers and/or polymer solutions comprised of polymers (e.g., protein polymers, such as silk) having molecular weights of at least about 200 kDa, and wherein no more than 30% of the silk fibroin has a molecular weight of less than 100 kDa. In some embodiments, high molecular weight polymers and/or polymer solutions have an average molecular weight of at least about 100 kDa or more, including, e.g., at least about 150 kDa, at least about 200 kDa, at least about 250 kDa, at least about 300 kDa, at least about 350 kDa or more. In some embodiments, high molecular weight polymers have a molecular weight distribution, no more than 50%, for example, including, no more than 40%, no more than 30%, no more than 20%, no more than 10%, of the silk fibroin can have a molecular weight of less than 150 kDa, or less than 125 kDa, or less than 100 kDa.

“Hydrolytically degradable”: As used herein, the term “hydrolytically degradable” is used to refer to materials that degrade by hydrolytic cleavage. In some embodiments, hydrolytically degradable materials degrade in water. In some embodiments, hydrolytically degradable materials degrade in water in the absence of any other agents or materials. In some embodiments, hydrolytically degradable materials degrade completely by hydrolytic cleavage, e.g., in water. By contrast, the term “non-hydrolytically degradable” typically refers to materials that do not fully degrade by hydrolytic cleavage and/or in the presence of water (e.g., in the sole presence of water).

“Hydrophilic”: As used herein, the term “hydrophilic” and/or “polar” refers to a tendency to mix with, or dissolve easily in, water.

“Hydrophobic”: As used herein, the term “hydrophobic” and/or “non-polar”, refers to a tendency to repel, not combine with, or an inability to dissolve easily in, water.

“Low Molecular Weight Polymer”: As used herein, the term “low molecular weight polymer” refers to polymers and/or polymer solutions, such as silk, comprised of polymers (e.g., protein polymers) having molecular weights within the range of about 3 kDa-about 200 kDa. In some embodiments, low molecular weight polymers (e.g., protein polymers) have molecular weights within a range between a lower bound (e.g., about 10 kDa, about 20 kDa, about 30 kDa, about 40 kDa, about 50 kDa, about 60 kDa, or more) and an upper bound (e.g., about 200 kDa, about 175 kDa, about 150 kDa, about 125 kDa, about 100 kDa, or less). In some embodiments, low molecular weight polymers (e.g., protein polymers such as silk) are substantially free of polymers having a molecular weight above about 200 kD. In some embodiments, the highest molecular weight polymers in provided hydrogels are less than about 100-about 200 kD (e.g., less than about 200 kD, less than about 175 kD, less than about 150 kD, less than about 125 kD, less than about 100 kD, etc). In some embodiments, a low molecular weight polymer and/or polymer solution can comprise a population of polymer fragments having a range of molecular weights, characterized in that: between 0 and 15% (e.g., at least 1% 5%, 10%) of the total moles of polymer fragments in the population has a molecular weight exceeding 200 kDa, and at least 50% of the total moles of the silk fibroin fragments in the population has a molecular weight within a specified range, wherein the specified range is between about 3 kDa and about 150 kDa or between about 5 kDa and about 125 kDa.

“Matrix”: As used herein, the term “matrix” refers to a biomaterial comprising silk fibroin or collagen or combinations of these two as well as with ECM components, on or in which cells will grow.

“Nucleic acid”: As used herein, the term “nucleic acid,” in its broadest sense, refers to any compound and/or substance that is or can be incorporated into an oligonucleotide chain. In some embodiments, a nucleic acid is a compound and/or substance that is or can be incorporated into an oligonucleotide chain via a phosphodiester linkage. In some embodiments, “nucleic acid” refers to individual nucleic acid residues (e.g., nucleotides and/or nucleosides). In some embodiments, “nucleic acid” refers to an oligonucleotide chain comprising individual nucleic acid residues. As used herein, the terms “oligonucleotide” and “polynucleotide” can be used interchangeably. In some embodiments, “nucleic acid” encompasses RNA as well as single and/or double-stranded DNA and/or cDNA. Furthermore, the terms “nucleic acid,” “DNA,” “RNA,” and/or similar terms include nucleic acid analogs, i.e., analogs having other than a phosphodiester backbone. For example, the so-called “peptide nucleic acids,” which are known in the art and have peptide bonds instead of phosphodiester bonds in the backbone, are considered within the scope of the present invention. The term “nucleotide sequence encoding an amino acid sequence” includes all nucleotide sequences that are degenerate versions of each other and/or encode the same amino acid sequence. Nucleotide sequences that encode proteins and/or RNA may include introns. Nucleic acids can be purified from natural sources, produced using recombinant expression systems and optionally purified, chemically synthesized, etc. Where appropriate, e.g., in the case of chemically synthesized molecules, nucleic acids can comprise nucleoside analogs such as analogs having chemically modified bases or sugars, backbone modifications, etc. A nucleic acid sequence is presented in the 5′ to 3′ direction unless otherwise indicated. The term “nucleic acid segment” is used herein to refer to a nucleic acid sequence that is a portion of a longer nucleic acid sequence. In many embodiments, a nucleic acid segment comprises at least 3, 4, 5, 6, 7, 8, 9, 10, or more residues. In some embodiments, a nucleic acid is or comprises natural nucleosides (e.g., adenosine, thymidine, guanosine, cytidine, uridine, deoxyadenosine, deoxythymidine, deoxyguanosine, and deoxycytidine); nucleoside analogs (e.g., 2-aminoadenosine, 2-thiothymidine, inosine, pyrrolo-pyrimidine, 3-methyl adenosine, 5-methylcytidine, C-5 propynyl-cytidine, C-5 propynyl-uridine, 2-aminoadenosine, C5-bromouridine, C5-fluorouridine, C5-iodouridine, C5-propynyl-uridine, C5-propynyl-cytidine, C5-methylcytidine, 2-aminoadenosine, 7-deazaadenosine, 7-deazaguanosine, 8-oxoadenosine, 8-oxoguanosine, O(6)-methylguanine, and 2-thiocytidine); chemically modified bases; biologically modified bases (e.g., methylated bases); intercalated bases; modified sugars (e.g., 2′-fluororibose, ribose, 2′-deoxyribose, arabinose, and hexose); and/or modified phosphate groups (e.g., phosphorothioates and 5′-N-phosphoramidite linkages). In some embodiments, the present invention is specifically directed to “unmodified nucleic acids,” meaning nucleic acids (e.g., polynucleotides and residues, including nucleotides and/or nucleosides) that have not been chemically modified in order to facilitate or achieve delivery.

“Physiological conditions”: The phrase “physiological conditions”, as used herein, relates to the range of chemical (e.g., pH, ionic strength) and biochemical (e.g., enzyme concentrations) conditions likely to be encountered in the intracellular and extracellular fluids of tissues. For most tissues, the physiological pH ranges from about 6.8 to about 8.0 and a temperature range of about 20-40 degrees Celsius, about 25-40° C., about 30-40° C., about 35-40° C., about 37° C., and atmospheric pressure of about 1. In some embodiments, physiological conditions utilize or include an aqueous environment (e.g., water, saline, Ringers solution, or other buffered solution); in some such embodiments, the aqueous environment is or comprises a phosphate buffered solution (e.g., phosphate-buffered saline).

“Polypeptide”: The term “polypeptide” as used herein, refers to a string of at least three amino acids linked together by peptide bonds. In some embodiments, a polypeptide comprises naturally-occurring amino acids; alternatively or additionally, in some embodiments, a polypeptide comprises one or more non-natural amino acids (i.e., compounds that do not occur in nature but that can be incorporated into a polypeptide chain; see, for example, www.cco.caltech.edurdadgrp/Unnatstruct.gif, which displays structures of non-natural amino acids that have been successfully incorporated into functional ion channels) and/or amino acid analogs as are known in the art may alternatively be employed). For example, a polypeptide can be a protein. In some embodiments, one or more of the amino acids in a polypeptide may be modified, for example, by the addition of a chemical entity such as a carbohydrate group, a phosphate group, a farnesyl group, an isofarnesyl group, a fatty acid group, a linker for conjugation, functionalization, or other modification, etc.

“Polysaccharide”: The term “polysaccharide” refers to a polymer of sugars. Typically, a polysaccharide comprises at least three sugars. In some embodiments, a polypeptide comprises natural sugars (e.g., glucose, fructose, galactose, mannose, arabinose, ribose, and xylose); alternatively or additionally, in some embodiments, a polypeptide comprises one or more non-natural amino acids (e.g. modified sugars such as 2′-fluororibose, 2′-deoxyribose, and hexose).

“Porosity”: The term “porosity” as used herein, refers to a measure of void spaces in a material and is a fraction of volume of voids over the total volume, as a percentage between 0 and 100%. A determination of porosity is known to a skilled artisan using standardized techniques, for example mercury porosimetry and gas adsorption (e.g., nitrogen adsorption).

“Protein”: As used herein, the term “protein” refers to a polypeptide (i.e., a string of at least two amino acids linked to one another by peptide bonds). Proteins may include moieties other than amino acids (e.g., may be glycoproteins, proteoglycans, etc.) and/or may be otherwise processed or modified. Those of ordinary skill in the art will appreciate that a “protein” can be a complete polypeptide chain as produced by a cell (with or without a signal sequence), or can be a characteristic portion thereof. Those of ordinary skill will appreciate that a protein can sometimes include more than one polypeptide chain, for example linked by one or more disulfide bonds or associated by other means. Polypeptides may contain L-amino acids, D-amino acids, or both and may contain any of a variety of amino acid modifications or analogs known in the art. Useful modifications include, e.g., terminal acetylation, amidation, methylation, etc. In some embodiments, proteins may comprise natural amino acids, non-natural amino acids, synthetic amino acids, and combinations thereof. The term “peptide” is generally used to refer to a polypeptide having a length of less than about 100 amino acids, less than about 50 amino acids, less than 20 amino acids, or less than 10 amino acids. In some embodiments, proteins are antibodies, antibody fragments, biologically active portions thereof, and/or characteristic portions thereof.

“Scaffold”: As used herein, the term “scaffold” refer to a three dimensional architecture that is generated from a matrix. Non-limiting examples include tubes, films, fibers, foams, gels, particles (e.g., microsphere, nanosphere, etc), ring shaped structures, and combinations thereof.

“Small molecule”: As used herein, the term “small molecule” is used to refer to molecules, whether naturally-occurring or artificially created (e.g., via chemical synthesis), having a relatively low molecular weight and being an organic and/or inorganic compound. Typically, a “small molecule” is monomeric and have a molecular weight of less than about 1500 g/mol. In general, a “small molecule” is a molecule that is less than about 5 kilodaltons (kD) in size. In some embodiments, a small molecule is less than about 4 kD, 3 kD, about 2 kD, or about 1 kD. In some embodiments, the small molecule is less than about 800 daltons (D), about 600 D, about 500 D, about 400 D, about 300 D, about 200 D, or about 100 D. In some embodiments, a small molecule is less than about 2000 g/mol, less than about 1500 g/mol, less than about 1000 g/mol, less than about 800 g/mol, or less than about 500 g/mol. In some embodiments, a small molecule is not a polymer. In some embodiments, a small molecule does not include a polymeric moiety. In some embodiments, a small molecule is not a protein or polypeptide (e.g., is not an oligopeptide or peptide). In some embodiments, a small molecule is not a polynucleotide (e.g., is not an oligonucleotide). In some embodiments, a small molecule is not a polysaccharide. In some embodiments, a small molecule does not comprise a polysaccharide (e.g., is not a glycoprotein, proteoglycan, glycolipid, etc.).

“Solution”: As used herein, the term “solution” broadly refers to a homogeneous mixture composed of one phase. Typically, a solution comprises a solute or solutes dissolved in a solvent or solvents. It is characterized in that the properties of the mixture (such as concentration, temperature, and density) can be uniformly distributed through the volume. In the context of the present application, therefore, a “silk fibroin solution” refers to silk fibroin protein in a soluble form, dissolved in a solvent, such as water. In some embodiments, silk fibroin solutions may be prepared from a solid-state silk fibroin material (i.e., silk matrices), such as silk films and other scaffolds. Typically, a solid-state silk fibroin material is reconstituted with an aqueous solution, such as water and a buffer, into a silk fibroin solution. It should be noted that liquid mixtures that are not homogeneous, e.g., colloids, suspensions, emulsions, are not considered solutions.

“Stable”: The term “stable,” when applied to compositions herein, means that the compositions maintain one or more aspects of their physical structure and/or activity over a period of time under a designated set of conditions. In some embodiments, a period of time is at least about one hour; in some embodiments, the period of time is about 5 hours, about 10 hours, about one (1) day, about one (1) week, about two (2) weeks, about one (1) month, about two (2) months, about three (3) months, about four (4) months, about five (5) months, about six (6) months, about eight (8) months, about ten (10) months, about twelve (12) months, about twenty-four (24) months, about thirty-six (36) months, or longer. In some embodiments, the period of time is within the range of about one (1) day to about twenty-four (24) months, about two (2) weeks to about twelve (12) months, about two (2) months to about five (5) months, etc. In some embodiments, the designated conditions are ambient conditions (e.g., at room temperature and ambient pressure). In some embodiments, the designated conditions are physiologic conditions (e.g., in vivo or at about 37° C. for example in serum or in phosphate buffered saline). In some embodiments, the designated conditions are under cold storage (e.g., at or below about 4° C., −20° C., or −70° C.). In some embodiments, the designated conditions are in the dark.

“Substantially”: As used herein, the term “substantially”, and grammatical equivalents, refer to the qualitative condition of exhibiting total or near-total extent or degree of a characteristic or property of interest. One of ordinary skill in the art will understand that biological and chemical phenomena rarely, if ever, go to completion and/or proceed to completeness or achieve or avoid an absolute result.

“Sustained release”: The term “sustained release” is used herein in accordance with its art-understood meaning of release that occurs over an extended period of time. The extended period of time can be at least about 3 days, about 5 days, about 7 days, about 10 days, about 15 days, about 30 days, about 1 month, about 2 months, about 3 months, about 6 months, or even about 1 year. In some embodiments, sustained release is substantially burst-free. In some embodiments, sustained release involves steady release over the extended period of time, so that the rate of release does not vary over the extended period of time more than about 5%, about 10%, about 15%, about 20%, about 30%, about 40% or about 50%. In some embodiments, sustained release involves release with first-order kinetics. In some embodiments, sustained release involves an initial burst, followed by a period of steady release. In some embodiments, sustained release does not involve an initial burst. In some embodiments, sustained release is substantially burst-free release.

### DETAILED DESCRIPTION OF CERTAIN EMBODIMENTS

Prior to the present invention, there has been no acceptable, physiologically relevant model of human brain tissue or substantial portions thereof for use in a research, and/or therapeutic setting. In part, this is because no previously known artificial brain composition was able to allow for physiologically relevant interactions between human nerve cells, glia, and a biocompatible and/or biodegradable scaffold, provide sustained function in vitro, support electrophysiological function, and sustain structure for extended time frames, to a satisfactory degree. In addition, various embodiments of the present invention also allow for integration of aspects of the immune system, vascular system, lymphatic system and even the endocrine system with the nervous system cells. Further, and in accordance with several embodiments, provided compositions comprise all human cells (e.g., human primary cells and/or human induced pluripotent stem cells). Even more, aspects of the present invention may include one or more electrical devices, optical devices, and/or optical tools that may be functionally associated with provided compositions.

The Brain

The brain is one of the most complex organs in the body, made up of billions of cells and trillions of connections. The brain, along with the spinal cord, make up the central nervous system. For vertebrates, and many invertebrates, the brain is the central control center, coordinating receipt and perception of stimuli, as well as motor and other responses thereto. In humans, the brain is made up of three major components: the telencephalon/forebrain, the mesencephalon/midbrain, and the rhombencephalon/hindbrain. Major brain components include the medulla, pons, hypothalamus, thalamus, cerebellum, hippocampus, basal ganglia, olfactory bulb, inferior colliculus, superior colliculus, and the cerebral cortex. Surprisingly, despite all of the complex structures in the brain, as well as all of the enormous diversity of signals a brain can interpret, and exert, brains are made up principally of just two major types of cells neurons and glia.

Because the brain is so important to the proper functions of an animal (e.g., a human), dysfunction in the brain can have devastating consequences including motor dysfunction (including partial or total paralysis), memory impairment or loss, pain, seizure, difficulty speaking, behavioral abnormalities and even death.

A variety of diseases, disorders, or conditions are known to affect the brain and/or spine, including Autism, Alzheimer's disease, Parkinson's disease, Huntington's disease, Creutzfeldt-Jakob disease, multiple sclerosis, meningitis, encephalitis, Lou Gehrig's disease, Tourette's, attention deficit hyperactivity disorder (ADHD), lysosomal storage disorders, Guillain-Barre syndrome, Bell's palsy, Schizophrenia, Obsessive compulsive disorder (OCD), Tay-Sachs disease, Niemann-Pick disease, chronic traumatic encephalopathy CTE), Multiple system atrophy, Stroke, certain cancers (e.g., medulloblastoma), and traumatic brain injury, among others, and it is contemplated that certain embodiments would be useful in the study and/or treatment of each.

Provided Compositions

In accordance with a variety of aspects, the present invention provides compositions comprising physiologically relevant brain-like compositions, as well as methods for making and using those compositions. In particular, the present invention provides, inter alfa, compositions including a plurality of human nerve cells, a plurality of glial cells, and silk fibroin. In some embodiments, provided compositions may include at least one extracellular matrix (ECM) agents, at least one active agent, and/or at least one additional cell type.

In accordance with some aspects of the present invention, certain provided compositions may be capable of performing certain functions and/or having certain structures classically associated with a blood brain barrier. By way of non-limiting example, in some embodiments, the present invention also provides compositions including a porous, three dimensional silk fibroin matrix comprising a plurality of pores, a conduit within the matrix sufficient to support flow of at least one fluid, a plurality of brain microvascular endothelial cells, and at least one ECM agent. In some embodiments, such compositions may further comprise at least one active agent and/or at least one additional cell type.

In some embodiments, provided compositions may be biocompatible suitable for in vivo use (e.g., as a transplant). In some embodiments, provided compositions may be used in vitro (e.g., to study certain diseases and/or test therapeutic candidates, for example, in cancer, brain hemorrhage, or traumatic brain injury contexts).

Nerve Cells/Neurons

Provided methods and compositions include a plurality of nerve cells/neurons. Neurons are commonly defined as a cell that is electrically excitable and that processes and transmits information via electrical and/or chemical signals. Neuronal structure generally consists of a cell body including a plurality of dendrites, an axon, and one or more axon terminals. Typically, neuronal communication involves a neuron receiving an input at the cell body/dendrites, and then proceeding to transmit a signal (also known as an action potential) down its axon, and to the axon terminal, where electrical and/or chemical signals are released to another neuron or effector cell. This communication of signal and release of electrical and/or chemical signal is often referred to as an action potential.

There are several known types of neuronal cell and each is specifically contemplated as useful in at least some embodiments. Neuronal cells include, but are not limited to, sensory neurons, motor neurons, neural stem cells, induced pluripotent stem cells, and combinations thereof.

Depending upon the specific structures of each component, there are a variety of different neuronal subtypes including unipolar (where a dendrite and axon emerge from the same process), bipolar (where an axon and a single dendrite are present on opposite sides of the cell body), multipolar (having two or more dendrites which are separate from the axon), and anaxonic neurons (where the axon cannot be distinguished from the dendrites). In addition to the above structural classifications, several additional neuronal subtypes are known and contemplated to be useful in some embodiments including, but not limited to basket cells, Betz cells, Lugaro cells, Purkinje cells, pyramidal cells, granule cells, and spindle cells.

Depending upon how particular neurons process information, they may be referred to as afferent neurons (convey information from tissues and/or organs to the brain or spinal cord (e.g. sensory neurons)), efferent neurons (transmit signals from the brain or spinal cord to the effector cells (e.g., motor neurons)), or interneurons (connect two neurons with specific regions of the brain or spinal cord).

Provided compositions include a plurality of mammalian nerve cells (e.g. human nerve cells, including primary human nerve cells). In some embodiments, at least some of a plurality of the nerve cells are functional (e.g., capable of firing a plurality of action potentials or differentiating into one or more cell types). In some embodiments, substantially all of the nerve cells are functional. Depending upon the specific embodiment, any of a variety of measures of functionality may be used including, but not limited to, the ability to fire an action potential, differentiation into one or more cell types, axonal formation, axonal elongation, active growth cone activity, synaptic formation, neurotransmitter production, resting membrane potential, gene expression, protein expression, and combinations thereof. In some embodiments, mammalian nerve cells may include rat, mouse, dog, primate, or other neurons.

In some embodiments, the function of at least some of the plurality of nerve cells may be assessed via any known method. For example, in some embodiments, the function of at least some of the plurality of nerve cells may be assessed via real time calcium imaging, electrophysiological recordings (either by whole-cell patch clamping or local field potential recordings), magnetic resonance imaging, assays of metabolic function including neurotransmitter production, and measurement of local field potential, among others.

As is known in the art, in order for neurons to function efficiently or, in some cases, at all, the axon must be myelinated by one or more glial cells. In some embodiments, at least some of a plurality of nerve cells are at least partially myelinated. In some embodiments, at least some of the plurality of nerve cells are fully myelinated (e.g., with a pattern of myelination substantially similar to those found in vivo). In some embodiments, the degree and/or quality of myelination may be assessed using any known method (e.g., via CARS laser).

Glial Cells

Provided methods and compositions also include a plurality of at least one type of mammalian glial cells. In some embodiments, glial cells are human glial cells (e.g., human primary glial cells). In general, glial cells are non-neuronal cells that provide support and protection for neurons. In some cases, glial cells myelinate one or more neurons. Some glial cells may function primarily as physical supports for neurons, while others provide nutritional support and/or maintain homeostasis in a local area, and still other types provide immune protection to the brain (e.g., by acting as scavenger cells, and removing harmful and/or foreign material from the tissue). In some embodiments, mammalian glial cells may include rat, mouse, dog, primate, or other glia.

There are several known types of glial cell and each is specifically contemplated as useful in at least some embodiments. Glial cells useful in at least some embodiments include, but are not limited to astrocytes, oligodendrocytes, Schwann cells, glial progenitor cells, ependymal cells, NG2 cells, microglia, tanycytes, and combinations thereof. Glial cells may have any of a variety of roles including myelination of one or more axons, clearance of neurotransmitter from a synapse, encouragement or inhibition of axonal repair, formation of scar in response to injury, immune protection, and contribute to formation and/or mechanical support of the blood-brain barrier.

Silk (e.g., Silk Fibroin)

Compositions provided herein include compositions comprising nerve cells, glial cells, and silk fibroin. In some embodiments, provided compositions may comprise a plurality of layers, compartments to represent the different regions of the brain or layers of specific compartments, each containing silk fibroin and/or in combination with other matrix components. Silk fibroin, such as that derived from Bombyx mori silkworm cocoons, for example, is a biocompatible material that degrades slowly in the body, is readily modified into a variety of formats, and generates mechanically robust materials.

As used herein, the term “fibroin” includes, but is not limited to, silkworm fibroin and insect or spider silk protein. In some embodiments, fibroin is obtained from a solution containing a dissolved silkworm silk or spider silk. In some embodiments silkworm silk protein is obtained, for example, from Bombyx mori, and spider silk is obtained from Nephila clavipes. In some embodiments, silk proteins suitable for use in the present invention may be obtained from a solution containing a genetically engineered silk, such as from bacteria, yeast, mammalian cells, transgenic animals or transgenic plants. See, for example, WO 97/08315 and U.S. Pat. No. 5,245,012. Genetically engineered silk can, for example, comprise a therapeutic agent, e.g., a fusion protein with a cytokine, an enzyme, or any number of hormones or peptide-based drugs, antimicrobials and related substrates.

In some embodiments, provided compositions comprising silk fibroin may be made using one or more silk solutions, which are known to be highly customizable and allow for the production of any of a variety of end products. As such, in some embodiments, provided compositions may be produced using any of a variety of silk solutions. Preparation of silk fibroin solutions has been described previously, e.g., in WO 2007/016524, which is incorporated herein by reference in its entirety. The reference describes not only the preparation of aqueous silk fibroin solutions, but also such solutions in conjunction with bioactive agents.

In accordance with various embodiments, a silk solution may comprise any of a variety of concentrations of silk fibroin. In some embodiments, a silk solution may comprise 0.1 to 30% by weight silk fibroin. In some embodiments, a silk solution may comprise between about 0.5% and 30% (e.g., 0.5% to 25%, 0.5% to 20%, 0.5% to 15%, 0.5% to 10%, 0.5% to 5%, 0.5% to 1.0%) by weight silk fibroin, inclusive. In some embodiments, a silk solution may comprise at least 0.1% (e.g., at least 0.5%, 1%, 2%, 3%, 4%, 5%, 6%, 7%, 8%, 9%, 10%, 15%, 20%, 25%) by weight silk fibroin. In some embodiments, a silk solution may comprise at most 30% (e.g., at most 25%, 20%, 15%, 14%, 13%, 12% 11%, 10%, 5%, 4%, 3%, 2%, 1%) by weight silk fibroin.

In accordance with various embodiments, the compositions disclosed herein can comprise any amount/ratio of silk fibroin to the total volume/weight of the overall composition (e.g., an individual composition or more complex provided composition such as a multi-layered composition or composition including multiple scaffold types). In some embodiments, the amount of silk fibroin in the solution used for making a provided silk fibroin composition itself can be varied to vary properties of the end silk fibroin composition. By way of specific example, in some embodiments, silk fibroin comprises at least 1% of a provided composition by weight (e.g., at least 2%, 3%, 4%, 5%, 6%, 7%, 8%, 9% 10%, 15%, 20%, 25% or more). In some embodiments, silk fibroin comprises at most 35% of a provided composition by weight (e.g., at most 30%, 25%, 20%, 15%, 10%, 5% or less). In some embodiments, silk fibroin comprises between 1-35% of a provided composition by weight (e.g., between 1-30%, 1-25%, 1-20%, 1-15%, 1-10%, 1-5%, 5-25%, 5-20%, 5-15%, 5-10%).

In accordance with various embodiments, silk used in provided methods and compositions is degummed silk (i.e. silk fibroin with at least a portion of the native sericin removed). Degummed silk can be prepared by any conventional method known to one skilled in the art. For example, B. mori cocoons are boiled for a period of pre-determined time in an aqueous solution. Generally, longer degumming time generates lower molecular silk fibroin. In some embodiments, the silk cocoons are boiled for at least 60 minutes, at least 70 minutes, at least 80 minutes, at least 90 minutes, at least 100 minutes, at least 110 minutes, at least 120 minutes, or longer. Additionally or alternatively, in some embodiments, silk cocoons can be heated or boiled at an elevated temperature. For example, in some embodiments, silk cocoons can be heated or boiled at about 101.0° C., at about 101.5° C., at about 102.0° C., at about 102.5° C., at about 103.0° C., at about 103.5° C., at about 104.0° C., at about 104.5° C., at about 105.0° C., at about 105.5° C., at about 106.0° C., at about 106.5° C., at about 107.0° C., at about 107.5° C., at about 108.0° C., at about 108.5° C., at about 109.0° C., at about 109.5° C., at about 110.0° C., at about 110.5° C., at about 111.0° C., at about 111.5° C., at about 112.0° C., at about 112.5° C., at about 113.0° C., 113.5° C., at about 114.0° C., at about 114.5° C., at about 115.0° C., at about 115.5° C., at about 116.0° C., at about 116.5° C., at about 117.0° C., at about 117.5° C., at about 118.0° C., at about 118.5° C., at about 119.0° C., at about 119.5° C., at about 120.0° C., or higher. In some embodiments, such elevated temperature can be achieved by carrying out at least portion of the heating process (e.g., boiling process) under pressure. For example, suitable pressure under which silk fibroin fragments described herein can be produced are typically between about 10-40 psi, e.g., about 11 psi, about 12 psi, about 13 psi, about 14 psi, about 15 psi, about 16 psi, about 17 psi, about 18 psi, about 19 psi, about 20 psi, about 21 psi, about 22 psi, about 23 psi, about 24 psi, about 25 psi, about 26 psi, about 27 psi, about 28 psi, about 29 psi, about 30 psi, about 31 psi, about 32 psi, about 33 psi, about 34 psi, about 35 psi, about 36 psi, about 37 psi, about 38 psi, about 39 psi, or about 40 psi.

In some embodiments, the aqueous solution used in the process of degumming silk cocoons comprises about 0.02M Na₂CO₃. The cocoons are rinsed, for example, with water to extract the sericin proteins. The degummed silk can be dried and used for preparing silk powder. Alternatively, the extracted silk can dissolved in an aqueous salt solution. Salts useful for this purpose include lithium bromide, lithium thiocyanate, calcium nitrate or other chemicals capable of solubilizing silk. In some embodiments, the extracted silk can be dissolved in about 8M-12 M LiBr solution. The salt is consequently removed using, for example, dialysis.

In some embodiments, the silk fibroin is substantially depleted of its native sericin content (e.g., 5% (w/w) or less residual sericin in the final extracted silk). In some embodiments, the silk fibroin is entirely free of its native sericin content. As used herein, the term “entirely free” (i.e. “consisting of” terminology) means that within the detection range of the instrument or process being used, the substance cannot be detected or its presence cannot be confirmed. In some embodiments, the silk fibroin is essentially free of its native sericin content. As used herein, the term “essentially free” (or “consisting essentially of”) means that only trace amounts of the substance can be detected, is present in an amount that is below detection, or is absent.

If necessary, a silk solution may be concentrated using, for example, dialysis against a hygroscopic polymer, for example, PEG, a polyethylene oxide, amylose or sericin. In some embodiments, the PEG is of a molecular weight of 8,000-10,000 g/mol and has a concentration of about 10% to about 50% (w/v). A slide-a-lyzer dialysis cassette (Pierce, MW CO 3500) can be used. However, any dialysis system can be used. The dialysis can be performed for a time period sufficient to result in a final concentration of aqueous silk solution between about 10% to about 30%. In most cases dialysis for 2-12 hours can be sufficient. See, for example, International Patent Application Publication No. WO 2005/012606, the content of which is incorporated herein by reference in its entirety. Another method to generate a concentrated silk solution comprises drying a dilute silk solution (e.g., through evaporation or lyophilization). The dilute solution can be dried partially to reduce the volume thereby increasing the silk concentration. The dilute solution can be dried completely and then dissolving the dried silk fibroin in a smaller volume of solvent compared to that of the dilute silk solution. In some embodiments, a silk fibroin solution can optionally, at a suitable point, be filtered and/or centrifuged. For example, in some embodiments, a silk fibroin solution can optionally be filtered and/or centrifuged following the heating or boiling step. In some embodiments, a silk fibroin solution can optionally be filtered and/or centrifuged following the dialysis step. In some embodiments, a silk fibroin solution can optionally be filtered and/or centrifuged following the step of adjusting concentrations. In some embodiments, a silk fibroin solution can optionally be filtered and/or centrifuged following the step of reconstitution. In any of such embodiments, the filtration and/or centrifugation step(s) can be carried out to remove insoluble materials. In any of such embodiments, the filtration and/or centrifugation step(s) can be carried out to selectively enrich silk fibroin fragments of certain molecular weight(s).

In some embodiments, provided silk compositions described herein, and methods of making and/or using them, may be performed in the absence of any organic solvent. Thus, in some embodiments, provided compositions and methods are particularly amenable to the incorporation of labile molecules, such as bioactive agents or therapeutics, and can, in certain embodiments, be used to produce controlled release biomaterials. In some embodiments, such methods are performed in water only.

In some embodiments, the silk fibroin solution can be produced using organic solvents. Such methods have been described, for example, in Li, M., et al., J. Appl. Poly Sci. 2001, 79, 2192-2199; Min, S., et al. Sen'I Gakkaishi 1997, 54, 85-92; Nazarov, R. et al., Biomacromolecules 2004 5,718-26, contents of all which are incorporated herein by reference in their entireties. An exemplary organic solvent that can be used to produce a silk solution includes, but is not limited to, hexafluoroisopropanol (HFIP). See, for example, International Application No. WO2004/000915, content of which is incorporated herein by reference in its entirety. In some embodiments, the silk solution is entirely free or essentially free of organic solvents, i.e., solvents other than water.

In some embodiments, biocompatible polymers can also be added to a silk solution to generate composite materials in accordance with various methods and processes of the present invention. Exemplary biocompatible polymers useful in some embodiments of the present invention include, for example, polyethylene oxide (PEO) (U.S. Pat. No. 6,302,848), polyethylene glycol (PEG) (U.S. Pat. No. 6,395,734), collagen (U.S. Pat. No. 6,127,143), fibronectin (U.S. Pat. No. 5,263,992), keratin (U.S. Pat. No. 6,379,690), polyaspartic acid (U.S. Pat. No. 5,015,476), polylysine (U.S. Pat. No. 4,806,355), alginate (U.S. Pat. No. 6,372,244), chitosan (U.S. Pat. No. 6,310,188), chitin (U.S. Pat. No. 5,093,489), hyaluronic acid (U.S. Pat. No. 387,413), pectin (U.S. Pat. No. 6,325,810), polycaprolactone (U.S. Pat. No. 6,337,198), polylactic acid (U.S. Pat. No. 6,267,776), polyglycolic acid (U.S. Pat. No. 5,576,881), polyhydroxyalkanoates (U.S. Pat. No. 6,245,537), dextrans (U.S. Pat. No. 5,902,800), and polyanhydrides (U.S. Pat. No. 5,270,419). In some embodiments, two or more biocompatible polymers can be used.

Various embodiments may include provided compositions comprising a plurality of pores of various sizes (i.e., porous silk scaffolds). In some embodiments, pores in a three dimensional silk scaffold have a diameter between about 1-1,000 μm, (e.g., between about 1-100, 20-100, 30-100, 40-100, 50-100, 60-100, 70-100, 80-100, 90-100, 50-1,000, 100-1,000, 200-1,000, 300-1,000, 400-1,000, 500-1,000, 600-1,000, 700-1,000, 800-1,000, or 900-1,000 μm) inclusive. In some embodiments, pores in a three dimensional silk scaffold have a diameter between about 500-1,000 μm, inclusive. In some embodiments, pores in a three dimensional silk scaffold have a diameter between about 500-600 μm, inclusive. In some embodiments, pores in a three dimensional silk scaffold have a diameter between about 200-300 μm, inclusive. In some embodiments, pores may be interconnected. In some embodiments, pores may be substantially random. In some embodiments, pores may be substantially aligned.

In accordance with various embodiments, provided embodiments may comprise any of a variety of levels of porosity. In some embodiments, provided compositions may comprise between 0 and 99% porosity. By way of specific example, in some embodiments, provided compositions may comprise between 5% and 90% porosity (e.g., 10 and 90%, 10 and 80%, 10 and 70%, 10 and 60%, 10 and 50%).

In some embodiments, provided compositions may be made porous through the use of one or more porogens. It is contemplated that any known porogen may be suitable for use according to various embodiments. In some embodiments, a porogen may be or comprise crystals (e.g., sodium chloride crystals), micro- and/or nano-spheres, polymers (such as polyethylene oxide, or PEO), ice crystals, and/or a laser. In some embodiments a porogen may comprise mechanical introduction of pores (e.g., using a needle or other article or device to pierce a composition or portion thereof one or more times, or using stress to introduce one or more tears in composition or portion thereof).

In accordance with various embodiments, provided compositions, for example, compositions comprising one or more silk-containing layers (e.g., porous silk layers) may be of a variety of different thicknesses. In some embodiments, a provided composition (e.g., silk layer) is less than or equal to 100 μm thick. In some embodiments, a provided composition (e.g., silk layer) is between 1 and 100 μm thick (e.g., 5-100, 10-100, 10-90, 10-80, 10-70, 20-70, 30-70, 40-70 μm thick). In some embodiments, provided composition (e.g., silk layer) is about 50-70 μm thick, inclusive. In some embodiments, a silk layer is of a substantially uniform thickness. In some embodiments, provided composition (e.g., silk layer) varies in thickness across a particular length (e.g., a 1 cm).

In some embodiments, provided compositions (and/or silk solutions from which a provided composition is made) may comprise, for example, low molecular weight silk fibroin fragments (e.g., fragments of silk fibroin between 3 kDa and 150 kDa, in some embodiments, with at least some fragments having molecular weights in excess of 200 kDa), though any molecular weight silk may be used in accordance with various embodiments. In any of the embodiments described herein, silk fibroin fragments can include one or more mutations and/or modifications, relative to a naturally occurring (e.g., wild type) sequence of silk fibroin. Such mutation and/or modification in the silk fibroin fragment can be spontaneously occurring or introduced by design. For example, in some embodiments, such mutation and/or modification in the silk fibroin fragment can be introduced using recombinant techniques, chemical modifications, etc.

In some embodiments, the silk fibroin matrix or scaffold may include one or more silk fibroin structures. In some embodiments including multiple silk fibroin structures, at least two of the structures are different from one another. In some embodiments, the silk fibroin comprises a three dimensional matrix. In some embodiments, the three dimensional matrix is or comprises a scaffold. In some embodiments, a matrix or scaffold may be or comprise a tube, film, fiber, foam, gel, particle (e.g., microsphere, nanosphere, etc), ring shaped structure, concentric rings, and combinations thereof. In some embodiments, a matrix or scaffold is not a tube. In some embodiments, a matrix or scaffold is not a film. In some embodiments, a matrix or scaffold is not a fiber. In some embodiments, a matrix or scaffold is not a foam. In some embodiments, a matrix or scaffold is not a gel. In some embodiments, a matrix or scaffold is not a particle (e.g., microsphere, nanosphere, etc.). In some embodiments, a matrix or scaffold is not a ring shaped structure. In some embodiments, a matrix or scaffold is not concentric rings. In some embodiments, provided methods comprise the provison or introduction of compositions comprising a plurality of layers. In some embodiments, compositions comprising a plurality of layers do not include an adhesive added to the composition to hold the layers together.

Silk—Conformational Changes

In some embodiments, a conformational change can be induced in the silk fibroin to control the solubility of the silk fibroin composition. In some embodiments, the conformational change can induce the silk fibroin to become at least partially insoluble. Without wishing to be bound by a theory, an induced conformational change may alter the crystallinity of the silk fibroin, e.g., Silk II beta-sheet crystallinity. In accordance with various embodiments, conformational change can be induced by any methods known in the art, including, but not limited to, alcohol immersion (e.g., ethanol, methanol), water annealing, shear stress, ultrasound (e.g., by sonication), pH reduction (e.g., pH titration and/or exposure to an electric field) and any combinations thereof. For example, the conformational change can be induced by one or more methods, including but not limited to, controlled slow drying (Lu et al., Biomacromolecules 2009, 10, 1032); water annealing (Jin et al., 15 Adv. Funct. Mats. 2005, 15, 1241; Hu et al., Biomacromolecules 2011, 12, 1686); stretching (Demura & Asakura, Biotech & Bioengin. 1989, 33, 598); compressing; solvent immersion, including methanol (Hofmann et al., J Control Release. 2006, 111, 219), ethanol (Miyairi et al., J. Fermen. Tech. 1978, 56, 303), glutaraldehyde (Acharya et al., Biotechnol J. 2008, 3, 226), and 1-ethyl-3-(3-dimethyl aminopropyl) carbodiimide (EDC) (Bayraktar et al., Eur J Pharm Biopharm. 2005, 60, 373); pH adjustment, e.g., pH titration and/or exposure to an electric field (see, e.g., U.S. Patent App. No. US2011/0171239); heat treatment; shear stress (see, e.g., International App. No.: WO 2011/005381), ultrasound, e.g., sonication (see, e.g., U.S. Patent Application Publication No. U.S. 2010/0178304 and International App. No. WO2008/150861); and any combinations thereof. Contents of all of the references listed above are incorporated herein by reference in their entireties.

In some embodiments, the conformation of silk fibroin can be altered by water annealing. Without wishing to be bound by a theory, it is believed that physical temperature-controlled water vapor annealing (TCWVA) provides a simple and effective method to obtain refined control of the molecular structure of silk biomaterials. The silk materials can be prepared with control of crystallinity, from a low beta-sheet content using conditions at 4° C. (a helix dominated silk I structure), to higher beta-sheet content of ˜60% crystallinity at 100° C. (β-sheet dominated silk II structure). This physical approach covers the range of structures previously reported to govern crystallization during the fabrication of silk materials, yet offers a simpler, green chemistry, approach with tight control of reproducibility. Water or water vapor annealing is described, for example, in PCT application no. PCT/US2004/011199, filed Apr. 12, 2004 and no. PCT/US2005/020844, filed Jun. 13, 2005; and Jin et al., Adv. Funct. Mats. 2005, 15: 1241 and Hu et al., Biomacromolecules, 2011, 12(5): 1686-1696, contents of all of which are incorporated herein by reference in their entireties.

In some embodiments, alteration in the conformation of the silk fibroin can be induced by immersing in alcohol, e.g., methanol, ethanol, etc. The alcohol concentration can be at least 10%, at least 20%, at least 30%, at least 40%, at least 50%, at least 60%, at least 70%, at least 80%, at least 90% or 100%. In some embodiment, alcohol concentration is 100%. If the alteration in the conformation is by immersing in a solvent, the silk composition can be washed, e.g., with solvent/water gradient to remove any of the residual solvent that is used for the immersion. The washing can be repeated one, e.g., one, two, three, four, five, or more times.

Alternatively, alteration in the conformation of the silk fibroin can be induced with shear stress. The shear stress can be applied, for example, by passing the silk composition through a needle. Other methods of inducing conformational changes include applying an electric field, applying pressure, or changing the salt concentration.

In some embodiments, alteration in the conformation of the silk fibroin can be induced by horseradish peroxidase (HRP) and hydrogen peroxide (H₂O₂). As is known in the art, HRP facilitates crosslinking of the tyrosines in silk fibroin via the formation of free radical species in the presence of hydrogen peroxide. Exemplary methods may be found in Partlow et al., Highly tunable elastomeric silk biomaterials, 2014, Adv Funct Mater, 24(29): 4615-4624.

The treatment time for inducing the conformational change can be any period of time to provide a desired silk II (beta-sheet crystallinity) content. In some embodiments, the treatment time can range from about 1 hour to about 12 hours, from about 1 hour to about 6 hours, from about 1 hour to about 5 hours, from about 1 hour to about 4 hours, or from about 1 hour to about 3 hours. In some embodiments, the sintering time can range from about 2 hours to about 4 hours or from 2.5 hours to about 3.5 hours.

When inducing the conformational change via solvent immersion, treatment time can range from minutes to hours. For example, immersion in a solvent can be for a period of at least about 15 minutes, at least about 30 minutes, at least about 1 hour, at least about 2 hours, at least 3 hours, at least about 6 hours, at least about 18 hours, at least about 12 hours, at least about 1 day, at least about 2 days, at least about 3 days, at least about 4 days, at least about 5 days, at least about 6 days, at least about 7 days, at least about 8 days, at least about 9 days, at least about 10 days, at least about 11 days, at least about 12 days, at least about 13 days, or at least about 14 days. In some embodiments, immersion in the solvent can be for a period of about 12 hours to about seven days, about 1 day to about 6 days, about 2 to about 5 days, or about 3 to about 4 days.

After the treatment to induce the conformational change, silk fibroin can comprise a silk II beta-sheet crystallinity content of at least about 5%, at least about 10%, at least about 20%, at least about 30%, at least about 40%, at least about 50%, at least about 60%, at least about 70%, at least about 80%, at least about 90%, or at least about 95% but not 100% (i.e., all the silk is present in a silk II beta-sheet conformation). In some embodiments, silk is present completely in a silk II beta-sheet conformation, i.e., 100% silk II beta-sheet crystallinity.

In some embodiments, the silk fibroin may comprise a protein structure that substantially includes β-turn and β-strand regions. Without wishing to be bound by a theory, the silk (3 sheet content can impact gel function and in vivo longevity of the composition. It is to be understood that composition including non-0 sheet content (e.g., e-gels) can also be utilized. In some embodiments, the silk fibroin has a protein structure including, e.g., about 5% β-turn and β-strand regions, about 10% β-turn and β-strand regions, about 20% β-turn and β-strand regions, about 30% β-turn and β-strand regions, about 40% β-turn and β-strand regions, about 50% β-turn and β-strand regions, about 60% β-turn and β-strand regions, about 70% β-turn and β-strand regions, about 80% β-turn and β-strand regions, about 90% β-turn and β-strand regions, or about 100% β-turn and β-strand regions. In other aspects of these embodiments, the silk fibroin has a protein structure including, e.g., at least 10% β-turn and β-strand regions, at least 20% β-turn and β-strand regions, at least 30% β-turn and β-strand regions, at least 40% β-turn and β-strand regions, at least 50% β-turn and β-strand regions, at least 60% β-turn and β-strand regions, at least 70% β-turn and β-strand regions, at least 80% β-turn and β-strand regions, at least 90% (3-turn and β-strand regions, or at least 95% β-turn and β-strand regions. In yet other aspects of these embodiments, the silk fibroin has a protein structure including, e.g., about 10% to about 30% β-turn and β-strand regions, about 20% to about 40% β-turn and β-strand regions, about 30% to about 50% β-turn and β-strand regions, about 40% to about 60% β-turn and β-strand regions, about 50% to about 70% β-turn and β-strand regions, about 60% to about 80% β-turn and β-strand regions, about 70% to about 90% β-turn and β-strand regions, about 80% to about 100% β-turn and β-strand regions, about 10% to about 40% β-turn and β-strand regions, about 30% to about 60% β-turn and β-strand regions, about 50% to about 80% β-turn and β-strand regions, about 70% to about 100% β-turn and β-strand regions, about 40% to about 80% β-turn and β-strand regions, about 50% to about 90% β-turn and β-strand regions, about 60% to about 100% β-turn and β-strand regions, or about 50% to about 100% β-turn and β-strand regions. In some embodiments, silk (3 sheet content, from less than 10% to −55% can be used in the silk fibroin compositions disclosed herein.

In some embodiments, the silk fibroin has a protein structure that is substantially-free of α-helix and random coil regions. In aspects of these embodiments, the silk fibroin has a protein structure including, e.g., about 5% α-helix and/or random coil regions, about 10% α-helix and/or random coil regions, about 15% α-helix and/or random coil regions, about 20% α-helix and/or random coil regions, about 25% α-helix and/or random coil regions, about 30% α-helix and/or random coil regions, about 35% α-helix and/or random coil regions, about 40% α-helix and/or random coil regions, about 45% α-helix and/or random coil regions, or about 50% α-helix and/or random coil regions. In other aspects of these embodiments, the silk fibroin has a protein structure including, e.g., at most 5% α-helix and/or random coil regions, at most 10% α-helix and/or random coil regions, at most 15% α-helix and/or random coil regions, at most 20% α-helix and/or random coil regions, at most 25% α-helix and/or random coil regions, at most 30% α-helix and/or random coil regions, at most 35% α-helix and/or random coil regions, at most 40% α-helix and/or random coil regions, at most 45% α-helix and/or random coil regions, or at most 50% α-helix and/or random coil regions. In yet other aspects of these embodiments, the silk fibroin has a protein structure including, e.g., about 5% to about 10% α-helix and/or random coil regions, about 5% to about 15% α-helix and/or random coil regions, about 5% to about 20% α-helix and/or random coil regions, about 5% to about 25% α-helix and/or random coil regions, about 5% to about 30% α-helix and/or random coil regions, about 5% to about 40% α-helix and/or random coil regions, about 5% to about 50% α-helix and/or random coil regions, about 10% to about 20% α-helix and/or random coil regions, about 10% to about 30% α-helix and/or random coil regions, about 15% to about 25% α-helix and/or random coil regions, about 15% to about 30% α-helix and/or random coil regions, or about 15% to about 35% α-helix and/or random coil regions.

ECM Agents

In accordance with various embodiments, provided compositions may include at least one of any of a variety of ECM agents. In some embodiments, a provided composition comprises at least two ECM agents. In accordance with various embodiments, an ECM agent may be or comprise any ECM component of native fetal ECM in a mammal (e.g., a human, rat, mouse, dog, primate, or other mammal). In accordance with various embodiments, an ECM agent may be or comprise any ECM component of adult ECM in a mammal (e.g., a human, rat, mouse, dog, primate, or other mammal). In some embodiments, ECM agents may be provided by decellularizing at least one of fetal or adult mammalian tissue, and extracting one or more ECM agents.

In some embodiments, an ECM agent may include one or more of fibronectin, laminin, positively charged polymers (e.g., poly-lysine), poly-ornithine, polysaccharide, collagen, elastin, serum, serum albumin, proteoglycan, glucosaminoglycan and cell adhesion molecule. In some embodiments, the cell adhesion molecule is selected from the group consisting of cadherin, immunoglobulin, selectin, mucin, integrin and combinations thereof. In some embodiments, the at least one ECM agent comprises collagen I, collagen IV and fibronectin. In some embodiments, the at least one ECM agent facilitates and/or promotes growth and/or attachment of cells on or within the matrix.

In accordance with various embodiments, any of a variety of concentrations of ECM agent(s) are contemplated. For example, in some embodiments, the at least one ECM agent is present at a concentration of at least 1 ng/ml (e.g., at least 10 ng/ml, 100 ng/ml, 1 μg/ml, 10 μg/ml, 100 μg/ml, 1 mg/ml, 10 mg/ml, 100 mg/ml or more). In some embodiments, the at least one ECM agent is present at a concentration between lng/ml and 100 mg/ml. In some embodiments, the at least one ECM agent is present substantially uniformly throughout the silk fibroin matrix. By way of non-limiting example, in some embodiments, provided compositions may include at least 10 μg/ml laminin, at least 20 μg/ml poly-ornithine, and/or at least 3 mg/ml Collagen I.

In some embodiments, provided methods and compositions include the expression or administration of one or more matrix metalloproteinases (“MMPs”). MMPs are calcium-dependent zinc-containing endopeptidases that exist in vertebrates, invertebrates, and plants. MMPs have a common domain structure including a pro-peptide, catalytic domain, and haemopexin-like C-terminal domain linked to the catalytic domain via a flexible hinge. MMPs are distinct from other endopeptidases for several reasons, including, e.g. their dependence on metal ions as cofactors and their ability to degrade extracellular matrix (“ECM”) proteins. They are also capable of processing many bioactive molecules and can participate in, e.g. chemokine/cytokine inactivation. MMPs are thought to be major players in certain cell behaviors, e.g. proliferation, migration, differentiation, apoptosis, etc.

Immune Cells

In some embodiments, provided methods and compositions include a plurality of at least one type of immune cells. In some embodiments, the at least one type of immune cells includes at least one of macrophages, microglia, leukocytes, and/or mast cells. In some embodiments at least some of the plurality of immune cells are functional immune cells (e.g., capable of carrying out their function substantially as if in vivo). In some embodiments, an immune cell or population of immune cells may be considered functional if the expression of one or more known markers of immune cell activation are detectable (e.g., pro-inflammatory cytokine [e.g. TNFα] and/or anti-inflammatory cytokine [e.g. IL-10] expression). In some embodiments, expression may be or comprise mRNA expression. In some embodiments, expression may be or comprise protein expression. In some embodiments, at least some of the plurality of immune cells are present in at least two discrete portions of a provided composition (e.g., in at least two layers or areas of a scaffold).

In accordance with a variety of embodiments, the at least one type of immune cell may be exposed to provided compositions via any known route of association. For example, in some embodiments, the at least one type of immune cells is exposed to the composition as a gel or hydrogel. By way of additional example, in some embodiments, the at least one type of immune cells is exposed to the composition via perfusion.

Endothelial Cells

In some embodiments, provided methods and compositions may further include a plurality of at least one type of endothelial cell. In some embodiments, endothelial cells are human endothelial cells (e.g., primary human endothelial cells, immortalized human endothelial cells, and hPSC-derived endothelial cells). In some embodiments, the endothelial cells comprise a monolayer. In some embodiments, a monolayer may be in the shape of a tube or portion of a tube. In some embodiments, endothelial cells may perform some or all of the functions of a functional vasculature (e.g., transporting nutrients and/or energy, removing wastes, providing cooling). In some embodiments, at least some of the plurality of endothelial cells are present in at least two discrete portions of a provided composition (e.g., in at least two layers or areas of a scaffold).

In some embodiments, the monolayer is characterized in that it is able to impede the flow of at least one fluid to or from an area substantially devoid of the plurality of human nerve cells and the plurality of glial cells into or from an area comprising the substantial majority of the plurality of human nerve cells and the plurality of glial cells. In some embodiments, the at least one fluid is a liquid or a gas. Additionally or alternatively, in some embodiments, provided compositions may include a plurality of brain microvascular endothelial cells.

In some embodiments, endothelial cells may be introduced to provided compositions as preformed/pregrown conduits. In some embodiments, such preformed conduits may interact with one or more of the other cell types in a provided composition (e.g. nerve cells, glial cells, immune cells, or others). In some embodiments, endothelial cells may be introduced to provided compositions via seeding or co-seeding the endothelial cells onto a partially or fully formed multi-layer silk composition.

In some embodiments, the at least one type of endothelial cell is exposed to the composition as a gel or hydrogel. In some embodiments, the at least one type of endothelial cell is exposed to the composition via perfusion.

Other Cells

In some embodiments, provided methods and compositions may further include at least one additional cell type. In some embodiments, provided methods and/or compositions include one or more modified stem cells. In some embodiments, provided methods and/or compositions include one or more transfected cells (e.g., comprising channel rhodopsin, or another optogenetic protein).

Human Induced Pluripotent Stem Cells and Differentiation in 3D

Without wishing to be bound by any theory, three dimensional in vitro cell culture models offer important advances within the field of tissue engineering, facilitating an understanding of mechanisms of organ development, cellular interactions, and disease progression within defined environments. The central nervous system in particular, when combined with the use of human iPS cells, can benefit from these approaches to improve the understanding of this biological structure. In some aspects, the present invention describes development and characterization of a three-dimensional tissue model that promotes the differentiation and long-term survival of neural networks. In some embodiments, these cultures show diverse cell populations including neurons and glial cells (astrocytes) interacting in 3D with spontaneous neural activity based on electrophysiogical recordings and calcium imaging. This approach allows for direct integration of pluripotent stem cells into a 3D construct, bypassing early neural differentiation steps (embryoid bodies and neural rosettes), streamlining the process and expediting the culture. These neural tissue models also show promise for use in the study of neurodegenerative disorders, such as, e.g., Alzheimer's or Parkinson's disease.

Understanding human neurodevelopment and neurological diseases represent some of the most challenging areas of biological sciences, due to the fragile and intricate nature of the human brain in vivo. With billions of neurons interconnected by trillions of synapses, parsing out accurate and specific information can prove difficult. The reverence for this intricacy is apparent in the practice of ‘awake brain surgery’, in which patients are asked to converse or perform a motor task to ensure that there is minimal disruption to normal brain function while undergoing tissue resection for, e.g. tumor, epilepsy, etc. When studying the brain in vivo, data may be derived from a variety of recording techniques including electroencephalogram (EEG) or magnetoencephalography (MEG), which both record bulk electrical activity with a high level of temporal resolution, but are largey limited to surface cortical activity. Another approach is use of functional magnetic resonance imaging (fMRI), which measures relative blood flow as a readout of activity. This method allows for better spatial resolution than either the EEG or MEG, but is temporally limited due to the lag time between synaptic activity and changes in blood oxygen level-dependent (“BOLD”) contrast. Even with these methods in combination, attaining single-cell level resolution is currently unattainable without more invasive techniques, such as the implantation of neurotrophic electrodes. Because of their invasive nature, these techniques are limited to severe cases such as patients suffering from “locked-in” syndrome. Due to these limitations, alternative models for the study and experimentation of neural cell functions in the context of complex 3D brain-related niches are required to further our understanding of the human brain.

Much of our knowledge surrounding the human brain has been gained through the use of animal (in vivo) models. Without wishing to be bound by any theory, while animal studies have significantly helped in understanding general brain development and function, they do not fully mimic human conditions of complexity and sentience, or disease states. Using animals as preclinical test models has come under scrutiny in many areas including their validity as a model for pharmacologic testing. A comprehensive survey of rates of clinical trial success for investigational drugs showed a 9.4% likelihood of acceptance (LOA) with neurological drugs in phase 1. The limitations present in animal models also include drug candidates discarded due to inefficacy observed within non-native neurological models.

An alternative to in vivo animal studies has been the use of patient tissue explants in the form of brain slices or hanging drop cultures. These culture methods have limitations including sample acquisition, damage to tissue during harvesting, limited time in culture, and inaccessibility to experimental intervention as they are often closed systems. There have been recent improvements in culture methods for ex vivo human tissue, such as microfluidic devices, but even with these advances, healthy human neurological tissues are rarely removed from a patient and even diseased tissue is often only available post-mortem. Culturing neurons, regardless of the source, can be problematic due to their post-mitotic nature, which can limit ability to expand in culture. This problem has previously been circumvented through use of pluripotent cells and the finding that mature somatic cells can be induced back into a pluripotent state (induced pluripotent stem cells-iPSCs), including for later use in controlled differentiation experiments. These pluripotent cells are able to be exponentially expanded, and, under controlled conditions can be directed to differentiate into specific multipotent cells and, finally, to terminally differentiated cell types, including neurons. There have been multiple advances in systems to support the use of pluripotent cells to differentiate into functioning neurons in three dimensions, including human and murine cerebral organoids and human cortical spheroids. These approaches generated neurospheres from hiPSCs, which were then used to create developmental brain models. The spheres were grown in spinning bioreactors, free floating, under minimal direction and formed organized structures that mimicked those found in the human brain with varying levels of consistency. The structures developed by these cells in an undirected environment demonstrated an ingrained ability to self-organize. While impressive, these models have limitations, in particular, in the absence of active media diffusion, the density and size of the spherical cultures results in necrosis and loss of function over longer time courses. Other cell and tissue models have been developed using neural stem cells or immortalized neural progenitor cells, and while these models explore development of the organoid as a whole, they are not well-suited to study network development and growth.

As exemplified herein, provided compositions bridge the gap between the accessibility of in vitro models and in vivo accuracy. In vitro, engineered models of human brain tissue can provide stable long-term systems to mimic brain tissue structure and function to support the study of neurological processes; brain development, degenerative disorders, drug interactions and related needs. As described herein, in order to achieve this goal, it is necessary to generate consistent cultures that support the growth of biologically relevant cells. The combination of defined 3D culture systems and patient derived iPS cells provides a platform that allows for personalized pharmacologic testing, and the study of the progression of numerous neurological diseases.

Active Agents

In some embodiments, provided methods and compositions may include one or more active agents. In some embodiments, wherein a provided composition comprises multiple layers or scaffold types for example, one or more active agents may be present in all layers or scaffolds of a provided composition. In some embodiments, one or more active agents may be present in fewer than all layers or scaffolds (e.g., absent from at least one layer or scaffold). In some embodiments at least one active agent is present only in a single layer or scaffold. In some embodiments, at least one active agent is distributed substantially homogenously throughout at least one layer or scaffold. In some embodiments, at least one active agent is distributed substantially as a gradient in at least one layer or scaffold. In some embodiments, at least one active agent is present only in a discrete portion of the composition (e.g., a discrete portion of one layer).

In some embodiments, the at least one active agent comprises one or more of proteins, peptides, antigens, immunogens, vaccines, antibodies or portions thereof, antibody-like molecules, enzymes, nucleic acids, siRNA, shRNA, aptamers, viruses, bacteria, small molecules, cells, hormones, antibiotics, therapeutic agents, diagnostic agents, and any combinations thereof. In some embodiments the at least one active agent comprises an anti-inflammatory agent. In some embodiments, the at least one active agent modulates the production and/or function of at least one neurotransmitter.

In some embodiments, an active agent may be or comprise at least one of collagen (e.g., Collagen I, Collagen II, Collagen III, Collagen IV, Collagen V, Collagen XI), laminin, fibronectin, hyaluronic acid, fibrinogen, sulfated glycosaminoglycans, and/or one or more growth factors. In some embodiments, the one or more growth factors are or comprise nerve growth factor, brain derived neurotrophic factor, fibroblast growth factor, insulin-like growth factor, vascular endothelial factor (VEGF), or tumor necrosis factor-β, and combinations thereof. In some embodiments, an active agent may be or comprise one or more anti-oxidants and/or one or more polyphenols.

Electrical Devices

In some embodiments, provided compositions may comprise one or more electrical devices. In some embodiments, provided methods and compositions may further include one or more electrical devices that are functionally connected to at least some of the plurality nerve cells in a provided composition. In some embodiments, activation of one or more such electrical devices results in the firing of one or more neurons and/or the activation of a plurality of cells in or on a provided composition. In some embodiments, the one or more electrical devices are or comprise at least one electrode. In some embodiments, the one or more electrical devices comprise silk fibroin.

In some embodiments, the one or more electrical devices is/are functionally connected to at least some of the plurality of nerve cells. In some embodiments, activation of the one or more electrical devices results in the firing of one or more neurons. In accordance with various embodiments, one or more electrical devices may be introduced above, below, and/or within any particular layer or scaffold of a provided composition.

In some embodiments, provided compositions may comprise one or more optical devices (e.g., optical fibers) or optical tools (e.g., optogenetic tools, for example, any of a variety of rhodopsin analogs, genetically, encoded calcium indicators, etc). In accordance with various embodiments, one or more optical devices and/or optical tools may be introduced above, below, and/or within any particular layer of a provided composition. In some embodiments, the one or more optical devices and/or optical tools are biodegradable. In some embodiments, the one or more optical devices and/or optical tools comprise silk fibroin. In some embodiments, the one or more optical devices and/or optical tools are non-biodegradable. In some embodiments, electrical devices are incorporated in the scaffolds and/or in the bioreactors to measure the transendothelial electrical resistance (TEER) to evaluate BBB function.

Diseases, Disorders, and Conditions

In accordance with various embodiments, provided methods and compositions may be used as models of diseases, disorders, or conditions or, in some embodiments, as diagnostic and/or therapeutics in one or more diseases, disorders, and conditions. Certain exemplary diseases disorders, and conditions are described below, but these are not intended to be limiting, rather, they are intended to provide specific examples of the general concept, as would be appreciated by one of skill in the art.

Traumatic Brain Injury

Traumatic Brain Injury (TBI) is defined as head trauma that occurs when brain physiology and/or morphology is disrupted either focally or diffusely, from mechanical impact applied to the head. TBIs are heterogeneous in nature, and can manifest in a variety of clinical symptoms, due to the activation of neural markers and molecular pathways, depending on the nature of the mechanical impact. Due to the complexity of the brain's structure and function, the relationships between specific molecular pathways or specific marker activation and the resulting symptoms are poorly understood, making TBI diagnosis and treatment challenging. TBIs are one of the top causes of injury and death in the US. Dysfunction of cognitive and motor abilities are significant deficits that result from TBI, with many patients experiencing significant memory loss. Despite the prevalence of TBI associated injuries, no treatments exist that are able to effectively mediate the observed immune response nor restore normal brain function.

Depending on the severity of the impact to the head, brain injuries can be divided into three groups, (1) mild injury which includes concussions, (2) moderate contusions or diffuse axonal injury, or (3) severe injury which results in hemorrhage and brain death. Until recently, brain injuries were considered acute trauma, however accumulated evidence over the last decade points toward chronic injuries, or “secondary damage”, that can manifest years after the primary impact. Secondary damage is associated with neuroinflammation, where glial and immune cells become activated, and subsequently trigger long-term degenerative mechanisms, resulting in neuropathologies such as chronic traumatic encephalopathy. In some studies these chronic effects were associated with development of Alzheimer's disease, Parkinson's disease and other dementias. Neuroiflammation has a dual effect after brain injury, at the initial stages, glial and immune cells secrete pro-inflammatory cytokines, such as tumor necrosis factor alpha (TNFα), a variety of interleukins, such as IL-1 beta, IL-13, iNOS and other molecules, that are responsible for clearing the inflammation. After a few days other anti-inflammatory cytokines (e.g., IL-4, IL-10) are released to help regenerate and restore tissue functions. However, in the case of chronic inflammation the switch towards regenerative activation of glial cells does not happen, which leads to adverse reactions and neurodegeneration. However, specific molecular mechanisms triggered after injury depend on the severity of injury, and the type of injury. For example, hemorrhagic injuries trigger a different response mechanism than contusion type injuries. In hemorrhagic, neurons undergo necroptosis due to hypoxia and blood toxicity, whereas in contusion injuries, a primary effect is activation of microglia and astrocytes. This variability in tissue response to different types of trauma underscores the importance of understanding each type of brain cell and its role in the various aspects and types of TBI.

Currently, TBI research is often conducted with in vivo rodent models, however, the inability to manipulate cell composition in these models makes it more difficult to understand the mechanisms underlying brain injury. In vitro brain tissue models can help address the limitations of in vivo models, however, it is challenging to design brain tissue models in vitro that recapitulate the complexity of native brain tissue. The brain is the most complex and sophisticated organ in the human body, and is comprised of two major cell groups: neurons and glia. To understand how the brain works in response to TBI, it is crucial to understand the function of each cell type, both separately and in combination, in healthy and pathological conditions.

The human brain contains approximately 100 billion neurons and, neurons are arguably the most important cell type of the brain, with a distinctive role in brain function.

Neurons form an extensive network, which is responsible for sensing environmental changes and regulating physiological responses. Glial cells (e.g., astrocytes, oligodendrocytes, ependymal cells) outnumber neurons tenfold and play crucial roles in maintenance of neural functions. Glial cell activities include regulation of ion balances in the extracellular space, axonal myelination, and neuronal nourishment. Microglia, the resident immune cells of the brain, are the only cell type that originates from myelomonocytic lineage, and migrate into the brain from the periphery during early embryonic development. Changes in neuronal morphology, activity, and organization have been studied in different brain pathologies, such as, e.g. TBI, Alzheimer's disease, brain cancers, etc. The contribution and/or role of glial cells in these diseases was underestimated for decades.

Without wishing to be bound by any theory, microglial responses to TBI may have both positive and negative effects on patient outcomes. In general, after the acute response to TBI has subsided, chronic microglial activation has been associated with increased severity of injury. More specifically, one study showed that through the administration of minocycline, an anti-inflammatory antibiotic, a decrease in microglial-induced inflammation and lesion severity was observed. Furthermore, active microglia produce reactive oxygen species (ROS), resulting in increased severity of injury. Despite negative outcomes observed in patients with chronically activated microglia, there are many benefits to the acute inflammatory response. The acute response is vital in regulating cell death, clearing debris, and maintaining the integrity of the blood-brain barrier (BBB). Previous studies have shown that immediate administration of an immunosuppressant can lead to increased risk of death, as well as less tissue regeneration 6 months after injury. Because of these diverse responses and complex impact of microglia after injury, there are no clinically available treatments to reduce the impact of TBI.

Similar to the microglial response to TBI, the inhibition of astrocyte responses may have both positive and negative impacts on patient outcomes. Many published studies remain unclear in the role of astrocytes in TBI responses. Depending on the injury model being studied, effects of astrocyte response can vary and serve multiple purposes. For example, one study showed that the astrocyte pathway associated with interferon (IFN)-γ, a pro-inflammatory cytokine, resulted in increased gliosis accompanied by reduced hippocampal neurodegeneration after CCI injury. Interestingly, astrocyte-specific overexpression of the pro-inflammatory cytokine IL-6 also resulted in increased wound healing concomitant with increased reactive gliosis. It has been shown that increased astrocyte activation in an in vitro oxygen-glucose deprivation model influenced the production of anti-inflammatory mediators and suppressed microglial activation. Reactive astrocytes secrete pro-inflammatory cytokines, chemokines, and matrix metalloproteinases, which may responsible for neural degeneration and blood-brain barrier disruption.

Overall, all brain cells can have a significant effect on the outcome of brain injury, thus it is crucial to create a tissue model that recapitulates the complexity of the native brain in structure and cell composition and it is contemplated that certain embodiments have improved and expanded upon our prior brain-tissue models to increase cellular complexity by incorporating microglia and astrocytes with the neurons. One or more aspects of the present invention may include a 3D model that incorporates more than one category of brain cell (e.g. neurons, astrocytes and/or microglia), and models different types of brain injuries (e.g. hemorrhage or contusion). In some embodiments, contemplated compositions are useful in vitro tools to gain insight into mechanisms and develop new treatments.

Brain Tumors

Brain tumors are the leading cause of pediatric tumor-related deaths with medulloblastoma being the most common embryonic tumors (tumors arising from cells at their earliest developmental stage). Furthermore, though highly invasive, none of the malignant primary brain tumors in pediatric and adult patients, including the most aggressive subtype, glioblastoma multiforme (GBM), have been known to metastasize outside of the central nervous system (CNS). Without being bound to any theory, this suggests the presence of a highly specialized microenvironment/extracellular matrix (ECM) in brain tumors, which is known to play an important role in, e.g., cell proliferation, migration, and invasion. Abnormally high levels of hyaluronic acid (HA) have been documented in regions around gliomas, and overexpression of certain chondroitin sulfate proteoglycans (CSPGs) has been seen, which supports that specific ratios of various brain ECM components can change in diseased states. Although, proteoglycans including CSPGs and heparin sulfate proteoglycans (HSPGs) are known to be mostly upregulated in highly invasive and lethal brain tumors such as, e.g., glioblastoma; it has been shown that certain CSPGs in particular may act as organizers of tumor microenvironment, preventing diffusive infiltration in non-invasive tumors, and being largely absent from infiltrative types.

Current platforms for investigating brain tumor biology include 2D monolayer cultures, organotypic cultures, and animal models, which have limitations such as, e.g., the lack of a 3D ECM environment, limited tunability for mechanistic studies, and cost/time intensity, low-throughput/need for immunocompromised animal models, respectively. Moreover, previous tumor studies using 3D models have predominantly focused on the mechanical effects of matrix on the growth of established tumor cell lines for glioblastoma and medulloblastoma.

In some embodiments, to determine the role of ECM in tumor development, a novel 3D model of tumor may be used, including cultured primary tumor cells from patient-excised brain tumors and a 3D in vitro bioengineered model of brain. Without wishing to be bound by any theory, one hypothesis is that fetal brain ECM, which has been found to be rich in chondroitin sulfate (CS), might be impeding the growth of medulloblastoma cells, as CS has previously been found to inhibit invasion in non-aggressive tumor microenvironments. In some embodiments, the present studies will elucidate the biochemical effects of introducing native brain-derived ECM in 3D cultures of primary tumor cells in order to, inter alia, development a representative brain tumor model that is physiologically and clinically relevant.

Without wishing to be bound by any theory, the inclusion of brain-derived ECM in provided 3D cultures is predicted to provide a physiologically relevant environment for long-term growth of the primary tumors in vitro, with resulting endpoints comparable to that observed in the histopathological findings of the respective tumor. The controlled modification of the microenvironmental cues, particularly CSPGs, presented to the primary tumor cells both in the stromal and soluble forms, will provide insight into the novel roles of these ECM molecules towards tumor progression and inhibition. It is possible that CSPGs, when presented in stromal versus soluble forms, could have opposing effects on tumor cell growth. Such information will help guide the future targeting of these ECM molecules as markers of tumor invasiveness and as potential therapeutic targets, e.g., by slowing down diffuse infiltrative tumor growth.

Examining and understanding the complex interactions between brain tumor niches and cells has been quite limited using standard 2D cultures or animal models. The novel platform presented in this application is the first known study to combine the tunability of a 3D bioengineered brain model with native brain-derived ECM from different developmental stages in conjunction with patient-derived brain tumor cells. The enhanced microenvironmental biomimicry and tunabilty for mechanistic studies will provide a model system and potentially resolve whether or not providing a CSPG rich microenvironment to an already aggressive tumor can impede its growth. In some embodiments, provided compositions may also help unravel the identity and composition of ECM deposited by tumor cells when cultured long-term in physiologically relevant microenvironments.

Brain Grafts

Brain damage is one of the most devastating pathologies that leads to massive neuron extinction and chronic brain inflammation, resulting in serious patient impairment, long-term disabilities, a significant decrease in quality of life, and in many cases, death. Annually, 10 million people are diagnosed with stroke (Collaborators, 2016), 10 million with various traumatic brain injuries (TBIs) (Wagner, 2013; Wong and Langley, 2016), and about 1 million with brain cancer (Collaborators, 2016). Combined, brain damage is the most frequent cause of death among individuals under the age of 45. Annual estimated cost for the treatment of brain injuries and rehabilitation in the United States is around 50 billion dollars (Humphreys et al., 2013; Melton, 2017). A recent study showed that nearly half of head trauma patients experienced symptoms from dizziness, tremors, or poor coordination, to complete paralysis. Most of these patients will be affected by the secondary impact of brain damage, such as from hypoxia, ischemia, brain atrophy, and demyelination (Humphreys et al., 2013; Kochanek et al., 2013). Further, many patients experience social problems in terms of integrating back into society, and as a consequence this leads to depression and associated increased risks of suicide (Humphreys et al., 2013). One reason for such devastating consequences with brain damaged patients may be due to the limited ability of the brain to self-regenerate and repair after these types of injuries. Without wishing to be bound by any theory, in part, this may be due to presence of only two small areas in the brain that are known to maintain neural progenitor cells: the subventricular zone (SVZ) and the subgranular zone (SGZ) (Gage and Temple, 2013). These progenitor cells are essential for brain damage repair and regeneration, however, while these progenitor cells play a critical role in replacement of small damaged areas, they cannot restore tissue architecture and function in large regions or in those very far away from the SVZ or SGZ (Arvidsson et al., 2002). Unfortunately, there is currently no therapy to fully restore brain architecture and lost functions after brain trauma. Most currently available treatments are focused on mitigating symptoms, but do not treat or repair the underlying cause of these symptoms, brain damage.

Brain restoration using external sources of cells, or other supplements, is a young field. Currently two major approaches are being pursued, internal stimulation to trigger brain self-regeneration, and transplantation of cells or more complex tissue-like grafts to restore brain architecture and function (Chen et al., 2016). The first approach is based on the activation of the self-regeneration capacity of the brain using growth factors, drugs or electrical stimuli. A variety of studies have shown increased neurogenesis, reduced stroke area, and improved functional outcomes after administration of different growth factors, such as erythropoietin (Gonzalez et al., 2007), vascular endothelial growth factor (VEGF) (Ryu Matsuo et al., 2013; Yang et al., 2014), brain derived neurotrophic factor (BDNF) (Fantacci et al., 2013; Grade et al., 2013), insulin-like growth factors (IGF) and others (Larpthaveesarp et al., 2015). Deep brain stimulation (DBS) (Sironi, 2011) is becoming a more common procedure to treat patients with brain diseases (Guggenmos et al., 2013), including Parkinson's, major depression (Williams, 2015), TBI and others (Rezai et al., 2016), and this approach is mostly focused on implanting electrodes as a bridge to restore communication between brain areas that were affected post injury (Rezai et al., 2016; Satzer et al., 2014; Williams, 2015). However, as mentioned earlier, the brain appears to have a limited regeneration capacity, which further limits the impact of this approach for the regeneration of large size brain defects. Thus the second approach, based on cellular injections or engineered brain-like tissue implantation have also been pursued (Chen et al., 2016). These approaches potentially allow the restoration of larger size brain defects, but also have limitations, such as suitable cell sources, fabrication of grafts that mimic native brain architecture, and management of acute inflammation post-implantation (Chen et al., 2016).

Early studies showed high integration and survival of injected human fetal neural progenitors (Jensen et al., 2013) or mature neurons (Weick et al., 2011) into rodent brains, however this approach was associated with ethical issues when considering human studies. The discovery of induced pluripotent stem cells (iPSCs) eliminates some of the ethical concerns, and iPSCs are currently being widely studied for brain tissue fabrication and regeneration (Lopez-Leon et al., 2014). Proper brain function is associated with an intact brain architecture. Thus, the successful implantation of cells or engineered brain grafts with simple architectures (Jgamadze et al., 2012), can be limiting depending upon which functions need to be restored.

Recent advances in tissue engineering allow the fabrication of brain-like tissue constructs in vitro to recapitulate the complexity of the native brain architecture, and offer new options for transplantation of brain tissue to restore defects (Bouyer et al., 2016; Jgamadze et al., 2012; Lancaster et al., 2013). Tissue engineering as a subfield of regenerative medicine combines principles of engineering, medical biology and material science to restore or replace damaged tissues or organs (Hopkins et al., 2015). Currently, two promising approaches to fabricate functional brain grafts include scaffold-free approaches, based on the principal of cells self-organization into more complex tissue, brain spheroids and organoids (Dingle et al., 2015; Lancaster et al., 2013); and scaffold-based approaches, where biomaterials are utilized to control tissue architecture and cell organization (Bouyer et al., 2016; Hopkins et al., 2015; Lozano et al., 2015; Tang-Schomer et al., 2014a). Both of these approaches are promising as research tools and could facilitate development of fundamental understanding of tissue grafting, however, application of brain organoids for transplantation is challenging.

Organoids and spheroids are prone to develop necrotic cores due to uncontrollable cell growth and poor mass transfer related to oxygenation and nutrients to the central regions of the structures, potentially triggering acute inflammation and consequently transplant rejection. Thus, engineered scaffold-based brain grafts with controlled tissue architecture, such as those provided herein in some embodiments of the present invention, may be a useful alternative for brain tissue transplantation, by avoiding the complications above and providing improved control of brain-like tissue structure and function.

Regardless of the approach utilized, neuroinflammation remains a major limitation after cell, biomaterial or tissue graft transplantation. Earlier studies with human cell sources (embryonic or induced pluripotent stem cells (iPSCs)) were performed on immunosuppressed rodents to eliminate transplant rejection and post-operative complications (Falkner et al., 2016). Neuroinflammation is a critical defense mechanism in the mammalian brain, but also plays an irreplaceable role in wound healing and tissue regeneration, thus complete immune suppression might delay or prevent implant integration and tissue regeneration and thus, be detrimental to treatment. The inflammatory response is activated by immune cells in the brain (microglia and macrophages) (Boche et al., 2013; Hu et al., 2015; Prinz and Priller, 2014; Saijo and Glass, 2011; Witcher et al., 2015). These cells display both pro-inflammatory (M1) and anti-inflammatory (M2) functions, upon contact with specific inflammatory markers, such as tumor necrosis factor alpha (TNF alpha) and lipopolysaccharide (LPS) for pro-inflammatory/M1 phenotypes, and interleukin-4 (IL-4) and interleukin-10 (IL-10) for anti-inflammatory/M2 phenotypes (Boche et al., 2013; Hu et al., 2015; Prinz and Priller, 2014; Saijo and Glass, 2011; Witcher et al., 2015). Neuronal damage or blood-brain barrier disruption after brain trauma triggers initial inflammatory responses associated with inflammatory cytokine, including burst release of TNF-alpha, ILI-beta, and other cytokines (Hu et al., 2015; Witcher et al., 2015). This response activates the M1 stage of immune scavengers, and increases the amount of secreted pro-inflammatory cytokines. Normally, in few days the microglia shift toward the M2 stage to clear damaged tissue, and trigger tissue regeneration (Hu et al., 2015; Prinz and Priller, 2014). However, in a severely damaged brain the amount of secreted pro-inflammatory cytokines can worsen the inflammation and not allow the microglia to shift to the regeneration phase; resulting in chronic inflammation and secondary brain damage (Witcher et al., 2015).

During post-mortem tissue analysis of severe TBI patients, a pronounced increase of pro-inflammatory cytokine mRNAs was documented (Kochanek et al., 2013). In the case of graft implantation, immune cells are not capable of easily eliminating the source of activation that triggers overproduction of inflammatory cytokines, and as a result, chronic inflammation, graft scarring, and rejection ensue (Boche et al., 2013; Hu et al., 2015; Prinz and Priller, 2014; Saijo and Glass, 2011; Witcher et al., 2015). Thus, successful brain grafts should mimic native brain architecture and be composed of inert or low immunogenic materials, neural progenitors or mature neurons, and factors that can mitigate inflammation and improve graft survival. Prior to the present invention, these challenges have yet to be met in the field and required new strategies to orchestrate proper graft design and function both in vitro and in vivo.

3D Intracerebral Hemorrhage

Intracerebral hemorrhage (ICH) is the deadliest form of stroke and accounts for 15% of all strokes in the United States. However, there are no effective treatments for ICH and the treatments that are available must be administered within hours of injury, which, for various reasons is not possible or does not happen in many cases. ICH brain models are invaluable tools in the search for new therapies, but all models currently available are lacking in at least one aspect. The most common models used are in vivo mouse models, which, among other challenges and criticisms, do not always translate to human physiology. Other models attempt to replicate the in vivo environment, but they are either two-dimensional co-cultures, or three-dimensional mono-cultures. Because the three-dimensional aspect affects the growth of neurons and glial cells, which regulate the inflammatory response, both aspects should be present in an ideal ICH model. In some aspects, the present application demonstrates a novel three-dimensional tri-culture model of ICH using primary murine striatal neurons, astrocytes, and microglia grown on a silk scaffold.

In the United States, approximately 795,000 hemorrhagic strokes occur every year. Intracerebral hemorrhage (ICH), a particularly fatal form of stroke, occurs in 15% of all strokes (King et al. 2014). High blood pressure, a common health problem in the United States today, increases the risk of spontaneous intracerebral hemorrhage 2-6 fold (Yadav et al. 2007). However, intracerebral hemorrhage is particularly hard to diagnose as its symptoms are easily confused with other neurological conditions such as seizures (Magistris, Bazak, and Martin 2013). ICH occurs when there is a stroke in a striatal artery and that artery then ruptures, starting a hemorrhage in the surrounding brain tissue. While the damage to the artery is significant, the aspect of the injury that affects disease outcome the most is the post-rupture inflammatory response of the surrounding tissue to the influx of blood (Magistris, Bazak, and Martin 2013). Expansion of the hematoma affects permeability of the blood-brain barrier which then allows potential toxins to enter the brain tissue. If the hematoma reaches a size of 30 mL or larger, mortality rates increase even further (Magistris, Bazak, and Martin 2013). Early detection of ICH is essential for limiting long-term brain damage and is usually done with brain imaging such as CT or MRI (Magistris, Bazak, and Martin 2013).

ICH patient outcome is estimated with an intracerebral hemorrhage score (ICHS) (Masotti et al. 2016). The ICHS is calculated by summing the scores for each of the patient characteristics found in Table 1 (Panchal, Shah, and Shah 2015). Scores range from 0 to 6, where 0 corresponds to a 0% mortality risk and 6 nears 100% mortality risk (Masotti et al. 2016). Patients taking antithrombotic agents, a common medication used for heart conditions or post-operative care, have a higher mortality rate from ICH due to the larger initial hematoma from the medicated and thinned blood (Masotti et al. 2016).

Current treatment methods focus on two different areas: stopping bleeding in the brain and managing other symptoms of ICH. To stop bleeding, surgeries such as endovascular coiling or placement of surgical clips are employed (Magistris, Bazak, and Martin 2013). Management of intracranial pressure and rehabilitation for severe brain injury are used to help patients regain function and independence (Magistris, Bazak, and Martin 2013). However, there are no treatments on the market which are effective at preventing neuronal injury and minimizing the inflammatory response in the brain itself (Murray, Parry-Jones, and Allan 2015). Medications currently being researched need to be administered in a time frame of hours in order to be effective (Murray, Parry-Jones, and Allan 2015). However, patients cannot always get to the hospital within a few hours if they are incapacitated by a stroke or if they do not have a caretaker available at the moment of the stroke.

To develop new treatments, different ICH models are being used to gather preclinical data. Prior to the present invention, mouse in vivo models were the dominant system (King et al. 2014, Su et al. 2015). These can be used to model many different diseases, as disease-causing mutations can easily be introduced into their genomes. This allows researchers to analyze the impact of hemorrhage in a wide range of patients, as well as observe any reactions the drug may have to common mutations.

As described in the Examples below, both porosity and cell types were verified by a scanning electron microscope and immunocytochemistry, respectively. Scaffold porosity was then optimized for striatal neuron growth. The models were then challenged with a blood injection to simulate a burst blood vessel. Assays were chosen to measure the viability of the cells, cell numbers, changes in their metabolism and proliferation, and release of reactive species. Necrostatin-1 (Nec-1), a drug currently researched for treatment of ICH, inhibits the RIP1/RIP3 pathways of necroptosis which are regulated by microglia (Su et al. 2015). This model was selected to detect necroptosis in our samples because a reduction in cell death or release of reactive species in the nec-1 treated groups would indicate that the drug is successfully inhibiting necroptosis.

Exemplary Methods of Making

In some embodiments, the present invention provides methods of making compositions including the step of providing a plurality of human nerve cells, providing a plurality of glial cells, and associating the plurality of human nerve cells and plurality of glial cells with a silk fibroin matrix or scaffold.

In some embodiments, the silk fibroin matrix or scaffold may include one or more silk fibroin structures. In some embodiments including multiple silk fibroin structures, at least two of the structures are different from one another. In some embodiments, the silk fibroin comprises a three dimensional matrix. In some embodiments, the three dimensional matrix is or comprises a scaffold. In some embodiments, a matrix or scaffold may be or comprise a tube, film, fiber, foam, gel, particle (e.g., microsphere, nanosphere, etc), ring shaped structure, concentric rings, and combinations thereof. In some embodiments, a matrix or scaffold is not a tube. In some embodiments, a matrix or scaffold is not a film. In some embodiments, a matrix or scaffold is not a fiber. In some embodiments, a matrix or scaffold is not a foam. In some embodiments, a matrix or scaffold is not a gel. In some embodiments, a matrix or scaffold is not a particle (e.g., microsphere, nanosphere, etc.). In some embodiments, a matrix or scaffold is not a ring shaped structure. In some embodiments, a matrix or scaffold is not concentric rings. In some embodiments, provided methods comprise the provison or introduction of compositions comprising a plurality of layers. In some embodiments, compositions comprising a plurality of layers do not include an adhesive added to the composition to hold the layers together.

In accordance with various embodiments, any variety of silk fibroin may be used. For example, in some embodiments, the silk fibroin is selected from the group consisting of silkworm silk fibroin, spider silk fibroin, silk tropoelastin blends, one or more genetically engineered variants of silk fibroin, and combinations thereof.

In some embodiments, provided methods further comprise introducing a plurality of pores into the matrix or scaffold. In some embodiments, at least some of the pores are interconnected. In some embodiments, substantially all of the pores are interconnected. In some embodiments, the arrangement of the plurality of pores is substantially random. In some embodiments, at least some of the plurality of pores are substantially aligned. In some embodiments, the pores are about 50 um to about 1000 um in size. In some embodiments, pore sizes of 500-600 uM may be particularly advantageous for encouraging interconnectivity and cell adhesion. In some embodiments, pore sizes of 200-300 uM may be particularly advantageous for encouraging interconnectivity and cell adhesion.

In accordance with various embodiments, the plurality of nervous system cells may be or comprise any of a variety of nervous system cells. For example, in some embodiments, the plurality of human nerve cells is selected from the group consisting of neurons, neural stem cells, neural progenitor cells, induced pluripotent stem cells, and combinations thereof. In some embodiments, the plurality of neurons comprise at least one of motor neurons and sensory neurons.

In accordance with various embodiments, any of a variety of glial cells may be used. In some embodiments, the plurality of glial cells are human glial cells. In some embodiments, the plurality of glial cells is selected from the group consisting of glial progenitor cells, ependymal cells, NG2 cells, astrocytes, microglia, oligodendrocytes, Schwann cells, tanycytes, and combinations thereof. In some embodiments, at least some of the plurality of glial cells myelinate at least one neuron in a provided composition. In some embodiments, provided compositions comprise neuronal and glial cells present in a ratio such that both cell types receive appropriate nourishment and do not substantially inhibit the function and/or proliferation of each other. In some embodiments, the presence of glial cells as described herein may supplement the exchange of neurotransmitters and contribute to chemical/enzymatic modification of said neurotransmitters.

In some embodiments, provided methods further comprise introducing a plurality of endothelial cells. In some embodiments, at least some of the plurality of endothelial cells are human endothelial cells. In some embodiments, substantially all of the plurality of endothelial cells are human endothelial cells. In some embodiments, the endothelial cells comprise a monolayer. In some embodiments, the monolayer is characterized in that it is able to impede the flow of at least one fluid to or from an area substantially devoid of the plurality of human nerve cells and the plurality of glial cells into or from an area comprising the substantial majority of the plurality of human nerve cells and the plurality of glial cells. In some embodiments, the at least one fluid is a liquid or a gas. Additionally or alternatively, in some embodiments, provided methods or compositions may include a plurality of brain microvascular endothelial cells. In some embodiments, this endothelial cell layer also serves a function for selective transport of molecules from one side of the monolayer to the other, regulating what gets into different compartments of provided compositions such as drugs, signaling factors, inflammatory compounds and others.

In accordance with some aspects of the present invention, certain provided compositions may be capable of performing certain functions and/or having certain structures classically associated with a blood brain barrier. Accordingly, in some embodiments, provided methods may include the provision of a blood brain barrier-like structure (e.g., a monolayer of endothelial cells). By way of non-limiting example, in some embodiments, provided methods further comprise introducing at least one extracellular matrix (ECM) agent (e.g., to the matrix or scaffold). In some embodiments, at least two ECM agents are introduced.

In some embodiments, the at least one ECM agent may include one or more of fibronectin, laminin, fibrin, poly-lysine, poly-ornithine, polysaccharide, collagen, elastin, serum, serum albumin, proteoglycan, melanin and cell adhesion molecule. In some embodiments, the cell adhesion molecule is selected from the group consisting of cadherin, immunoglobulin, selectin, mucin, integrin and combinations thereof. In some embodiments, the at least one ECM agent comprises collagen I, collagen IV and fibronectin. In some embodiments, the at least one ECM agent facilitates and/or promotes growth and/or attachment of cells on or within the matrix.

In accordance with various embodiments, any of a variety of concentrations of ECM agent(s) are contemplated. For example, in some embodiments, the at least one ECM agent is present at a concentration of at least 1 ng/ml (e.g., at least 10 ng/ml, 100 ng/ml, 1 μg/ml, 10 μg/ml, 100 μg/ml, 1 mg/ml, 10 mg/ml, 100 mg/ml or more). In some embodiments, the at least one ECM agent is present at a concentration between 1 ng/ml and 100 mg/ml. In some embodiments, the at least one ECM agent is present substantially uniformly throughout the silk fibroin matrix.

In accordance with various embodiments, provided methods may further comprise introduction of at least one active agent. In some embodiments, the at least one active agent comprises one or more of proteins, peptides, antigens, immunogens, vaccines, antibodies or portions thereof, antibody-like molecules, enzymes, nucleic acids, siRNA, shRNA, aptamers, viruses, bacteria, small molecules, cells, hormones, antibiotics, therapeutic agents, diagnostic agents, and any combinations thereof. In some embodiments the at least one active agent comprises an anti-inflammatory agent.

In some embodiments, provided methods further comprise providing at least one electrical device, optical device, and/optical tool (e.g. to a provided composition, for example, in functional communication therewith). In some embodiments, provided compositions may include at least one electronic device. In some embodiments, the at least one electronic device comprises at least one electrode. In some embodiments, provided methods and/or compositions further include at least one of an optical device and an optical tool.

In some embodiments, provided methods may further comprise associating at least some of the plurality of nerve cells and/or glial cells with a differentiation media or other differentiation supplement. In some embodiments, provided methods occur in a two dimensional culture. In some embodiments, provided methods occur in a three-dimensional culture.

In some embodiments, provided methods may be used to create a model of nervous tissue (e.g. brain tissue) that is or approximates such tissue when suffering from a disease, disorder or condtion such as Alzehimer's disease, cancer, etc (or any other disease, disorder or condition described herein). Thus, in some embodiments, at least some of the plurality of nervous system cells and/or glial cells may include at least one genetic or other abnormality as compared to normal healthy tissue. By way of specific example, in some embodiments, at least some of the plurality of nerve cells may exhibit abnormal expression and/or processing of a tumor suppressor such as p53, a growth factor or related protein such as epidermal growth factor receptor (EGFR), amyloid precursor protein, presenilin 1, or presenilin 2 at the genetic and/or protein level.

Exemplary Methods of Using

While contemplating a wide variety of uses for provided compositions, certain exemplary uses are described below in order to provide a clearer picture of some of the benefits of certain embodiments. For example, in some embodiments, provided compositions may be useful for studying diseases of nervous tissue, such as the brain. In some embodiments, provided compositions may be useful in repairing damage to nervous tissue, such as that due to injury or disease. By way of specific example, in some embodiments, provided compositions may also be useful in studying certain diseases including Alzheimer's disease, Parkinson's disease, Huntington's disease, traumatic brain injury, fibrosis, multiple sclerosis, certain cancers (e.g., an astrocytoma such as glioblastoma, a glioma, metastatic brain tumor, meningioma, pituitary tumors, etc), and other nervous system diseases due to the longevity of sustained cultivation and greater physiological relevance of provided compositions as compared to previously known compositions. It is specifically contemplated that certain provided compositions may be useful in stdying and/or treating any of the conditions described herein and not just those examples listed in this paragraph.

In some embodiments, provided compositions may be useful in identifying and/or characterizing therapeutic compositions for treating diseases, disorders, or conditions or the central and/or peripheral nervous systems. In some embodiments, provided compositions may be used to assess the efficacy of certain drugs (e.g., formulated for intrathecal or other brain delivery including intravenous, if appropriate). In some embodiments, the present invention provides methods including the steps of providing a composition according to any of the compositions described herein, associating one or more active agents with the composition, and characterizing the effect of the one or more active agents on the composition. In some embodiments, an effect may be or comprise: alteration of the expression of one or more gene or proteins, alteration of the production of one or more meurotransmitters, alteration of cell viability, and/or oxygen and/or glucose metabolism, relative to a control composition not associated with the active agent. In some embodiments, the present invention provides methods including the steps of providing a composition according to any of the compositions described herein, associating one or more active agents with the composition, and determining if the one or more active agents modulates the production of at least one neurotransmitter.

In some embodiments, provided compositions may be maintained for a long period of time. In some embodiments, a long period of time is at least one week (e.g., at least 2 weeks, 3 weeks, 4 weeks, 5, weeks, 6 weeks, 7 weeks, 8 weeks, 9 weeks, 10 weeks, 11 weeks, 12 weeks, or more). In some embodiments, provided compositions may be maintained indefinitely. In some embodiments, such long useful life may be achieved through use of a bioreactor type design.

### EXAMPLES

### Example 1: Silk Scaffolds and ECM Gels

Materials and Methods

Unless otherwise specified, the below examples each use the following materials and/or methods, as applicable:

Silk Scaffold and ECM Gel Preparation

Silk solution and porous scaffolds were prepared from Bombyx mori cocoons as described previously (See Tang-Schomer et al., Bioengineered functional brain-like cortical tissue, 2014, PNAS, 111(38): 13811-13816, the disclosure of which is hereby incorporated by reference in its entirety). A biopsy punch with concentrically arranged rings was used to punch out layered donut shapes. These structures were separated to prepare for cell seeding and reassembled back into the original structure. The scaffolds were autoclaved and coated with poly-L-lysine (10 μg/mL). The scaffolds were immersed in concentrated neuron suspension (20-30 million cells/mL) for 24 hours, washed extensively to remove unattached cells, and prepared for hydrogel infusion. Collagen gel was prepared from rat tail type I collagen I (3-4 mg/mL; BD Biosciences), 10×199 media (Gibco), and 1 M NaOH by mixing at a ratio of 88:10:2. Fibrin gel was prepared by mixing fibrinogen (20 mg/mL; EMD Millipore) and thrombin (10 U/mL; Sigma) at a volume ratio of 2:5. Matrigel was purchased from BD Biosciences.

To make a scaffold-gel composite structure, the scaffolds were washed with ECM gels in liquid form to replace the media within the scaffold followed by incubation at 37° C. for 30 min before media immersion.

### Example 2: Cell Culture: Rodent Sources

In this Example, cells were first isolated and studied in two dimensional models and later adapted and studied in 3D models. Primary neurons and glia were cultured from rodents (rats and mice). Fetal neurons were isolated according to previously used procedures and used in preparation of a 3D scaffold model as previously described (see Tang-Schomer, 2014, PNAS).

Glial Cell Culture and Isolation

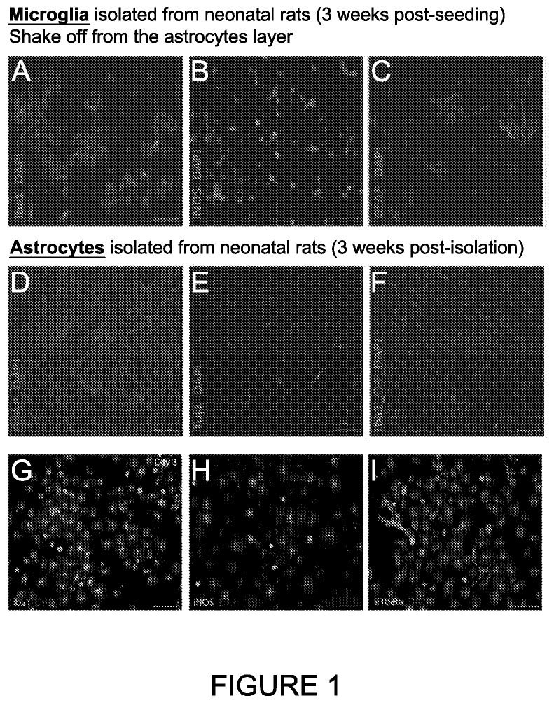

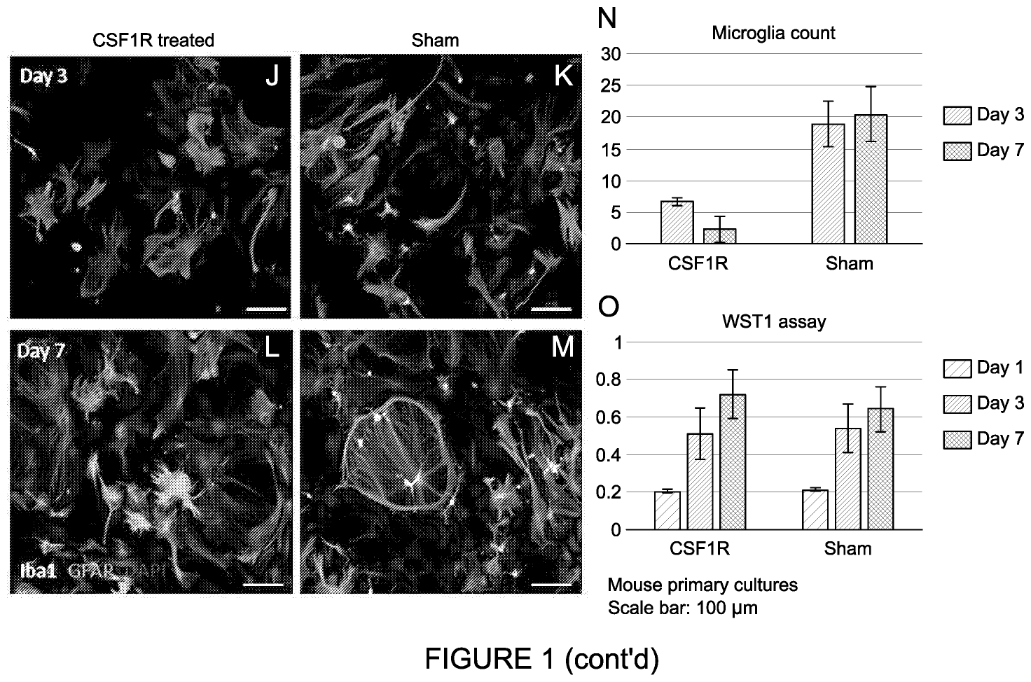

Glial cells were isolated according to available protocols (Tamashire, T. T., 2012; Mingwei, N., 2010). In brief, cortical tissue was collected from neonatal cerebellum, dissociated and plated in T75 flasks and allowed to grow for 2-3 weeks in media composed of 75% glial media and 25% neuronal media, until there were sufficient numbers of microglia in the cultures. After a 2-3 week period, a sufficient number of microglia were growing on top of a confluent astrocytic layer. Microglia were then shaken off of the cultures by shaking the flasks at 100 rpm for 1 hour at 37° C. Media was then collected from the flasks and spun at 1000 rpm for 5 minutes to collect cells. Cells were counted following spinning and used either for expansion of additional microglial cultures or used in subsequent experiments. Following removal of microglia, many astrocytes remained. These astrocytes were either frozen or used in subsequent experiments. The results in FIG. 1 show that primary microglia and primary astrocytes were successfully isolated from rat and mouse tissue. Cells were characterized to determine purity of cultures and to assess presence of specific phenotypic markers. Cells were stained according to the protocol “Immunocytochemistry (ICC) and immunofluorescence (IF) protocol”, Abcam, available on the world wide web at abcam.com/protocols/immunocytochemistry-immunofluorescence-protocol.

As shown in FIG. 1, panels A-F, cells were identified with the following molecular markers: neurons—beta-III tubulin (clone tuj1) and microtubule-associated protein 2 (MAP2); astrocytes—glial fibrillary acidic protein (GFAP); oligodendrocytes—O4; and microglia—iba1. DAPI was used as a nuclear counterstain. Microglia were also assessed for their ability to activate upon or following treatment with lipopolysaccharide (LPS) (FIG. 1, panel G), and further analyzed post-treatment for expression of inducible nitric oxide synthase (iNOS) (FIG. 1, panel H) and interleukin 1-beta (IL1-beta) (FIG. 1, panel I). Microglia activation ability with LPS (lipopolysaccharide) treatment was performed with 100 ng/mL of LPS to activate microglia cells (20,000 cells per well, 96 well plate). Cells were treated for 3 days, which was efficient to activate microglia.

During initial cell culture processes, it was noted that glial cultures contained a greater proportion of microglia relative to astrocytes, therefore alternative cell culture methods were considered to suppress microglial activity in some cultures in order to obtain pure astrocyte populations. Among those conditions considered but found unacceptable were clodronic acid, to suppress microglial growth and activity. In addition to primary microglia, an immortalized microglial cell line (N9) was also used. It was noted that this line has a fast proliferation rate and appeared to suppress neuronal cultures, so primary cell lines were maintained as a source of microglia. In general, it is thought that microglia inhibit growth of astrocytic populations. Microglia proliferation is strictly regulated by colony-stimulating factor 1 and its receptor on microglia membranes. Without wishing to be held to a particular theory, it was contemplated that if the receptor for CSF1 growth factor was inhibited, the growth of microglia in astrocytic population would be inhibited. Accordingly, a 2 week experiment was performed in two dimensional (2D) culture conditions to see if microglial growth could be inhibited. For this experiment, the glial media that was supplied with luM supplement of CSF1R inhibitors was used. Media was changed every second day in 96 well plates with 10.000 mice primary astrocytic cultures. As can be seen in FIG. 1, panels J-M, cells were cultures and fixed after 3 or 7 days, then immunostained with specific markers: GFAP for astrocytes (red, panels J-M), ibal for microglia (green, panels J-M), and DAPI (blue) as a nuclear counterstain. Number of microglia was determined in cells treated with CSF1R inhibitor or control (FIG. 1, panel N) and further evaluated via WST-1 assay (FIG. 1, panel 0).

Co-Cultures of Neurons and Glia

As stated above, initial experiments were conducted under 2D conditions before moving to three dimensional (3D) conditions. Conditions for optimal co-culturing of neurons with astrocytes and/or microglia were evaluated, including media composition and cell ratio. Media composition may have a significant impact on neurons and/or glia. In standard culture procedures neuronal media was serum free and had a high concentration of glutamine-containing supplements (e.g. GLUTAMAX®) and other growth factors, specifically approximately 480 ml of neurobasal media, 10 ml of B27 supplement, 5 ml antibiotics, 5 ml GLUTAMAX®. In standard culture procedures glial media has serum and a basic growth factor composition, specifically, approximately 450 ml Dulbecco's Modified Eagle Medium, supplemented with approximately 50 ml heat inactivated fetal bovine serum and 5 ml of antibiotics. In considering culture composition, it was thought that presence of serum in primary neuronal cultures may boost proliferation of other cell types (e.g., glia), such that the ratio of cells in samples would change to include more glia and fewer neurons, whereas growth factors typically used in neuronal cultures may cause astrogliosis/microgliosis.

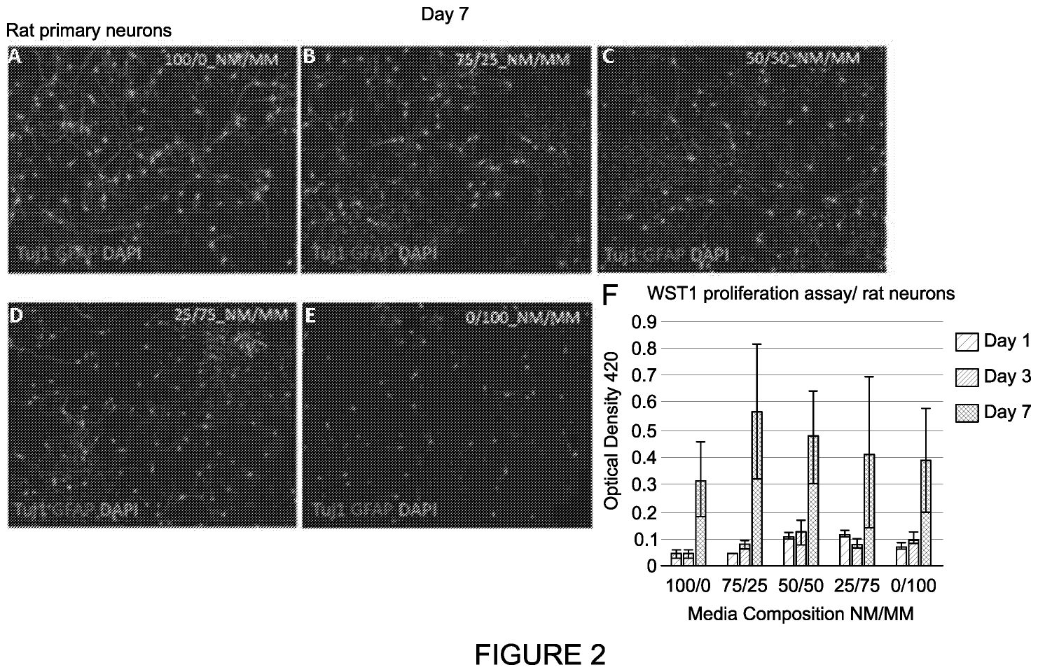

As shown in FIG. 2, panels A-F, effect of culture conditions on neuronal and glial growth and proliferation was evaluated using differing media conditions (e.g. varied ratios of neuronal media (NM) and glial media (MM)), phenotypic markers (GFAP—astrocytes; tuj1—neurons; DAPI—nuclear counterstain) (FIG. 2, panels A-E) and a Cell Proliferation Reagent WST-1 (WST1) proliferation assay to evaluate numbers of neurons over the course of one week (FIG. 2, panel F). Media ratios used were as follows: FIG. 2, panel A: 100:0 NM to MM; FIG. 2, panel B: 75:25 NM to MM; FIG. 2, panel C: 50:50 NM to MM; FIG. 2, panel D: 25:75 NM to MM; FIG. 2, panel E: 0:100 NM to MM.

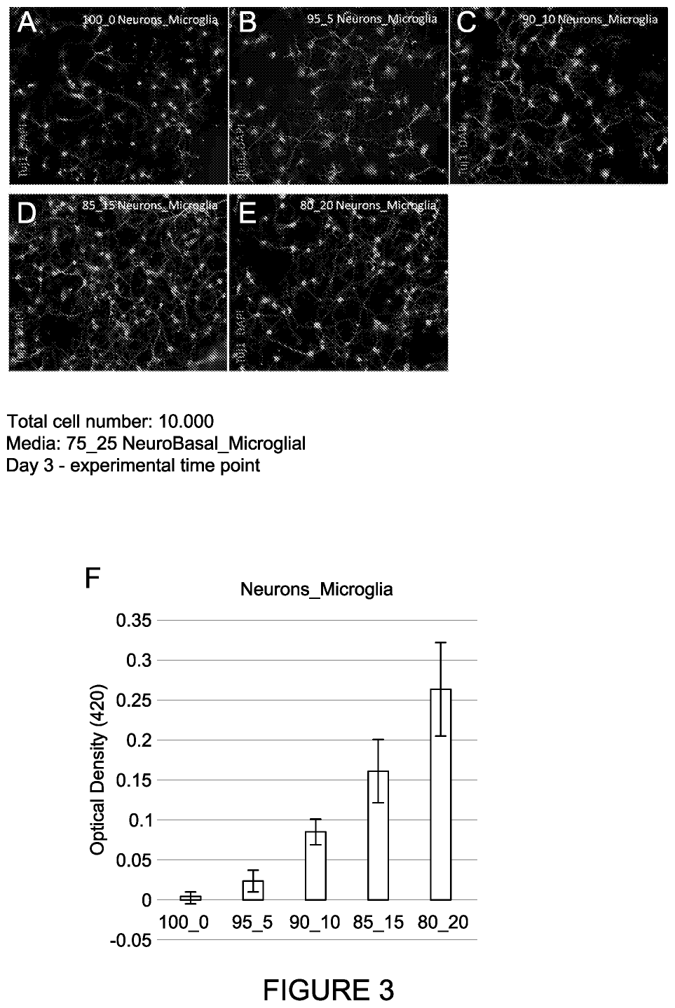

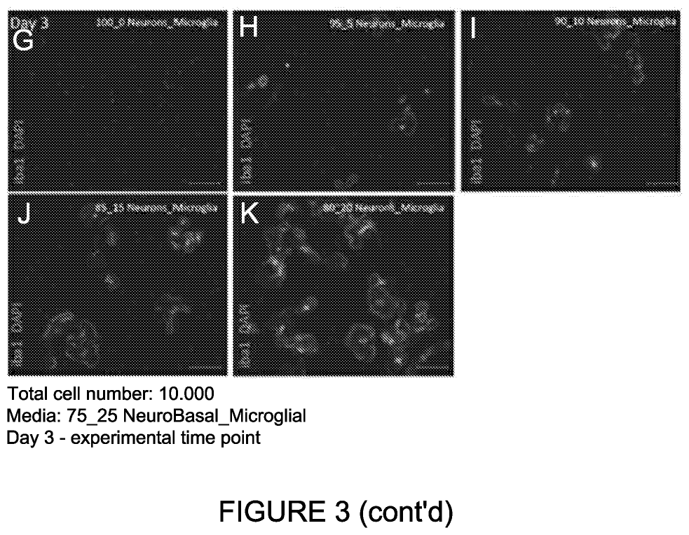

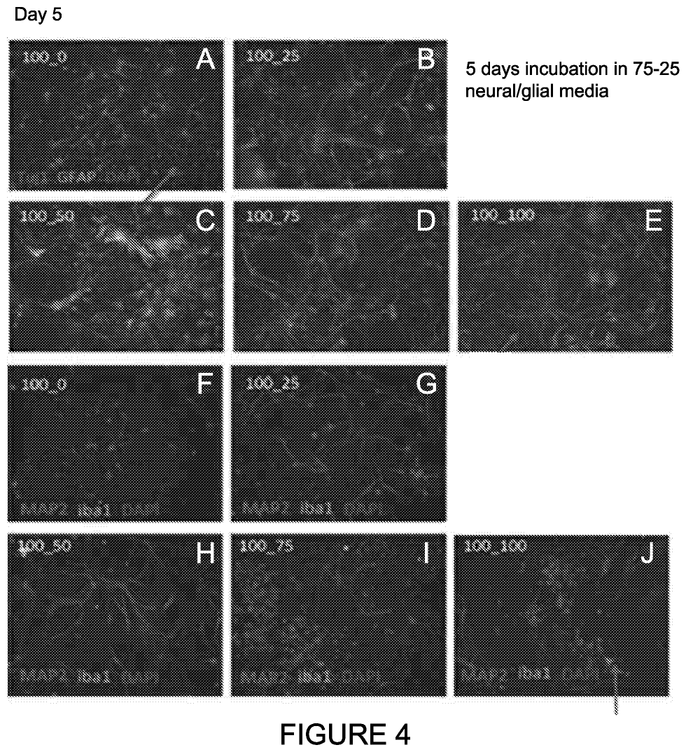

Ratio of neurons to glia was also evaluated. Initially, we aimed to mimic an in vivo brain cell ratio, but also explored ratios that were higher and lower than in vivo conditions. Neurons and microglia were co-cultured in 75% neuronal media and 25% glial at varied ratios of neurons to microglia for 3 days (FIG. 3, panels A-K) and neurons to astrocytes for 5 days (FIG. 4, panels A-J) and then fixed and immunostained for phenotypic markers of microglia (ibal) and neurons (tuj1, MAP2). Cell ratios used for the 3 day time point were 100:0 neurons to microglia (FIG. 3, panels A and G); 95:5 neurons to microglia (FIG. 3, panels B and H); 90:10 neurons to microglia (FIG. 3, panels C and I); 85:15 neurons to microglia (FIG. 3, panels D and J) and 80:20 neurons to microglia (FIG. 3, panels E and K).

2D co-cultures of neurons and astrocytes were established using different ratios of neurons and astrocytes (FIG. 4, panels A-J). Cells were fixed and immunostained at day 5 with specific markers: ibal (FIG. 4, panels F-J)—microglial; GFAP—astrocytic (FIG. 4, panels A-E); Tuj1—early neuronal (FIG. 4, panels A-E); MAP2—late neuronal markers (FIG. 4, panels F-J) and DAPI for nuclei counterstaining (FIG. 4, panels A-J). Culture media was 75:25 neural to glial media.

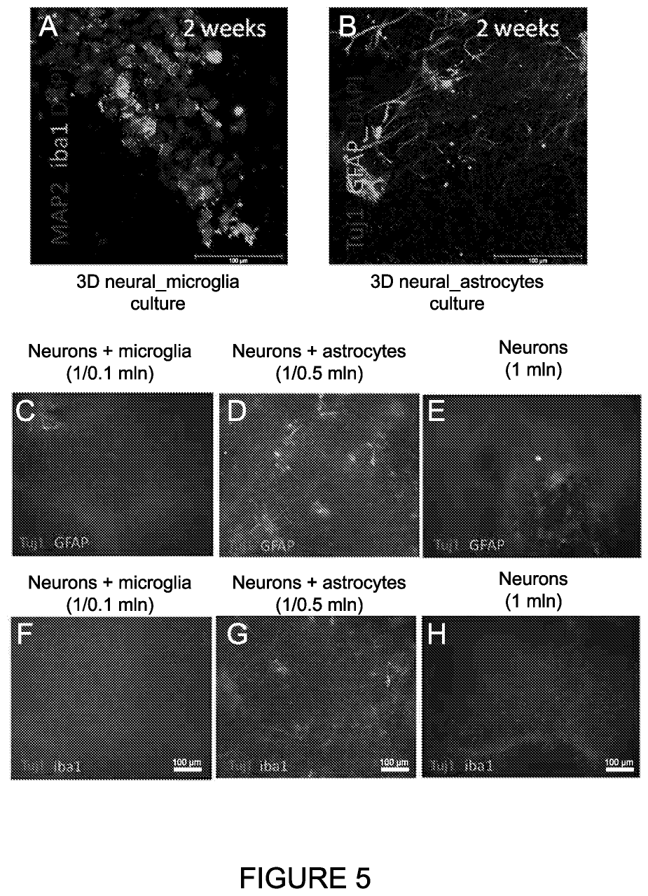

Next, 2D culture experiments were adapted to 3D co-cultures with media composition and cell ratio based on findings from the 2D experiments (FIG. 5, panels A-H). Cells were cultured and then fixed and immunostained after 1 week (FIG. 5, panels C-H) or 2 weeks (FIG. 5, panels A and B). Cells were stained with ibal (FIG. 5, panels A andC-H), GFAP (FIG. 5, panels B and C-E), tuj1 (FIG. 5, panels B-H), and MAP2 (FIG. 5, panel A). It was noted that at a ratio of 75% neuronal to 25% glial medium, there was an extremely high rate of glial cell proliferation and significant suppression of neuronal growth. As shown in FIG. 5, panels C-H, after 7 days in 100% neuronal media, which supported neuronal growth, glial cells grew but did not over-proliferate to the detriment of neurons.

### Example 3: Human Induced Pluripotent Stem Cells (hiPSCs)

As described above, silk scaffolds were prepared for use in growth of hIPSCs. Several different modifications and experiments were conducted to study growth of hIPSCs on silk scaffolds.

hIPSCs Differentiated to Neurospheres and Seeded in Fibroin Hydrogel

To begin, hiPSCs were differentiated to neurospheres and seeded in fibroin hydrogel prepared using an HRP/H₂O₂ crosslinking method as described in Partlow et al., Highly tunable elastomeric silk biomaterials, 2014, Adv Funct Mater, 24(29): 4615-4624. This process included seeding intact neurospheres into silk hydrogel (30 minute and 60 minute boil at 3% and 6% w/v). Cell survival was confirmed via Live/Dead staining. The 3% hydrogel did not completely gel, whereas the 6% gel did, however, the cells did not remodel the fibroin hydrogel, as evidenced by the lack of axonal penetration through the hydrogel and did not appear to show nearly the amount of growth as observed in parallel 2D cultures (data not shown).

Dissociated Neurospheres Seeded in Fibroin Hydrogel

It was hypothesized that neurospheres were inhibitory to neurite outgrowth due to close proximity of cells within the spheroids. To examine dissociated cell growth in fibroin hydrogel, neurospheres generated from hIPSC cells were dissociated with one of several reagents (e.g. trypsin, typ-LE, accutase, collagenase, dispase, etc.) and subsequently seeded into a fibroin hydrogel. Seeded cells survived, but no neuron-like extensions were observed.

Dissociated Neurospheres in a Fibroin Substituted Hydrogel

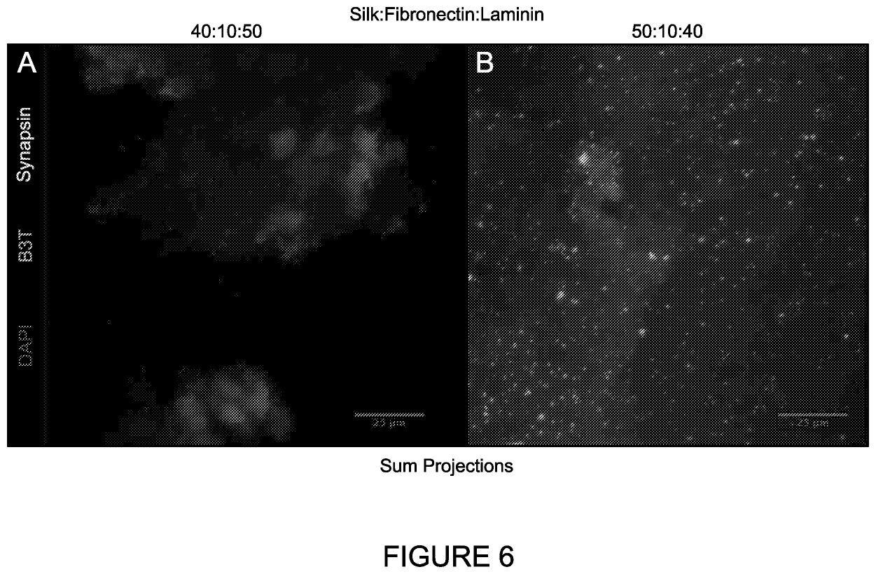

Here, hiPSCs were cultured and directed to form neurospheres, which were then dissociated. To mimic a more native environment for cells to grow and form a network, fibroin hydrogel was substituted with hyaluronic acid that had been substituted with tyramide at 1%, 3%, or 5% and mixed at a 1:1 ratio with the silk. Hyaluronic acid did not readily dissolve and increased neuron growth and/or neurite extension was not observed in this setup. In addition to hyaluronic acid, other native extracellular matrix components (e.g. fibronectin, laminin, collagen IV) were tested as additives to fibroin hydrogel. 2D tests showed neuronal differentiation and neurite extension and, as seen in FIG. 6, panels A and B, cells in the 3D models expressed the neuronal marker beta-III tubulin (red) and synapsin (green) in a combination hydrogel of silk:fibronectin:laminin at ratios of 40:10:50 (panel A) and 50:10:40 (panel B).

Dissociated Neurospheres in a Salt-Leached Fibroin Scaffold

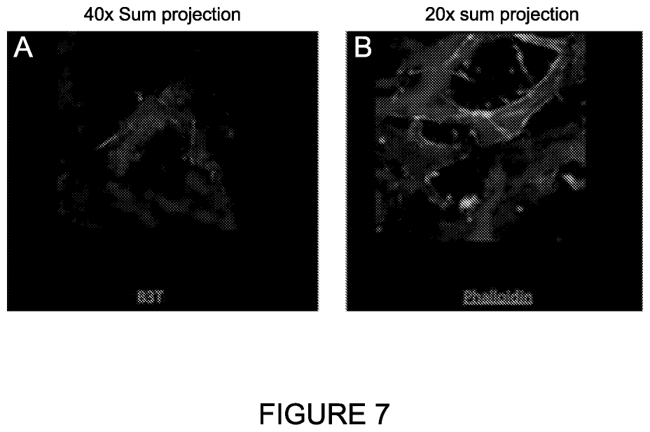

As described above, hiPSCs were cultured to form neurospheres, which were then dissociated. The growth of the dissociated neurospheres was evaluated in a salt-leached/porous silk fibroin scaffold filled with a collagen-I hydrogen that was added following cell seeding and attachment. As seen in FIG. 7, panels A andB, results showed the presence of beta-III tubulin-positive cells (panel A) and phalloidin-positive (panel B) staining confirmed cell attachment and growth along the scaffold/pore walls.

Undifferentiated hiPSCs Seeded into a Salt-Leached Fibroin Scaffold

To determine if adaptation of the hiPSCs to a non-adherent culture, such as in neurospheres, was hindering subsequent cell attachment within the scaffolds undifferentiated hiPSCs were seeded into the salt-leached scaffolds, culture time was extended and extent of neuronal differentiation was examined. In brief, undifferentiated human induced pluripotent stem cells (hiPSCs) were grown on a matrigel coated plate and passaged as cell clusters using ReLeSR (Stem Cell Technologies). A sample of this cell suspension was then treated with trypsin in order to obtain a single cell suspension, necessary to obtain a more reliable cell count. The remaining cell clusters were diluted (in mTeSR1) to a concentration of 5×10⁶ cells per mL based off of the trypsinized sample. This concentrated cell solution was then seeded into the prepared scaffold at 100 uL (5×10⁵ cells). The scaffolds were then given daily media changes and moved to fresh wells in order to remove any dead cells and limit the media usage by cells settled to the bottom of the plate. On the 5^{th} day of growth in the scaffolds the media was aspirated from the scaffolds and the scaffolds were filled with a collagen-I hydrogel. Once the hydrogel was able to solidify (approximately 30 minutes at 37° C.) the scaffolds were transferred to a 24 well plate and given 1 mL of final neuro media+B27, N2, ITS-A (insulin, transferrin, selenium-A) (Final neuro media: Neuro basal media+antibiotic/antimycotic+glutamax). This media was changed every 3 to 4 days. Neurite extension/network formation was visible in the central hole by 8 weeks.

Alternative Method: Neural Rosettes Selectively Seeded into Scaffold.

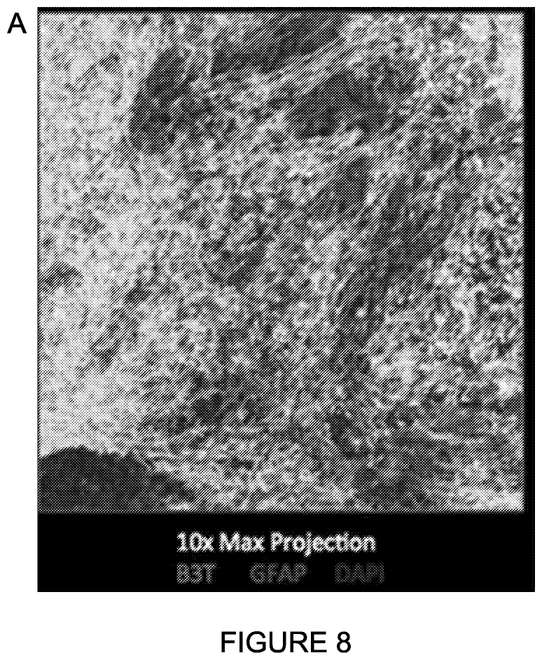

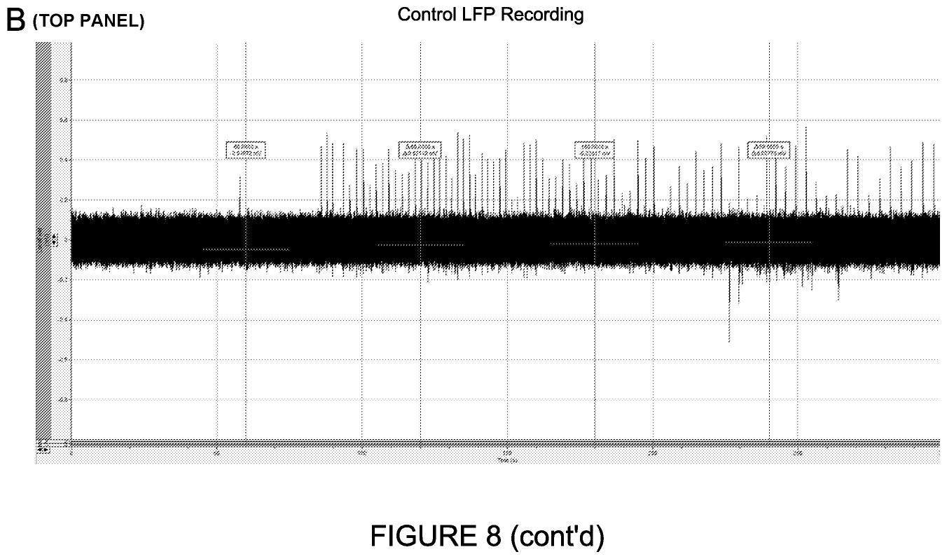

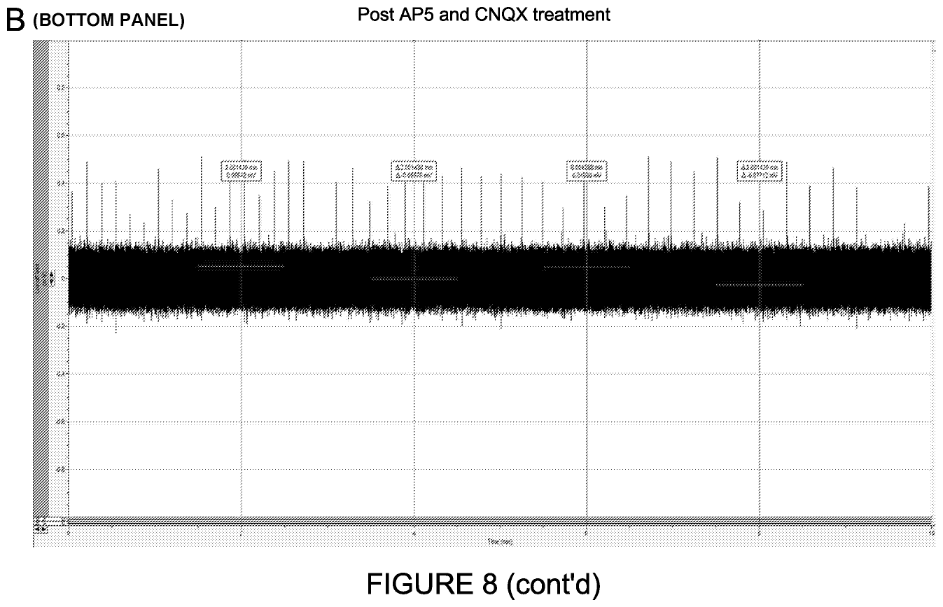

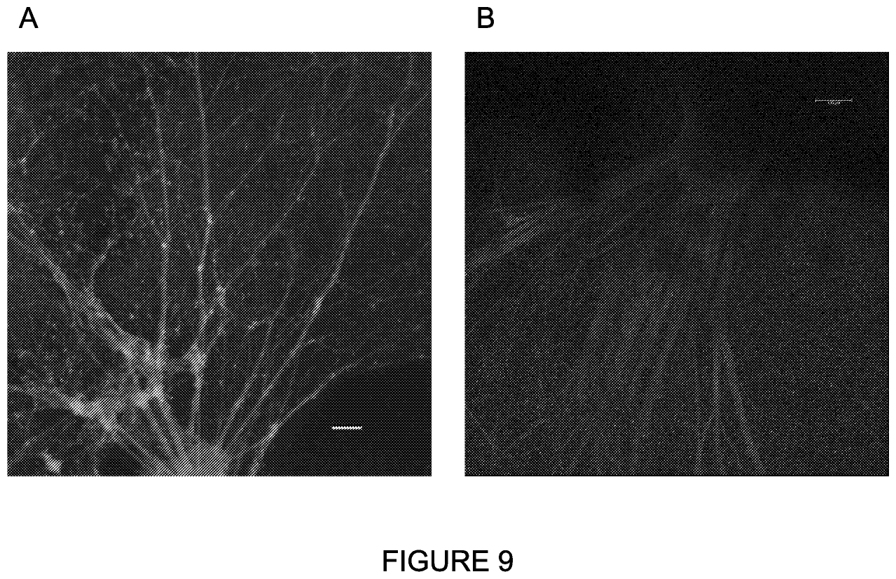

Undifferentiated hiPSCs were collected using gentle cell dissociation reagent (GCDR) and seeded at 3×10⁶ cells per well in an Aggrewell™ 800 plate in neural induction medium (full protocol described in Stem Cell Technologies StemDiff™ manual). This method produced uniform embryoid bodies (EBs) that were grown in the Aggrewell™ plates for 5 days. The EBs were then replated to a matrigel coated plate and over the next 6 days neural rosettes were observed forming. After day 11 (or day 6 post-plating for rosette formation) the rosettes can be selectively removed from the plate using StemDiff™ Neural Rosette Selection Reagent. These rosette clusters will then be treated as mentioned above and seeded similarly (5×10⁵ cells per scaffold for a period of 5 days prior to collagen-I hydrogel filling). Growth is observed over the subsequent weeks and analysis as previously described will be performed to identify cell populations present within the cultures as well as their relative neuronal maturity and network formation. As FIG. 8, panel A and FIG. 9, panel B show, a much more dense network of neurons and astrocytes was observed as compared to other types of scaffolds/gels described above in this Example, as well as electrical activity as measured by local field potential recordings (FIG. 8, panel B), and synapses, as shown by immunocytochemistry (FIG. 9, panel A).

### Example 4: Human Neural Stem Cells

Human ECM and Effects on Differentiation of hNSCs

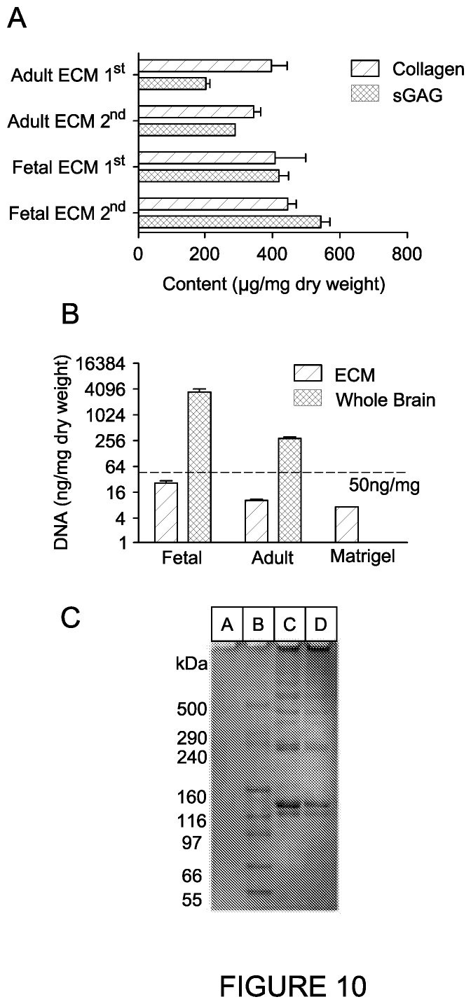

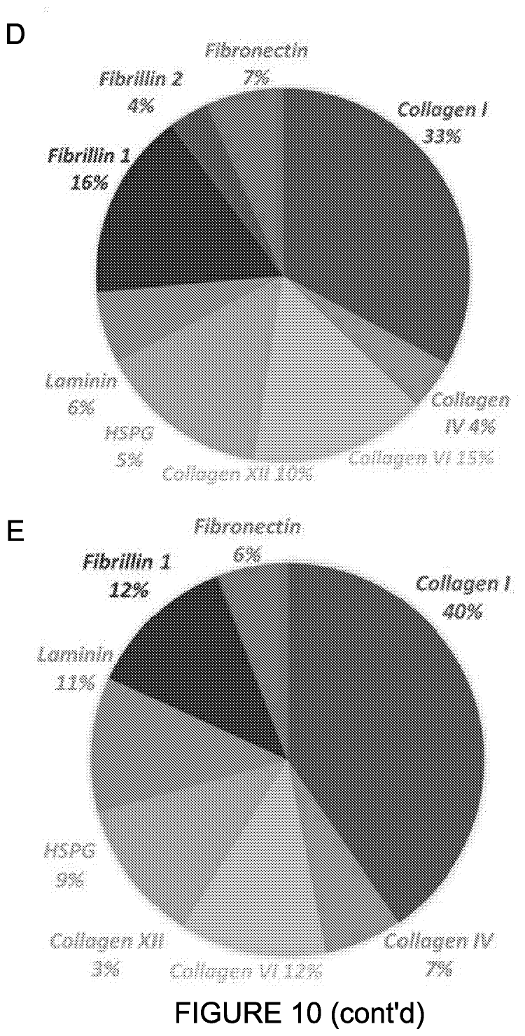

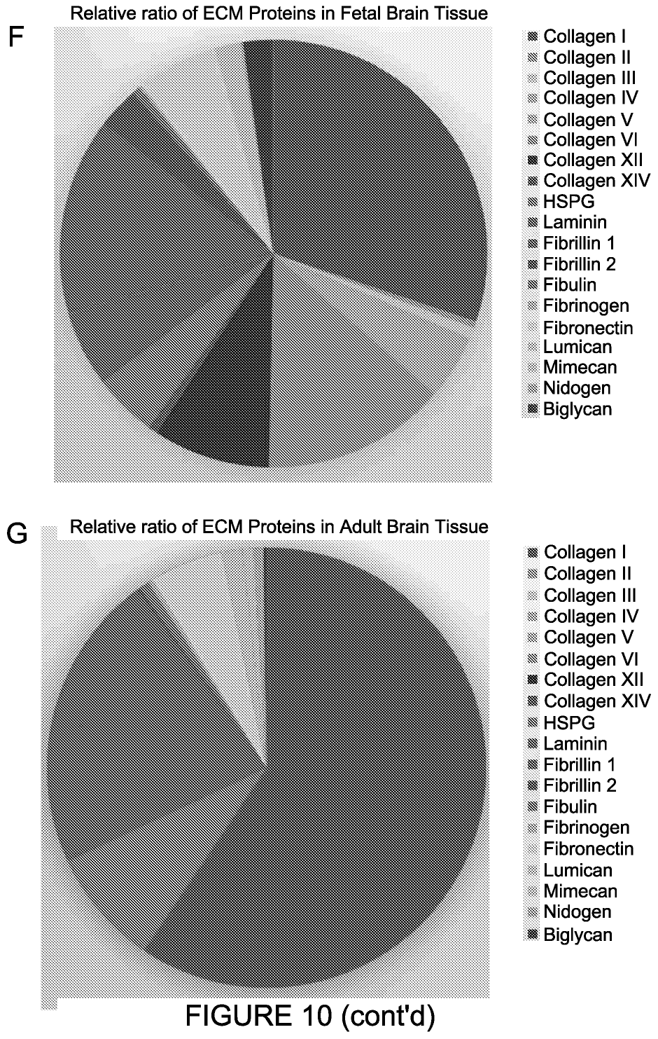

A comparison and characterization of fetal and adult-brain derived ECM was conducted using porcine fetal and adult brain ECM. As shown in FIG. 10, panels A-G characterization and comparison of decellularized fetal and adult-brain derived ECM shows sGAG and collagen content of the fetal and adult porcine brain ECM over multiple extractions, with a consistently higher ratio of collagen to sGAGs found in fetal ECM, while higher ratio of sGAG to collagen was found in adult ECM (FIG. 10, panel A). DNA content in the decellularized brain tissues versus adult and fetal whole brains was quantified by Pico green assay and seen to be on par with commercially available matrigel (FIG. 10, panel B). An exemplary protein gel shows bands in fetal and adult brain ECM (FIG. 10, panel C). Compositional differences were detected between fetal (FIG. 10, panel D) and adult (FIG. 10, panel E) porcine brain-derived ECM. Relative percentages of the major ECM proteins found in urea-digested brain ECM were quantified by LC/MS. An exemplary comprehensive list of the ECM proteins detected via LC/MS in decellularized fetal and adult brain ECM is shown in FIG. 10, panels F and G.

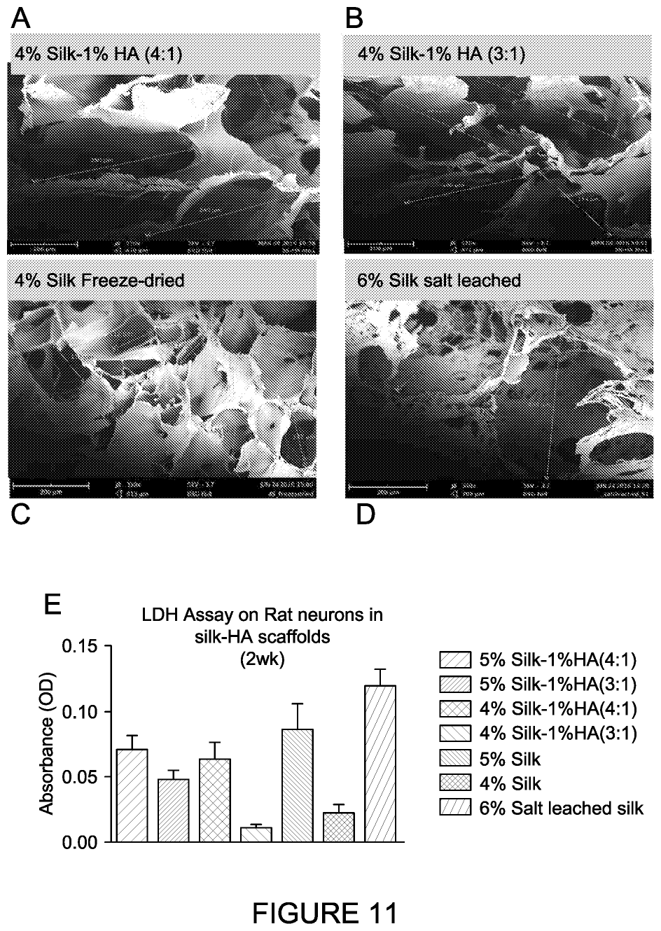

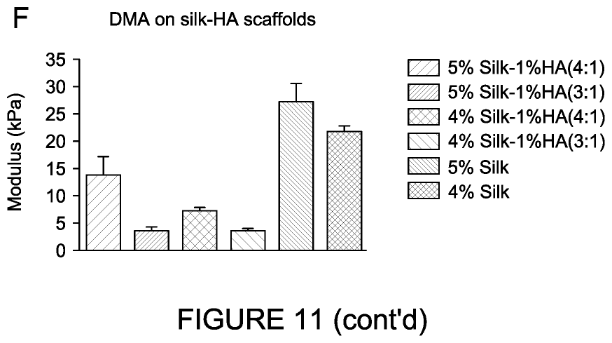

Characterization of silk-HA combination scaffolds was performed. As seen in FIG. 11, panels A-F SEM images show the pore size differences in Panel A) 4% silk—1% HA freeze-dried scaffold at a ratio of 4:1; Panel B) 4% silk—1% HA freeze-dried scaffold at a ratio of 3:1; Panel C) 4% silk freeze-dried scaffold; and Panel D) 6% silk salt-leached scaffold. An exemplary LDH assay (toxicity comparison) on 2 week primary rat neuronal cultures grown on freeze-dried silk alone and silk-HA scaffolds alongside the salt leached scaffolds show differences in substrates used (FIG. 11, panel E), while FIG. 11, panel F shows dynamic mechanical analysis (DMA) performed on silk-HA scaffolds to determine the modulus via unconfined compression testing.

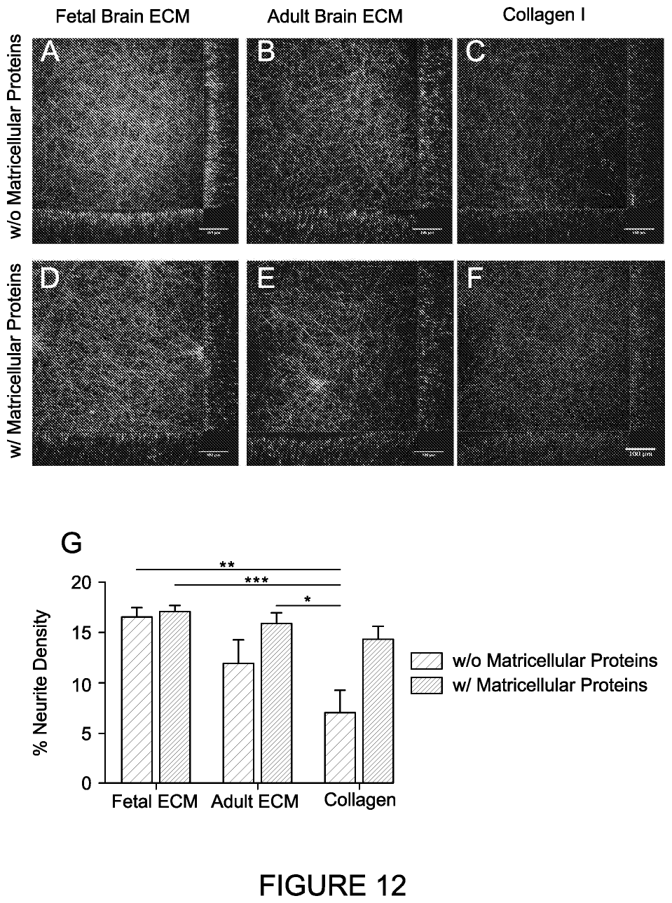

A comparison of primary rat cortical neurons (embryonic day 18) cultured in a 3D silk scaffold infused with collagen I gel (panels C and F) or collagen supplemented with 1000 ug/mL fetal brain ECM (FIG. 12, panel A and D) or 1000 ug/mL adult brain ECM (FIG. 12, panel B and E) either with (FIG. 12, panels D-F) or without (FIG. 12, panels A-C) astrocyte-released matricellular proteins (SPARC, Hevin, TSP2) was analyzed. FIG. 12, panel G shows average % neurite density per 2D plane of corresponding 3D Z-stack as quantified by using a custom image analysis code; ***p=0.001, **p<0.005, *p<0.007; n=5; collagen I as control

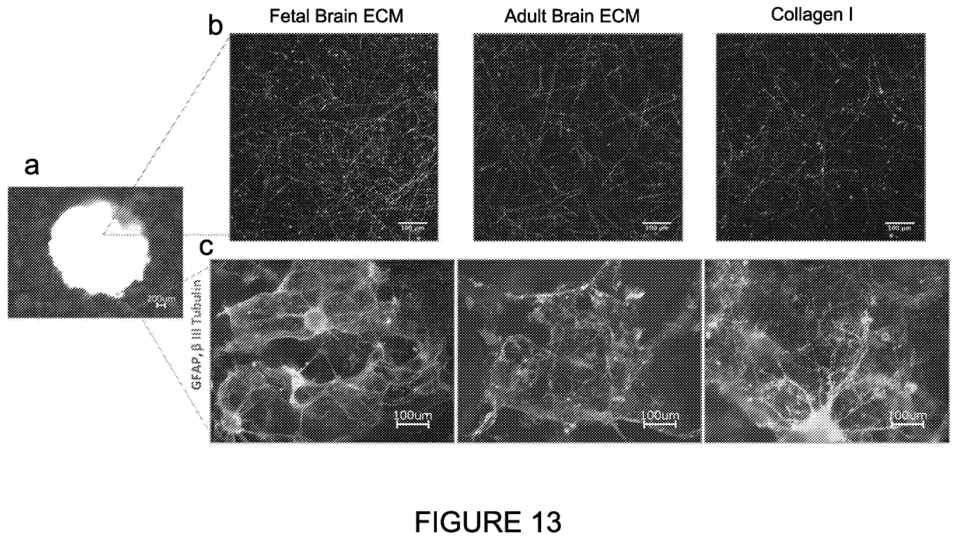

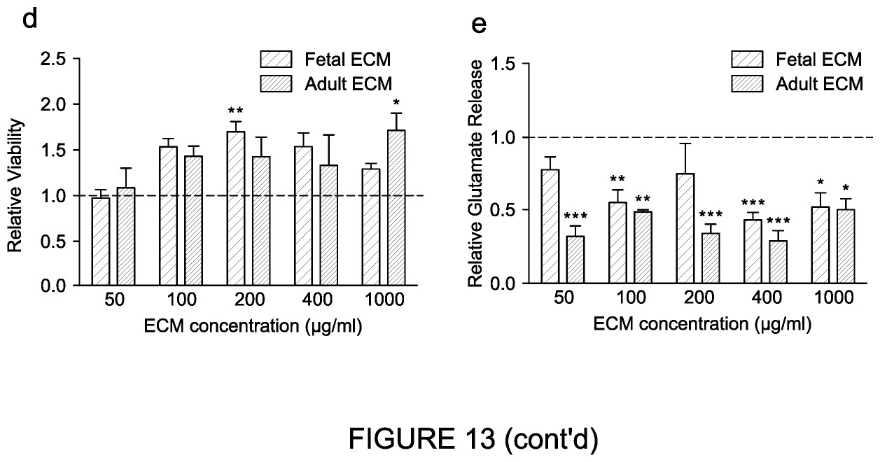

The effect of brain-derived fetal and adult ECM on rat cortical neurons was initially studied using an in vitro silk-collagen type I 3D bioengineered model of cortical brain tissue. 3D silk scaffolds were supplemented with either fetal or adult brain ECM, single component collagen I hydrogels, as well as Collagen I, collagen I+fetal brain ECM, HystemC and HyStemC+fetal brain ECM (see FIGS. 12 and 13). Fetal brain ECM induced superior neural network formation and spontaneous spiking activity over adult brain ECM or single component collagen I matrix. FIG. 13 shows growth of primary cortical rat neurons (embryonic day 18) (FIG. 13, panel a) in the middle window of the 3D silk scaffold (FIG. 13, panel b) infused with collagen I gel or collagen I supplemented with fetal or adult brain ECM at a concentration of 1000 μg/mL and neurites stained by β-III Tubulin at a 2 week time point and imaged within the donut-shaped portion (FIG. 13, panel c); Growth of primary cortical rat neurons in 3D silk scaffold infused with collagen I gel or collagen I supplemented with fetal or adult brain ECM at 1000 μg/mL of gel and culture media additionally supplemented with astrocyte-released matricellular proteins (SPARC, Hevin, TSP2) with neurites stained by β-III Tubulin at 2 week time point (as shown in FIG. 12 and as shown in FIG. 5 of Sood, D., Chwalek, K., DeBaun, E., Pouli, D Du, C., Tang-Schomer, M., & Kaplan, D. L. (2015). Fetal brain extracellular matrix boosts neuronal network formation in 3D bioengineered model of cortical brain tissue. ACS Biomaterials Science & Engineering, the disclosure of which is hereby incorporated in its entirety). FIG. 13, panel d shows relative viability of cells was measured in fetal or adult ECM at ECM concentrations of 50 μg/mL, 100 μg/mL, 200 μg/mL, 400 μg/mL and 1000 μg/mL and FIG. 13, panel e shows relative glutamate release was measured in fetal or adult ECM at ECM concentrations of 50 μg/mL, 100 μg/mL, 200 μg/mL, 400 μg/mL and 1000 μg/mL. The range of concentrations of fetal and adult brain ECM tested in the 3D model from 50 μg/ml to 1000 μg/ml. Solubilized fetal or adult brain ECM was mixed with 3 mg/mL Collagen I. In this entire range of ECM concentration, cell viability and glutamate release relative to collagen I-based model was determined for the following concentrations of ECM: 50, 100, 200, 400 and 1000 μg/mL. An overall increase in cell viability was observed for all concentrations of ECM, with the exception of 50 μg/mL fetal and adult ECM, which yielded similar level as collagen I alone. The ECM additive consistently lowered the level of glutamate release along the entire concentration range as compared to collagen I alone. (see FIG. 13, panel e) Fetal brain ECM is hypothesized to contain cues which promote neuronal differentiation of human neural stem cells (hNSCs). The effects of fetal and adult brain ECM on neural stem cells may differ, therefore a comparison of the two ECMs on neural stem cells was conducted. Fetal or adult ECM was derived from fetal or adult pigs, respectively. Effects of these two types of ECM on human neural stem cells (hNSCs) derived from skin fibroblasts were compared in a 3D donut-shaped silk scaffold model. The goal of this research was to investigate the composition of brain-derived ECM, to identify components relevant for neuronal differentiation and to study their effects towards the maturation of hNSCs. Delineating the basis for the divergence of cell responses on the two respective brain ECMs may have implications for brain regenerative medicine and for generating 3D in vitro disease models with more complete maturation of patient-derived neural stem cells.

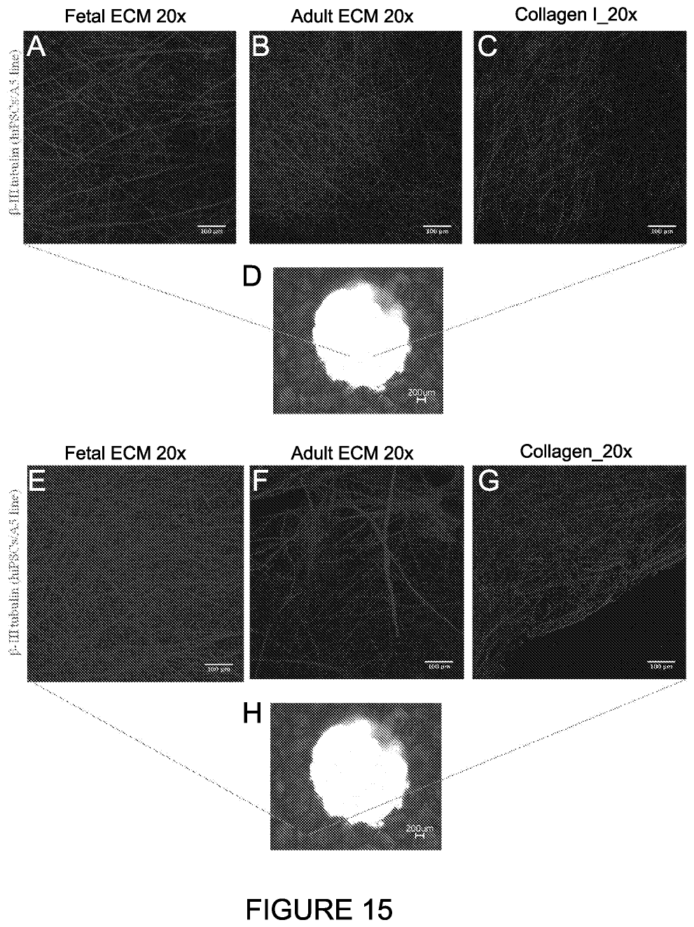

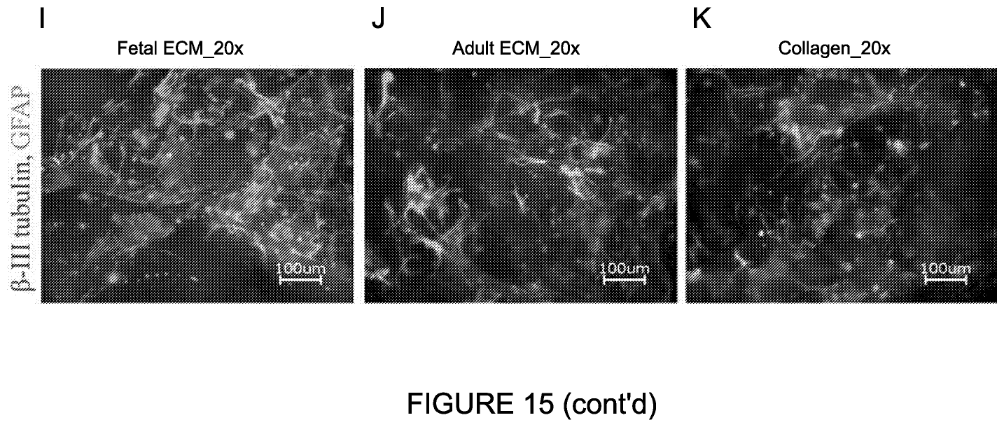

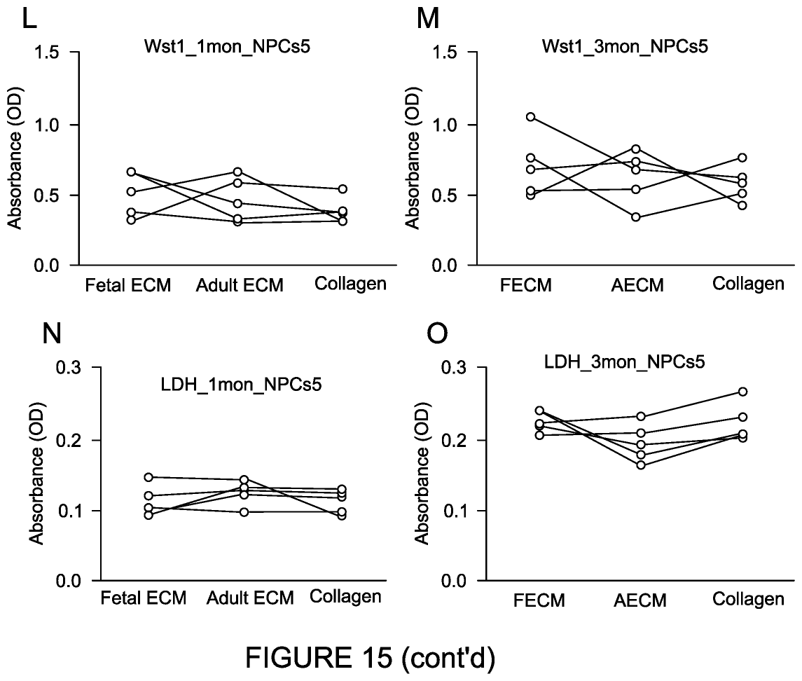

FIGS. 13 and 15 show that the higher neurite densities were observed in the fetal ECM condition and that the Collagen I+Brain ECM condition appeared to be best, overall, as evidenced by axonal ingrowth and functional assessments via calcium signaling and local field potential measurements (see Addendum).

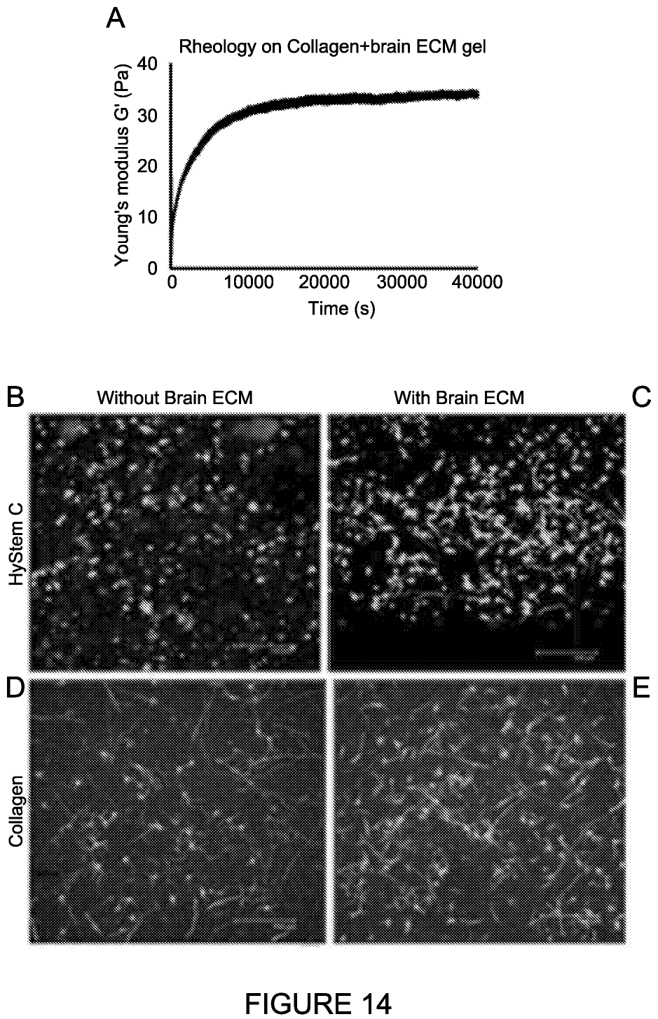

FIG. 14, panels A-E shows rheological characterization of collagen core hydrogel infused with brain ECM (Panel A); Panels B-E show primary embryonic rat neurons encapsulated in collagen I and Hystem C with (panels B and D) or without (panels C and E) brain ECM.

FIG. 15, panels A-O. Human neural stem cells were derived by direct reprogramming of skin fibroblasts in 3D silk scaffold supplemented with fetal (FIG. 15, panel A) and adult (FIG. 15, panel B) brain ECM or single component collagen I (FIG. 15, panel C) hydrogels, were imaged in the hydrogel window of the donut-shaped scaffold (FIG. 15, panel D) and were stained for neuronal maker, beta-III tubulin (red, FIG. 15, panels A-C). The highest axonal network density was observed in the fetal ECM condition (FIG. 15, panel B). The human neural stem cells in 3D silk scaffold were supplemented with fetal (FIG. 15, panel E) and adult (FIG. 15, panel F) brain ECM or single component collagen I (FIG. 15, panel G) hydrogels, imaged within the silk scaffold (panel H) and stained for neuronal maker, beta-III tubulin (red, FIG. 15, panels E-G). The highest axonal network density was observed in fetal ECM condition (FIG. 15, panel E). Differentiation of ReNCell neural stem cells in 3D silk scaffold supplemented with fetal (FIG. 15, panel I) and adult (FIG. 15, panel J) brain ECM or single component collagen I (FIG. 15, panel K) hydrogels were imaged at a 6 week time point within the scaffold (GFAP—astrocytes, green; β-III tubulin, red—neurons). Viability (FIG. 15, panels L-M) and toxicity (FIG. 15, panels N-O) assays were performed at 1 month (FIG. 15, panels L and N) and 3 month (FIG. 15, panels M and O) time points on skin fibroblast-derived human neural stem cell 3D cultures and showed no statistical difference in comparison to collagen I in viability or toxicity when the cells were grown in the presence of decellularized ECM; p>0.305. No increase in cell toxicity due to the presence of either fetal or adult brain-derived ECM was observed.

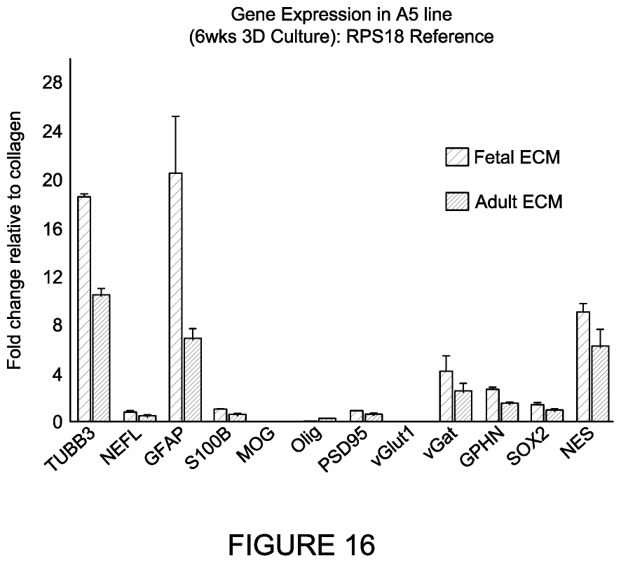

Differentiation of hNSCs was quantified after 6 weeks in 3D culture (FIG. 16) Cells were stained with beta-III tubulin and/or neurofilament light polypeptide (NEFL) to detect neurons, Glial fibrillary acidic protein (GFAP) and/or calcium binding protein B (S100B) to detect astrocytes, myelin oligodendrocyte glycoprotein (MOG) and/or oligodendrocyte transcription factor 1 (Olig1), postsynaptic density protein 95 (PSD95) and/or vesicular GABA transporter (vGlut1) to detect excitatory synapses, vGAT1 and/or Gephyrin (GPHN) to detect inhibitory synapses, and SOX2 and/or Nestin to mark stem cells. As is shown in this Example, show that the greatest amount of differentiation into neurons and astrocytes was observed in brain ECM conditions rather than collagen I conditions. Differentiation of ReNCell neural stem cells (from EMD Millipore) was also observed following 6 weeks of culture in a 3D silk scaffold supplemented with fetal brain ECM, adult brain ECM or collagen I hydrogels. Cells were stained with beta-III tubulin to detect neurons and GFAP to detect astrocytes.

### Example 5: In Vitro 3D Scaffold and Blood-Brain Barrier

### Materials and Methods

Fabrication of Tube and Salt-Leached Sponge Bioreactor

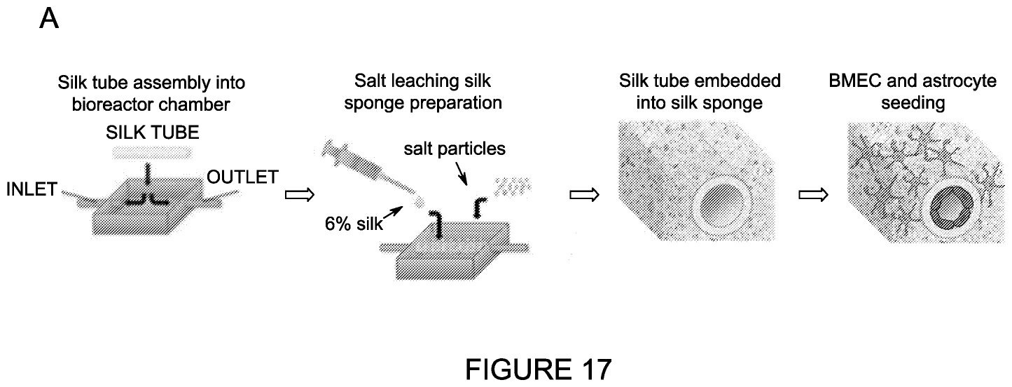

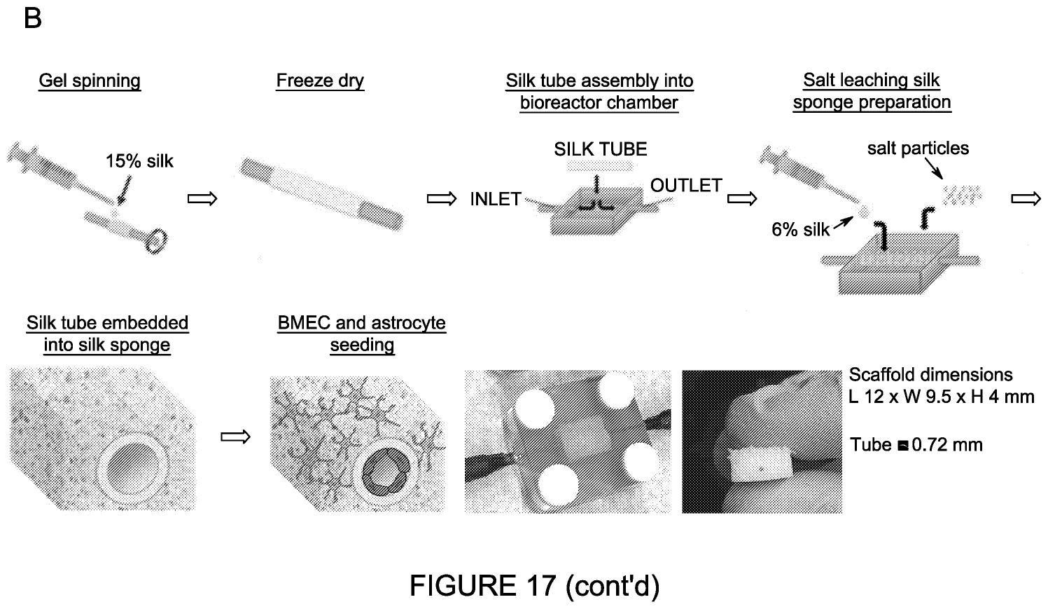

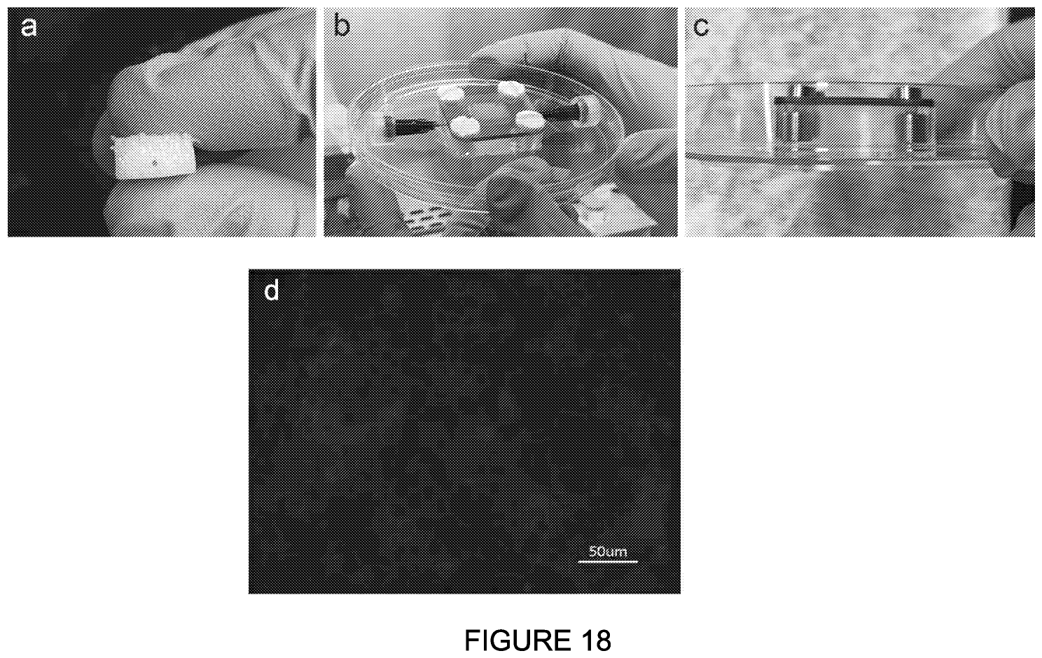

In this Example, a scaffold was created to model the blood-brain barrier, and was fabricated using a silk tube and salt-leached sponge (FIG. 17, panels A-B). Initially, a silk fibroin solution was prepared and concentrated to 18-20%. To support human umbilical vein endothelial cell (HUVEC) seeding, 50 μg/mL fibronectin was added to the silk solution. To support BMEC seeding, 40 μg/mL collagen IV and 10 μg/mL fibronectin was added to the silk solution. 22G needle was dip-coated in the Silk/ECM solution (for HUVEC, 50 μg/ml fibronectin; for BMEC, 40 μg/ml collagen IV and 10 μg/ml fibronectin), and this coating was immersed in 100% methanol to induce β-sheet formation, forming a silk tube. Two 22G blunt needles were inserted in a customized PDMS chamber and connected to the silk tube. To form a silk sponge around the tube, a 6% silk solution was poured into the chamber, salt particles with diameter 500-600 μm were added to the silk solution at a 1 mL-to-2 g ratio, and the mixture was cured at room temperature for 2 days (FIGS. 17 and 18, panel a). The salt particles were leached in water with several water exchanges. The bioreactor was closed with a lid secured by screws (FIG. 18, panel b) or magnets (FIG. 18, panel c).

Fabrication of Lyophilized Sponge Bioreactor

A Teflon-coated wire was placed in a 5% silk solution, and the silk was lyophilized to create a sponge with 20-90 μm pores. ECM solution (for HUVEC 1 mg/ml collagen I and 50 μg/ml fibronectin; for BMEC, 50 μg/ml collagen I, 400 μg/ml collagen IV, and 100 μg/ml fibronectin) was mixed with human astrocytes, hPSC-derived neural progenitor cells, hPSC-derived neurons, and/or hPSC-derived astrocytes, added to the sponge, and allowed to gel. The wire was removed to create a channel, and an endothelial cell (EC) suspension was injected into the channel. The channel was then connected to needles housed in a PDMS chamber to form a bioreactor unit. The bioreactor was connected to a peristaltic pump to induce physiological shear force on the ECs.

BMEC Culture on ECM Gel

The following gels were made in 24 well plates: 1) Matrigel, 2) collagen I (1 mg/mL) gel, and 3) collagen I (1 mg/mL)+collagen IV (400 μg/mL)+fibronectin (100 μg/mL) gel. Freshly thawed BMECs were cultured on gel in the wellplates at 1×10⁶ cells/cm² in complete media on the first day, followed by bFGF and RA withdrawal the next day. BMECs were fixed on day 2 to examine morphology and ZO-1 expression. An exemplary micrograph shows that BMECs express the tight junction protein ZO-1 when cultured on an ECM gel comprising collagen I (1 mg/mL), collagen IV (400 μg/mL), and fibronectin (100 μg/mL) (FIG. 18, panel d).

BMEC Culture in 3D Tube

Silk tubes were coated with 400 μg/mL collagen IV and 100 μg/mL fibronectin overnight in a 37° C. incubator. Frozen BMECs were thawed and labeled with Cell Tracker Green in suspension. BMECs were washed and resuspended at 10×10⁶ cell/mL in complete EC media (hESFM media supplemented with 1% platelet poor plasma derived serum, 20 ng/mL bFGF, 1 μM retinoic acid, and 1 μM Y-27632), 200 μL of cell solution was manually injected into the tube. The sponge was filled with complete EC media. The bioreactors were closed with needle caps and a lid and were placed on a roller for 0.5 rpm for 2 hrs or upside-down for 2 hrs. The bioreactors were returned to an upright position and the lid and caps removed. The bioreactors were placed in a 37° C. incubator overnight and the next day, media was exchanged to media without basic fibroblast growth factor (bFGF) and retinoic acid (RA). Scaffolds were fixed with 4% paraformaldehyde and cut open with a blade for imaging analysis.

HUVEC Culture in 3D Tube

Silk tubes were coated with 100 μg/mL fibronectin overnight. GFP-HUVECs were trypsinized and resuspended in EGM-2 (5×10⁶ cell/mL) and 200 μL of cell solution were injected into the tube. The bioreactors were closed with caps and a lid and placed on a roller at 0.5 rpm for 2 hours. Another 200 μL of GFP-HUVEC suspension was injected again, and the bioreactors were kept upside-down for 2 hours. The bioreactors were then returned to an upright position and the lid and caps were removed—The bioreactors were placed in a 37° C. incubator overnight, and media was refreshed the next day. The samples were cultured in a static condition for 2 days, then the scaffolds were fixed with 4% paraformaldehyde and cut open with a blade for imaging analysis.

### Results

Design of 3D In Vitro Blood-Brain Barrier (BBB) Bioreactor

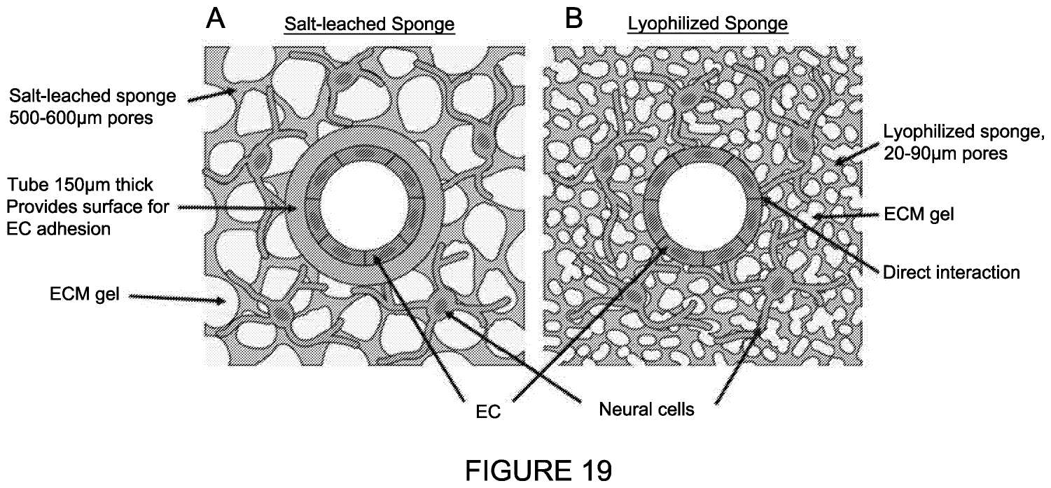

In this example, a 3D in vitro BBB culture scaffold is created that consists of a silk sponge with a hollow channel inside the sponge. Endothelial cells (BMECs) are seeded in the channel to mimic blood vessels. Neural cells, including astrocytes, neurons, and neural progenitor cells (NPCs), are seeded in the sponge to mimic brain tissues. The sponges are connected to a bioreactor and perfused to induce physiologically relevant shear stress on the ECs. Two types of sponges are tested: 1) a salt-leached sponge with a tube and 2) a lyophilized sponge (FIG. 19, panels A-B). The salt-leached sponge (FIG. 19, panel A) design was adapted from a 3D brain model (Tang-Schomer et al., 2014) and a bone marrow model described in Di Buduo et al (Di Buduo et al., 2015). A silk tube (diameter=0.7 mm, thickness≈150 μm) was embedded in a salt-leached sponge (pore size=500-600 μm). The tube provides a smooth surface for EC adhesion and monolayer formation. The lyophilized sponge (FIG. 19, panel B) was created by lyophilizing silk solution (pore size=20-90 μm). A wire was placed in the silk solution during lyophilization and then removed to create a hollow channel. The pores were filled with ECM gel to create a smooth surface for EC adhesion and monolayer formation.

GFP-HUVECs Formed a Monolayer in the Tube in the Salt-Leached Sponge

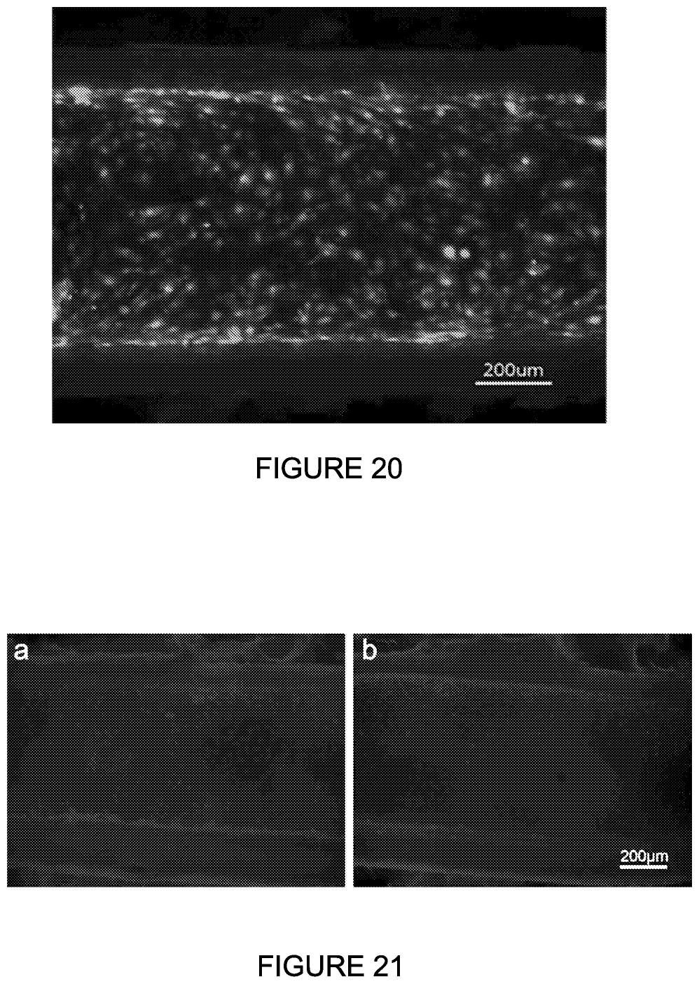

To demonstrate formation of a monolayer of cells using endothelial cells in the luminal wall of the tube embedded in a salt-leached sponge, GFP-HUVECs were seeded into the tube. The seeding protocol was as described above with certain optimization steps. Silk tubes were coated with 100 μg/mL fibronectin overnight. GFP-HUVECs were trypsinized and resuspended in EGM-2 (5×10⁶ cell/mL) and 200 μL of cell solution were injected into the tube. The bioreactors were closed with caps and a lid and placed on a roller at 0.5 rpm for 2 hours. Another 200 μL of GFP-HUVEC suspension was injected again, and the bioreactors were kept upside-down for 2 hours. The bioreactors were then returned to an upright position and the lid and caps were removed. The bioreactors were placed in a 37° C. incubator overnight and media was refreshed the next day. After 2 days of culture, fluorescence microscopy showed that the GFP-HUVECs had formed a 3D monolayer on the luminal wall of the tube (FIG. 20).

BMECs Adhered to the Tube

To create an in vitro model of a blood vessel with endothelial cells of brain origin, human iPSC-derived BMECs (Lippmann et al., 2012; Stebbins et al., 2015) were seeded in the luminal wall of the tube. BMECs (10×10⁶ cell/mL) were injected into the tube. The bioreactors were either 1) placed on a roller at 0.5 rpm for 2 hrs or 2) placed upside-down for 2 hrs, and then returned to an upright position to be cultured for 2 days. Scaffolds were fixed with 4% paraformaldehyde and cut open with a blade for imaging analysis. Hoechst staining of cell nuclei showed that the BMECs adhered to the tube (FIG. 21, panels a-b).

BMECs Morphology on ECM Gel

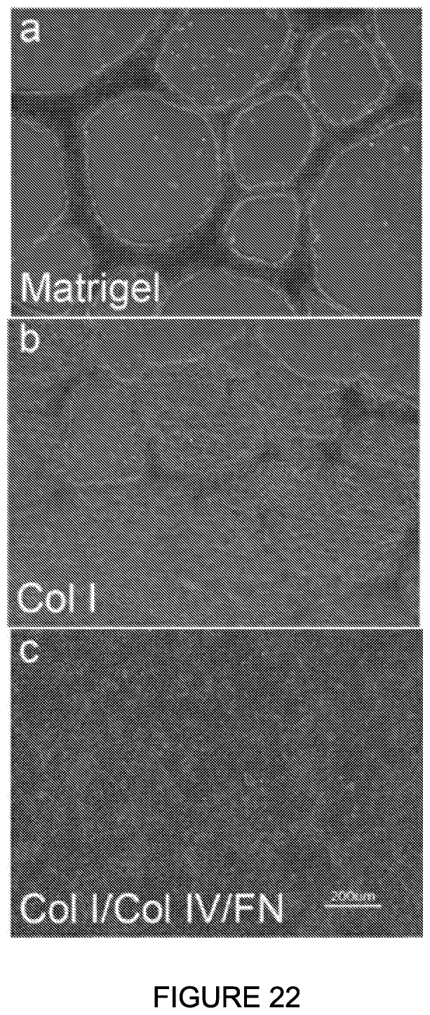

In the lyophilized sponge (FIG. 19, panels B, described above), ECs are in contact with ECM gels. ECM gels have an effect on EC adhesion and morphology, so in order to determine the composition of ECM gel that best supports BMEC monolayer formation, BMEC morphology was examined on 1) Matrigel, 2) collagen I gel, and 3) collagen I+collagen IV+fibronectin (100 μg/mL) gel. As shown in FIG. 22, panels a-c, BMECs formed a web-like morphology on Matrigel (panel a), patchy monolayer on collagen I gel (panel b), and monolayer on collage I+collagen IV+fibronectin gel (panel c).

### Example 6: Functionalization

### Materials and Methods

In this example, silk scaffolds were prepared via salt leaching with a pore size of less than 700 μm, and cut to final dimensions of 6 mm diameter by 2 mm height. Scaffolds were allowed to equilibrate in neurobasal media overnight before seeding with rat primary cortical neurons (embryonic day 18, 1 million cells/scaffold). Cells were seeded and the seeded scaffold was incubated overnight to allow cells to adhere prior to embedding sponges with, e.g. collagen I, fibroin, and/or methylprednisolone, and encapsulation.

The following day, fibrin hydrogel solutions were prepared from salmon fibrinogen (3 mg/ml) and thrombin (1.5 units/ml), and formulated with the water-soluble corticosteroid methylprednisolone (MP) hemisuccinate sodium salt (MW 496.53 g/mole), to final concentrations of 1.5, 3, 6, and 8.7 mM MP. 100 ul of Fibrinogen/Thrombin/MP solution was added to each scaffold, and scaffolds were subsequently incubated at 37 degrees Celsius for 30 minutes to promote cross-linking. Upon successful gelation, constructs were transferred to 24 well plates containing 1 mL of neurobasal media per well and incubated at 37 degrees Celsius for release studies.

At specified time points up to 14 days, media was sampled and frozen for MP quantification by HPLC, as well as cytotoxicity analysis by LDH assay. A media volume range of 0.8-1.0 mL was maintained throughout the course of the study. At the conclusion of the study constructs were stained with calcein/ethidium homodimer to visualize live and dead cells. MP was quantified via Agilent 1260 HPLC using an Agilent Zorbax Eclipse Plus C18 reverse phase column (4.6×75 mm, 3.5 um); running a gradient of 95% water+0.1% TFA/5% Acetonitrile→5% water+0.1% TFA/95% ACN over 4 minutes, followed by a 2 minute hold and subsequent re-equilibration. A standard curve of MP spiked in neurobasal media was produced over a concentration range of 500-0.39 ug/ml, with an R-squared value of 0.999.

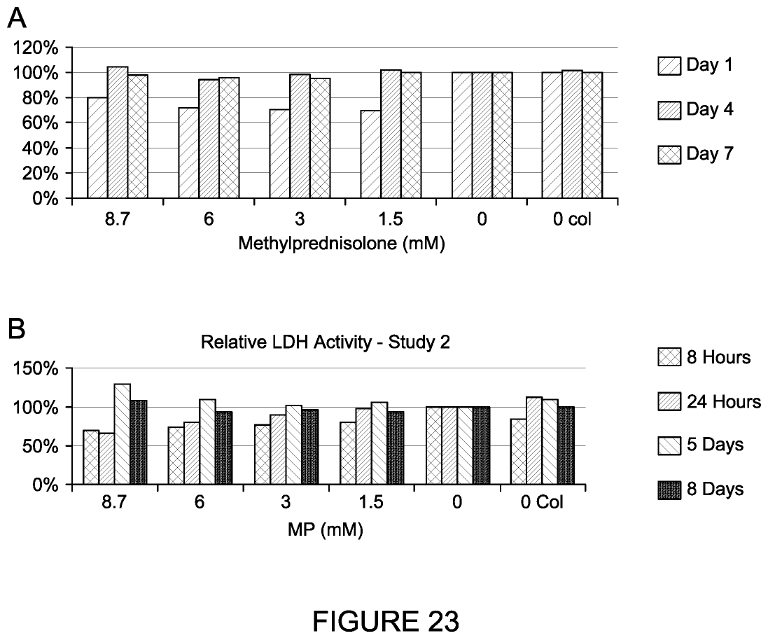

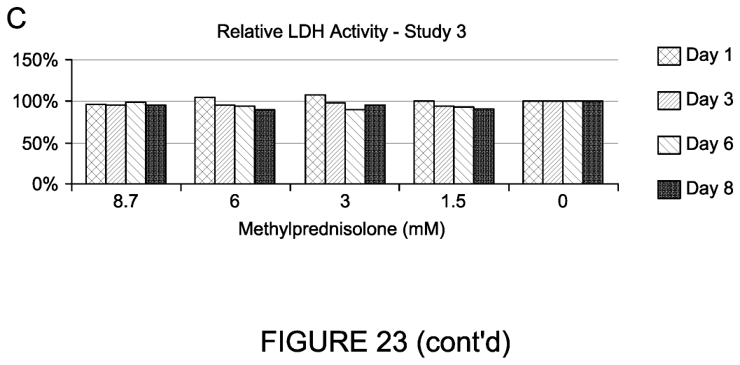

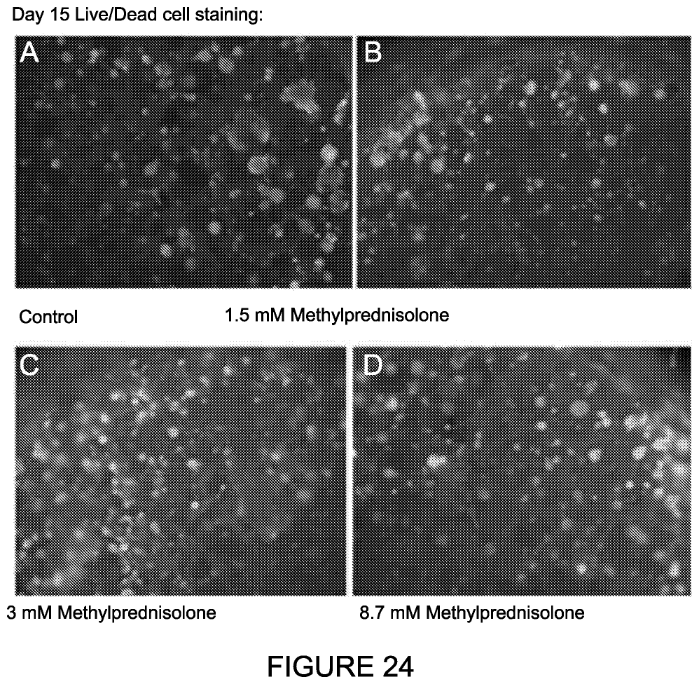

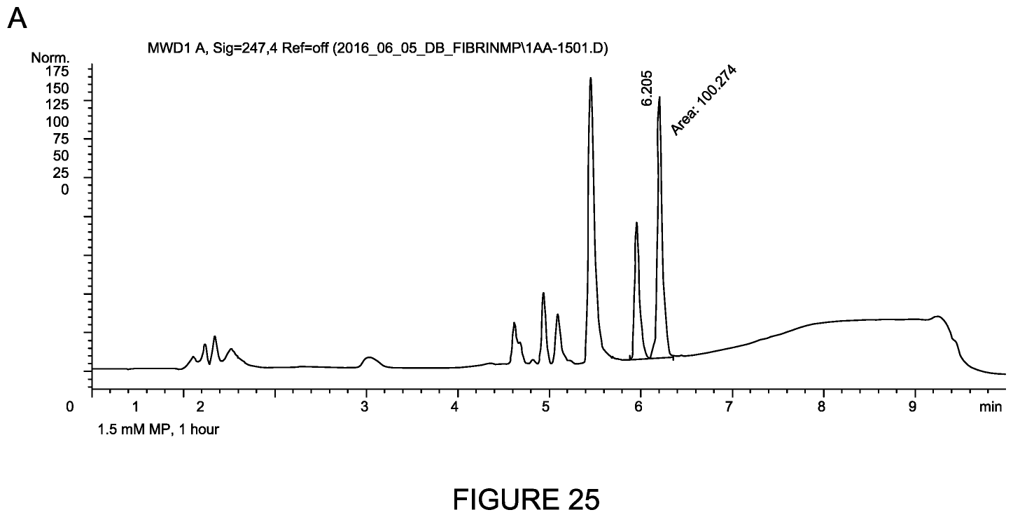

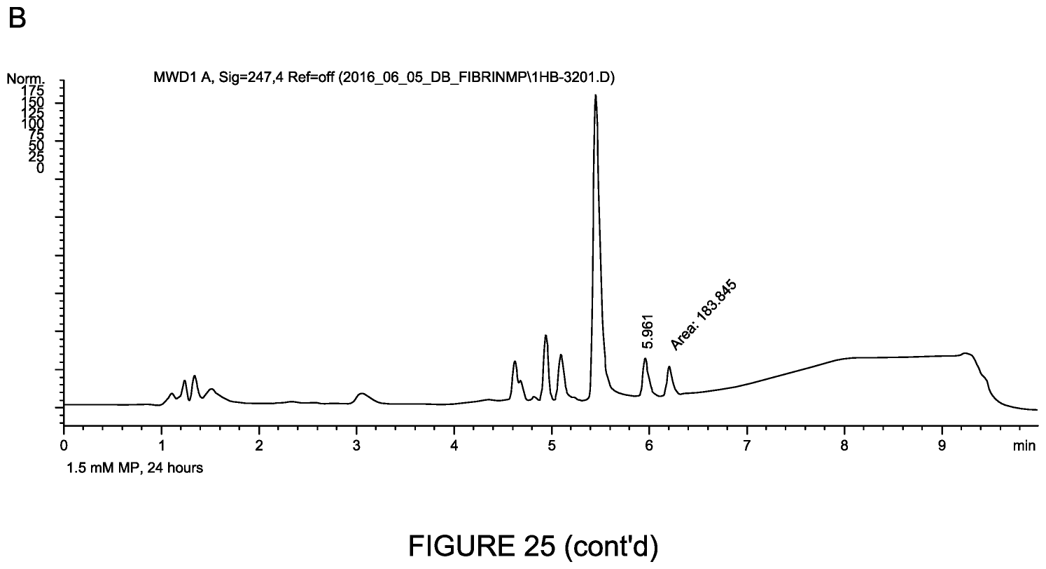

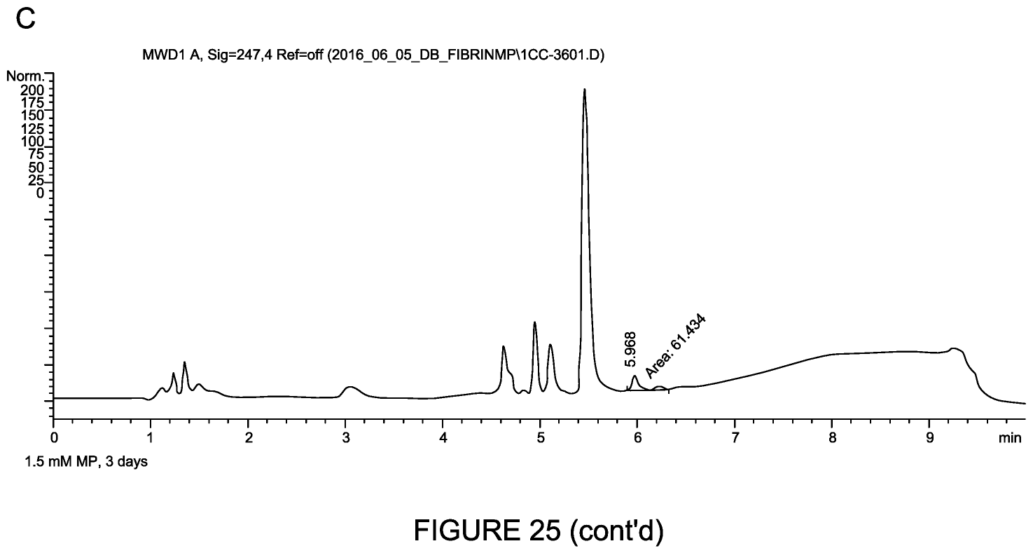

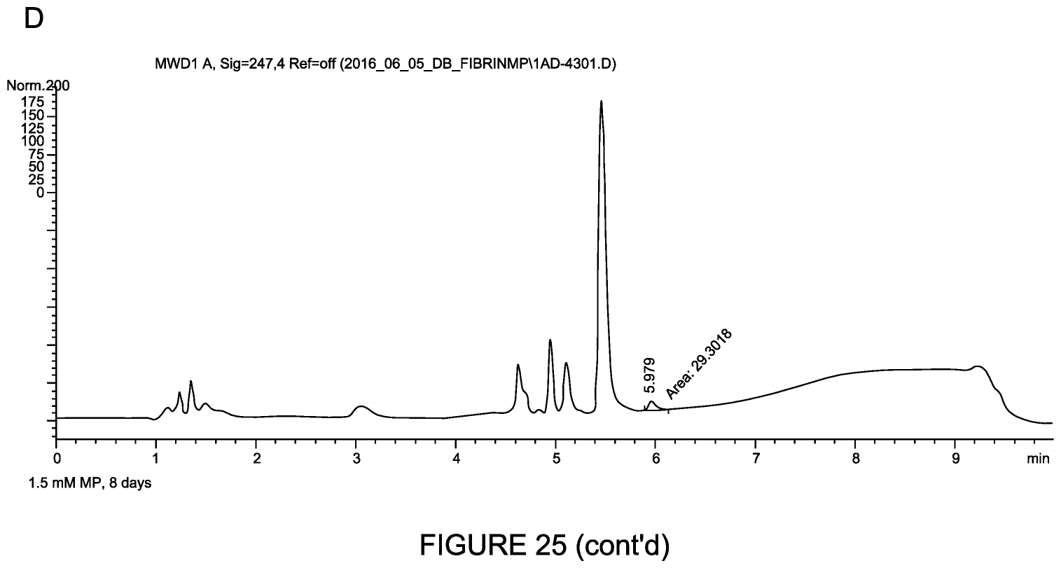

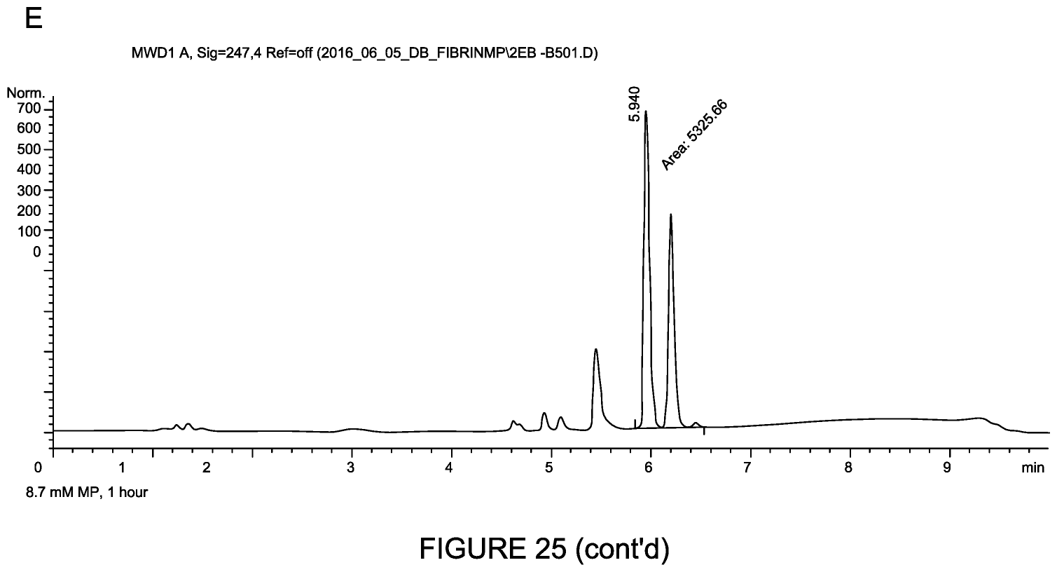

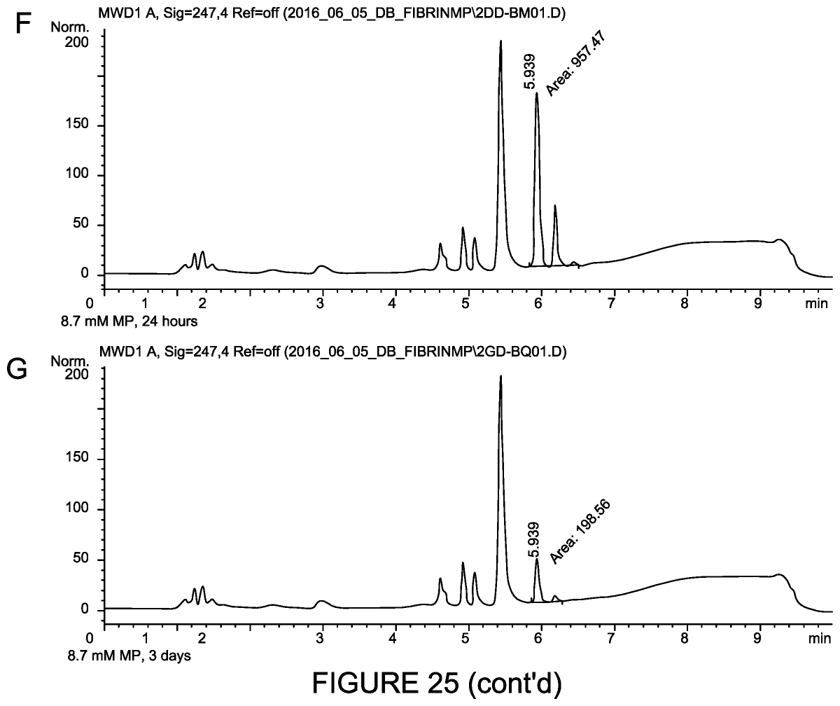

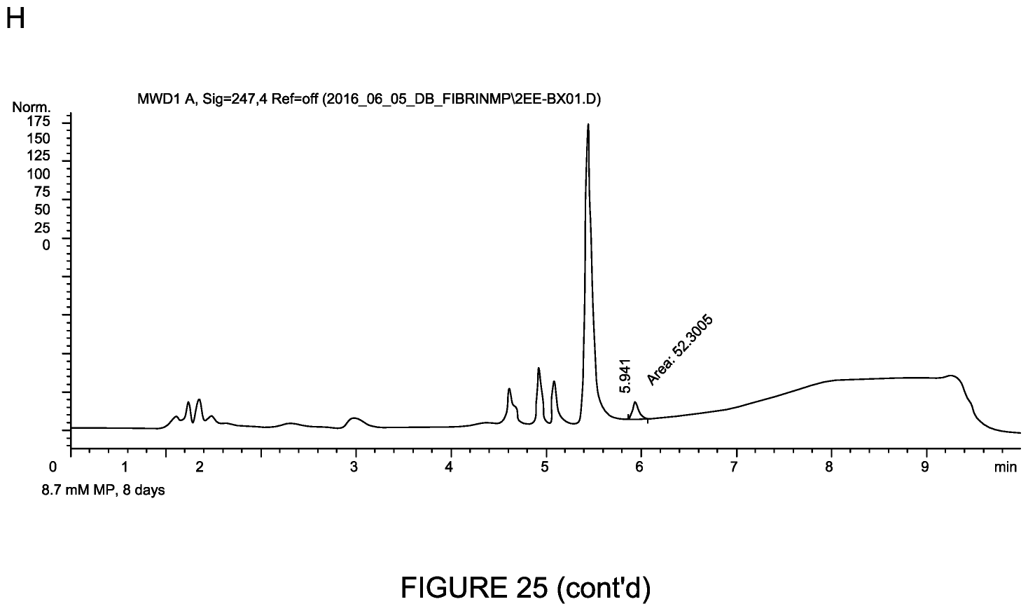

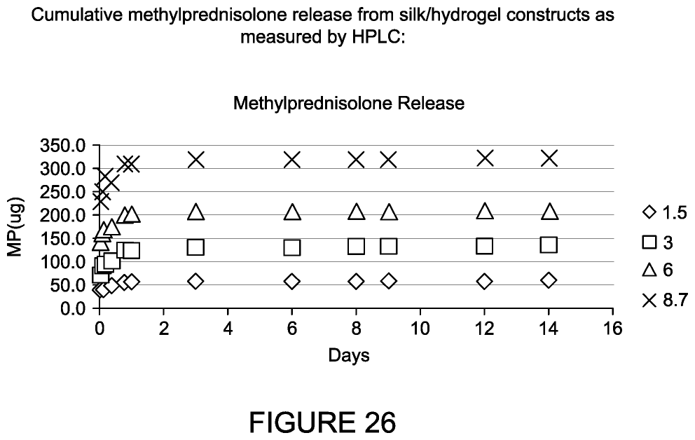

Results of the LDH assay indicate that cell survival was not adversely impacted by the concentrations of methylprednisolone loaded into the fibrin hydrogels (see FIG. 23, panels A-C) and representative Live/Dead cell staining in FIG. 24, panels A-D. Representative HPLC chromatograms (FIG. 25, panels A-H) are summarized in FIG. 26, which shows cumulative methylprednisolone release from silk/hydrogel constructs (as measured by HPLC) measured in micrograms by HPLC at doses of 1.5, 3, 6, and 8.7 mM of methylprednisolone over a course of up to 16 days. At all loading concentrations, the results suggest that MP appears to undergo a majority burst release within the first 24 hours, followed by low-level, microgram quantity release over the course of 1-1.5 weeks.

### Example 7: Instrumentation

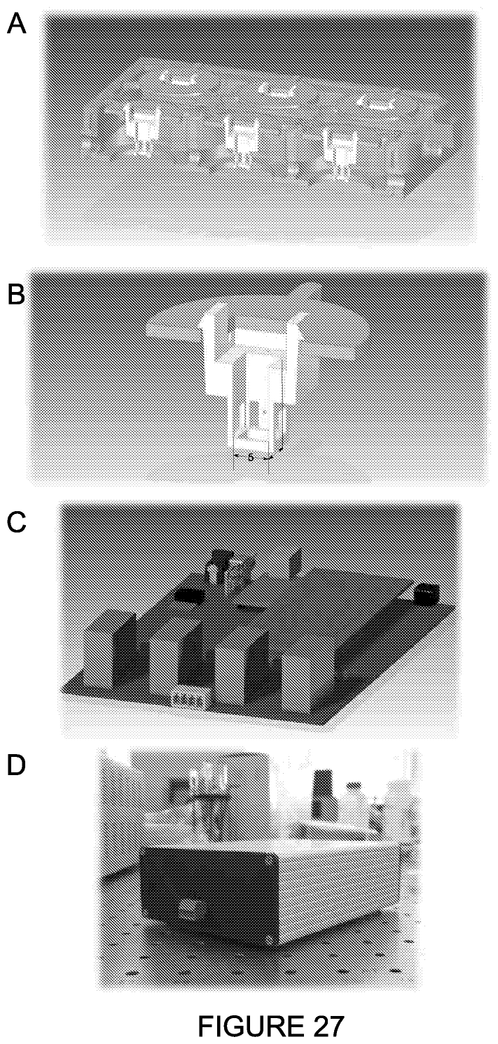

In some embodiments, instrumentation (e.g., with one or more electronic, optic, and/or optogenetics device, for example) of some compositions may be achieved by making use of a bioreactor interface that is capable of 3D electrical stimulation, coupled with media perfusion. By way of non-limiting example, schematics certain formats comprising provided compositions are shown in FIG. 27, panels A-D. In some embodiments, provided compositions comprising one or more electronic, optic, and/or optogenetics devices may (1) mimic endogenous neuro-oscillations of alpha, beta and/or gamma waves and/or (2) incorporate clinically relevant parameters of deep brain stimulation. Furthermore, in some embodiments, provided compositions may be coupled with, inter alia, a frequency generator to (1) construct unique wave-forms and/or (2) modify charge density on tissue engineered constructs. As shown in FIG. 27, an exemplary method of instrumentizing can be achieved using a well plate bioreactor (FIG. 27, panel A) within which electrostimulation inserts (white) may be placed containing tissue constructs (FIG. 27, panel B). This bioreactor may then be connected with an electrostimulation device (FIG. 27, panel C) and/or to a frequency generator (FIG. 27, panel D)

### Example 8: Disease Models

Traumatic Brain Injury

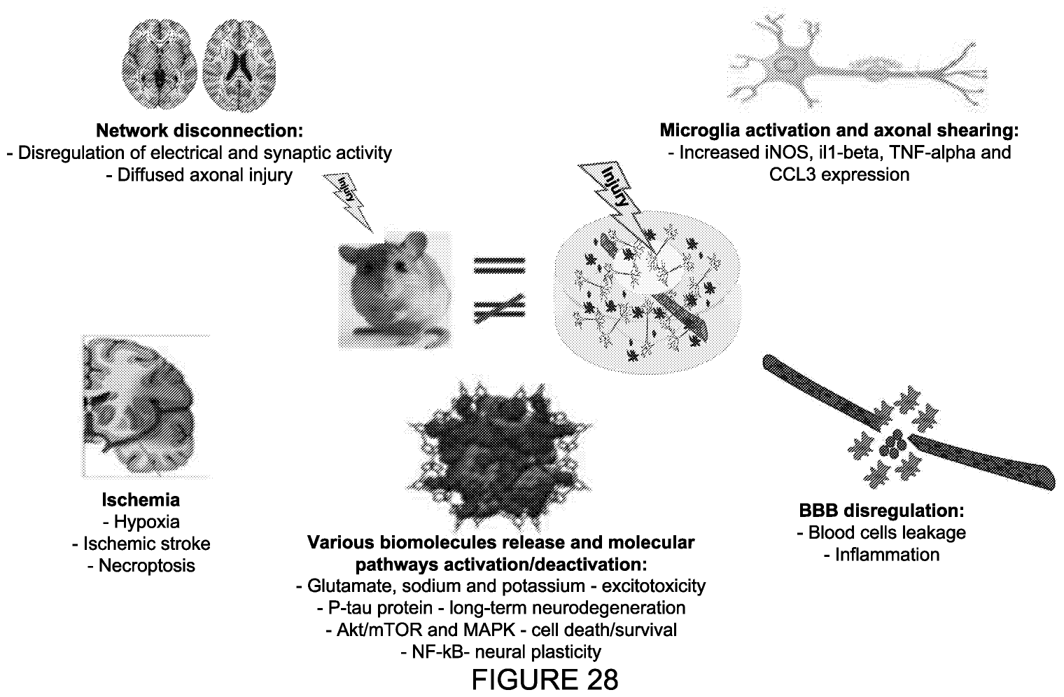

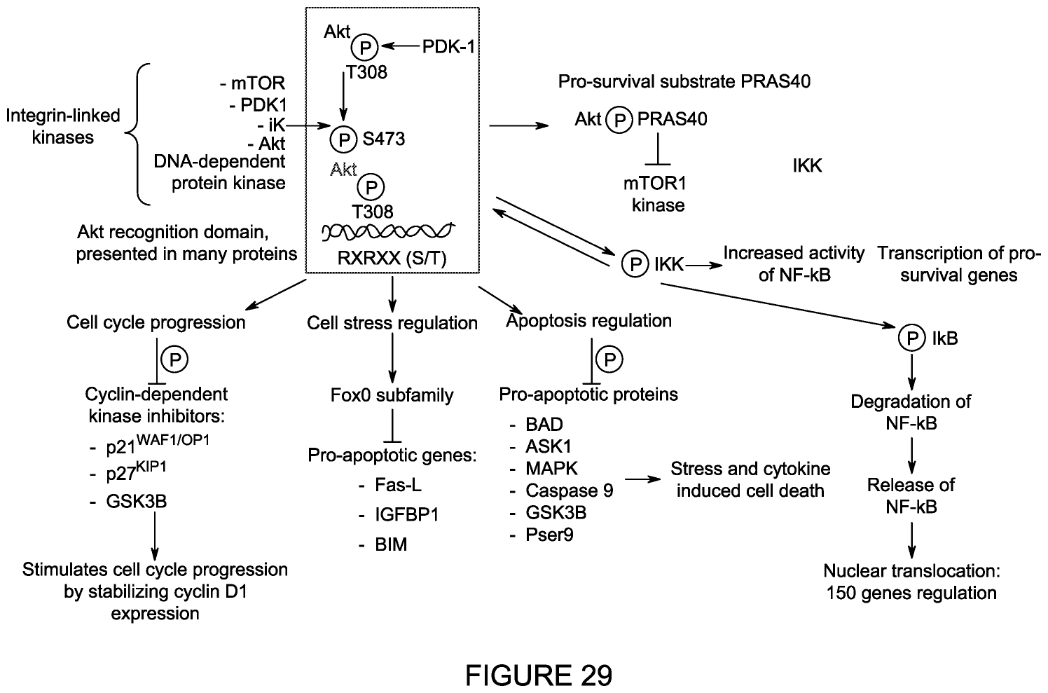

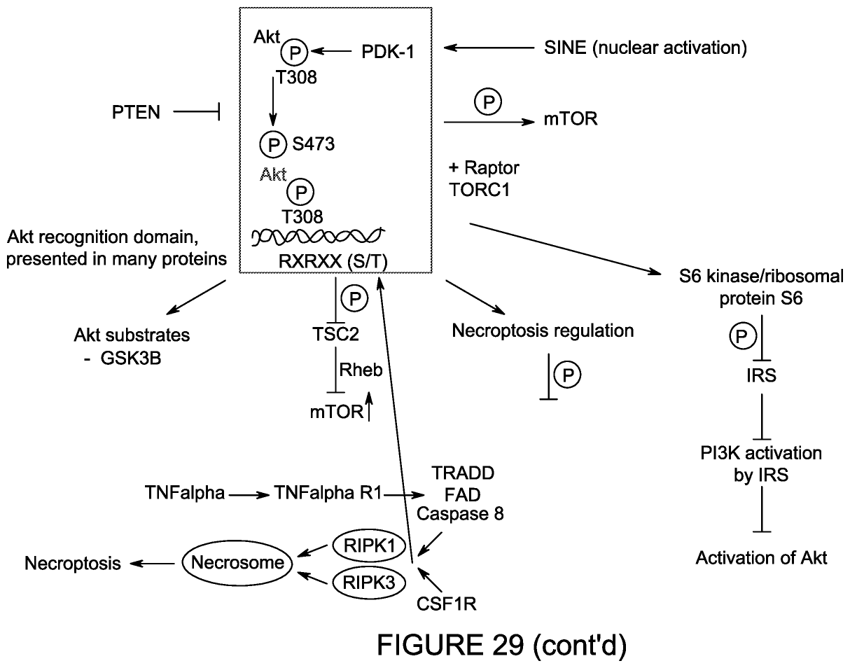

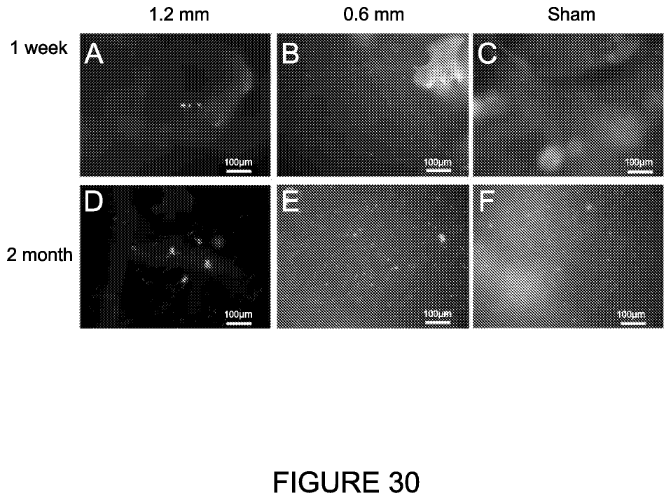

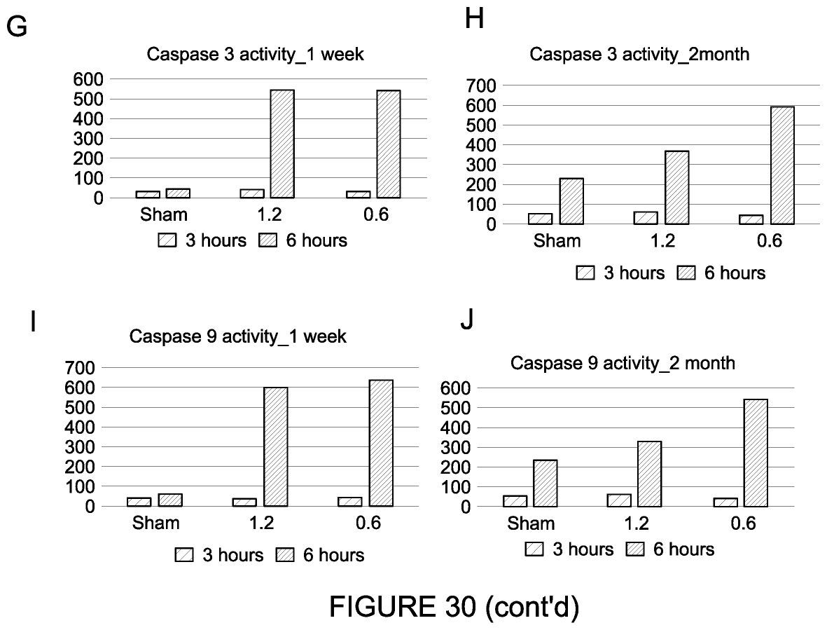

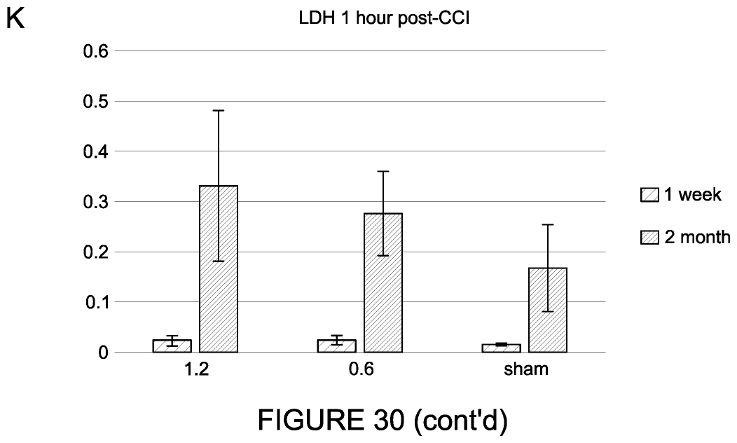

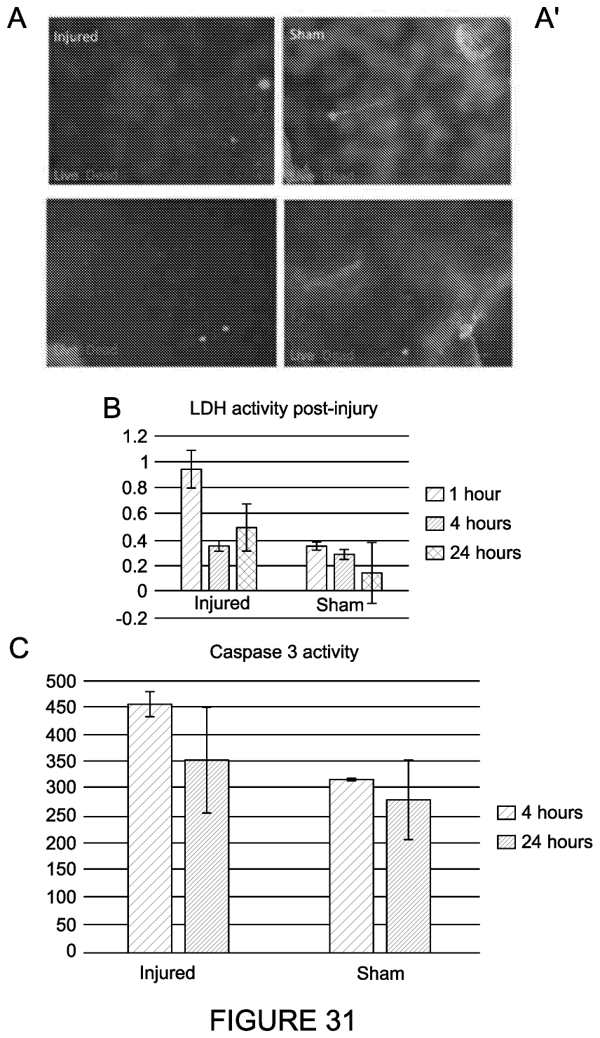

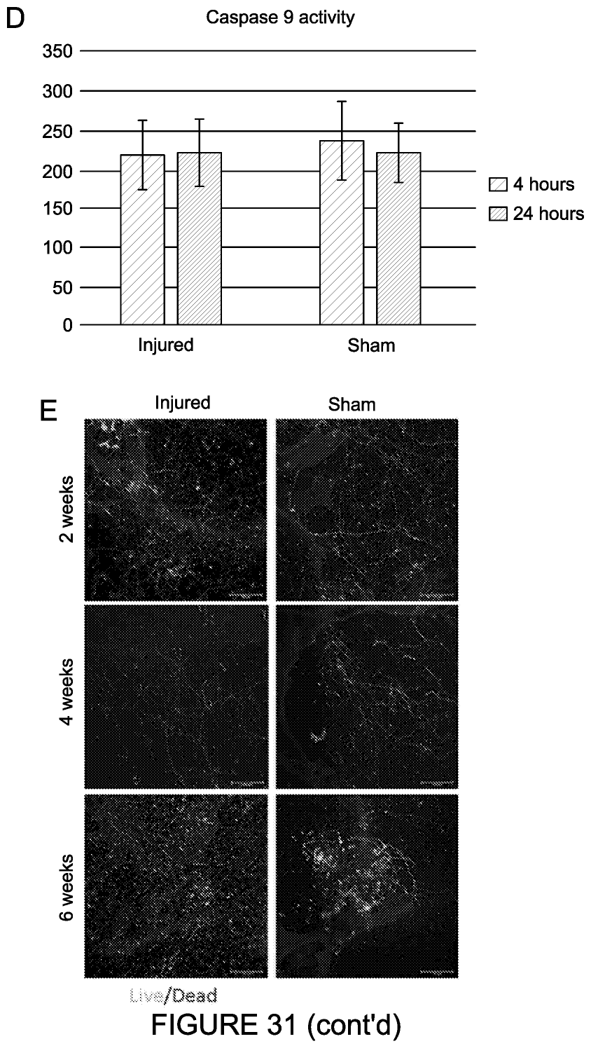

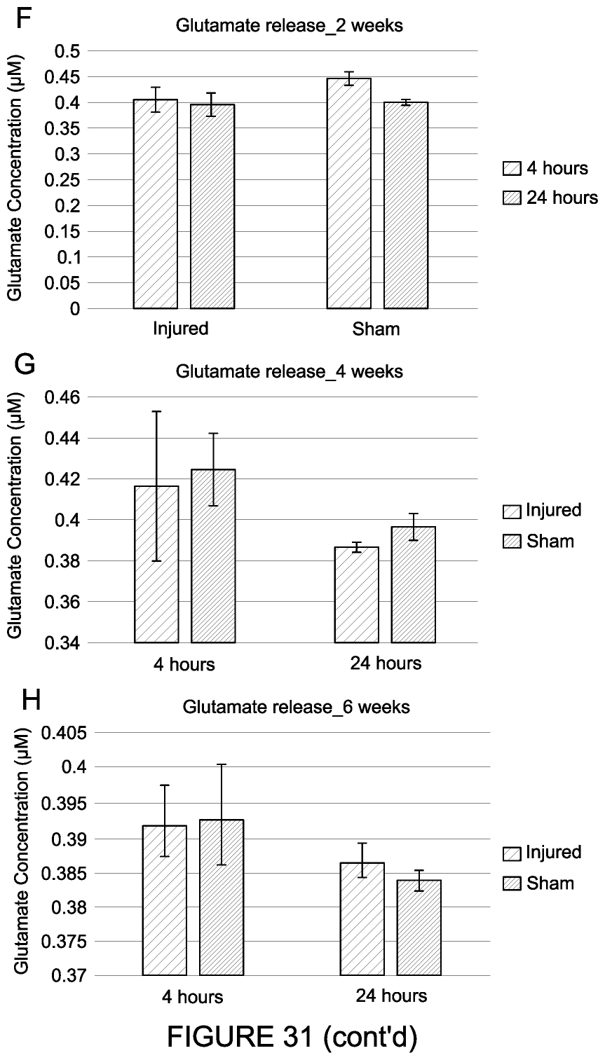

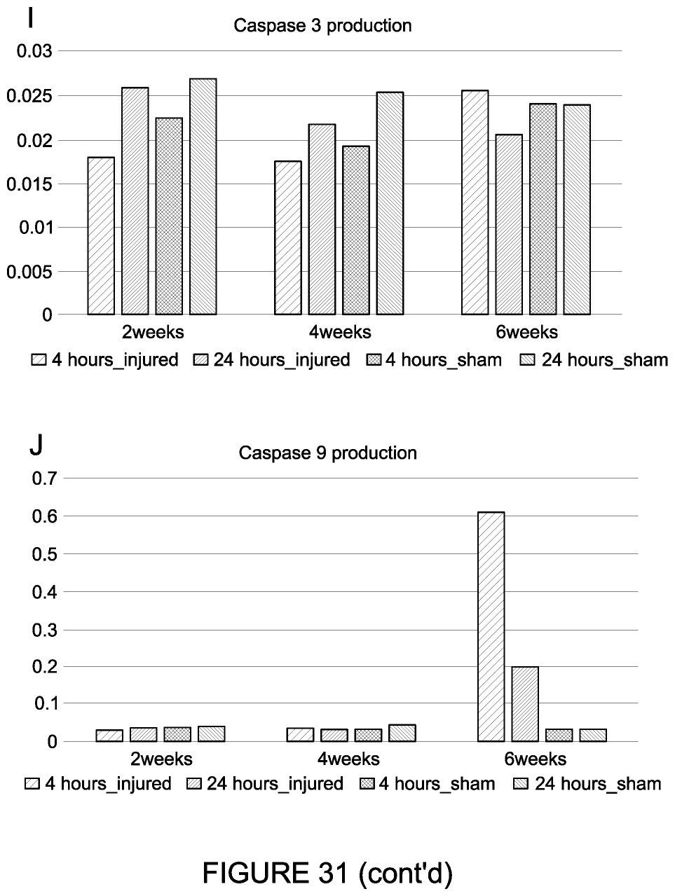

To study the molecular basis of traumatic brain injury (TBI), a variety of injuries can be performed to compare experimental data with in vivo read-outs on functional, cellular and molecular levels (FIG. 28). Also, an exemplary list of biomarkers and pathways that may participate in molecular responses post-TBI is shown in FIG. 29. In this Example, to study the effect of TBI, a controlled cortical impact (CCI) injury model was used. Normally, impact penetration of 0.6 mm depth is used in this model, with an impact depth of 1.2 mm as a positive-control for damage/injury. Injuries were performed on 1 week and 2 month old cultures (FIG. 30, panels A-K and 31, panels A-J). Post-injury, evaluation of cell viability was performed (using Live/Dead assay (FIG. 30, panel A-F and 31, panels A-A′), caspase 3 (FIG. 30, panels G-H and 31, panels C) and 9 (FIG. 30, panels I-J and 31, panel D) activity (early and late apoptotic markers, respectively), and LDH release assay (FIG. 30, panel K and 31, panel B; at 1, 4 and 24 hours post injury (FIG. 31, panel B) or with media collected 1 hour post-injury to evaluate cytotoxicity, post-injury (FIG. 30, panel K)). The study showed that impact penetration of 0.6 mm in vitro provided the best in vivo analog. Further evaluation was performed on 2, 4, and 6 week old mouse cultures and evaluated for cell viability (green—live cells; red—dead cells) in CCI-treated and sham control on mouse samples of 2, 4, and 6 weeks old (FIG. 31, panel E). Glutamate release (FIG. 31, panels F-H) and caspase production (FIG. 31, panels I and J) were also evaluated. As can be seen in FIG. 31, panels F-H, glutamate release after up to 24 hours was measured in 2, 4, and 6 week old cultures, respectively. Caspase 3 and Caspase 9 production were also measured up to 24 hours post injury in 2, 4 and 6 week old cultures (FIG. 31, panel I [caspase 3] and 31, panel J [caspase 6]).

Optimal incubation time for in vitro cultures prior to injury was also evaluated. It was noted that a significant difference in viability and LDH release was observed between cultures of different incubation times. Cells were cultured and evaluated every two weeks over a two month period. Viability of cells was evaluated with LD, LDH, caspase activity and western blots to examine Akt/mTOR signaling pathway.

Alzheimer's Disease

Using the donut-model and hIPSCs, cells derived from patients with and without Alzheimer's disease will be collected and used to studied incorporation of cells into 3D scaffolds and to gain an understanding of early disease progression.

Autism

Cells from patients with autism have primarily been studied in 2D cultures. Cells derived from patients with autism will be used in a 3D in vitro brain model, supplemented with brain-specific ECM. This will facilitate studies of the etiology of the disease, diagnostic and therapeutic targets in a way that has not previously been possible. Cells will be harvested or accessed from cell banks and studied in 3D in vitro matrix models

Parkinson's Disease

Parkinson's disease involves decline/death of cells in the substantia nigra and surrounding regions of the mesencephalon/midbrain. Studies may be undertaken with cells such as a conditionally immortalized dopaminergic cell line with a phenotype representative of mature neurons that are human relevant (e.g. Lund's Human Mesencephalon (LUHMES) cells, which have been used as a basis of comparison against previous models).

Materials and Methods

Neuromelanin-Silk Sponge Preparation

In this example, melanin extraction from Sepia Officinalis ink was adapted to be compatible with silk formulation and is referred to in this example as “neuromelanin” (NM) (Gou, 2013). Sepia melanin (Sigma) was sonicated using a tip probe in a basic solution of 5M NaOH (Sigma). Both aqueous (aq.) soluble and insoluble precipitates were found following sonication. Following pH equibration, the aq. extract was dialyzed, enriched and isolated into powder form by lyophilization. To construct ‘synthetic Lewy Bodies’ solutions of ferric & ferrous chloride iron salts were precipitated into ‘Iron-NM’ clusters when combined with the aq. NM extract. In combination with alpha-synuclein (Sigma) ‘Iron-NM’ an exterior protein coating was formed.

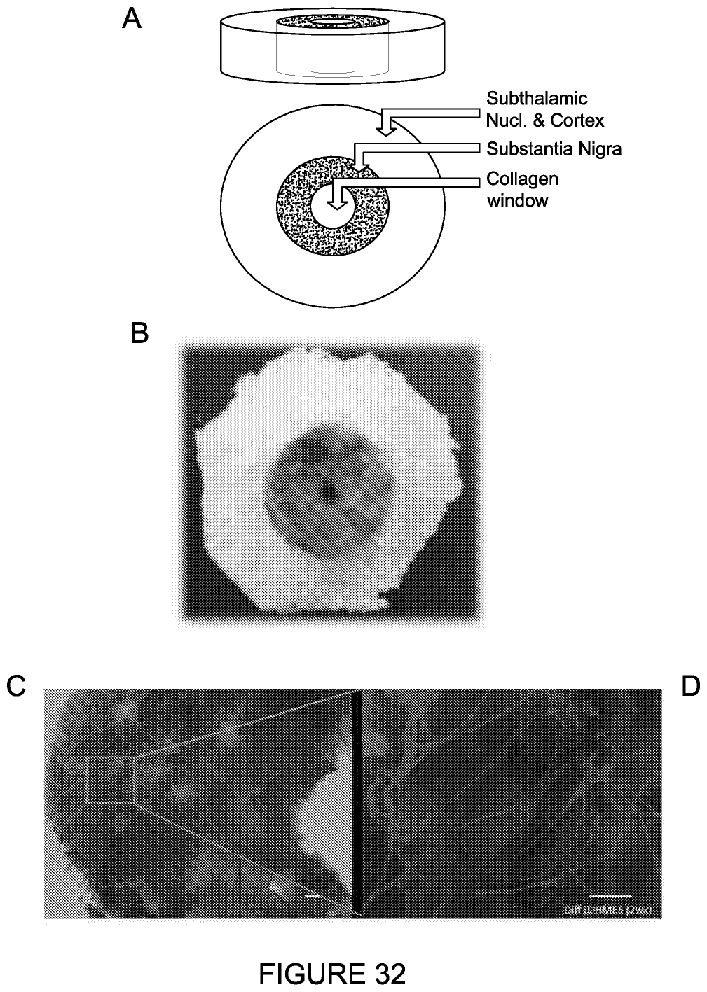

To create NM-Silk sponges, silk was extracted from cocoons of the Bombyx mori silkworm using a 30 minute extraction protocol. Silk salt leeched sponges were prepared as described in Tang-Schomer, 2014 using 500-600 μM sieved particles. Preparation of ‘NM-sponges’ was conducted by combining 10 mg of aq. NM per mL of silk solution (30 ME 6.5%). Control silk sponges were prepared without NM for regions of the subthalamic nucleus and to provide a basis of comparison. Silk sponges were treated with poly-ornithine (Sigma) overnight followed by human fibronectin (Roche) for 12 hours prior to cell seeding. FIG. 32, panel A shows an exemplary schematic of a neuromelanin-silk sponge preparation and FIG. 32, panel B shows an exemplary image of an NM silk sponge after 4 weeks of differentiation. LUHMES dopaminergic neurons expressed beta-III tubulin after 2 weeks of differentiation (FIG. 32, panels C and D).

Dopaminergic (LUHMES) Cell Culture

Primary cortical neurons were extracted from embryonic day-18 Sprague Dawley rats (Charles River). Ethical treatment and sacrifice of rats was conducted under the auspices of the Tufts University IACUC animal facility. Cortices were isolated and dissociated for 20 minutes with 0.25% trypsin (Fisher). Cells were centrifuged and resuspended in complete neurobasal media (GIBCO®) fortified with 1× GlutaMAX® (Fisher) and 1×B-27 supplement (GIBCO®). Cortical neurons were plated on 2D surfaces (100K cells/mL) and silk sponges (10M cells/mL) following coating.

Lund's Human Mesencephalon cells (LUHMES, ATCC: CRL-2927) (Lotharius, 2007) were grown to 80% confluence prior to seeding in silk sponges. LUHMES were seeded at a density of 1 million cells per scaffold (10M cells/mL with 100 μL per scaffold) on both NM-sponges and silk control sponges overnight. Collagen-I gels (2 mg/mL BD science) were combined with growth factors dibutyryl cAMP (Sigma) and GDNF (Sigma) prior to casting on the silk sponges the following day. Media was supplemented with tetracycline (50 μg/mL), GDNF (100 ng/mL), db-cAMP (50 mM) and bFGF (50 μg/mL).

Immunostaining & Microscopy

Silk control and NM-sponges were fixed using 4% paraformaldehyde (PFA) in DPBS for 30 min, after which sponges were washed extensively with 1×DPBS. Samples were blocked with 10% goat serum (Thermo) before permeabilizing with Triton X-100 (0.2% in DPBS). Primary antibodies included: (1) β-III-tubulin—B3T (Mouse 1:500) (2) tyrosine hydroxylase—TH (Mouse 1:500) (3) and dopamine receptor D2—DRD2 (Rabbit 1:500) (All Sigma). Samples were in cold conditions overnight (at 4° C.). Following washing out of the of the unbound primary antibodies secondary staining was conducted using DAPI and Alexa-568 (Goat-anti-mouse, 1:250) and Alexa-488 (Goat-anti-rabbit, 1:250) for 3-4 hrs at 37° C. Fluorescent microscopy was done using a Keyence BZ-X700.

RT-PCR Expression Profiling

Coding DNA (cDNA) was prepared from day-7 lysed cells from both differentiated and undifferentiated LUHMES cells and primary cortical neurons using Trizol extraction (Thermo) followed by RNeasy extraction kit (Qiagen). Transcription of cDNA was conducted with an iScript kit (BioRad) as described by the manufacturer using a PTC-100 thermocycler (MJ Research). cDNA concentrations were measured using a nanodrop-2000 spectrophotometer (Thermo). SYBR green (Applied Biosystems) was used to conduct analysis according to manufacturer guidelines on a Stratagene MX3000P. Primer sequences can be found in the supplemental section (Schildknecht, 2013 & Harvard Primer Bank).

Electrophysiology

Local Field Potential

Observation of spontaneous neural firing was conducted using local field potential (LFP) with pulled capillary electrodes and induced electrical stimulation. Extracellular bath solutions included: 140 mM NaCl, 2.8 mM KCl, 2 mM CaCl2, 2 mM MgCl2, 10 mM HEPES, and 10 mM D-glucose, with pH adjusted to 7.4 with NaOH. Spontaneous firing results were confirmed using Tetrodotoxin (TTX) (1 mM) to block depolarization following stimulation.

Patch Clamp Analysis

Voltage clamp recordings were conducted to assess if there was an appreciable difference between differentiated and undifferentiated LUHMES cells. Extracellular bath solutions were created as described above and intracellular bath solutions were 140 mM Potassium Gluconate, 140 mM NaCl, 2 mM MgCl, 10 mM HEPES, 1 mM EGTA, 4 mM MgATP, and 0.3 mM NaATP with pH adjusted to 7.3 with KOH. Following successful formation of a gigaseal the membrane potential was adapted using voltage steps of 10 mV between −70 to 160 mV.

Neurotransmitter Analysis

Silk sponges that were grown for several days in culture were removed for neurotransmitter extraction and analysis. Sponges were cut up using micro-scissors then individually immersed in a solution of 3M perchloric acid for 10 min. Following the initial extraction the lysate was exposed to a series of freeze thaw cycling and sonication to remove soluble protein before HPLC injection. The extracted lysate was separated using a QIAshredder kit (Qiagen) after which the falcon tube was centrifuged at 15K RPM to remove any residual precipitate. The solid protein precipitate was assessed using a Pierce BCA protein reagent kit in order to standardize the amount of NT per unit of mass (pM/mg). HPLC analysis was conducted using an Antec-Leyden Intro HPLC equipped with an electrochemical detector. Samples concentrations were diluted 1:50 with Nanopure water before injection to accommodate the sensitivity range of the instrument (refer to Cannazza, 2005 for further details). Neurotransmitters evaluated include Dopamine (DA), L-DOPA, Homovanilic Acid (HVA), Hydroxy indole acetic acid (SHIAA), Serotonin (5HT), Norepinephrine (NEPI) and epinephrine (EPI). Neurotransmitter processing enzymes evaluated include: TH—Tyrosine hydroxylase (2) AADC—Amino acid decarboxylase (3) PNMT—Phenylethanolamine-N-methyl transferase (4) DBH—Dopamine beta hydroxylase (5) MAO—Monoamine oxidase.

Protein Analysis

Sponges were lysed at desired time-points using NP-40 lysis buffer. The soluble protein content was isolated and further purified. NuPAGE (10% Bis-Tris) western blotting gels (Thermo) were used and loaded with NuPAGE antioxidant and loading buffer using morpholino-propanesulfonic acid (MOPS) running buffer. Following completion gels were transferred using the iBlot gel transfer device and corresponding cassettes using a PVDF membrane. Transfer was confirmed using Ponceau staining prior to sample confirmation with immunoblotting 1:500 concentration of primary antibodies in blocking buffer (deionized water with 4% goat serum. 0.01% Triton X-100).

Statistical & Computational Analysis

Samples were conducted in triplicates and data are expressed in means+/−SEM. Graphpad was used as a statistical analysis software package and in the preparation of graphs for T-test and ANOVA analysis. ImageJ and NeuronJ were used in the analysis of neurite length (NIH).

### Results

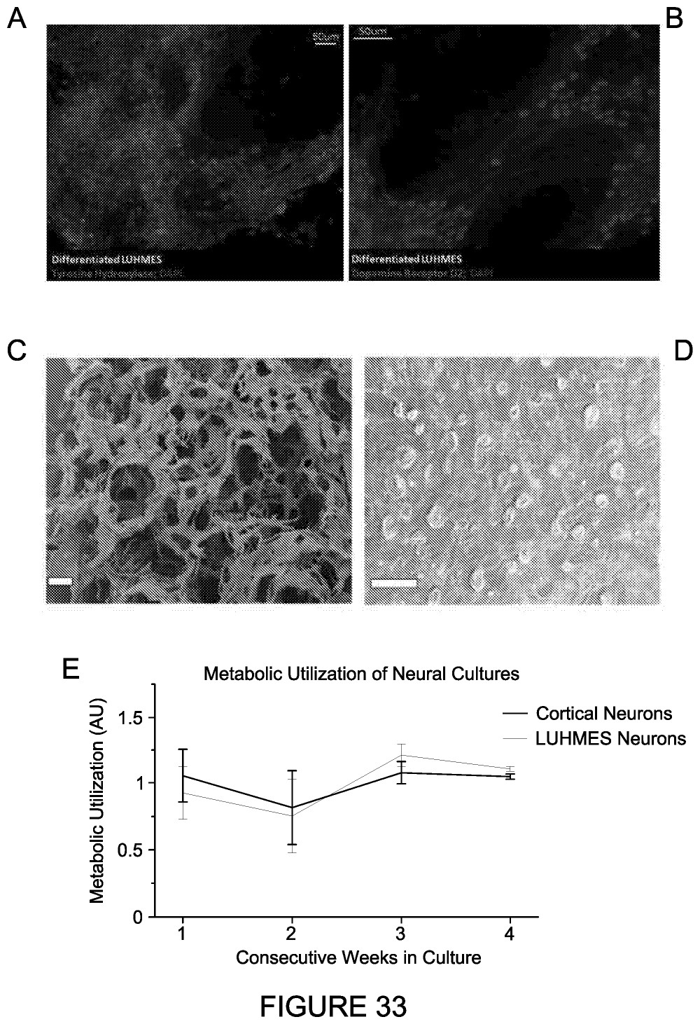

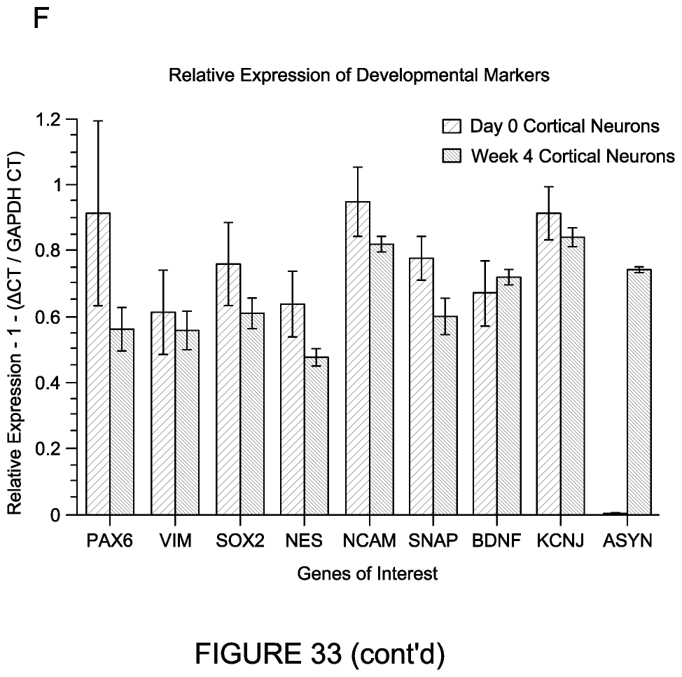

Following two weeks of differentiation using differentiation factors described in Lotharius 2007 LUHMES dopaminergic neurons showed extensive neural projections, as shown by beta-III tubulin-positive staining (FIG. 32, panels C-D). After roughly four weeks of differentiation the silk sponges became visibly brown (FIG. 32, panel B), possibly due to extensive production of dopamine and consequent formation of NM like side products. In addition to Dopamine (DA), additional neurotransmitters were studied including 3,4-Dihydroxyphenylacetic acid (DOPAC), L-3,4-dihydroxyphenylalanine (L-DOPA), 5-Hydroxyindoleacetic acid (5-HIAA) and 5-hydroxytryptamine (5-HT), norepinephrine (NEPI) and epinephrine (EPI). Presently the contributions of neurotransmitter enzymatic profile are not fully understood though the major enzymes implicated in NT production have been established. As can be observed in WST-1 cell viability studies, dopaminergic sponges survived for 4 weeks and showed comparable metabolism to rat primary cortical neurons (FIG. 33, panel E). 3D cultures of differentiated LUHMES cells were positive for dopaminergic markers DRD2 (not shown) and tyrosine hydroxylase (red, FIG. 33, panels A-B). SEM images of NM treated silk sponges show scaffolding on which cells adhere (FIG. 33, panel C) and synthetic lewy bodies (FIG. 33, panel D). Developmental markers expression was measured in cortical cultures of 0 and 4 weeks, and showed changes in several exemplary developmental markers (FIG. 33, panel F).

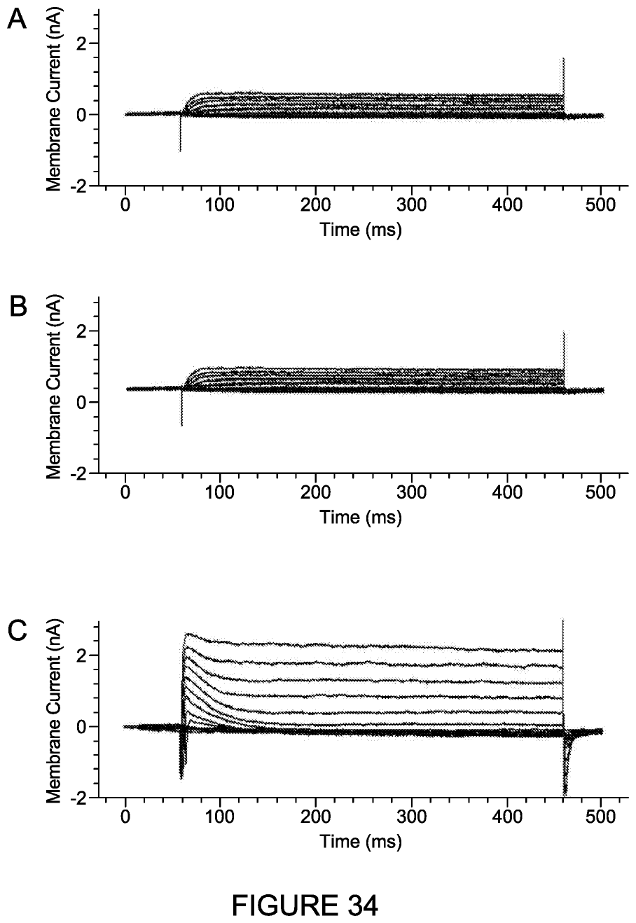

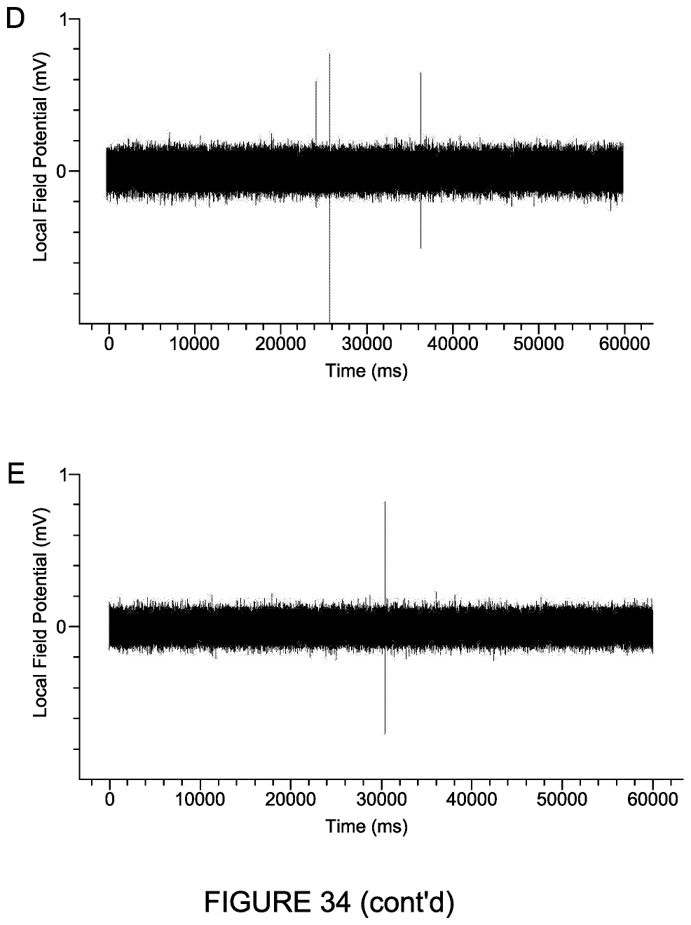

Patch clamping revealed a doubling in the density of potassium voltage gated channels following differentiation. However, inward voltage gated channels were not observed as they were with primary cortical neurons (FIG. 34, panel A-C). Cortical neurons exhibited a higher density of voltage gated currents under patch clamp. An abundance of inward currents is seen. Local Field Potentials show spontaneous action potential firing and electrically evoked spiking were observed with LUHMES (FIG. 34, panel D) and cortical neurons (FIG. 34, panel E).

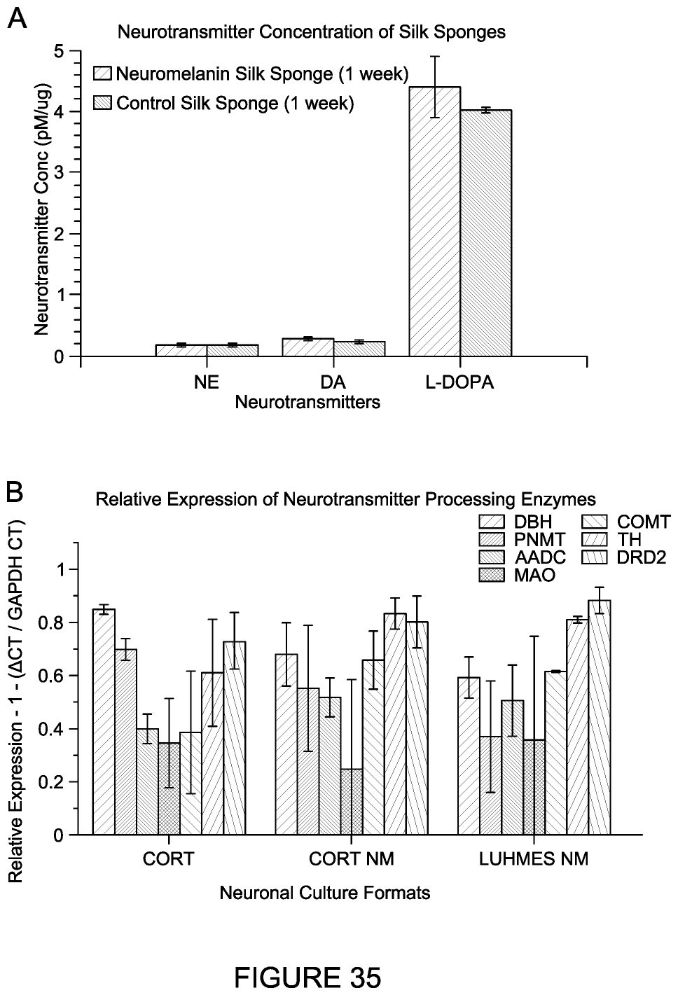

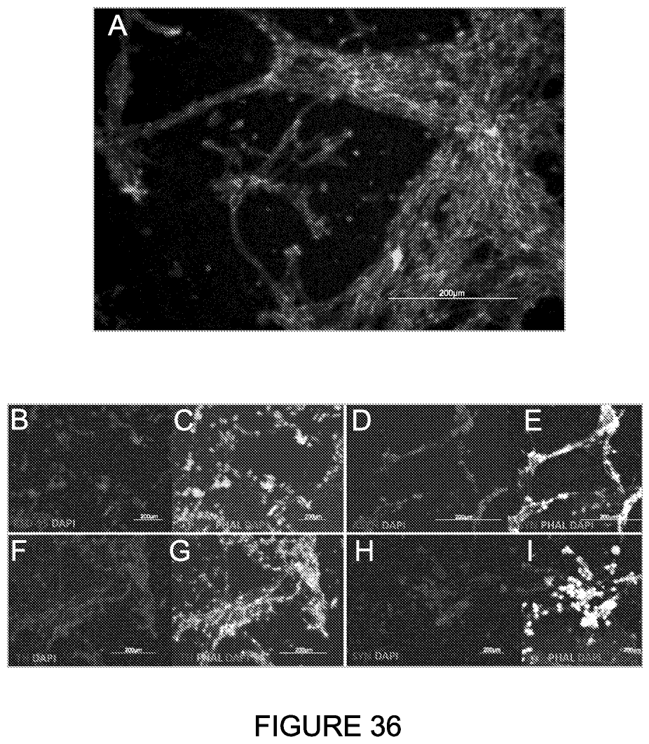

The phenotypic properties of dopaminergic neurons were observed and expressed according to IHC staining (FIG. 36, panels A-I), expression profiling (not shown), neutoransmitter concentration of silk sponges (FIG. 35, panel A), and neurotransmitter processing enzyme quantification (FIG. 35, panel B). FIG. 36, panels A-I show immunocytochemistry of LUHMES cells and staining for vesicle dye FM-1-43 in (red, beta-III tubulin, FIG. 36, panel A), and the excitatory post synaptic density marker (PSD-95, red, panels B and C), synaptophysin (SYN) (red, panels H and I), alpha-synuclein (ASYN; red, panels D and E), tyrosine hydroxylase (TH, red, panels F and G), phalloidin (green, panels C, E, G, and I) and DAPI nuclear counterstain (blue, panels A-H) were observed. Scale bars: 200 um (panel A); 50 um (panels B-H).

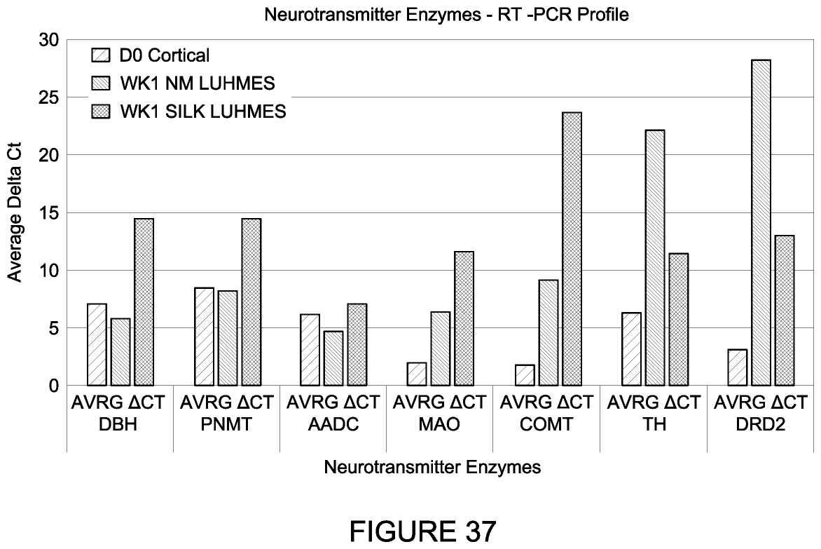

Enzymes implicated in NT metabolism were confirmed using RT-PCR (FIG. 37). This included Catachol-O-Methyl Transferase (COMT), Monamine oxidase (MAO), Amino acid decarboxylase (AADC), tyrosine hydroxylase (TH), Dopamine beta hydroxylase (DBH), and Phenylethanolamine-N-methyl transferase (PNMT) which revealed underlying complexity of NT metabolism. Some of the enzymes implicated in NT metabolism, namely COMT and MAO, are therapeutic targets for inhibitors which increase the available DA within the synaptic cleft. Other means of intervention could include the prevention of NT reuptake mechanisms allowing for a longer duration of activity.

### Example 9: Neuro-Developmental Models

Cerebellar Neuron Model The purpose of this model is to demonstrate an interface between cortical neurons and cerebellar neurons and to observe effects of master regulator genes during development. Previous models have emphasized rat primary cortical neurons whereas this model incorporates cells from both the mesencephalon (becomes the midbrain) and rhombencephalon (becomes the hindbrain). In some embodiments, provided compositions may be used to study cerebellar neurons for their role in epigenetic changes, e.g. DNA methylation and acetylation.

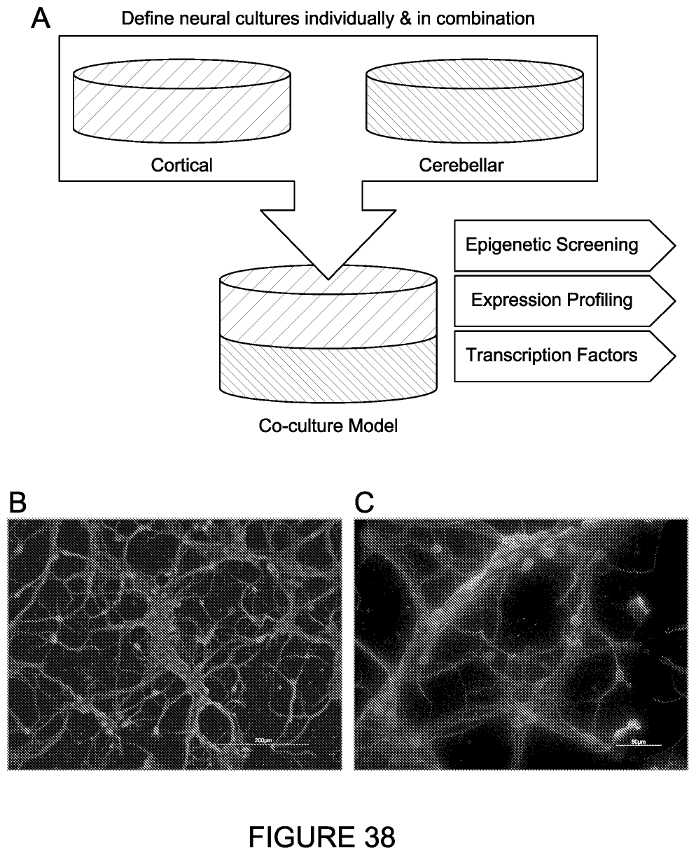

A schematic describing an exemplary provided method is shown in FIG. 38, panel A. This model allows observation of characteristic markers of, e.g., development and developmental stages. Cerebellar neurons were cultured for 2 weeks and show beta-III tubulin positive expression (green, FIG. 38, panels B and C).

### Example 10: 3D In Vitro Engineered Rat Brain Tissue for Transplantation

The objective of the work in this Example was to enrich a 3D in vitro cortical-like brain tissue model developed earlier in our group (Tang-Schomer et al., 2014b) with features to modulate the inflammatory cascades to be presented in vivo and to engineer brain grafts for transplantation. In this Example, a representative anti-inflammatory, methylprednisolone (MP) (used in clinical applications to mitigate the consequences of brain injuries), was integrated in the 3D brain tissue model. Both burst and prolonged release were achieved, with burst release of MP achieved in the first 48 hours, and prolonged release achieved over two weeks. This design was used to mitigate the initial acute immune response in vivo, followed by more sustained control over a reasonable time frame while not interfering with the subsequent M2 regeneration phase of the process. Moreover, in in vitro studies the release dose of MP over 14 days converted M1 (pro-inflammatory) activated stage of immune cells, microglia and monocytes into M2 (anti-inflammatory) in a suitable time frame, which is critical in application to treatment of brain recovery and transplant survival.

### Materials and Methods

Poly-L-lysine (PLL), 6α-Methylprednisolone 21-hemisuccinate sodium salt (MP), Lactate Dehydrogenase kit (MAK066), Propidium iodide, Bovine Serum Albumin, TritonX were purchased from Sigma-Aldrich (St Louis, Mo., USA). B-27 Supplement (50×), penicillin/streptomycin, Glutamax, Trypsin-EDTA (0.25%), Defined Trypsin Inhibitor, Dulbecco's Modified Eagle Medium (DMEM), NeuroBasal medium, CalceinAM, Paraformaldehyde, Goat serum; 4′,6-Diamidino-2-Phenylindole, Dihydrochloride (DAPI); Dulbescco's Phosphate-Buffered Saline (DPBS), Hank's Balanced Salt solution (HBSS) were obtained fromThermoFisher Scientific (Grand Island, N.Y., USA). DNAse was provided by Roche Applied Sciences (Indianapolis, Ind., USA). Salmon fibrinogen and Salmon thrombin were purchased from Sea Run Holdings (Freeport, Minn., USA).

Silk Scaffold Preparation.

Liquid silk and salt-leached scaffolds were prepared from Bombyx mori cocoons following our published protocols (Rockwood et al., 2011). Scaffolds were biopsy punched (McMaster-Carr, Princeton, N.J., USA) for in vitro experiments (6 mm diameter, 1-2 mm height) and in vivo experiments (2 mm diameter, 1-2 mm height). Prior to cell seeding scaffolds were autoclaved and coated with poly-L-lysine (10 ug/mL; Sigma) overnight in a CO₂ incubator. The next day scaffolds were extensively washed with PBS (ThermoFisher), and equilibrated for one hour in NeuroBasal medium (ThermoFisher) supplemented with 2% B27 (ThermoFisher), penicillin/streptomycin (100 U/mL and 100 ug/mL) (ThermoFisher) and 2 mM Glutamax (ThermoFisher). Concentrated cell suspensions (10 million cells/mL) were added to slightly dehydrated scaffolds and incubated for 24 hours, washed with PBS, and prepared for hydrogel embedding the next day. Scaffolds were embedded in fibrin hydrogels either without MP or loaded with MP for 30 min before immersion in media.

Microglia Isolation from Neonatal Brain Tissue

A neonatal brain tissue isolation protocol was approved by the Tufts University Institutional Animal Care and Use Committee and complies with the National Institutes of Health Guide for the Care and Use of Laboratory Animals (Institutional Animal Care and Use Committee M2015-151). Microglia isolation was performed following established protocols (Tamashiro et al., 2012). In brief, P2 (postnatal) Sprague-Dawley rats (Charles River) were utilized, all brains were removed, cleared from meninges and collected in cold HBSS (ThermoFisher). Collected brains were dissociated with trypsin (0.25%; Thermo Fisher) mixed with fresh DNase (1 mg/ml, Roche Applied Sciences), followed by incubation with trypsin inhibitors. Before centrifugation, cell suspensions were gently vortexed, and clumps were removed with 40 um filters. After centrifugation cells were resuspended in glial media (DMEM basal medium (ThermoFisher) supplemented with 10% FBS and penicillin/streptomycin (100 U/mL and 100 μg/mL) and plated in T75 flasks for 2-3 weeks, until microglia reached a high number. Microglia were isolated by shaking for 1 hour at 100 rounds/minute. Media with microglia in suspension was collected and spun down, counted and plated according to the experimental design.

Cortical Neuron Isolation

A fetal brain tissue isolation protocol was approved by the Tufts University Institutional Animal Care and Use Committee and complies with the National Institutes of Health Guide for the Care and Use of Laboratory Animals (Institutional Animal Care and Use Committee M2015-28). Cortices were isolated from embryonic day 18 (E18) Sprague-Dawley rats (Taconic). Cortices were dissociated using a mix of trypsin (0.25%; ThermoFisher) and DNase solution (0.2%; Roche Applied Sciences) and followed with incubation with trypsin inhibitors (ThermoFisher). Cells were collected in pellets by centrifugation, resuspended in fresh neural medium and counted.

3D Brain-Like Tissue Model Preparation with Methylprednisolone Loaded into the Hydrogel

Following the published protocol (Tang-Schomer et al., 2014b) 100 ul of cortical neurons were seeded in silk sponges (10 million cells/mL), and incubated at 37° C. overnight. The following day, the sponges were embedded in a fibrin hydrogel mixed with methylprednisolone. In the original work (Tang-Schomer et al., 2014b) collagen gel was used to support the formation of axon connections, in this study collagen was replaced with salmon fibrinogen and thrombin as it was shown to have a better support for neuronal growth and functional network formation (Ju et al., 2007). Methylprednisolone (Sigma) was mixed with thrombin to achieve a final concentration of MP in a range from 0 to 8.7 mM (Table 1) and after combined with fibrinogen. The upper limit for loading methylprednisolone into the fibrin hydrogel was determined to be 8.7 mM, based on the solubility limit of MP in water (10 mM), and the drug loading capacity of thrombin. After all components were combined, 100 ul of this solution was added to the seeded sponges and incubated at 37° C. for 30 minutes to promote gelation. After samples were transferred to a 24 well plates containing lml NB media per well for MP release measurements. Sampling for release of the drug included complete media replacement for more precise quantification and was carried out over the first 24 hours and up to 2 weeks. To study the viability with lactate dehydrogenase (LDH, Sigma) assay the media was collected at Days 1, 3, 7 and 14, and samples were stained for Live and Dead imaging.

Preparation of Fibrin Gel Loaded with Methylprednisolone.

Fibrin hydrogels were prepared to a final concentration of 3 mg/ml fibrinogen (Sea Run Holdings) and 1.5 U/ml thrombin (Sea Run Holdings), using water and/or 10 mM methylprednisolone (Sigma) in sterile water (ThermoFisher) as diluent, as indicated in Table 1. All steps were carried out on ice. To begin, 6 ul of thrombin stock (250 U/mL) was combined with variety of water volumes (0, 0.27, 0.57, 0.72, 0.87 mL) and methylprednisolone (0.87, 0.6, 0.3, 0.15, 0 mL) to achieve hydrogels with MP final concentrations of 8.7, 6, 3, 1.5, and 0 mM accordingly. Lastly, fibrinogen was added to pre-mixed solutions of thrombin with the drug and immediately pipetted on top of the cell seeded scaffolds. Samples were incubated for 30 minutes at 37° C. to allow for fibrin gelation, and afterwards were immersed in media.

TABLE 1
Fibrin gel preparation with incorporated methylprednisolone
at different concentrations.

| | | | | | |
|---|---|---|---|---|---|
| Prep volume (ml) | 1 | 1 | 1 | 1 | 1 |
| Fibrinogen stock | 25 | 25 | 25 | 25 | 25 |
| concentration (mg/ml) | | | | | |
| Thrombin stock | 250 | 250 | 250 | 250 | 250 |
| concentration (U/ml) | | | | | |
| MP stock concentration | 10 | 10 | 10 | 10 | 10 |
| (mM) | | | | | |
| Target MP concentration | 8.7 | 6 | 3 | 1.5 | 0 |
| (mM) | | | | | |
| Solution Components & | | | | | |
| Volumes: | | | | | |
| Volume Fibrinogen stock | 0.12 | 0.12 | 0.12 | 0.12 | 0.12 |
| (ml) | | | | | |
| Volume Thrombin stock | 0.006 | 0.006 | 0.006 | 0.006 | 0.006 |
| (ml) | | | | | |
| Volume MP stock (ml) | 0.87 | 0.6 | 0.3 | 0.15 | 0 |
| Volume water (ml) | 0 | 0.27 | 0.57 | 0.72 | 0.87 |
| Final volume (ml) | 1.00 | 1.00 | 1.00 | 1.00 | 1.00 |
| Final methylprednisolone | 8.7 | 6 | 3 | 1.5 | 0 |
| concentration (mM) | | | | | |

Drug Release Measurements with High Performance Liquid Chromatography (HPLC)

A reverse-phase HPLC method was employed to separate methylprednisolone from media components for quantitation (Tobar-Grande et al., 2013). Methylprednisolone was quantified via an Agilent 1260 HPLC using an Agilent Zorbax Eclipse Plus C18 reverse phase column (4.6×75 mm, 3.5 um; Agilent Technologies, Santa Clara, Calif., USA); flow rate of 1 ml/minute, running a gradient of 95% (water+0.1% TFA)/5% acetonitrile toward 5% (water+0.1% TFA)/95% ACN from 2 to 6 minutes, followed by a 2 minute hold and subsequent re-equilibration. A standard curve of methylprednisolone in neurobasal medium was produced over a concentration range of 500-0.39 ug/ml, with an R squared value of 0.999, by integrating the area under the curve for the peak corresponding to MP (retention time=5.9-6.2 minutes).

Cell Viability Assays

Cell viability was evaluated with calcein/propidium iodide staining (Live/Dead; ThermoFisher, Sigma) and LDH assay. In brief, samples were washed in PBS and after that incubated for 30 min at 37° C. in the solution of calcein and propidium iodide (PI) (2 μm calcein and 25 μm PI in PBS). After incubation samples were extensively washed with PBS, and then imaged with Leica SP8 CARS (Leica Microsystems) immediately. LDH release was measured and quantified following the manufacturers protocol (Sigma, MAK066). In brief, media was collected from drug-loaded samples according to the experimental design. Experimental values were compared to standard curves.

Immunostaining

Samples were fixed with 4% paraformaldehyde (PFA) (Thermo Fisher) for 30 minutes, washed and permeabilized with 0.1% TritonX supplemented with 10% donkey serum (Sigma), and 4% BSA (Sigma) for 1 hour. Primary antibody incubation for 2D cultures was for 1 hour at room temperature; for 3D cultures overnight at 4° C. Samples were washed three times with PBS with gentle shaking, and afterwards incubated with secondary antibodies for 1 hour at room temperature, followed by 5 minutes DAPI incubation and triple washing with PBS. Fluorescent images were taken on a Leica SP8 CARS (Leica Microsystems).

Primary antibodies included: anti-alpha tubulin (mouse; 1:200); anti-MAP2 (rabbit; 1:200); anti-iNOS (rabbit; 1:200); anti-il1beta (rabbit; 1:200) Abcam; anti-ibal (rabbit; 1:200) Wako; anti-TNF-alpha, anti-il4 or 10. , Secondary antibodies: goat anti-mouse or rabbit Alexa 488 and 568 (1:500; Thermo Fisher)

Activation of Microglia to M1 and M2 Stages

For the assays, 96-well plates were seeded with 20,000 microglial cells per well and allowed to equilibrate overnight in 100 ul 75%/25% Glial/Neurobasal media. The next day all media was replaced with either control media (75/25 Glial/NB); control media+20 ng/ml interferon gamma (M1 activator); control media+20 ng/ml interleukin 4 (M2 activator). Cells were incubated for 24 hours, the media was again replaced with the same activators plus varying concentrations of MP based on the release measurements from 3D culture. The MP concentrations in the media were decreased at subsequent feedings according to the release data from previous experiment, while the activator concentrations were maintained. At days 3, 5 and 7 after first adding MP, the media was replaced and the spent media was analyzed for TNF-alpha and interleukin 10 (IL-10) release, markers of M1 and M2 activation, respectively. Cells were fixed for immunological analysis on days 3, 5 and 7.

ELISA

Cytokine (TNF-alpha and IL-10) content in cell culture supernatants was measured using ELISA kits (TNF-alpha (ab46070), it-10 (ab100765), Abcam, Cambridge, Mass., USA) according to the manufacturer's protocol. The assays were performed at room temperature. The limits of detection were 15 pg/mL for TNF-alpha and less than 10 pg/mL for Il-10.

Statistical Analysis

Statistical analyses were performed with one way or two way analysis of variance using GraphPad Prism 7 software. A significant difference was defined as p<0.05. Tukey's post-hoc test was used to further evaluate significant differences between experimental and control groups.

### Results

3D Brain-Like Tissue Transplants Trigger an Acute Inflammation in the Native Brain.

We performed an in vivo motor cortex transplantation. Three experimental groups were evaluated to determine the brain response towards the transplants: no injury, delayed injury, and acute injury. In the first group (no injury), rats underwent a craniotomy and the graft was placed on top of undamaged and unaltered brain tissue. In the second group (delayed injury), an injury of 2 mm² was made and rats were allowed to recover for 7 days prior to transplantation of the graft. In the third group, a 2 mm² injury (acute injury) was made and transplantation of the graft occurred immediately thereafter All transplants were done with tissue containing GFP-labeled neurons, to distinguish them from the hostbrain cells. Three days after transplantation, brains were collected and analyzed for neuronal survival, and inflammatory responseAnalysis showed that only the no injury group (GFP—green; MAP2—red; DAPI nuclear stain—blue) showed high neuronal survival and transplant integration. Immunohistochemical analysis also showed the presence of ibal and CD68 positive immune cells, such as microglia and macrophages in the transplant/graft in both acute and delayed injury groups Subsequent studies were aimed at modulating the inflammatory response to support improved integration of the transplant one way that this was accomplished was by incorporating the anti-inflammatory drug, methylprednisolone, into the brain tissue model.

Methylprednisolone Release from 3D In Vitro Brain Model.

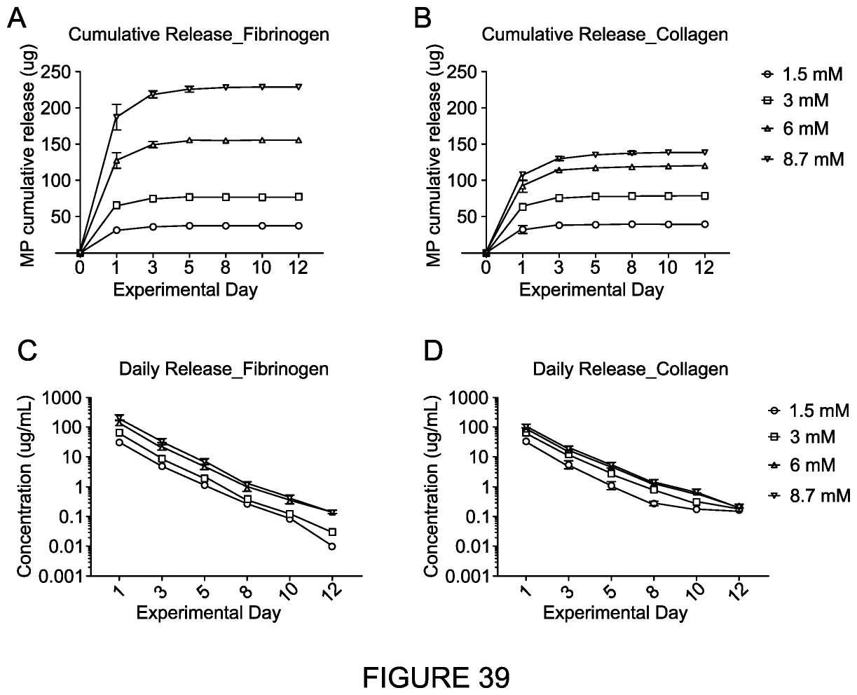

Two different hydrogels, fibrin and collagen were studied for methylprednisolone release in order to determine which material would have burst release in the first 2-3 days of acute inflammation following transplantation, and prolonged small dose releases in two weeks following transplantation, to support the transplant integration, but not enough to suppress immune cell activity at the regeneration step. All fibrin/methylprednisolone groups demonstrated a release of MP that was twice as high in first 48 hours as compared to collagen groups (FIG. 39, panels A and B). This initial release consisted of about 50-60% of the total amount of loaded drug and followed by slower release (20% more) out to day 8 in both groups. However, from Day 10 on, the release for all conditions was in the nanogram range, whereas the collagen groups showed twice as much release of MP as the fibrin groups (FIG. 39, panels A-D). The MP release data point towards application of fibrin for a drug loaded hydrogel, due to its desirable characteristics for transplantation release kinetics, with high doses during the initial days following transplant, and nanogram range doses at later time points.

High Doses of Methylprednisolone have No Negative Effect on Neuronal Viability

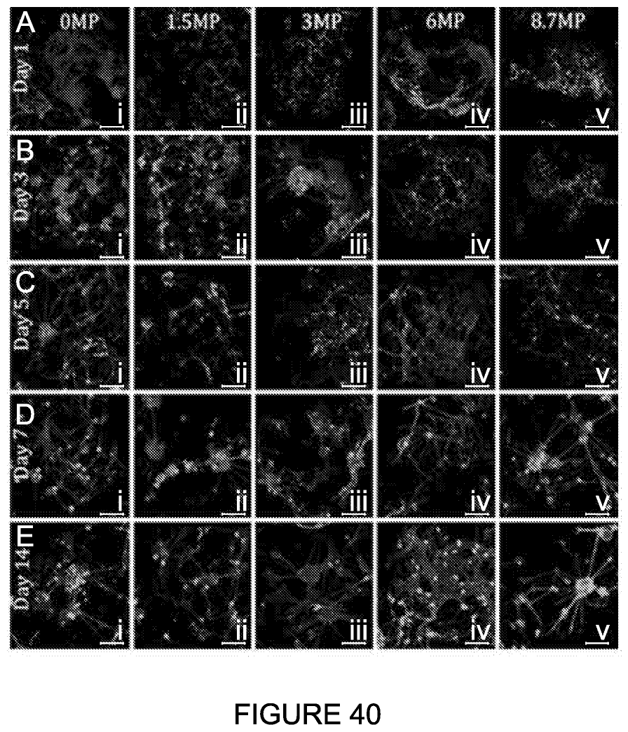

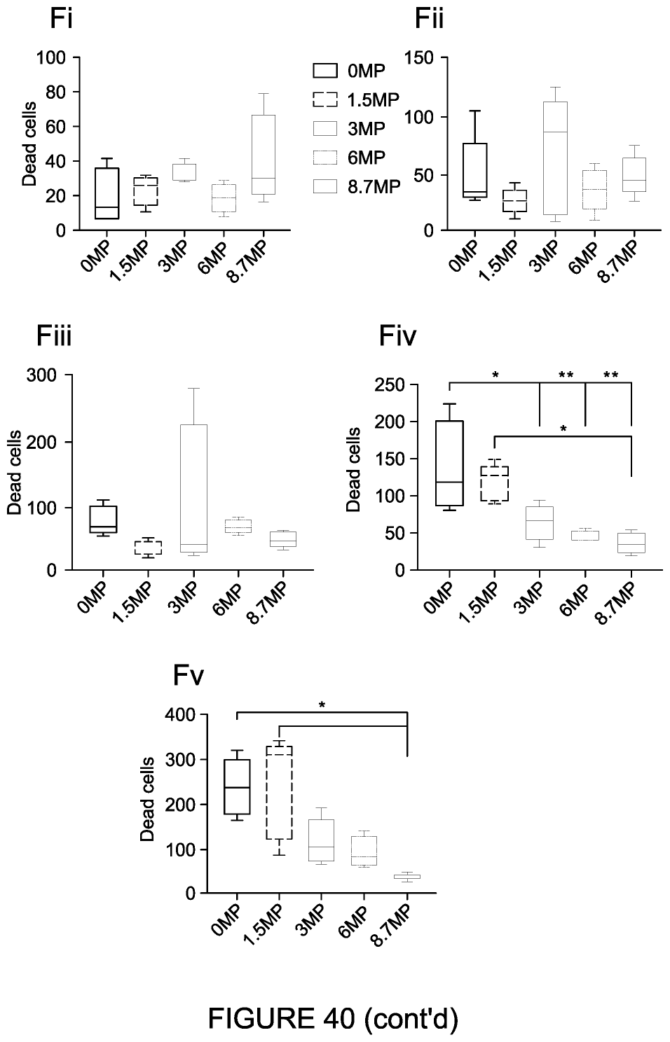

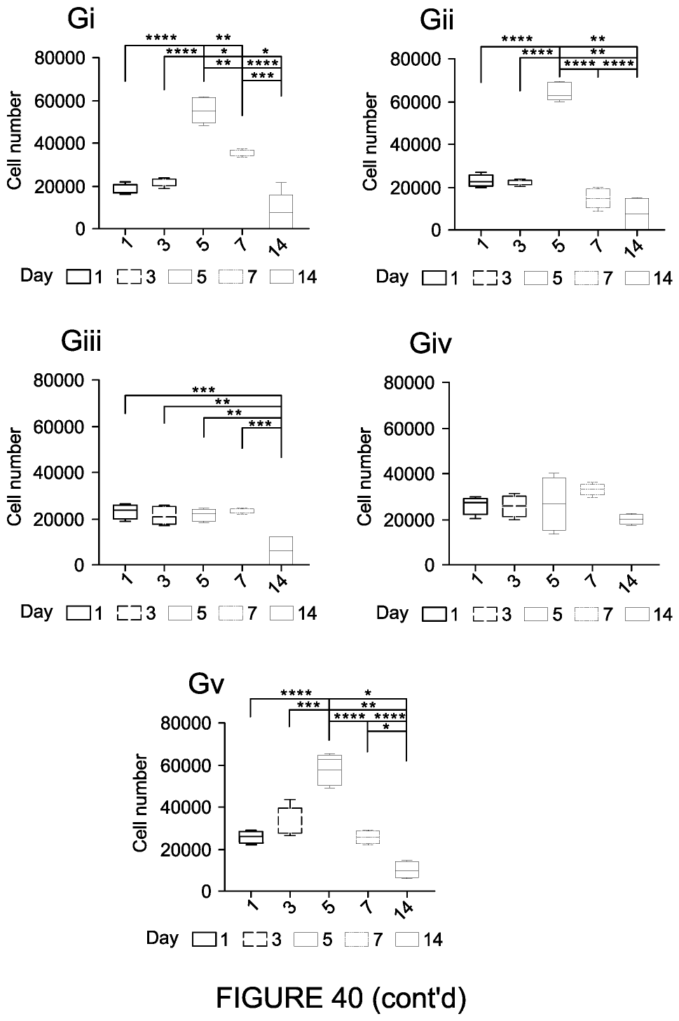

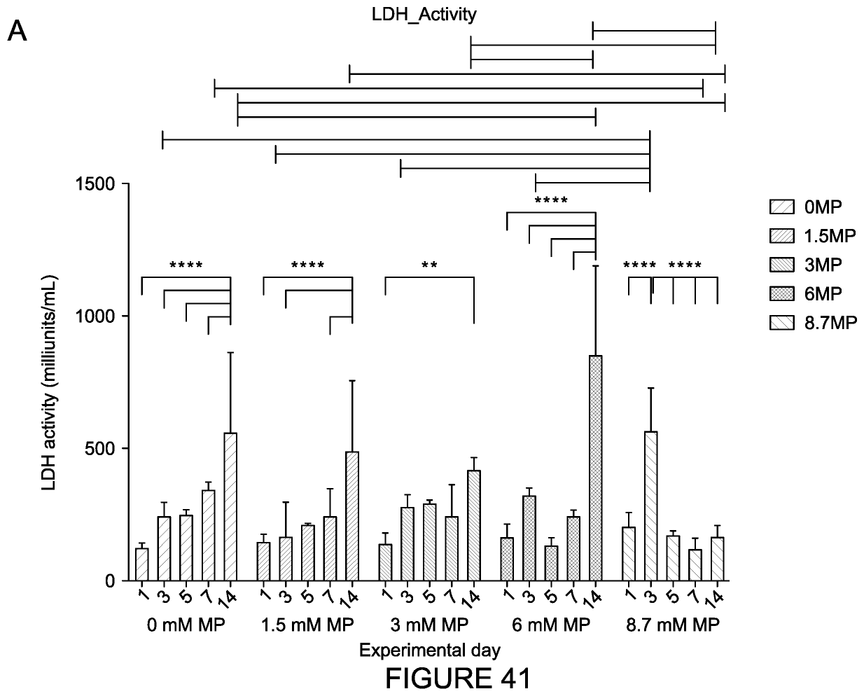

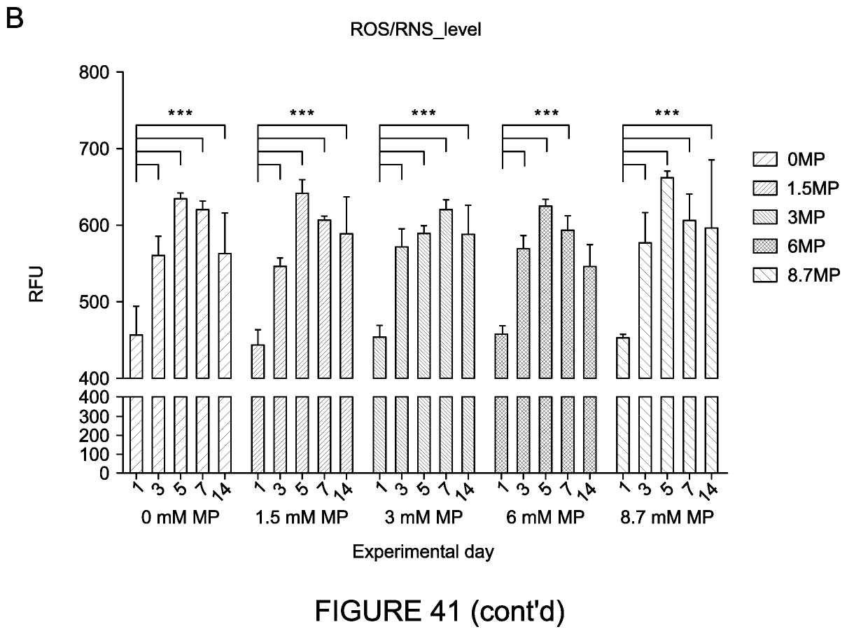

To determine if methylprednisolone (“MP”) effects neuronal viability we employed several different assays including a live/dead assay to analyze the neuronal network formation, dead cell quantification, DNA quantification to determine the total cell number, an LDH assay to evaluate release of cell death markers, and a ROS/RNS assay to eliminate the hypoxia in our model (FIG. 41, panel A and B, respectively). The live/dead assay (FIG. 40, panels A-C) showed viability and quantification of cells with and without MP treatment and demonstrated extensive neuronal network formation in control (0 mM MP) and all drug-loaded groups at later time points (7 and 14 days), however at early time points in 6 mM and 8.7 mM MP groups, obvious differences in neuronal cell body size was observed, as well as less network formation. There were no statistically significant differences in number of dead cells in control and MP-treated groups in the first three time points (0, 7, and 14 days), however at the latest time points a significantly lower number of dead cells was present in 8.7 MP group. This decrease was also supported by LDH release data showing. The total amount of DNA indicated a general decline in cell number at later time points. The collected data support that high doses of MP do not negatively affect neuronal viability, but do support that high doses of MP postpones neuronal maturation, which could be beneficial for transplantation, including because of potential for increased plasticity of neuronal progenitors as compared to mature neurons.

Anti-Inflammatory Effect of Methylprednisolone on Immune Cells in In Vitro.

Microglia and macrophages are considered the main target of MP in the brain. Microglia and macrophages were grown in 96 well plates in the presence or absence of cytokines known to induce either classic (M1) or alternative (M2) activation profiles (Goggin et al.).

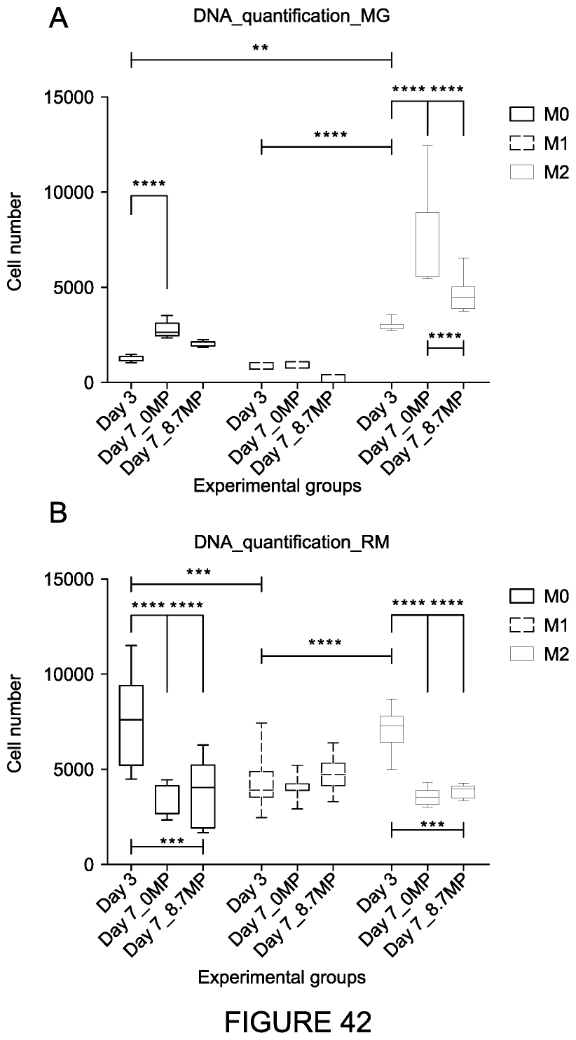

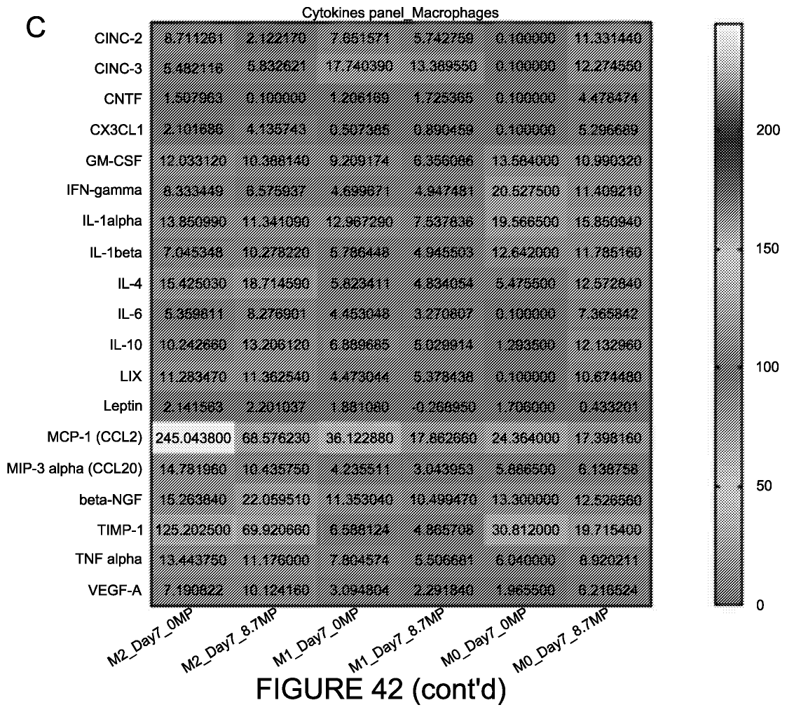

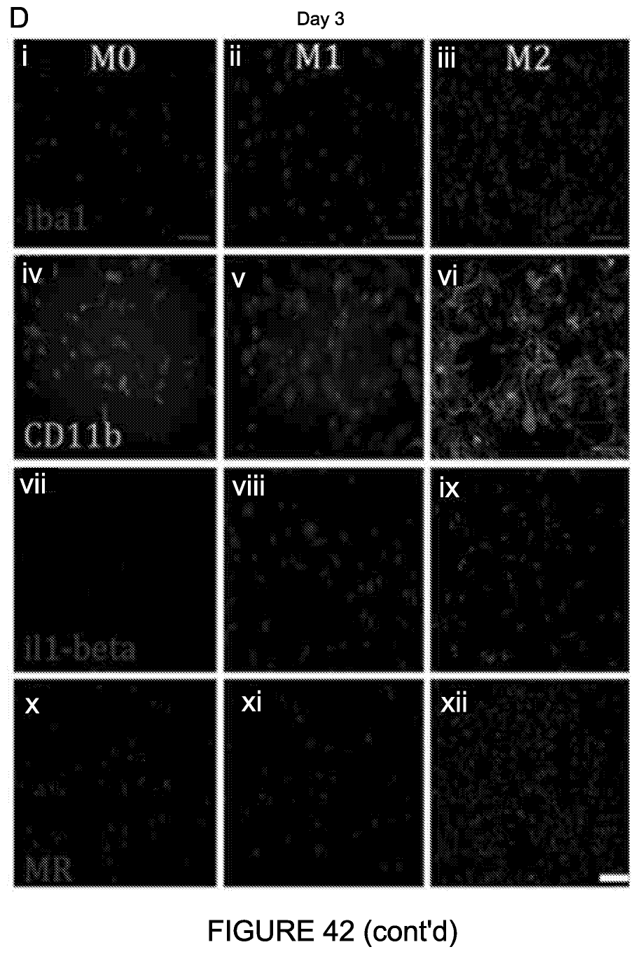

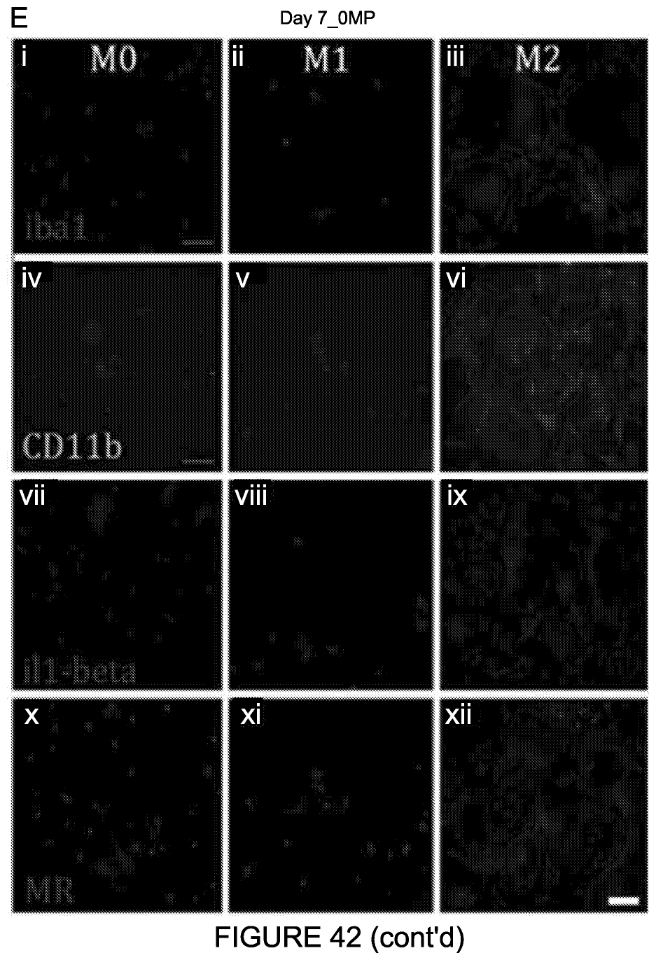

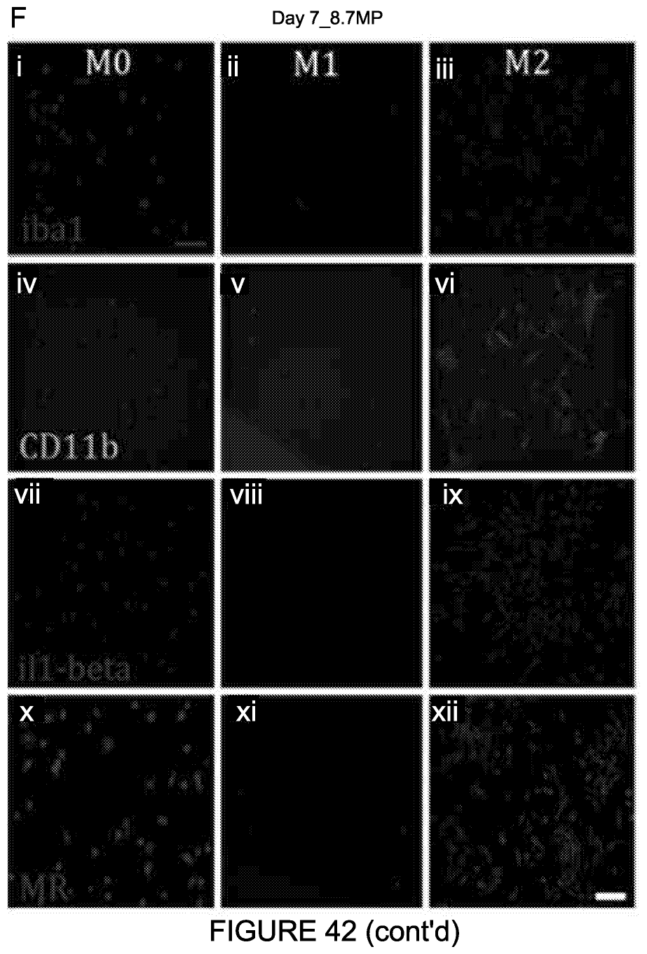

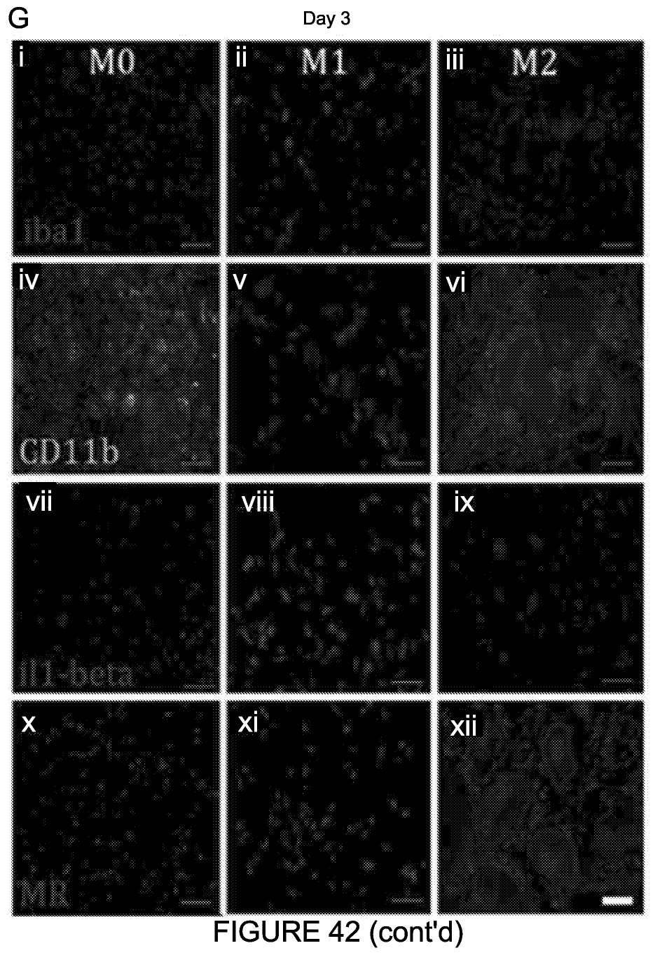

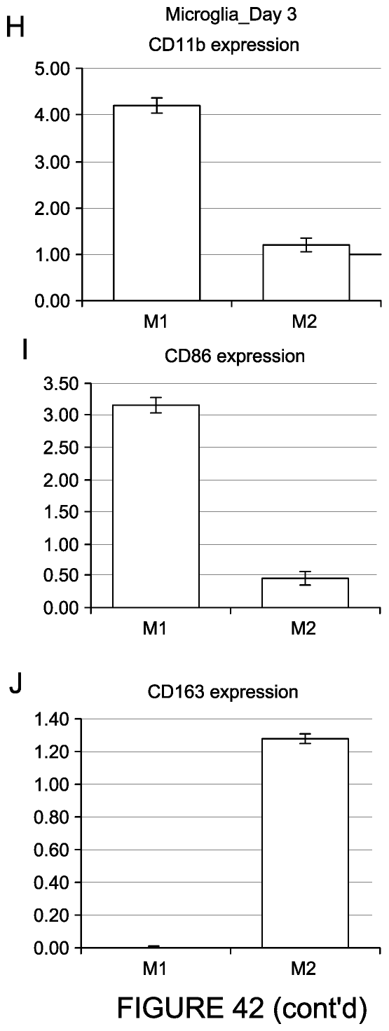

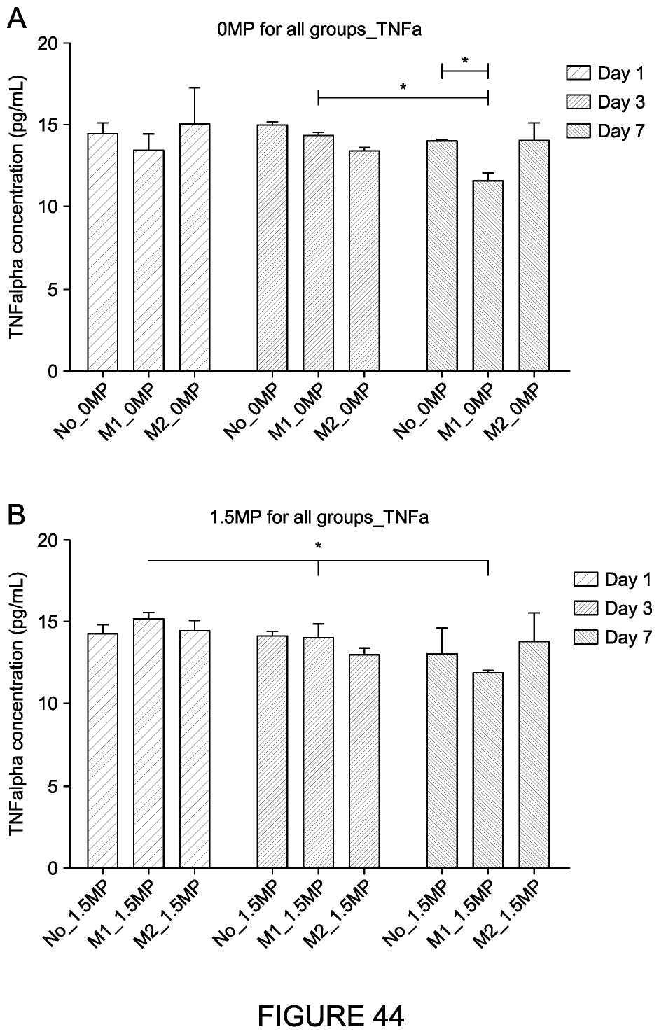

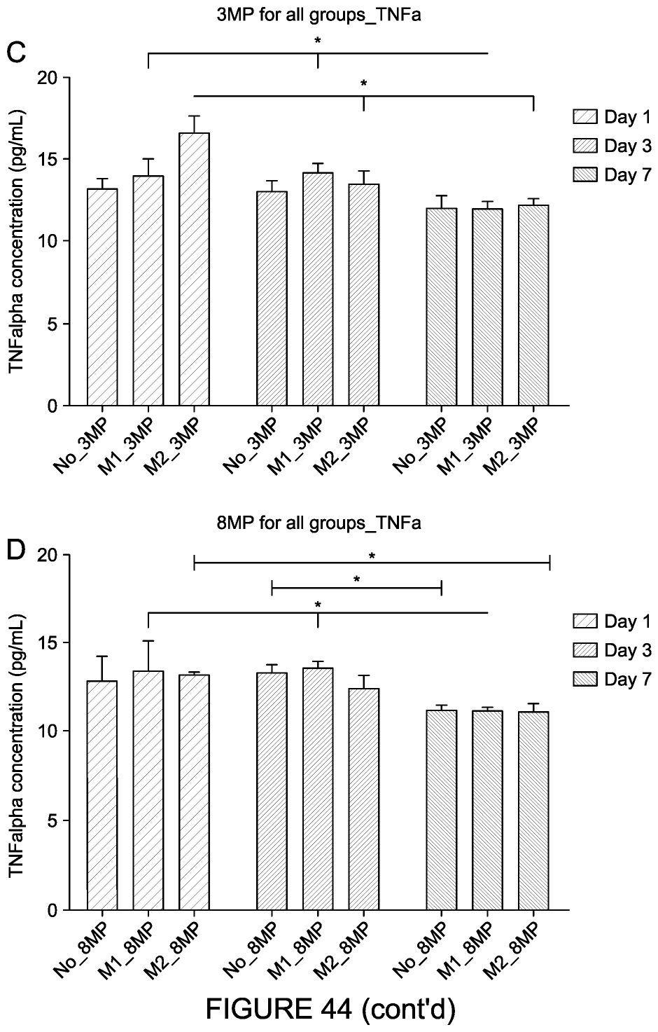

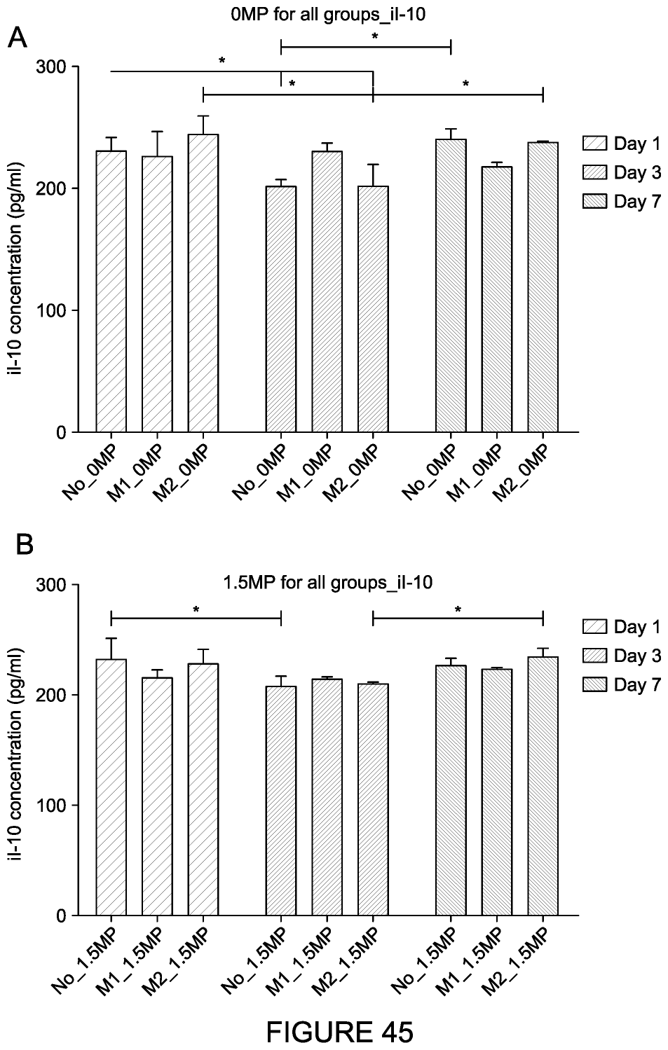

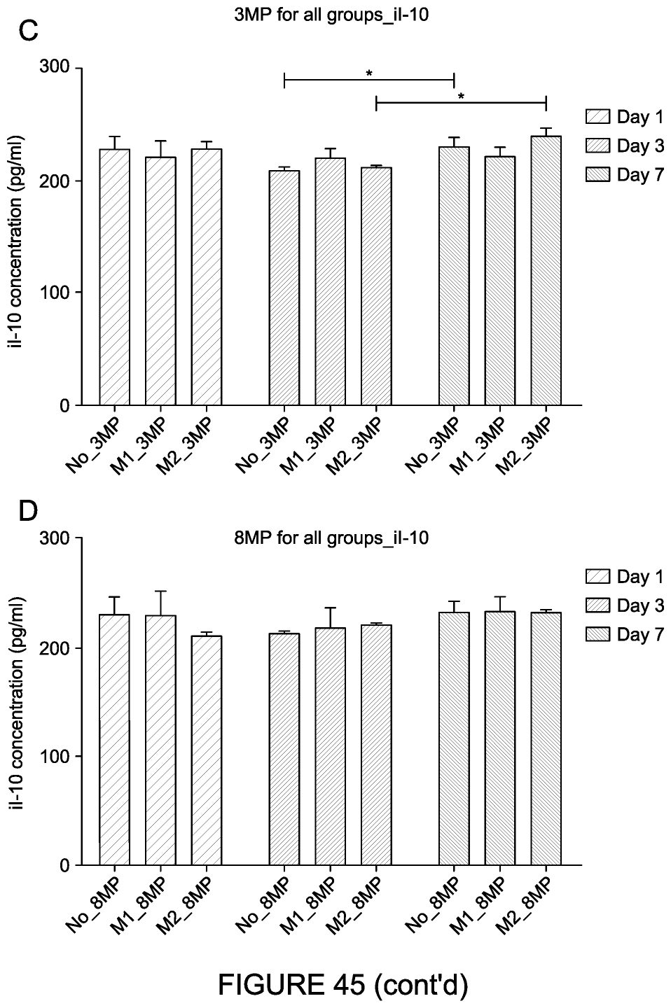

Additionally, after 24 hours of exposure to control (NB media) or activating (M1—20 ng/ml interferon gamma; M2—20 ng/mL interleukin-4)-conditions, cultures were then exposed to varying concentrations of MP. The MP concentrations in the media were decreased at subsequent feedings according to release data from previous experiments, while the activator concentrations were maintained. At three and five days after MP addition, the media was replaced; the removed was analyzed for presence and quantity of markers of M1 (TNF-alpha; FIG. 44, panels A-D) and M2 (interleukin-10; FIG. 45, panels A-D) activation. Cells were fixed on days 3, 5, and 7 for immunocytochemical analysis as showns in FIG. 42, panels A-J and 43, panels Ai-Cxii.

No Activation Group (Control)

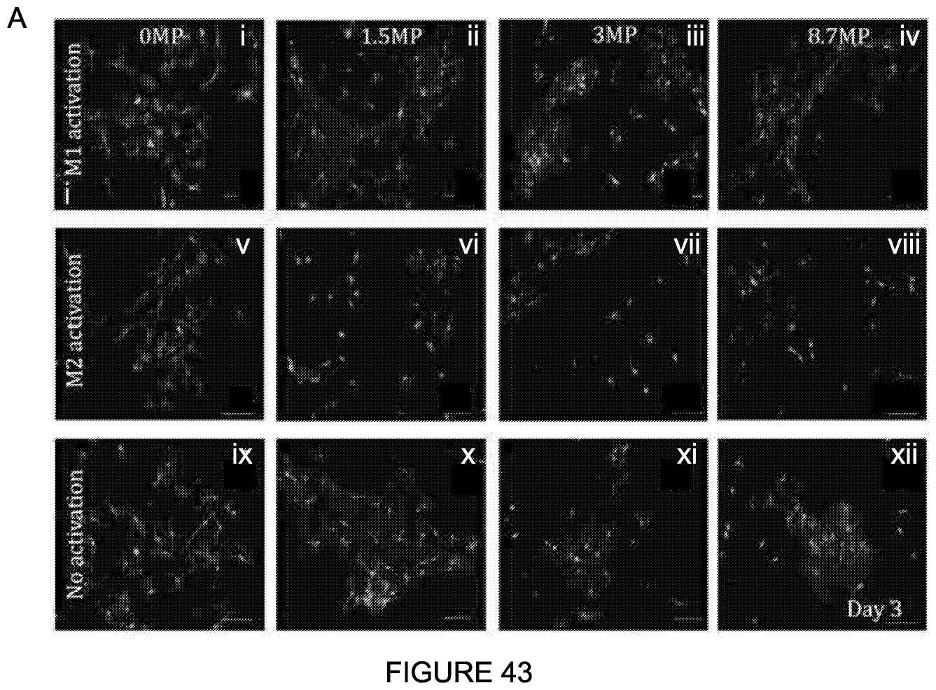

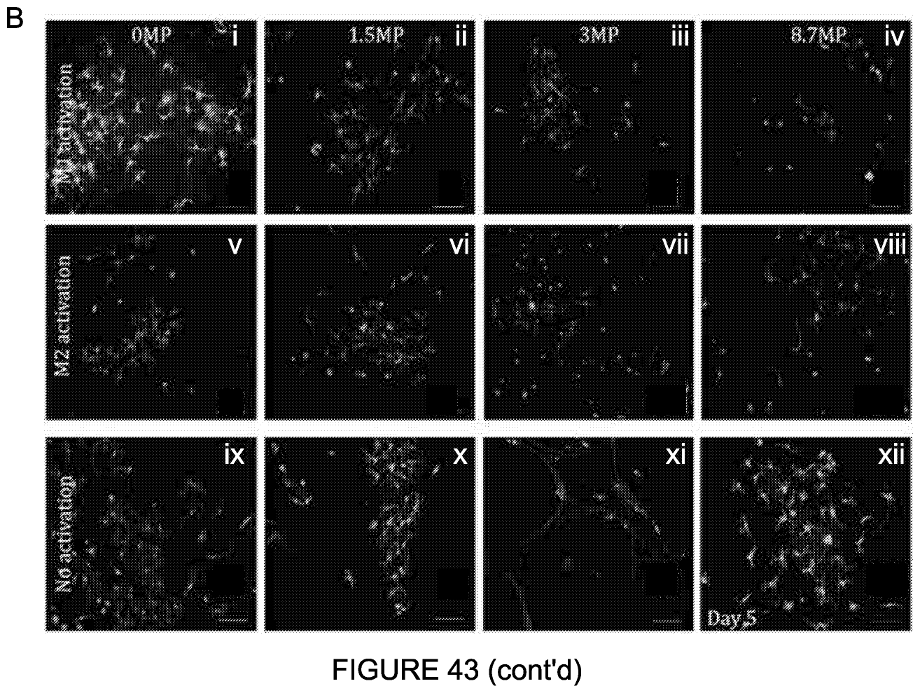

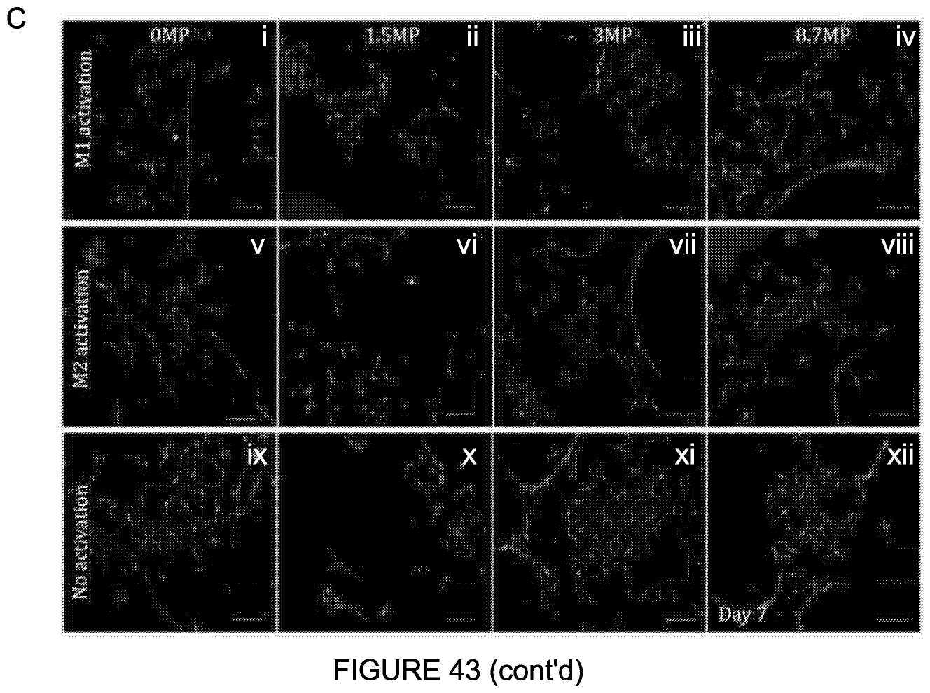

The no-activation, no-drug control group cells appeared have mostly ramifiedmorphologies at day 3 (FIG. 43, panels Ai-Axii), with some activation at day 5 (FIG. 43, panels Bi-Bxii), and a return to a more ramified morphology by day 7 (FIG. 43, panels Ci-Cxii). At day 5 the presence of MP appeared to maintain the cells in an intermediate phenotype, while the no-drug condition contained cells with both amoeboid and retracted phenotypes. By Day 7, the non-activated cells in all drug conditions appear primarily ramified.

M1 Activation

The M1-activated group without MP treatment showed an activated phenotype at Day 3. Upon the addition of MP the cells appeared to be reduced in terms of the number of amoeboid-shaped cells. At day 5 in the no drug group, there activation was still present, however cell morphology changed. There was a shift from primarily amoeboid cells to a more stellate shape, with large cell bodies and processes. In the 1.5 MP and 3 MP conditions, the cells exhibited a more ramified morphology, while in the 8.7 MP condition the majority of cells were round and retracted. At day 7, the cells returned to ramified shapes in both the control and drug groups. Cell death was observed in all conditions.

M2 Activation

At day 3, the M2-activated, no drug group showed a different morphology than both the M1- and no-activation groups. These cells were not amoeboid or stellate, but contained enlarged cell bodies and some projections. However, the presence of MP resulted in primarily small, retracted cells, with some amoeboid shapes. At day 5, the no-drug group continued to exhibit a similar morphology. Cells in the 1.5 MP condition appeared similar to the day 5, no-drug group. In both groups there were dead cells. In the 3 MP and 8 MP conditions there were a few cells showing similar morphology, but there are also a significant number of dead cells present. At day 7, cells in the no-drug group appeared more ramified. In the drug groups, the cells were primarily small and rounded, with some small processes. There appeared to be significant cell death in the drug-treated conditions as well.

To support the immunocytochemistry data, molecular analysis of secreted cytokines is crucial to support the selective activation of the immune cells. The effects of methylprednisolone on microglial morphology and cytokine production were examined. We discovered that methylprednisolone significantly downregulates pro-inflammatory cytokine production, without affecting proliferation potential of immune cells, which is supported by our findings that proliferation ability of M0, M1 and M2 polarized microglia (MG) and macrophages (RM) treated/non-treated with methylprednisolone (MP) was significantly different, as measured via DNA quantification assay. (FIG. 42, panels A-B). Further, cytokine production differed between M0, M1, and M2 polarized microglia and macrophages treated/non-treated with methylprednisolone. We also showed that methylprednisolone enhances the expression of anti-inflammatory markers in all groups, and significantly affects cell morphology using immunocytochemistry against the pan: ibal, CD11b; and polarization ill-beta and mannose receptor (MR) markers of microglia and macrophages at day 3 after administration of activation molecules, and Day 7 post-treatment with 8.7 mM methylprednisolone. We also confirmed the phenotype (judged by polarization), via RT-qPCR microglia and macrophages in M1 and M2 stages using CD11b (a pan-microglial/macrophagemarker; FIG. 42, panel H), CD86 (an M1 specific marker; FIG. 42, panel I) and CD163 (an M2 specific marker; FIG. 42, panel J). Gene expression was normalized to RSP18 gene, and the expression of the genes of interest was compared to the M0 non-activated control group.

### Example 11: 3D In Vitro Model of Primary Brain Tumor

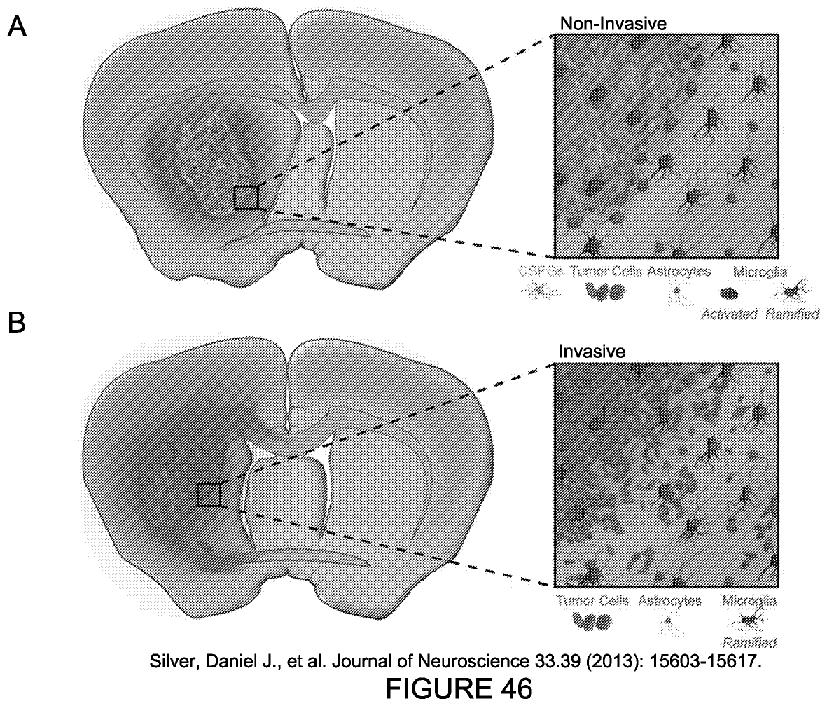

Tumor microenvironment/ECM in the brain, particularly proteoglycans, is known to play an important role in cell proliferation and migration. Many chondroitin sulfate proteoglycans (CSPGs) and heparin sulfate proteoglycans (HSPGs) are known to be upregulated in highly invasive and lethal brain tumors, such as glioblastoma; however, it has been shown that certain CSPGs might be largely absent from infiltrative type tumors or act as organizers of the microenvironment and prevent diffusive infiltration in non-invasive tumors (schematic FIG. 46).

To understand the role and content of ECM in brain tumor, primary tumor cells were cultured in a 3D in vitro model of brain that was enriched with native brain-derived ECM. The controlled modification of microenvironmental cues, such as CSPGs presented to the tumor cells both in the stromal and soluble forms, alongside the correlations between in vitro tumor models and original tumor tissue provide insight into the novel roles of these ECM molecules towards tumor progression and inhibition. This information may be used to guide appropriate targeting of ECM molecules as markers of tumor invasiveness and as a way to potentially slow down infiltrative tumor growth by modulation of these molecules. The present results already show improvement over previously known platforms for investigating brain tumor biology (e.g. 2D monolayer cultures, organotypic cultures, and animal models). Some challenges in such current models include, e.g., lack of in vitro 3D ECM environment, limited tunability for mechanistic studies, and cost/time intensity as well as low-throughput due to use of immunocompromised animal models, respectively. In addition to challenges with other platforms, previous tumor studies using 3D models predominantly focused on mechanical effects of matrix on the growth of established tumor cell lines for glioblastoma and medulloblastoma. Our studies expand and improve these models. Our in vitro 3D bioengineered silk brain model uses primary medulloblastoma tissue and results thus far suggest a microenvironment dependent tumor growth. CSPGs appear to be transiently upregulated and then downregulated in fetal/adult ECM conditions where healthy neuronal/astrocyte differentiation of hiNSCs was supported. This result is consistent with certain observations in the literature. However, consistently high levels of CSPGs were observed to be released in tumor cultures and pure collagen I constructs of hiNSCs, where reactive astrogliosis was observed through immunostaining. Without wishing to be held to a particular theory, we predict that future work with such provided compositions will show that fetal brain ECM, which is rich in chondroitin sulfate (CS), may impede or slow growth of certain brain tumor cells, because CS is known to inhibit invasion in non-aggressive tumor microenvironment. Until now, these studies were not possible, due to lack of proper models, including primary tumor cells.

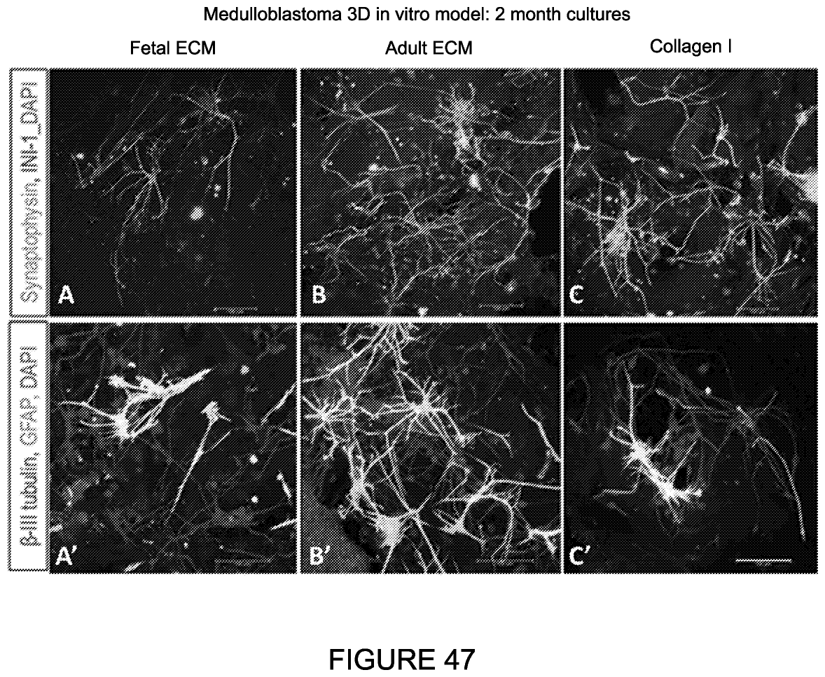

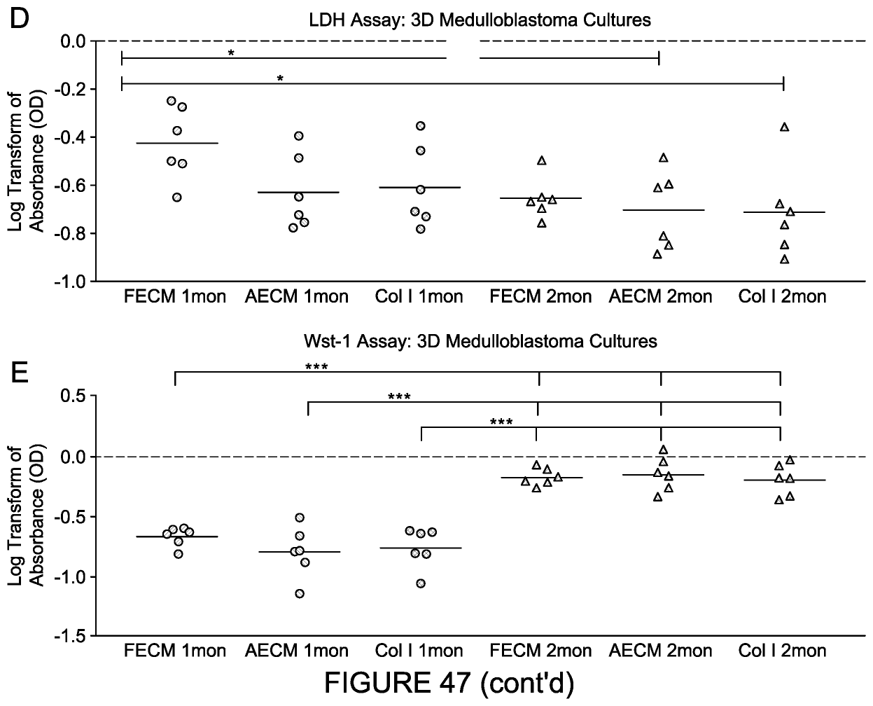

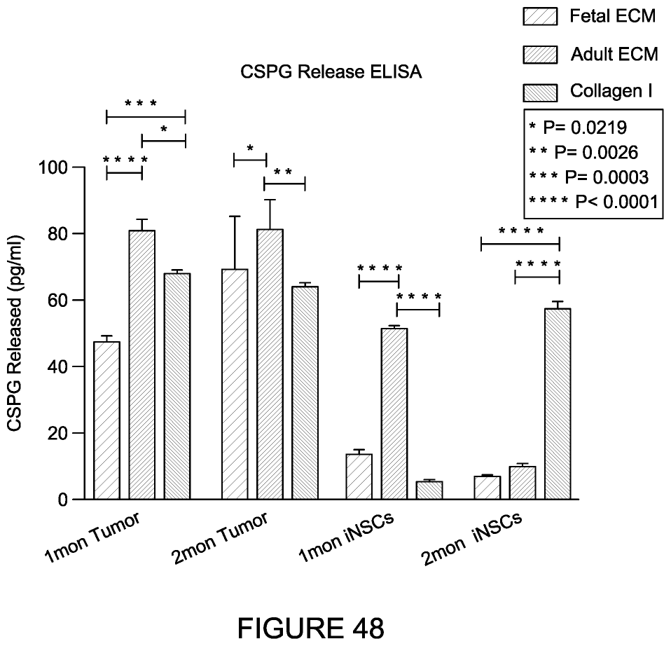

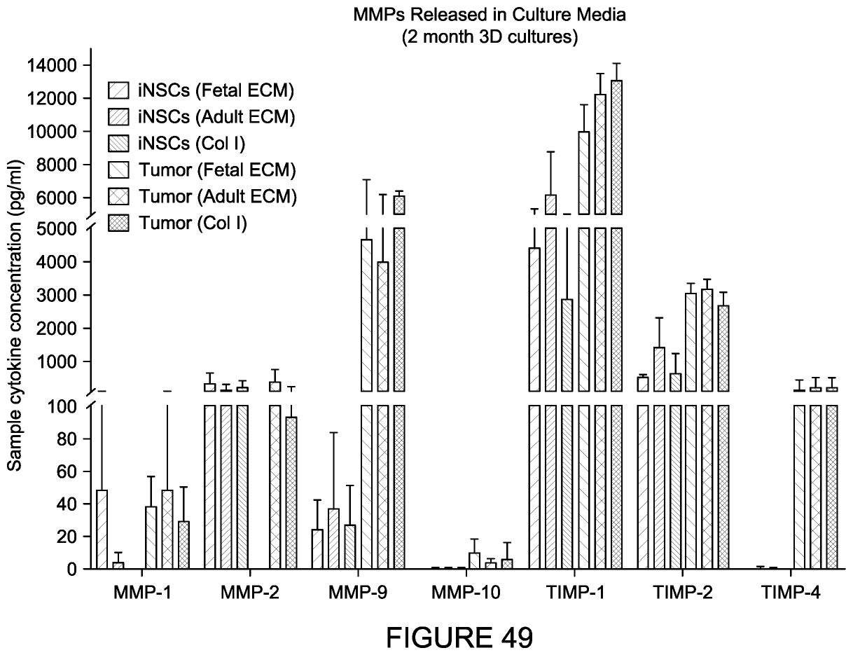

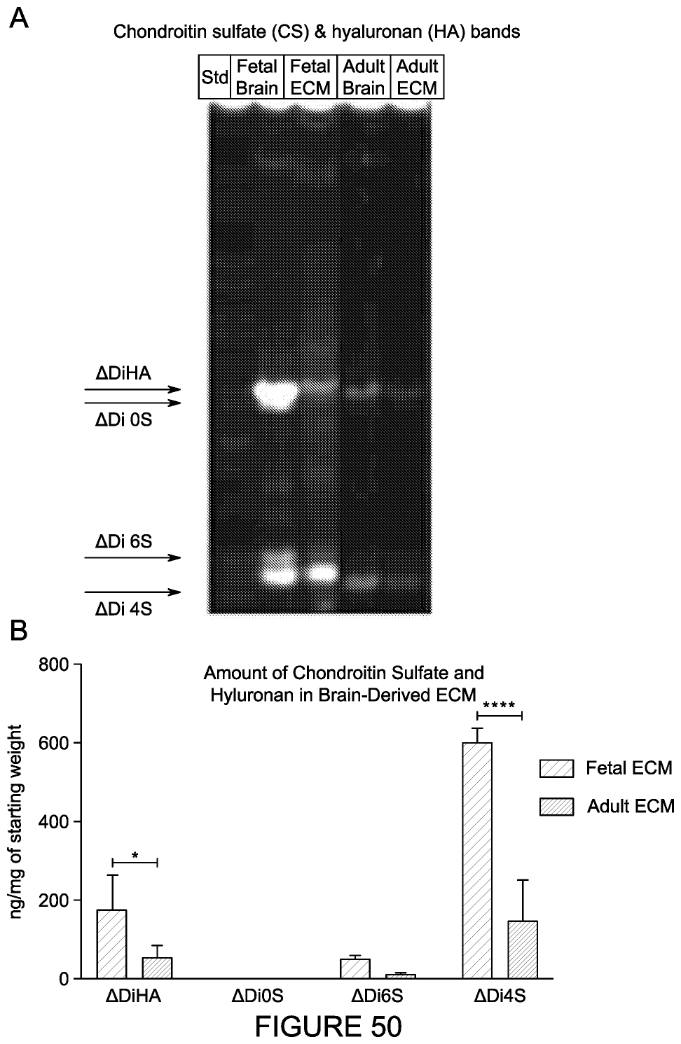

To create a 3D in vitro model of primary brain tumor and study the role of microenvironment on tumor growth, patient-excised medulloblastoma and ependymoma were cultured in the 3D brain model enriched with porcine brain-derived ECM. Viable medulloblastoma cells, as demonstrated by positive synaptophysin staining were maintained in all the 3D constructs for up to 2 months post seeding (FIG. 47, panels A-E). However, medulloblastoma growth seems to be hampered in the presence fetal brain ECM (FIG. 47, panels A and A′). Also, higher levels of lactate dehydrogenase (LDH) released in media indicated increased cytotoxicity of tumor cells in fetal ECM constructs, mainly during initial growth (FIG. 47, panels D and E). High levels of CSPGs (FIG. 48) and matrix remodeling molecules (MMPs; FIG. 49) were released in tumor cultures, indicative of increased remodeling of the microenvironment by the tumor cells, particularly in the adult ECM constructs (FIG. 47, panels B and B′). Although MMP 2 and 9 are overall upregulated in tumor microenvironment, MMP 2 was not released in fetal ECM constructs (FIG. 49). Similarly, high cytotoxicity levels in fetal ECM constructs are observed in the ongoing ependymoma cultures. We hypothesize that fetal brain ECM found to be rich in CS, through fluorescence assisted carbohydrate gel electrophoresis (FACE) (FIG. 50, panels A-B), could potentially be hampering the growth of tumor cells; CSPGs are known to inhibit invasion in non-aggressive tumor microenvironment and CS to hamper glioma spheroid invasion within composite matrices of collagen I and CS.

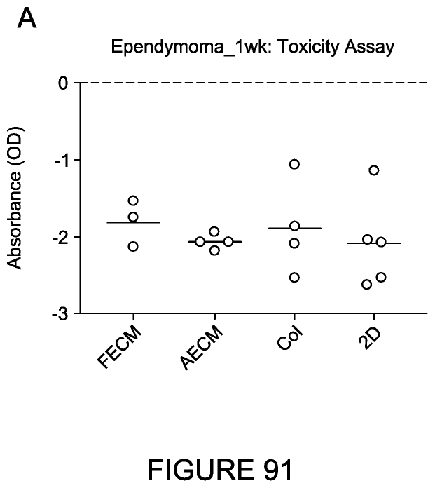

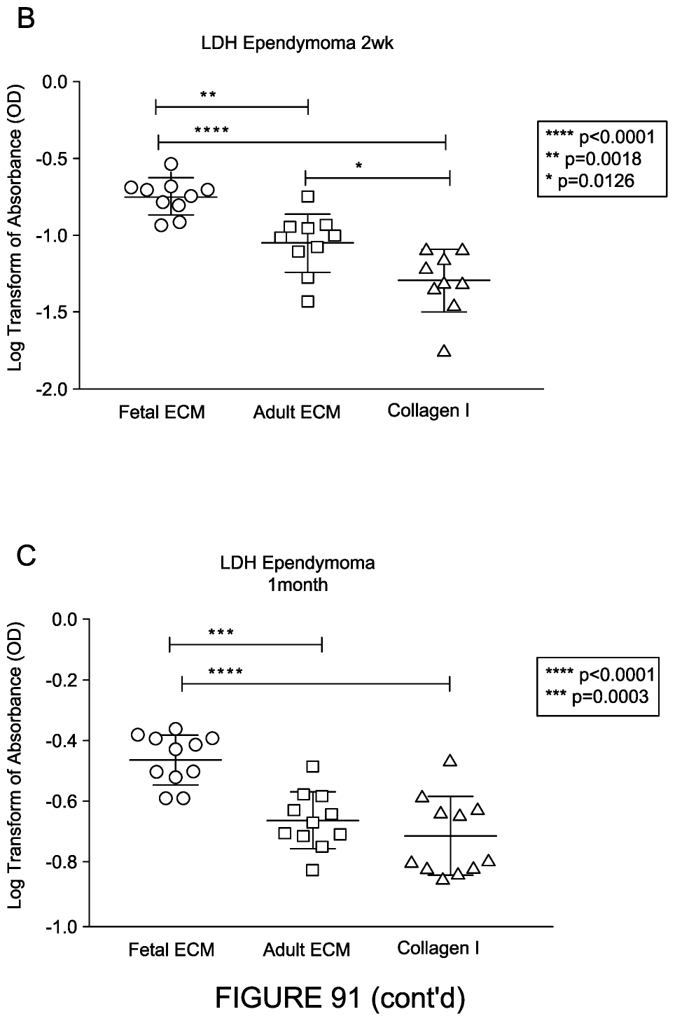

We also conducted preliminary metabolic analyses on anaplastic ependymoma cells. FIG. 91, panels A-D show a toxicity assay and LDH assays on Anaplastic Ependymoma cells cultured in Endothelial cell media (EGM 2MV) and Complete Neurobasal media (NBM). Toxicity data shows no differences between 3D cultures of fetal ECM (FECM), adult ECM (AECM) and Collagen (Col) in 3D cultures or any significant difference in toxicity as compared to 2D Cultures at very early time points (1 week cultures; FIG. 91, panel A). However, LDH assays after 2 weeks (FIG. 91, panel B) and 1 month (FIG. 91, panel C) in fetal ECM (FECM, green circles), adult ECM (AECM, blue squares) and Collagen (Col, red triangles), in 3D cultures showed significant differences between groups, according to the asterisks above the graphs.

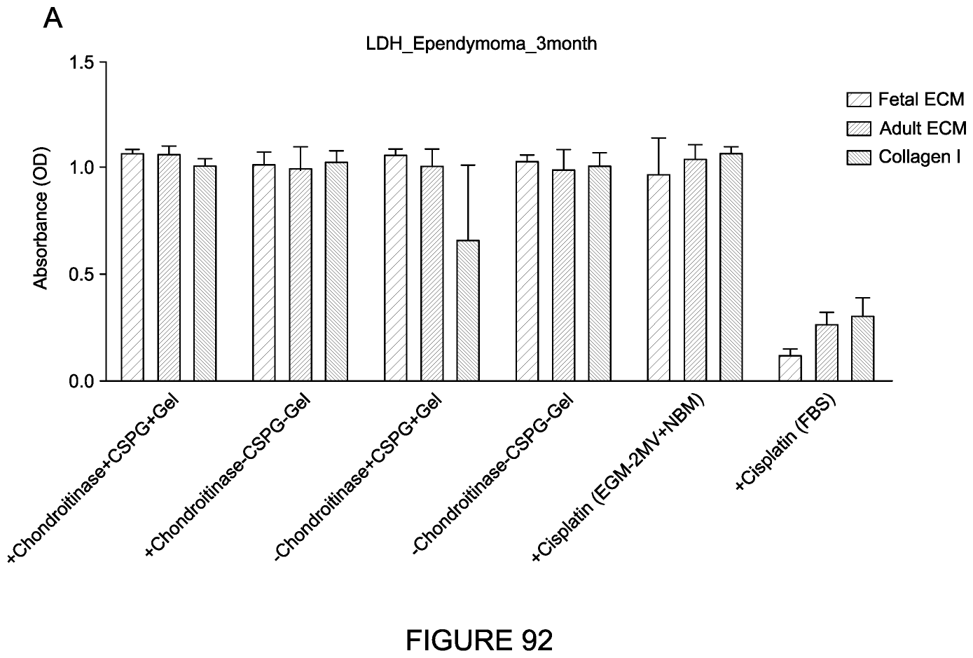

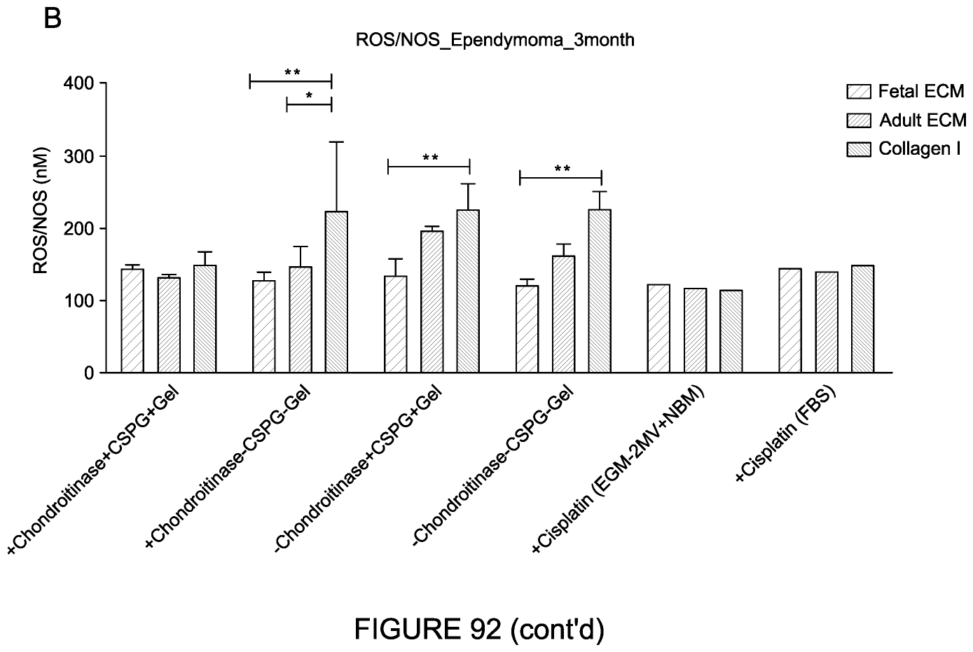

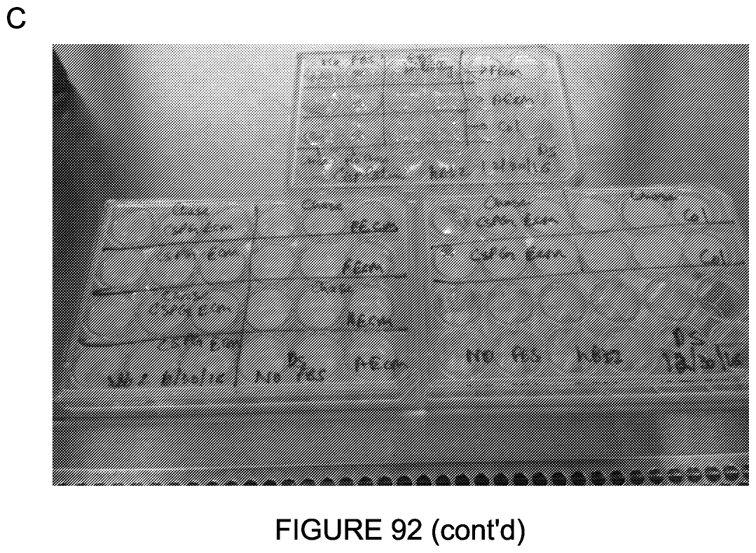

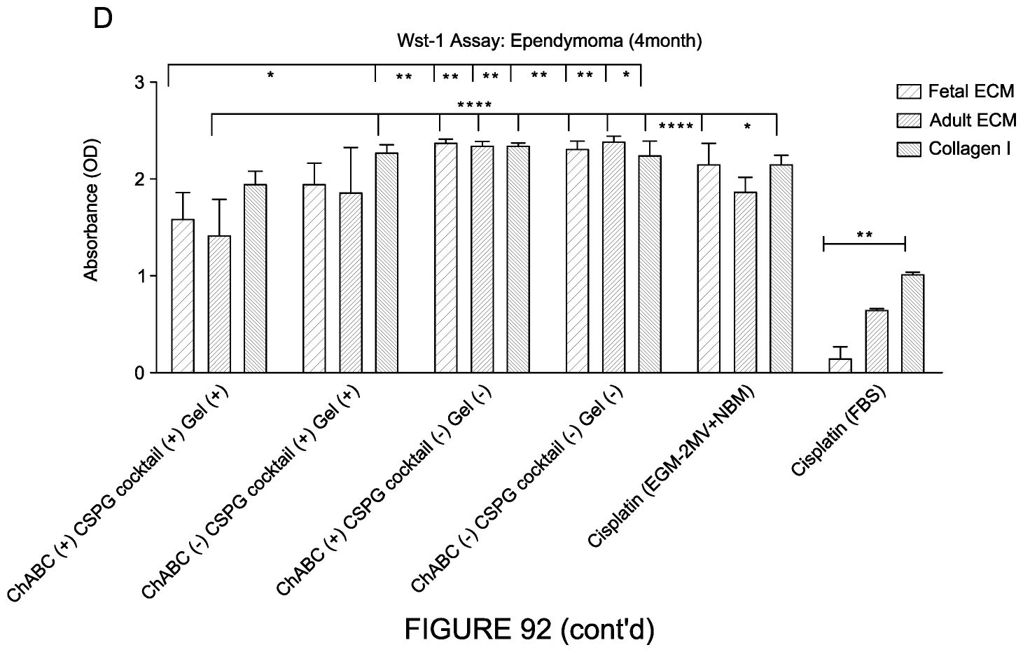

Metabolic measurements were taken from anaplastic ependymoma cells after three months in culture via LDH assay (FIG. 92, panel A), ROS/NOS assay (FIG. 92, panel B) and WST-1 assay (FIG. 92, panel D). Two different base media conditions were used: EGM-2MV+NBM (FBS free Endothelial cell media+complete neurobasal media) or FBS (10% FBS in DMEM) (FIG. 92, panel C). Ependymoma cells were overall able to better maintain their morphology and proliferative capacity in EGM-2MV+NBM media. Cells were cultured in FECM, AECM or Collagen, either with or without combinations of the enzyme chondroitinase ABC, soluble CSPG cocktail, and CSPG within a hydrogel added sequentially, or Cisplatin, a drug known for use in the treatment regime for ependymoma. Base medium was EGM-2MV+NBM, except in the FBS conditions. No significant differences were seen between conditions in LDH levels (FIG. 92, panel A), between or within conditions, except the constructs cultured in FBS media+cisplatin. In the ROS/NOS assay (FIG. 92, panel B), significant differences were observed within groups of several conditions, as noted by the asterisks above the bars on the graph in FIG. 92, panel B. Significant differences both within and between several groups were observed in a representative WST-1 assay, as shown by the asterisks and lines above the bar graph in FIG. 92, panel D.

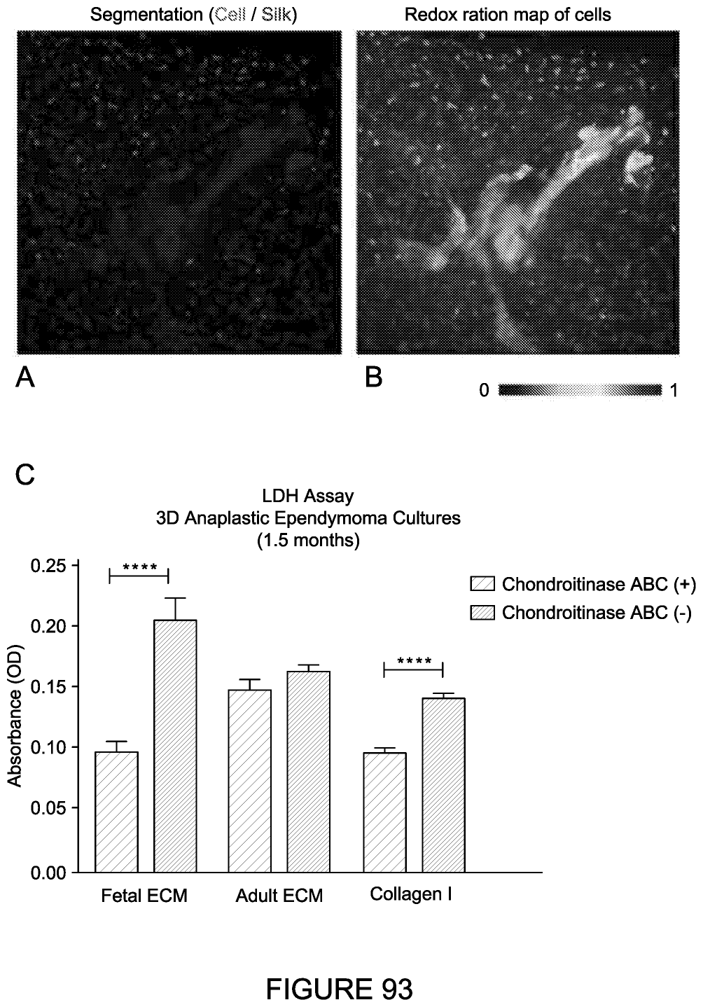

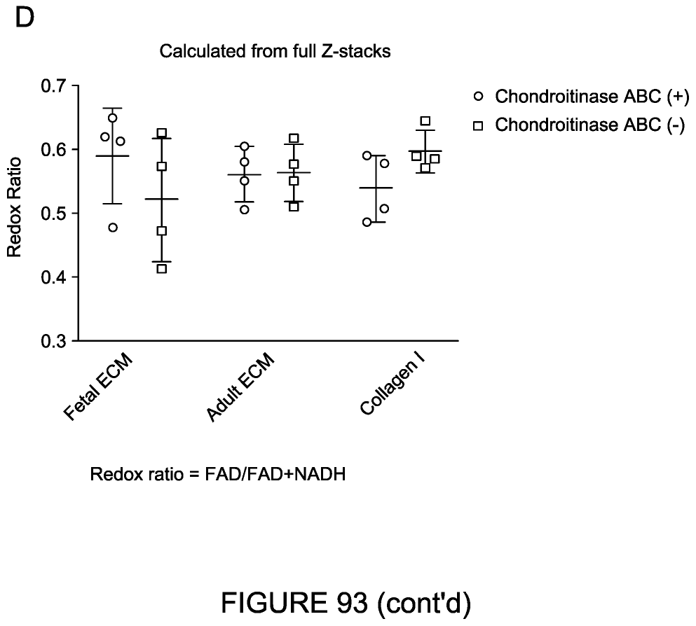

Metabolic imaging and analysis was also used to measure and determine redox ratios (FIG. 93, panels A-D). To conduct this analysis, cells and silk were separated (segmented), and marked by green or red, respectively, and cells were then color-coded using measured redox ratio values according to the scale shown below FIG. 93, panel B. FIG. 93, panel A shows segmentation with cells in green and silk in red and FIG. 93, panel B shows re-coloring to create a redox ratio map of the cells according to the colormetric scale below the image. FIG. 93, panels C and D show results of LDH assays (FIG. 93, panel C) and redox ratios (FIG. 93, panel D) following chondroitinase treatment. The redox ratio is determined as FAD/FAD+NADH. An increased redox ratio under normoxia is considered indicative of a higher metastatic potential in certain cancer cell lines as in Alhallak et al. (Alhallak, Kinan, et al. “Optical redox ratio identifies metastatic potential-dependent changes in breast cancer cell metabolism.” Biomedical Optics Express 7.11 (2016): 4364-4374).

### Research Methodology:

For continued studies, the required number of subject tumors (#4-6) were determined, taking into consideration inherent variability in tumor samples from individual patients, even when the tumor is of the same type/category. This potential heterogeneity/variability (of brain tumor samples) was also considered throughout the various steps undertaken to grow primary tumor cells in vitro. Variability may also exist in the 3D scaffolds prepared for tumor cell seeding or because of differential attachment of cells from different tumors within the scaffolds. For each patient-derived tumor tissue, the appropriate environment for long-term culture (2-4 months) of primary tumor cells will be/was identified through comparing porcine brain-derived extracellular matrix ad a single component collagen type I control matrix. The appropriate environmental conditions will be/were confirmed by comparison to patient histopathological report findings, such as markers for specific neuronal subtypes, reactive astrocytes, proliferation markers (e.g., KI67), tumor suppressors (e.g., p53). The effects of CSPGs on invasiveness of tumor cells, either as part of the ECM or in soluble form, will be compared by incorporating brain-derived ECM (containing CSPG constituents) and/or commercially available CSPG cocktails, within the developed 3D in vitro brain model, respectively. Specific roles of CSPGs in observed cellular phenotypes will be/were further determined by addition of an inactivation enzyme (e.g., chondroitinase ABC), to render ineffective any CSPGs present in the culture.

Primary study endpoints include representative tumor growth in vitro as compared to what would be expected in vivo by review of histopathological reports. These growth characteristics are assessed via, e.g., viability/toxicity assays, immunostaining/immunohistochemistry for tumor-related markers, levels of gene and protein expression for tumor specific genes as measured by PCR and western blotting, etc. Quantification of tumor-related markers, through genome profiling was used as an indicator for how closely an in vitro model can mimic in vivo tumor growth.

Secondary end points include results from ELISA-based microarrays for MMPs/TIMPs, growth factors and inflammatory cytokines (e.g., FGF2, EGF, TGF-β, etc.), and extracellular matrix (ECM) components (e.g., CSPGs) released into culture media; in order to determine the interaction of the tumor cells with their microenvironment. These results are correlated back to the primary endpoints.

If growth of primary glioblastoma tumor cells on salt-leached silk scaffolds is problematic, silk-HA freeze-dried scaffolds instead may be considered as a starting substrate for the primary tumor cells. HA is known to increase in tumor microenvironment and signal tumor cell migration and invasion by stimulating the secretion of MMPs.

If examination of overall levels of CSPGs across different patient brain tumors does not provide any clear trends due to inherent tumor heterogeneity, focus is placed only on GBM, where versican analysis may be specifically performed since it is a CSPG known to be highly upregulated in tumors. Immunohistochemistry against the different isoforms of the CSPG, versican is done using antibodies against specific isoforms of versican V0, V1 and V2.

### Example 12: Alternative In Vitro 3D Scaffold and Blood-Brain Barrier

To incorporate a blood-brain barrier (BBB) into our existing artificial brain tissue model, we introduced a channel into the brain model scaffold to model a blood vessel compartment. We have improved upon our earlier work by using hCMEC/D3 (brain endothelial cells), which are more physiologically relevant than HUVEC cells used in earlier work. We have also achieved better coverage by the cells (hCMEC/D3) on both the tube & salt leached sponge and the lyophilized channel sponge models. In addition, we have improved the design of our bioreactor and have succeeded in seeding astrocytes onto our scaffolds.

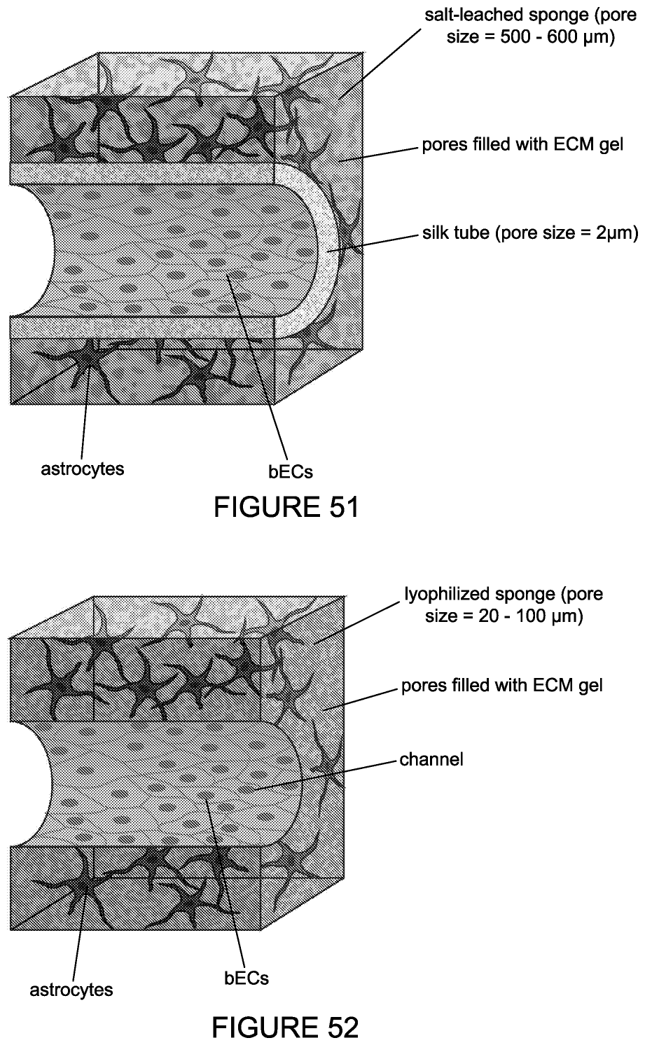

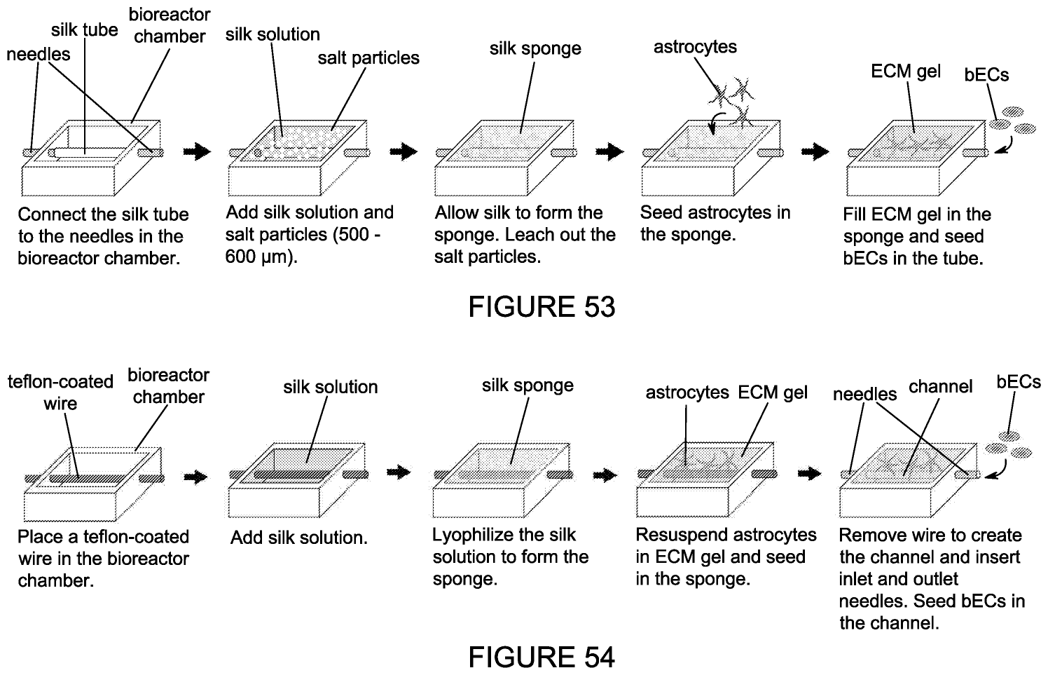

The two scaffold designs we investigated were 1) tube & salt-leached sponge (FIG. 51) and 2) lyophilized channel sponge (FIG. 52). We fabricated the scaffolds and seeded an immortalized human cerebral microvascular endothelial cell line (hCMEC/; “bECs”) in the luminal wall. Astrocytes have been shown to promote the barrier phenotype. In our model, astrocytes were seeded in the bulk space in the scaffolds. In the tube & salt-leached sponge design, a silk tube was inserted into the scaffold to provide a smooth 3D surface for bECs to adhere, mimicking brain microvasculature. The sponge provides support for 3D culture of astrocytes, to mimicking the extravascular brain tissue environment. The pores in the tube allow soluble factors and small cell processes to pass. In the lyophilized channel sponge design, the luminal surface of the channel was filled with ECM gels to provide a smooth 3D surface for bECs to adhere, mimicking the brain microvasculature. The sponge provides support for 3D culture of the astrocytes, mimicking the extravascular brain tissue. The pores in this design allow direct bEC-to-neural cell (astrocytes and neurons) contact.

### Methods

Brain Endothelial Cells and Astrocytes

The immortalized hCMEC/D3 cell line was purchased from Millipore, and the cells were cultured in EndoGroMV media supplemented with bFGF (1 ng/mL) according to the supplier's protocol. hCMEC/D3 cells were used as the model bECs. Primary human astrocytes were purchased from ScienCell Laboratories and were cultured in Astrocyte Growth Media according to the supplier's protocol.

Fabrication of Tube & Salt-Leached Sponge Scaffolds

To fabricate the tube & salt-leached sponge scaffold (FIG. 54), silk fibroin solution was concentrated to 18-20%. 5% PEO solution was mixed with silk fibroin solution at 1:10 ratio. Fibronectin was added to the mixture at a final concentration of 50 μg/mL. To make tubes, a 25G (diameter=500 μm) needle was coated with the silk/PEO/fibronectin solution by manual dipping, and the needle was immersed in 100% methanol to induce beta-sheet formation of the silk. This process was repeated 2-3 times to improve the strength of the tube. The tube was removed from the needle. Two blunt needles were inserted in the bioreactor chamber, and the silk tube was connected to the needles within the bioreactor chamber. 600 μL 6% silk solution followed by 1.2 g of salt particles (500-600 μm) was added to the chamber. The silk was allowed to dehydrate at room temperature for 48 hrs followed by 60° C. for 1 hr. Salt particles were washed out with water. Tubes in the tube & salt-leached sponges were coated with col I (150 μg/mL) prior to cell seeding. To fabricate the lyophilized channel sponge model (FIG. 54), the luminal surface of the channel was filled with ECM gels to provide a smooth 3D surface for bECs to adhere, mimicking the brain microvasculature. The sponge provides support for 3D culture of the astrocytes, mimicking the extravascular brain tissue. The pores in this design allow direct cell-to-cell contact. Lyophilized sponge were filled with col I gel (2 mg/mL).

For both models bECs were seeded in the luminal wall of the tube or the channel, respectively. For tube & salt-leached sponges, astrocytes were seeded directly into the the sponge. For lyophilized channel sponges, astrocytes were first suspended in the collagen solution and seeded in the sponge with collagen.

### Results

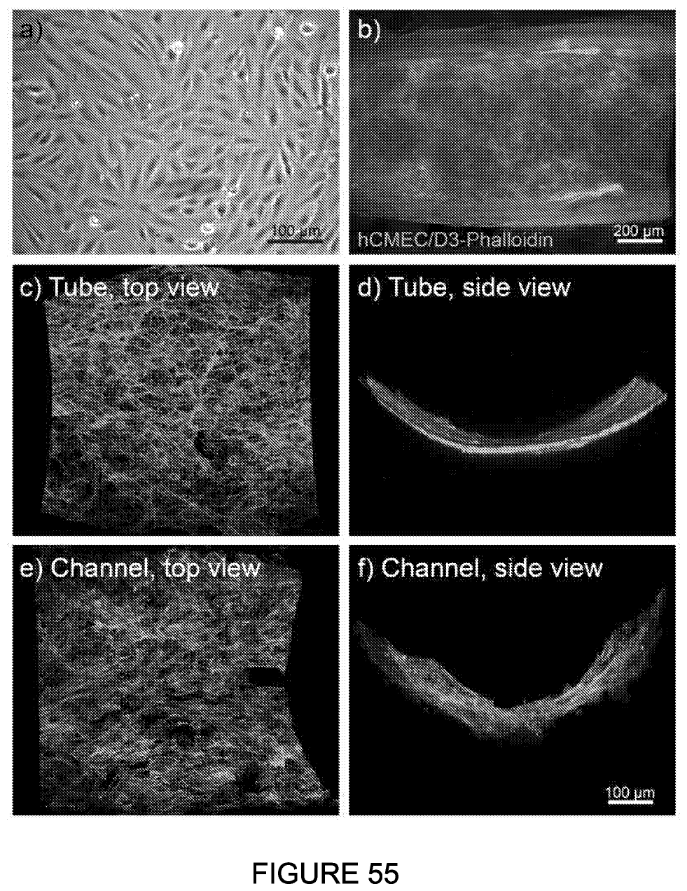

hCMEC/D3, an immortalized bECs, adhered to and formed a monolayer on the luminal wall of the silk tubes and channel (FIG. 55, panels A-D). The scaffolds were cut open and cells were stained with phalloidin (FIG. 55). hCMEC/D3 cells adhered to silk tubes (FIG. 55, panel B). FIG. 55, panels C-D show confocal images of phalloidin-stained hCMEC/D3 cells in the tube in salt-leached sponges and FIG. 55, panels E-F show hCMEC/D3 cells in the channel of lyophilized sponges. These results support that using this cell line is feasible for the improved 3D BBB model that we will continue developing and characterizing.

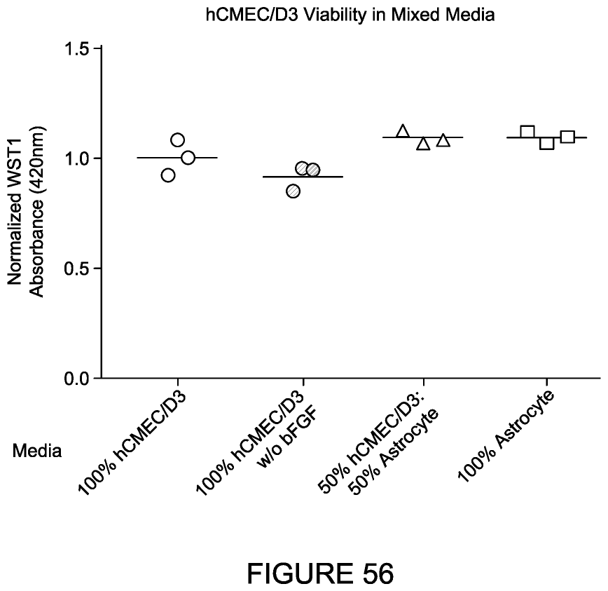

FIG. 56 shows that hCMEC/D3 cells survived in (50%:50% hCMEC/D3 media to astrocyte media and 100% astrocyte media). hCMEC/D3 media was EndoGroMV Media with bFGF (Millipore), and astrocyte media was Astrocyte Growth Media (ScienCell Laboratories). To screen media for use in hCMEC/D3-astrocyte co-cultures, hCMEC/D3 cell viability was first assessed using a WST1 assay when cultured in different combination of media for 7 days in 2D in 96 well plates: 100% hCMEC/D3 media, 100% hCMEC/D3 media without bFGF, 50%; 50% mix of hCMEC/D3 and astrocyte media, and 100% astrocyte media. hCMEC/D3 cell viability was not signifiantly different when cultured in 50% or 100% astrocyte media, or in hCMEC/D3 cell media without bFGF supplementation.

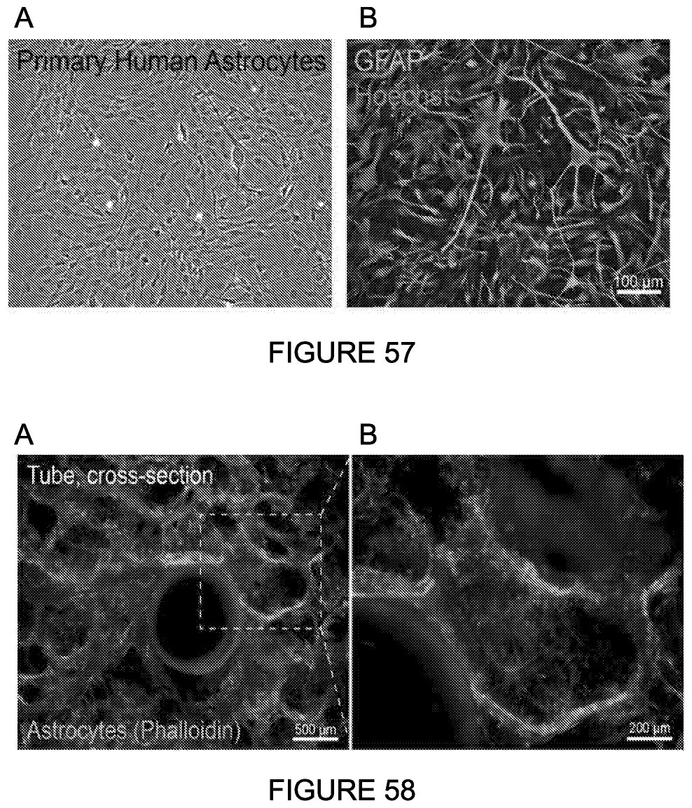

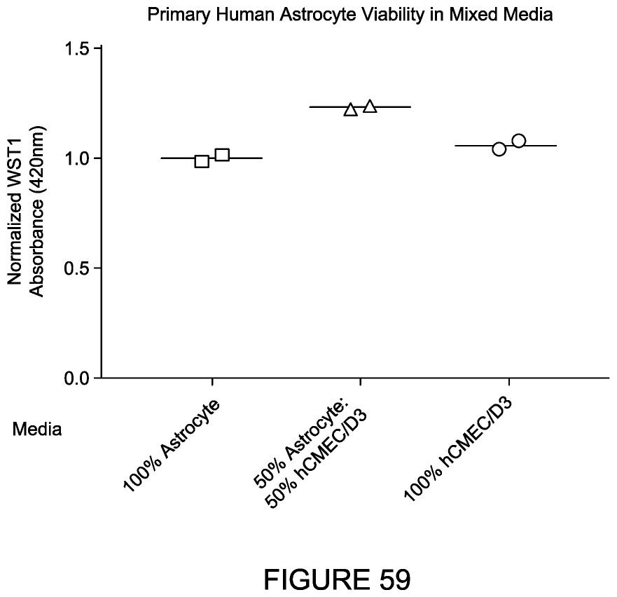

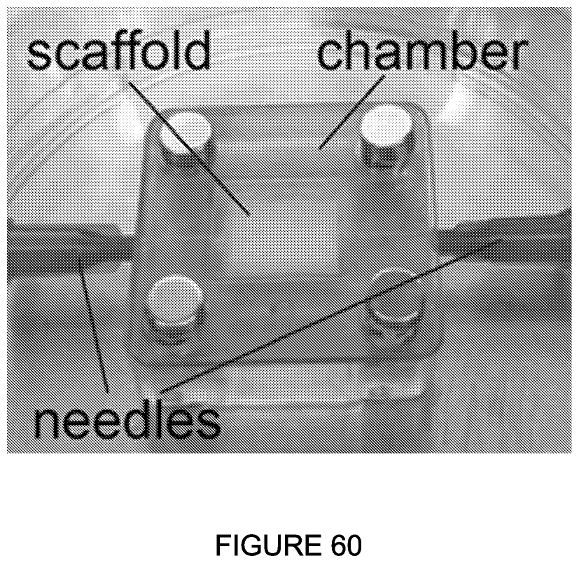

Since the model aims to replicate the BBB and include astrocytes, primary human astrocytes were purchased from ScienCell Laboratories and cultured in Astrocyte Growth Media and passaged according to the supplier's protocol. No astrocytes were used in this study/model that were older than passage 5. Cells were fixed with 4% paraformaldehyde, immunostained for GFAP, and imaged. Cells were visualized to determine relative GFAP expression within the population and FIG. 57 confirms that a majority of these cultured astrocytes expressed GFAP. Furthermore, FIG. 59 demonstrates that primary human astrocytes survived in co-culture media. As with hCMEC/D3 cells, above, to screen media for hCMEC/D3-astrocyte co-cultures, astrocyte viability was first assessed using a WST1 assay when cultured in different combination of media for 7 days in 2D in 96 well plates: 100% astrocyte media (Astrocyte Growth Media, ScienCell Laboratories), 100% hCMEC/D3 media (EngoGroMV Media with bFGF, Millipore), and 50%:50% mix of astrocyte media and hCMEC/D3 media. Astrocyte viability was not significantly different when cultured in 50% and 100% hCMEC/D3 media.

Cultured astrocytes were also seeded onto scaffolds with salt-leached sponges. The sponges were pre-coated with poly-L-lysine (50 μg/mL). The sponges were filled with col I gel (3 mg/mL) 2 days after astrocyte seeding. FIG. 58, panels A and B showfluorescent images of phalloidin-stained astrocytes, demonstrating that astrocytes adhered to and spread into the bulk space. This spreading supports that cultured primary human astrocytes are feasible for use in our 3D BBB models. Finally, in our models, we demonstrated that flow can be introduced to the cultured hCMEC/D3 endothelial cells using a bioreactor. Scaffolds of both designs were fabricated in a PDMS bioreactor chamber, and lids were secured with magnets. hCMEC/D3s were injected in the tube or channel and were allowed to adhere to the luminal wall for at least 24 hrs. The bioreactor was connected to a peristaltic pump with hCMEC/D3 media at a flow rate of 400 μL per min for a shear stress of 4 dyne/cm², and the cells were exposed to physiologically relevant shear stress.

### Example 13: Cortex & Cerebellar Interface Model

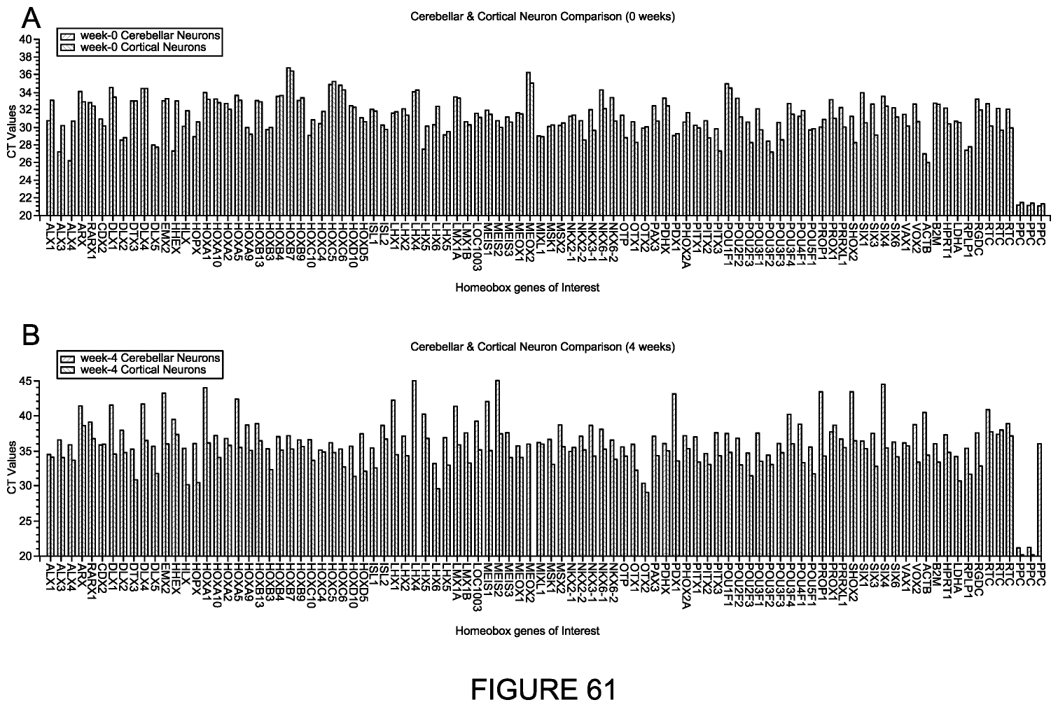

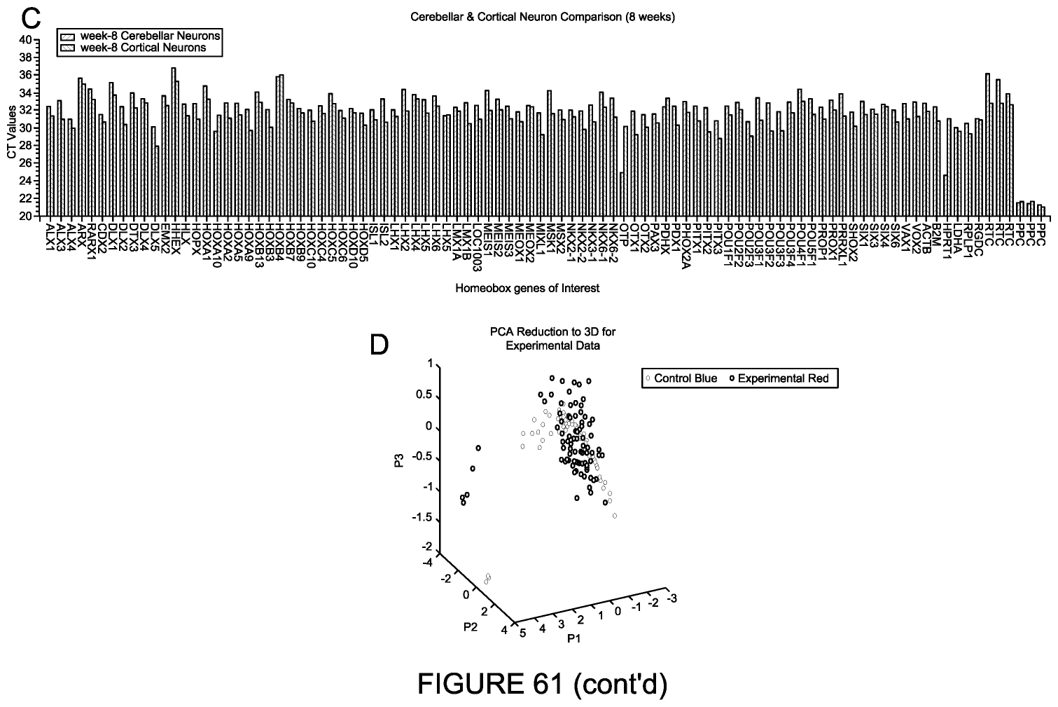

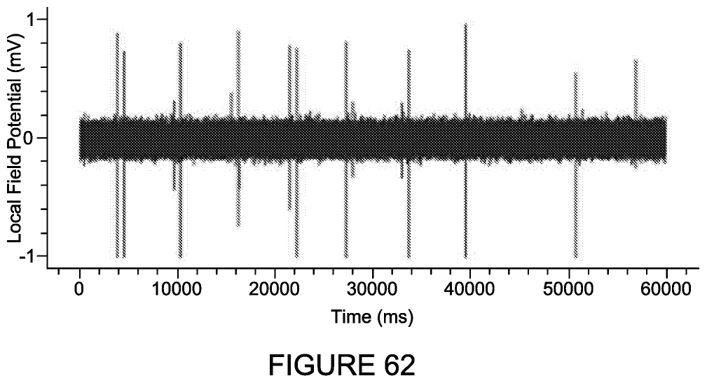

The cerebellum comprises the ‘old brain’ whereas the ‘cortex’ comprises the new brain. Numerous musculoskeletal and abnormalities of motor control (i.e., dyskinesia or involuntary movements) emerge from the interaction between these separate regions of the brain. However, it is impossible to discern the structure-function relationship (i.e., neural connectivity & activity) that makes these regions unique without first addressing morphological differences. This poses a developmental question regarding neural lineage by which the silk scaffolds can be used to address the role of surface topology on cell-fate. Perhaps the most immediate genes implicated in development include the ‘homeobox’ or HOX genes which immediately influence tissue development and structural morphology. However, it stands to mention that ECM components additionally contribute to cell lineage which may thereby confound any conclusions drawn regarding surface topography alone. To this end, we conducted a comparison of the existing cortical and cerebellar models in regards to surface topology with HOX genes as a lingeage specific, functional basis for variance. Furthermore, in order to more succinctly depict these comparisons over time multi-variate analysis was used to represent gene markers in a 3D space. As can be seen in FIG. 61, panels A-D, cortical or cerebellar rat neurons were cultured for 0, 4, and 8 weeks. Several homeobox genes were assayed for expression levels in rat cortex and cerebellum and any changes that occurred during this period of time. Cerebellar neurons were also assayed for electrical activity via measurement of local field potentials. To measure local field potentials, an electrode solution of KCl was placed into a pulled capillary electrode and the scaffold was submerged within a osmotic bath solution. Spontaneous action potentials were recorded. FIG. 62 demonstrates that cultured cerebellar neurons show evidence of spontaneous neural network depolarization.

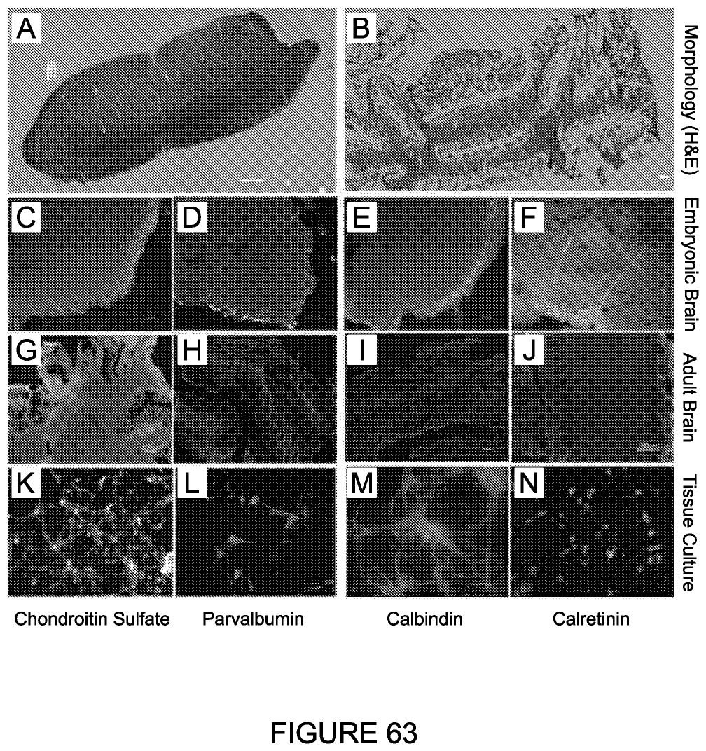

Phenotypic analyses of cerebellar cells was performed. FIG. 63, panels A-N shows H&E Staining and Immunostaining of the cerebellum. Unlike the cortex the cerebellum exhibits stratified layers to within its architecture. Over time the embryonic cerebellum (FIG. 63, panel A) becomes increasingly complex in its morphology during the progression to adulthood (FIG. 63, panel B). FIG. 63, panels C-J demonstrate that gross anatomical comparison of fetal (FIG. 63, panels C-F) and adult (FIG. 63, panels G-J) cerebellum reveals this stratification is also observed in components of the ECM.

Embryonic Day-14 rat cerebellum was surgically removed from a pregnant Sprague-Dawley rat. The meningeal layers were removed and the remaining portions of the brain were divided into quarters and digested with Accutase—enzyme detachment media. Cerebellar neurons were also cultured on glass slides and immunophenotyped using cerebellar neuron markers of the ECM to validate primary antibodies and substantiate presence of the markers contrasting the embryonic and fetal brain against those in cell culture. FIG. 63, panels K-N show that these cultured cerebellar neurons, as well as tissue slices of cerebellum (Panels C-J), identified by presence of beta-III tubulin (green), also co-express appropriate phenotypic markers including chondroitin sulfate (FIG. 63, panels C, G, and K), parvalbumin (FIG. 63, panels D, H, and L), calbindin (FIG. 63, panels E, I, and N), and calretinin (FIG. 63, panels F, J, and N) (all labeled with a secondary antibody to Texas Red; cell nuclei are identified using DAPI, blue).

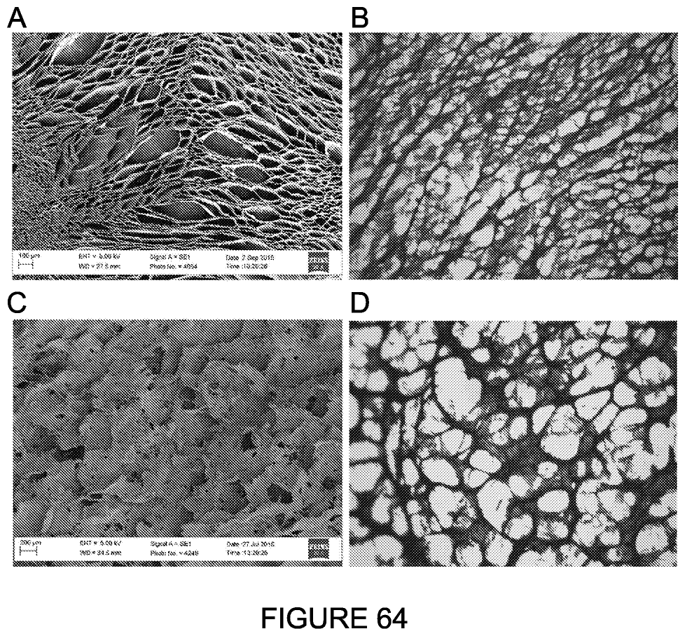

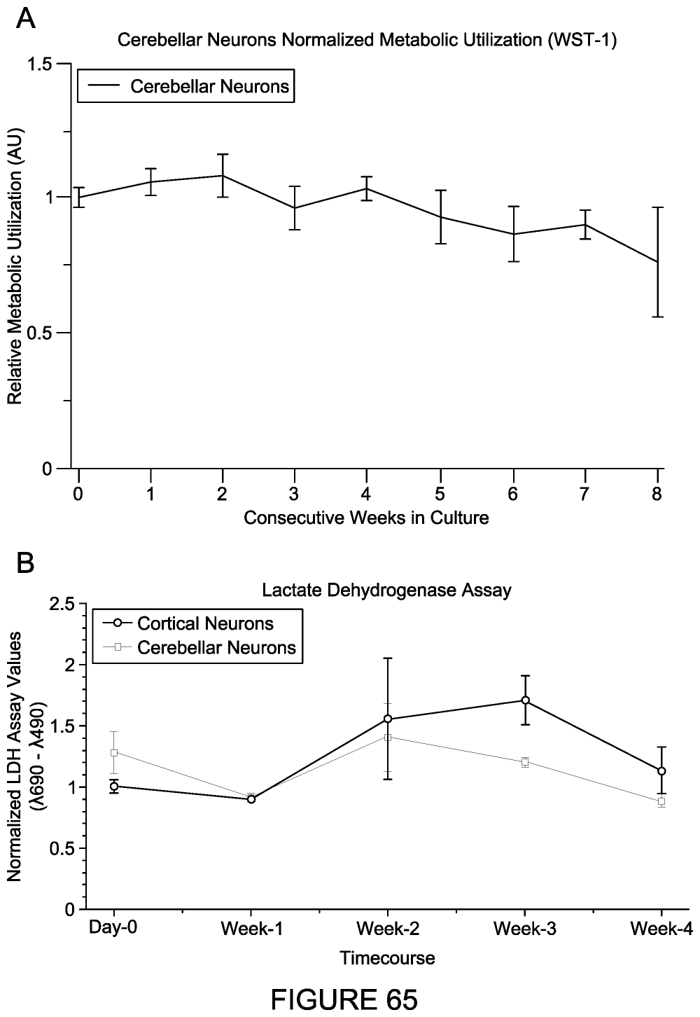

Scaffolds were evaluated using scanning electron microscopy (“SEM”) and cortical slices were evaluated using hematoxylin and eosin (“H&E”) staining, to compare morphology of silk sponges with that of cortex. FIG. 64, panels A and C show SEM of a traditional silk sponge and H&E staining of adult (FIG. 64, panel B) and embryonic (FIG. 64, panel D) cortices. The striated banding patterns observed in FIG. 64, panels B and D are representative of the histological patterns invagination of the surface of cerebellar tissue which thereby serves to increase the surface area to volume ratio (SA/V) of the adult brain as compared to the embryonic. Increase or decrease in the SA/V ratio thereby influences the degree of connectivity that can be achieved by altering the topology and influencing the proximity of individual cells. These figures demonstrate a resemblance between native tissue structure and that of the 3D silk scaffold, which supports that the silk scaffolds mimic in vivo brain structure/architecture. Cerebellar neurons were also cultured long-term (8 weeks) and evaluated for metabolic utilization using Wst-1 assays (FIG. 65, panel A) and LDH assays (FIG. 65, panel B). These assays demonstrated that cerebellar neuron scaffolds remained active for a duration of 8 weeks in culture. Our data support that cerebellar neurons have a comparable metabolic rate to cortical neurons.

### Example 14: Alternative Parkinson's Disease Model

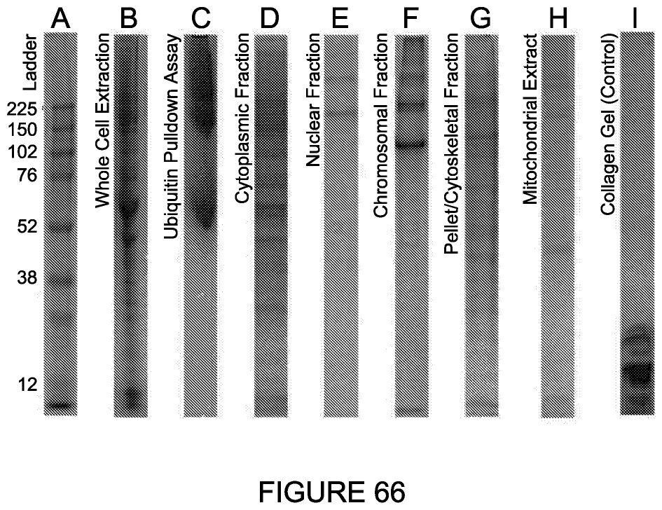

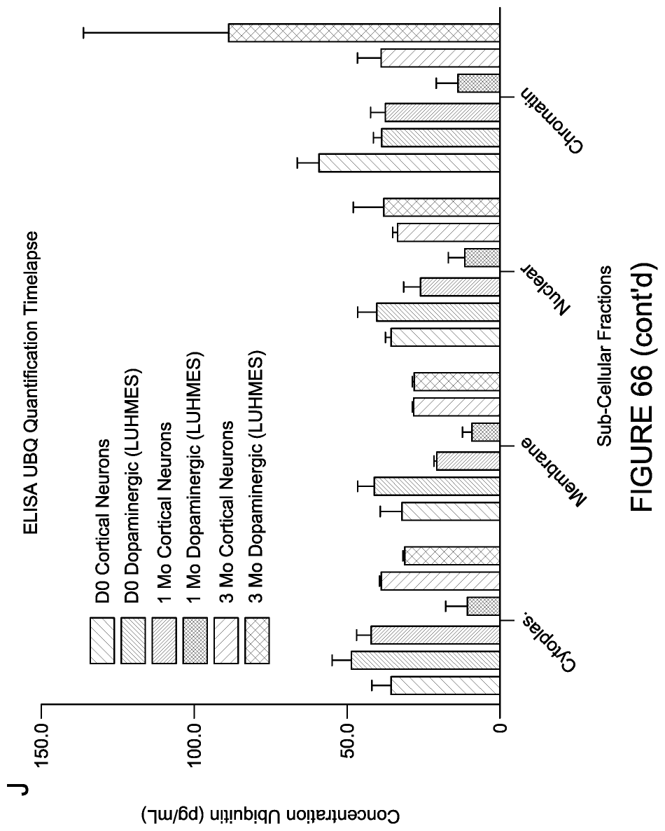

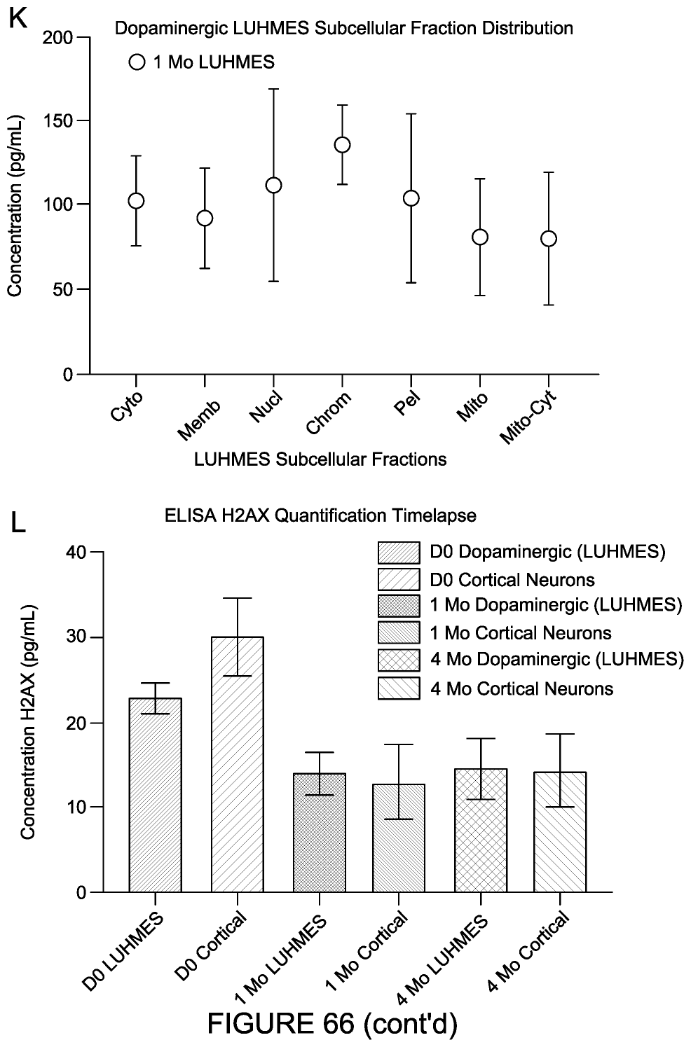

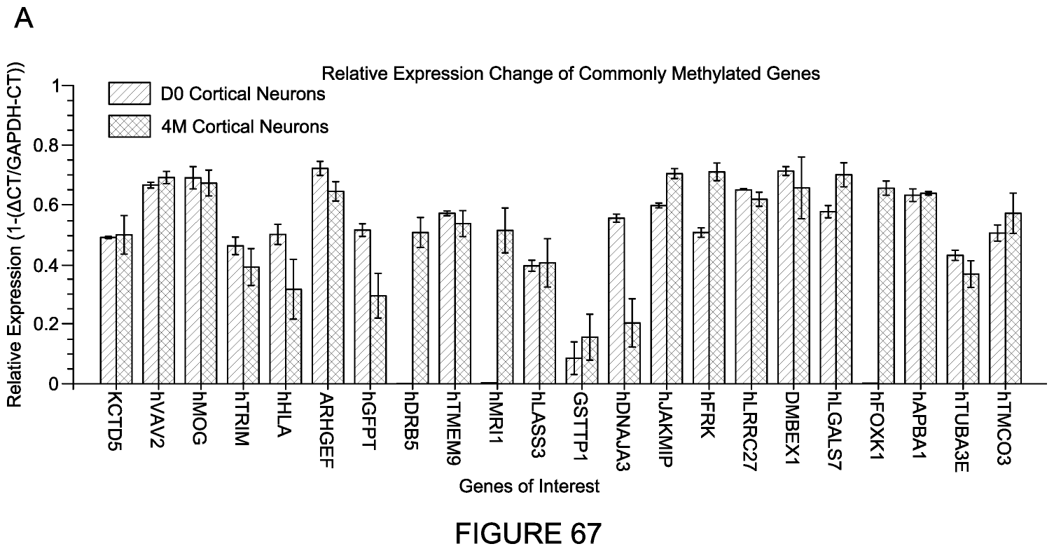

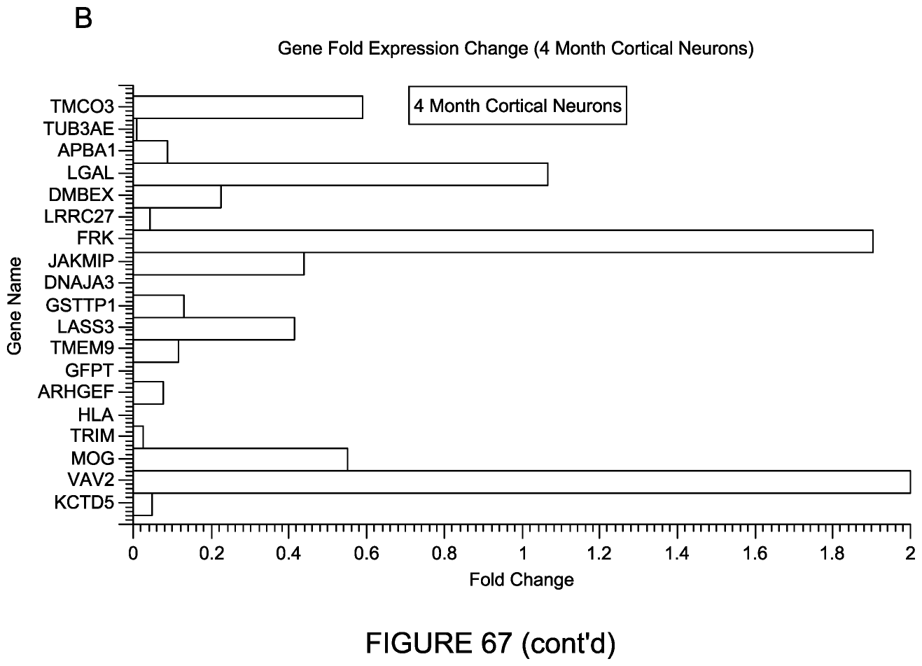

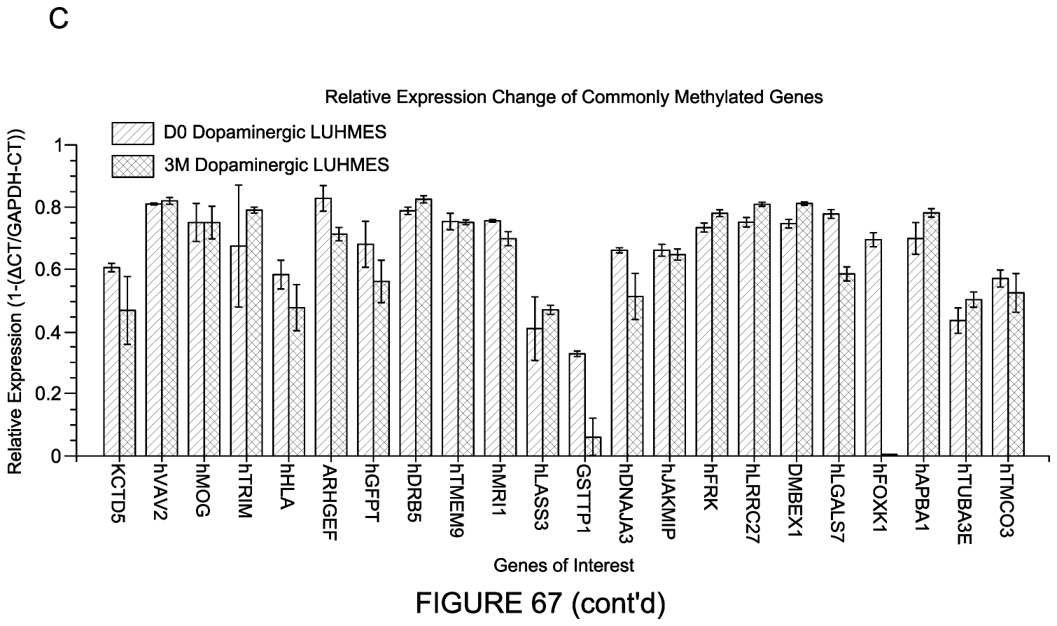

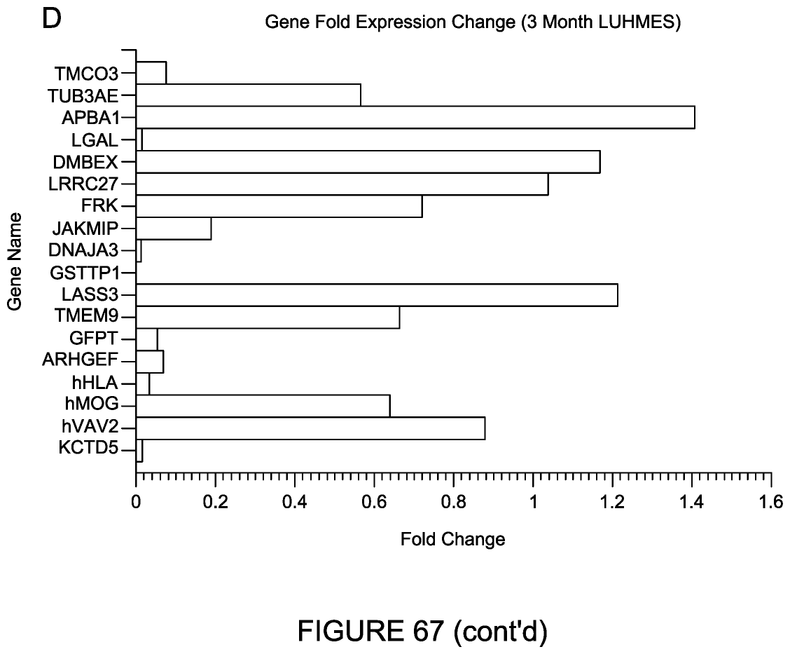

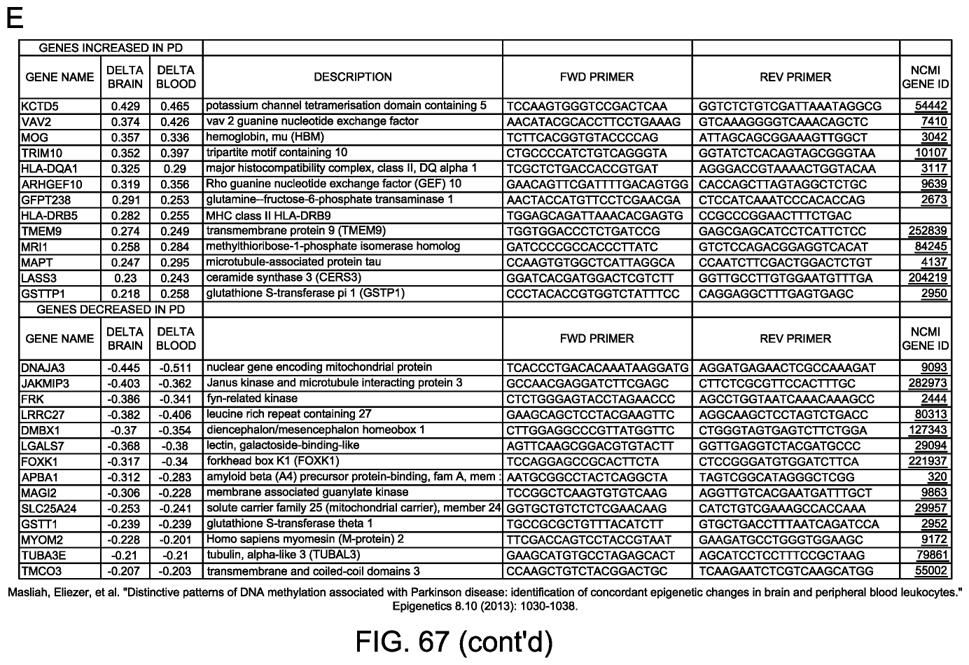

In this Example, proteomic analyses were conducted on LUHMES doparminergic neurons. Subcellular fractions (e.g. cytoplasm, nuclear, cytoskeletal, mitochondrial, etc.) were isolated from LUHMES cells that had been cultured for one or three months. Whole cell lysate, a ubiquitin pulldown assay, and subcellular fractions were all probed to demonstrate molecular weight of proteins within the fractions. Analysis of subcellular fractions (FIG. 66, panels A-K) showed a full breadth of protein fractions, however, the majority are heavier than the 52 kDA marker shown in the ladder (FIG. 66, panel A). Distribution of subcellular fractions in 1-month LUHMES cells is seen in FIG. 66, panel K. Mitochondrial fractions will be used to further explore protein turnover. ELISA assays (FIG. 66, panel J) were conducted on subcellular fractions of 0-day and 3-month LUHMES cells and cortical neurons. ELISAs were run to compare unbiquitin concentrations in several fractions (FIG. 66, panel J) and H2AX levels in LUHMES and cortical neurons at day 0, 1, and 4-month timepoints (FIG. 66, panel L).

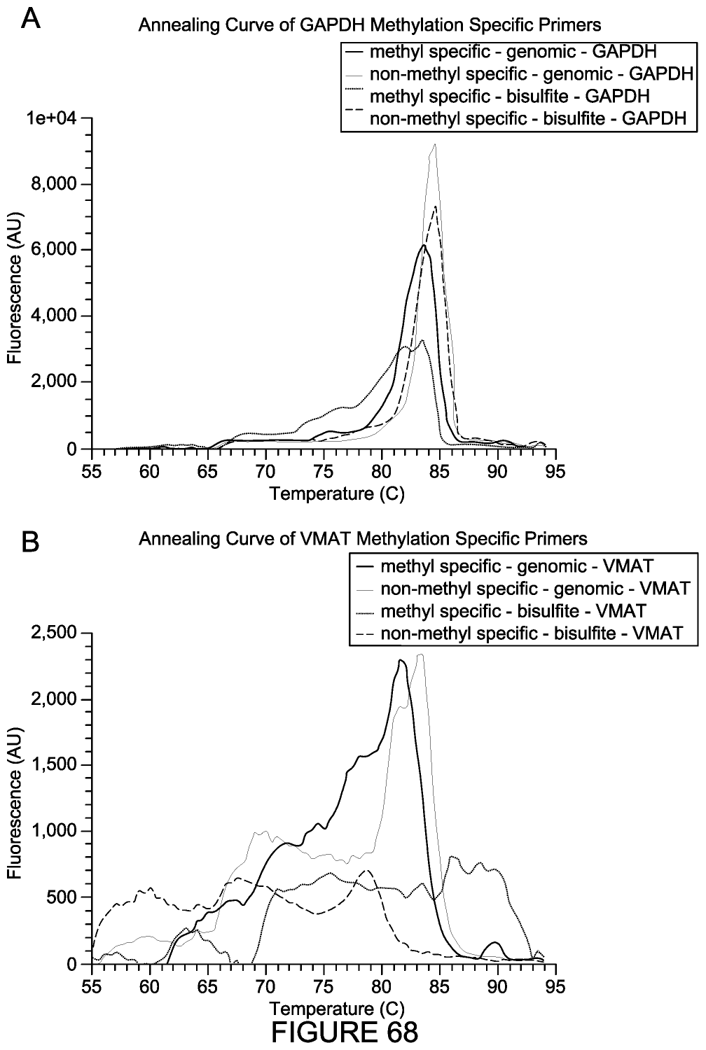

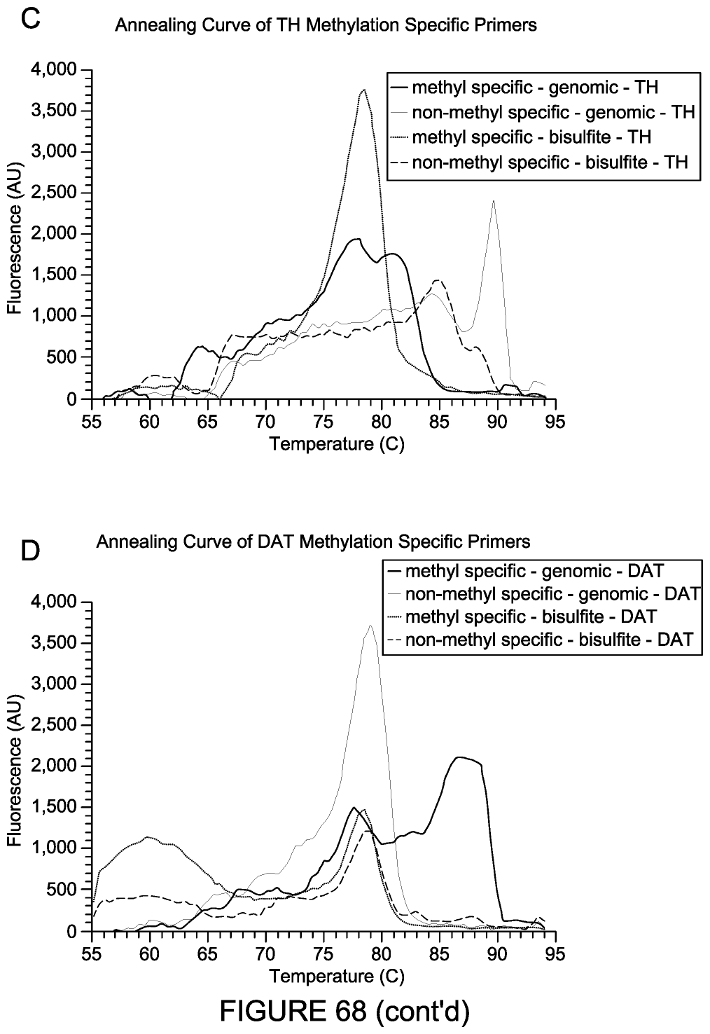

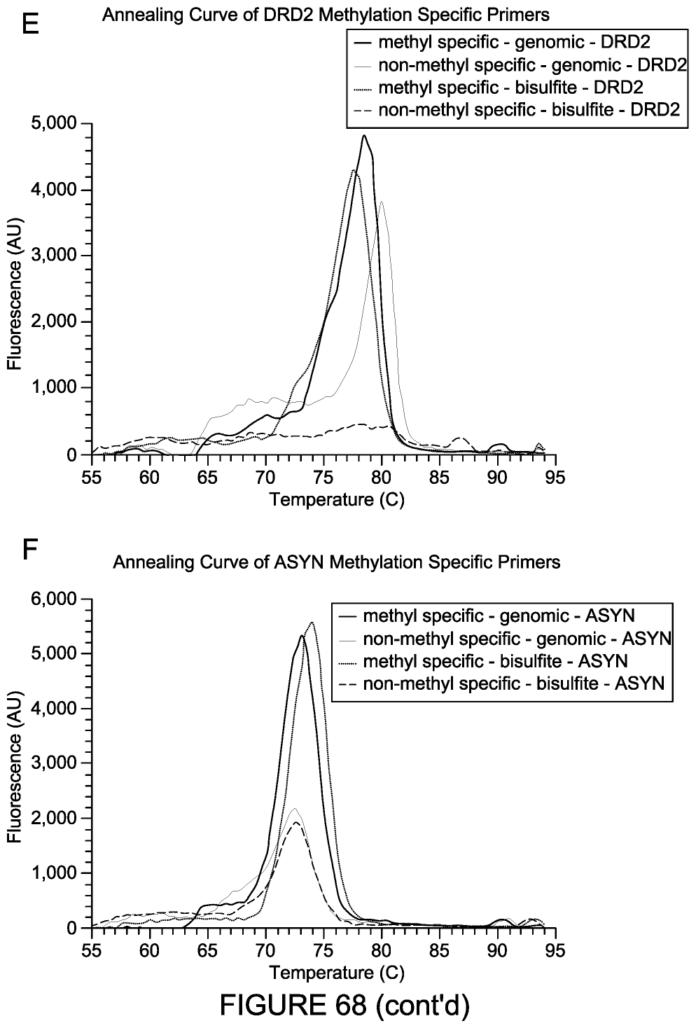

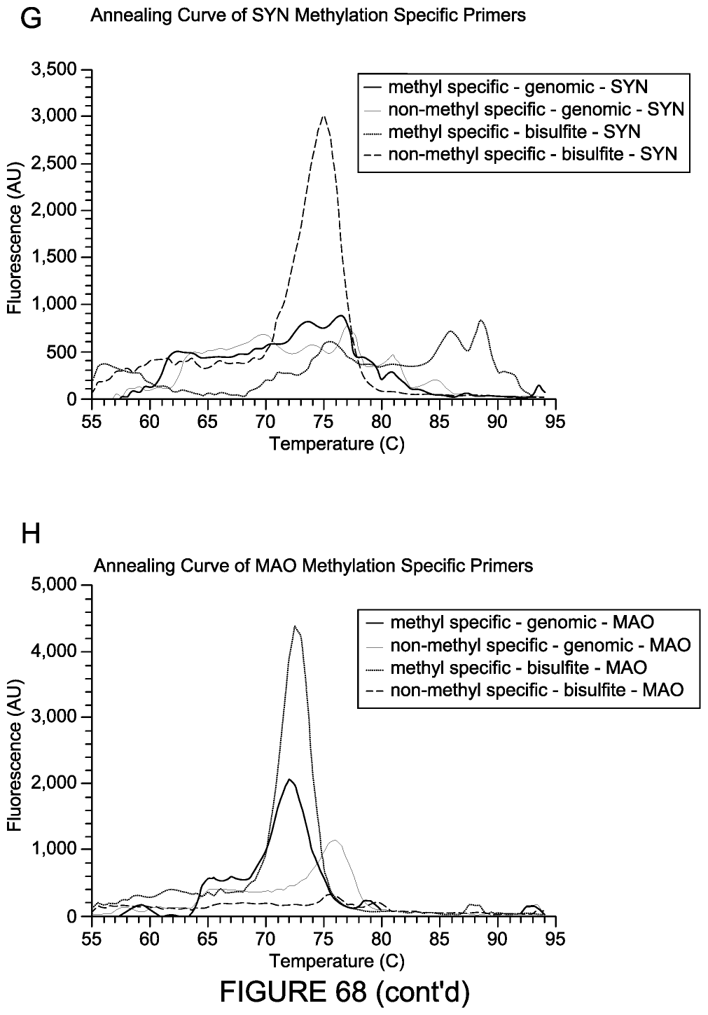

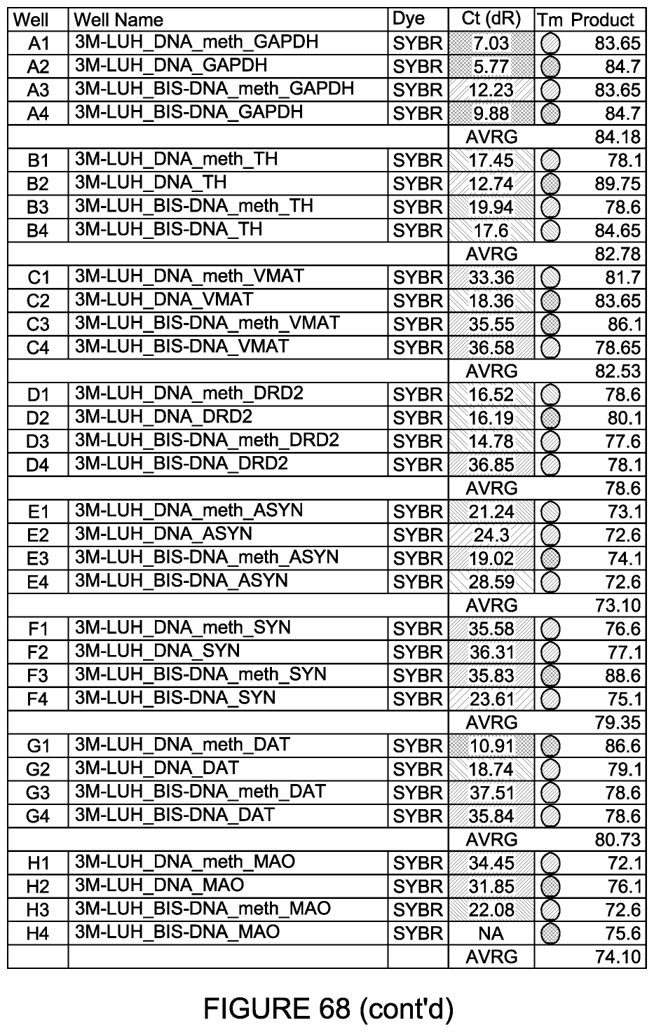

Methylation analyses were conducted on eight representative genes of interest (vesicular monamine transporter (VMAT; FIG. 68, panel B), tyrosine hydroylase (TH; FIG. 68, panel C), dopamine transporter (DAT; FIG. 68, panel D), dopamine receptor (DRD2; FIG. 68, panel E); alpha synuclein (ASYN; FIG. 68, panel F); synaptophysin (SYN; FIG. 68, panel H); 1-H monoamine oxidase (MAO; FIG. 68, panel H), which have been implicated in of Parkinson's disease. Methylation studies were conducted and results (annealing curves of each of the genes of interest) were compared against levels of a control, the housekeeping gene, GAPDH (FIG. 68, panels A-I). In FIG. 68, panels A-H, methyl-specific primers are indicated by black (genomic DNA template) or green (bisulfite DNA template) lines and non-methyl specific primers are indicated by red (genomic DNA template) or blue (bisulfate DNA template) lines. Shifts in annealing temperature (“Tm product”) were observed in TH (FIG. 68, panel C), DAT (FIG. 68, panel D), SYN (FIG. 68, panel H), and MAO (FIG. 68, panel H) indicating increased adenine-thymine (“AT”) bonding following bisulfite conversion, which supports that these genes experience genomic methylation. Bisulfite conversion pertains to the chemical conversion of cytosine (C) to uracil (U) which effectively acts to change the annealing temperature following PCR amplification. Evaluation of CT curves and Tm products in FIG. 68, panel I were used to confirm relative abundance and primer efficiency of the genes of interest. CT values were heat mapped for abundance/efficacy, with red indicating the lowest and green indicating the highest levels. The Tm product column compares annealing temperature values from curves in FIG. 68, panels A-H. Red circles indicate higher Tm (which signifies higher guanine-cytosine (“GC”) content), and green circles indicate lower Tm (which signifies higher AT content).

### Example 15: 3D In Vitro Brain Tissue Models to Study Traumatic Brain Injury

The goal of this example was to expand and improve upon our prior brain-tissue model by, inter alia, increasing cellular complexity including by incorporating microglia and astrocytes with the neurons and then characterize the responses of neurons, astrocytes, and microglia to different types of brain injuries, such as hemorrhage or contusion. This provides a platform and model for continued characterization and development of in vitro tools to study cellular mechanisms or, e.g. injury and healing, and to develop treatments.

### Materials and Methods

Preparation of Silk Scaffolds

Scaffolds were made of silk fibroin solution extracted from Bombyx mori cocoons. Scaffolds were cut with biopsy punches (McMaster-Carr, Princeton, N.J., USA) to outside and inside diameters of 6 mm and 2 mm respectively and 1-2 mm height for all in vitro experiments. Scaffolds with different porosities were prepared using the salt-leaching method, and NaCl salt particles in size varying from 100 μm to 700 μm.

Silk Scaffold Analysis

After scaffolds were cut into the proper shape and size, pores size was analyzed with scanning electron microscopy, and mechanical properties were analyzed using a compression test. High-resolution images of nanostructure were taken with a Zeiss Ultra55 SEM. Scaffolds were placed on SEM stubs (Ted Pella) using carbon tape (Ted Pella), and coated with a thin layer (10 nm) of gold to visualize the structure of silk scaffolds. Pores size was calculated after imagingusing ImageJ software. At least three images per sample were taken and at least three samples per condition were analyzed. A compression test was performed to evaluate the mechanical properties of silk scaffolds with different porosity. At least 8 samples per condition were.

Neuronal Cell Isolation

A fetal brain tissue isolation protocol was approved by the Tufts University Institutional Animal Care and Use Committee and complies with the National Institutes of Health Guide for the Care and Use of Laboratory Animals (Institutional Animal Care and Use Committee M2015-28A1). Cortices and striatum were isolated from embryonic day 16 (E16) C57 black mouse brains (Charles River). Isolated brain tissue was dissociated with 2 mL trypsin/EDTA (0.25%; Thermo Fisher) mixed with fresh DNase (0.3 mg/mL; Roche Applied Sciences), followed by inactivation of trypsin using an equal volume of PBS/hiFBS (10%) solution. Cells were pelleted by centrifugation, resuspended in fresh neuronal medium and counted.

Glial Cell Isolation from Neonatal Brain Tissue

A neonatal brain tissue isolation protocol was approved by the Tufts University Institutional Animal Care and Use Committee and complies with the National Institutes of Health Guide for the Care and Use of Laboratory Animals (Institutional Animal Care and Use Committee M2015-28A2). Microglia isolation was done following established protocols. In brief, P2 (postnatal day 2) C57 black mice (Charles River) was used for glial cells isolation. All brains were collected in cold HBSS (ThermoFisher) and cleared of meninges and dissected under a Zeiss light microscope. Isolated brain tissue was dissociated with 2 mL trypsin/EDTA (0.25%; Thermo Fisher) mixed with fresh DNase (0.3 mg/mL; Roche Applied Sciences), followed by inactivation of trypsin using an equal volume of PBS/hiFBS (10%) solution. Before centrifugation, the cell suspension was gently vortexed, and clumps were removed with 100 μm filters. After centrifugation, cells were resuspended in glial media (DMEM basal medium (ThermoFisher) supplemented with 10% FBS, and penicillin/streptomycin (100 U/mL and 100 μg/mL), plated in T75 flasks, and cultured for 2-3 weeks, until microglia reached a high number. Microglia were then isolated by shaking for 1 hour at 100 rpm on a shaker. Media with suspended microglia was then collected and centrifuged to pellet cells. Cells were resuspended in media, counted, and plated. Astrocytes were collected by trypsinization of the confluent cell layer remaining in the flask after microglia removal. In brief, the attached cell layer was washed once with PBS and incubated with 0.25% Trypsin/EDTA for 3-5 mins. Enzyme activity was then inhibited with an equal volume of glial media. The cell suspension was centrifuged to pellet cells, the pellet resuspended in glial media, and cells counted.

3D Brain Model Preparation

Neuronal and glial cells in monocultures were seeded in silk sponges following our published protocol. Shortly before in vitro experiments began, scaffolds were autoclaved in diH₂O, then coated with poly-D-lysine (PDL) (0.1 mg/mL) overnight in a CO₂ incubator. The following day, scaffolds were extensively rinsed with PBS (ThermoFisher), and equilibrated for one hour in NeuroBasal medium (ThermoFisher) supplemented with 2% B27 (ThermoFisher), penicillin/streptomycin (100 U/mL and 100 μg/mL) (ThermoFisher) and 2 mM Glutamax (ThermoFisher). Concentrated brain cell suspensions (10 million cells/mL, unless different seeding conditions were specifically noted) were seeded onto partly dehydrated scaffolds and incubated for 24 hours, washed with PBS prior collagen embedding the next day. Scaffolds were embedded in 3 mg/ml collagen type I for 30 minutes and after immersed in media, following incubation at 37° C. until the established experimental time points. In co-culture samples, neurons (10 million cells/mL) were seeded onto scaffolds, the next day different amounts of glial cells were added to scaffolds, and on the following day scaffolds were embedded in collagen.

Cell Viability Assays

Cellular viability was studied with the following assays: calcein/propidium iodide staining (live/dead; ThermoFisher, Sigma), DNA quantification (P11496, FisherScientific), MTS cell proliferation (PR-G3580, Promega), luminescent adenosine triphosphate (“ATP”) detection (ab113849, Abcam), caspase-3, 9 detection (BF3100 and BF10100 R&D systems) and lactate dehydrogenase (LDH) measurement (MAK066-1KT, Sigma). All assays were performed following the manufacturer's instructions. In brief, for Live/Deaddead assays, samples were washed once in PBS and then incubated for 30 mins at 37° C. in a solution of calcein and propidium iodide (“PI”) (2 μm calcein and 25 μm PI in PBS). After incubation, samples were extensively washed with PBS and then imaged with a Leica SP8 FLIM (Leica Microsystems) immediately. After imaging, DNA was extracted from samples by treating cells with 0.05% TritonX, followed by sonication for 10 minutes in sonication bath. DNA content was measured via PicoGreen assay using 96-well black plates and a plate reader. DNA concentration was calculated based on a calibration curve prepared using the DNA standard provided by the kit. For MTS cell proliferation assays, cells were mixed with cell culture media at a 1:10 ratio, and samples were incubated for 3 hours in the dark. Results were evaluated using a plate reader and normalizedby subtracting a blank sample from all experimental groups. ATP detection was performed using sample that had been previously frozen (−80° C.) in provided buffer to ensure ATPase enzyme inactivation and cellular ATP stability. Caspase 3 and caspase 9 production were evaluated with detection kits (R&D) using samples that were frozen at −80° C. post-injury in caspase lysis buffer. For LDH release measurement, media was collected at the established experimental time points, and frozen until all samples were collected. LDH activity of experimental groups was calculated by comparing LDH values with those of standard curves, and normalized against cell number to determine the overall LDH activity of single cells.

Immunostaining

2D and 3D samples were fixed with 4% paraformaldehyde (PFA) (Thermo Fisher) for 10 and 30 minutes respectively, washed, permeabilized with 0.1% TritonX supplemented with 10% donkey serum (Sigma), and blocked with 4% BSA (Sigma) for 1 hour. Primary antibody incubation for 2D cultures was 1 hour at room temperature and for 3D cultures, overnight at 4° C. After primary antibody incubation, samples were washed three times with PBS with gentle shaking then incubated with secondary antibodies for 1 hour at room temperature, followed by a 5 minute incubation with DAPI nuclear stain, followed by three additional washes PBS. Fluorescent images of 3D samples were taken on a Leica SP8 FLIM (Leica Microsystems) and images of 2D samples were taken on a Keyence fluorescent microscope.

Antibodies

Primary antibodies and dilutions used for immunostaining were as follows: anti-alpha tubulin); anti-MAP2; anti-VGLUT1, 2; anti-VGAT; anti-GFAP; and anti-S-100 all purchased from Abcam; anti-DARPP-32 (Cell Signaling); and anti-ibal (Wako). Secondary antibodies were goat anti-mouse, anti-rabbit, and anti-chicken Alexa 488 and 568 (1:500; Thermo Fisher).

Calcium Imaging

Neuronal activity was evaluated with calcium imaging following established protocols. Artificial cerebrospinal fluid (ACSF) contained 140 mM NaCl, 2.8 mM KCl, 2 mM CaCl2, 10 mM HEPES and 2 mM MgCl2*6H2O; pH of 7.4 pH was achieved using 200 μl NaOH. Samples were incubated with Fluoro4AM (diluted in Pluronic® F-127) (ThermoFisher) fluorescent probe, with 0.5M glucose added to ACSF. Samples were incubated in the dark for 1 hour with Fluoro4AM (1:1000 dilution) in ACSF. After incubation, samples were rinsed with PBS, and immersed in ACSF during imaging on a Leica SP8 FLIM. Time-lapse images were taken in 30-60 second intervals, with at least 2 seconds per.

### Protein and RNA Isolation

Total protein and RNA were isolated using Fisher BioReagents™ SurePrep™ RNA/Protein Purification Kit. Pure samples with high protein or RNA concentrations were obtained and analyzed via Western blot or real-time quantitative polymerase chain reaction (RT-qPCR), respectively. Lysis buffer was supplemented with protease and phosphatase inhibitors to ensure stability of phosphorylated proteins during isolation and analysis. Western blot analysis was performed following the a standard protocol.

### Results

Structure and Function of 3D Murine Cortical and Striatal Models

Cell viability and functions of the 3D brain tissue model were studied over time to assess changes. Both cortical and striatal neurons appeared healthy and were live and active for 2 weeks in the model system, with a gradual decrease in viability and activity after 4 weeks of culture (data not shown). Immunostaining of cultured cells showed immunoreactivity with neuronal cytoskeletal markers beta-III tubulin (clone tuj1) and MAP2, markers of inhibitory and excitatory synapses, dopaminergic striatal cells (data not shown), and displayed calcium-based activity as a marker of cellular function.

Optimization of the Matrix Conditions for Cortical and Striatal Brain Models

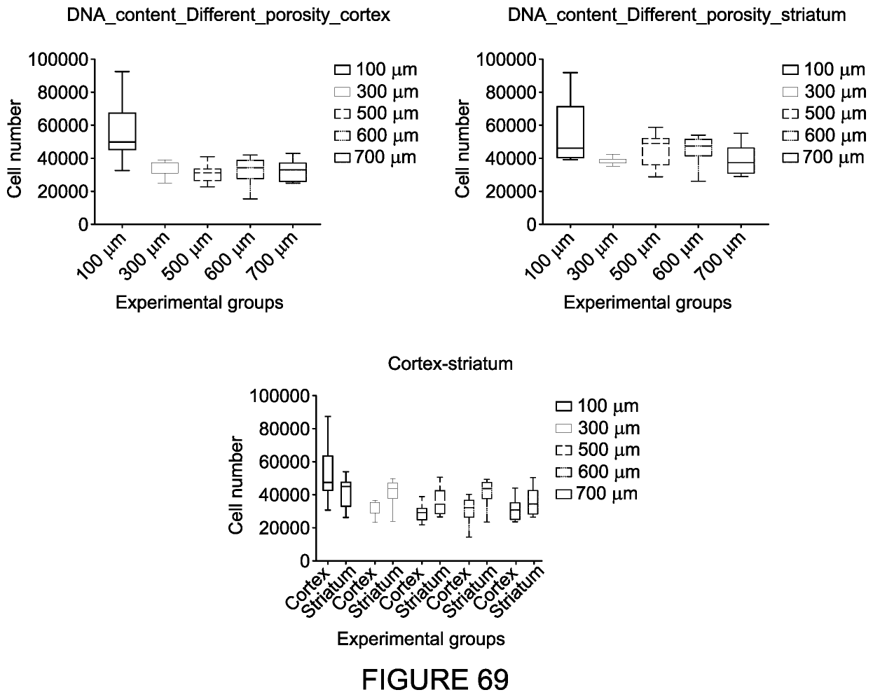

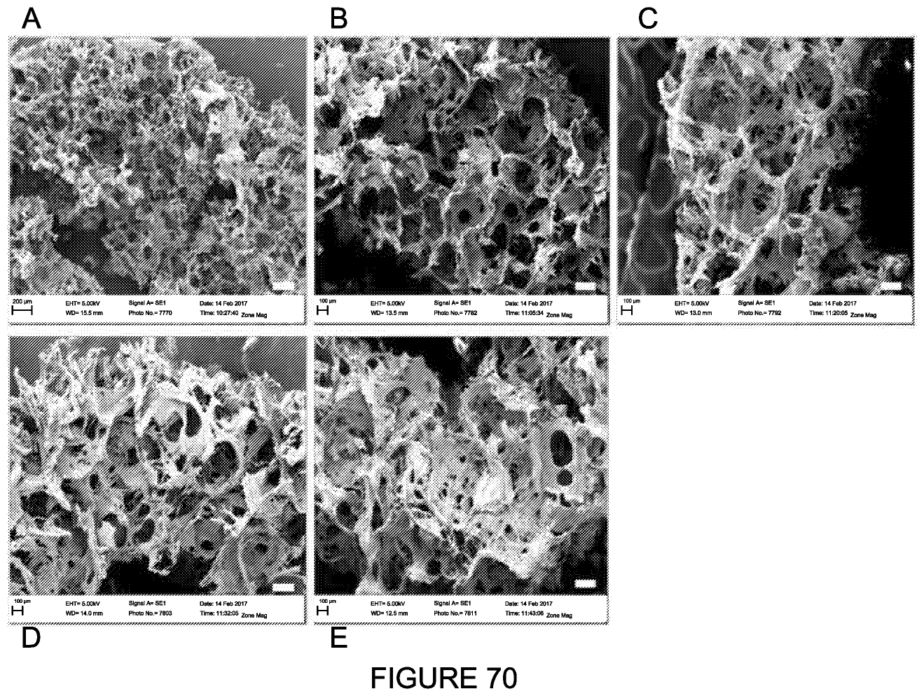

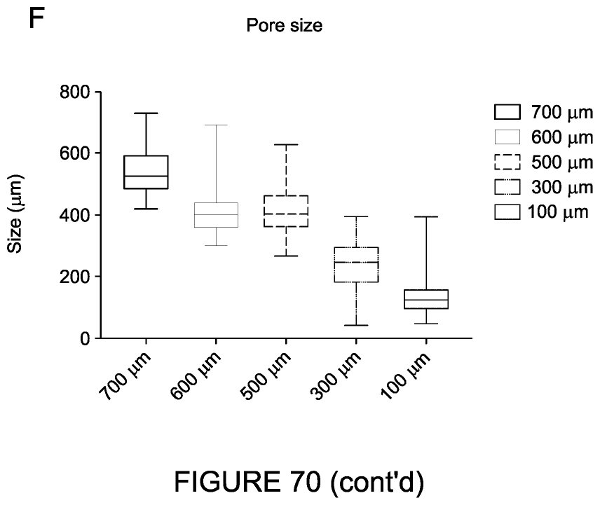

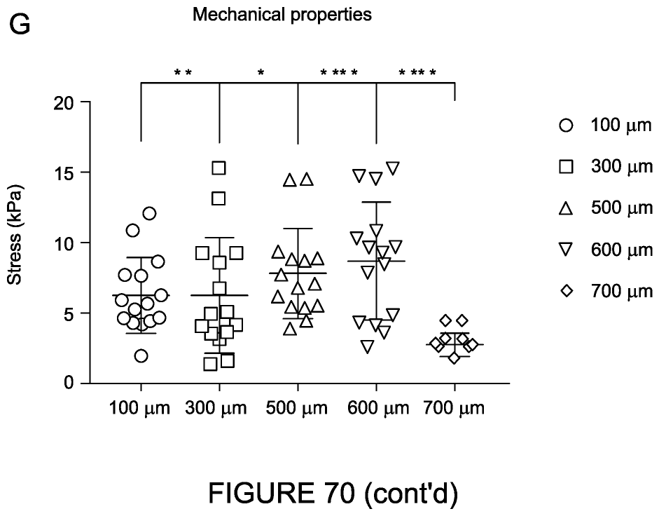

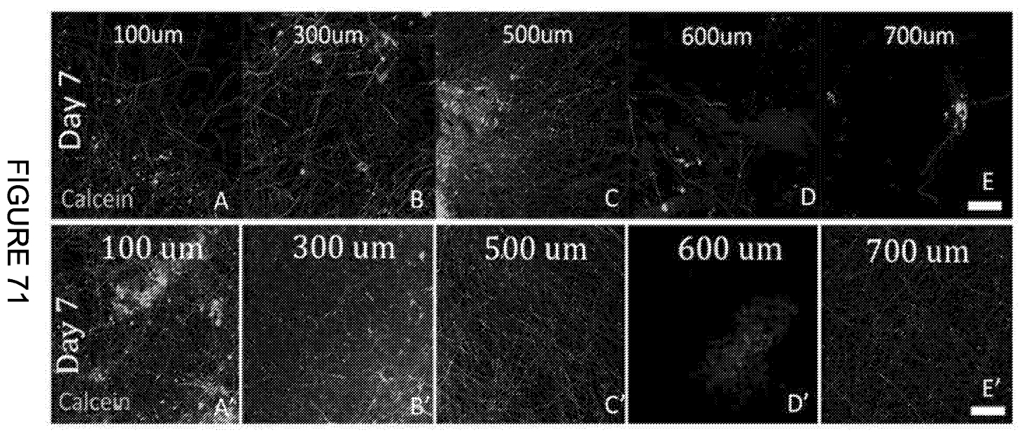

Our earlier work used silk protein sponges with porosities greater than 500 μm to prepare 3D rat cortical brain models. With the addition of cell types in the present study, a range of porosities from 100 μm to 700 μm was assessed for neuronal viability, activity, and function. Porosity was analyzed with SEM images (FIG. 70, panels A-E) and the quantified results (FIG. 70, panel F) showed that the pore size corresponded to the porogen size used to fabricate sponges (FIG. 70, panels A-G). A porosity of 300 μm supported the most dense neurite network formation and showed the best neuronal viability, while the electrical activity was highest scaffolds/sponges with 600 μm pores. The mechanical properties of samples were also analyzed to compare matrix stiffness with that of brain tissue in vivo (FIG. 70, panel G). We also observed that differences in porosity did not affect neuronal seeding efficiency. DNA quantification of cells that did not attach to silk sponges with porosities ranging from 100 μM-700 μm was measured in cortical (FIG. 69, panel A), striatal (FIG. 69, panel B), and both cortical and striatal (FIG. 69, panel C) neurons.

Characterization of Glial Cell Activity and Function in 3D Tissue Model

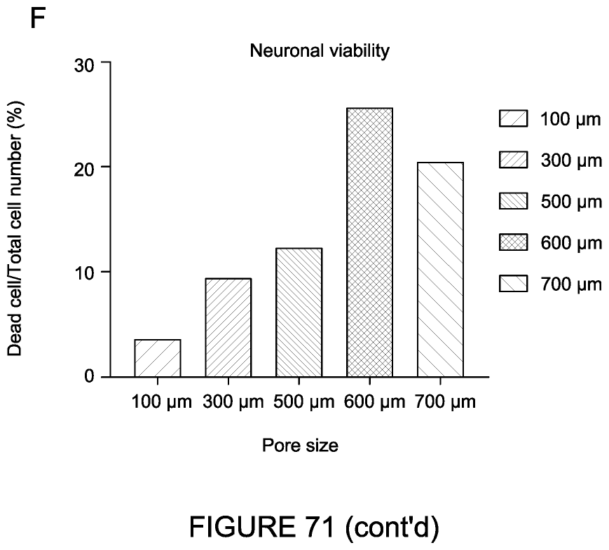

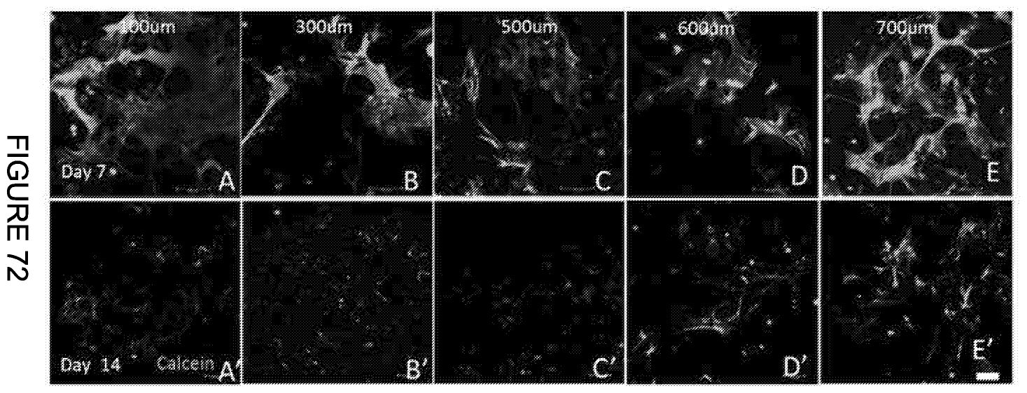

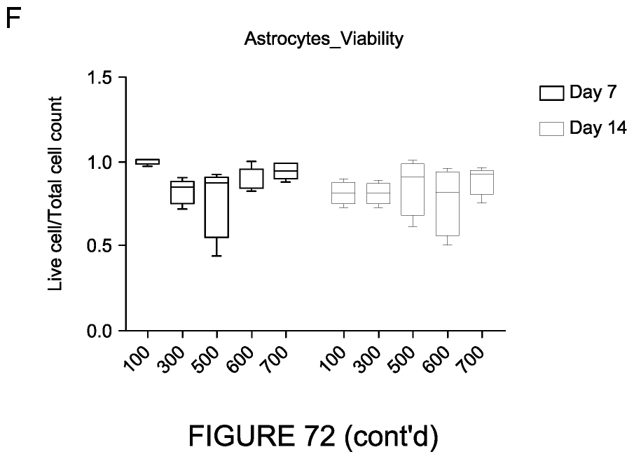

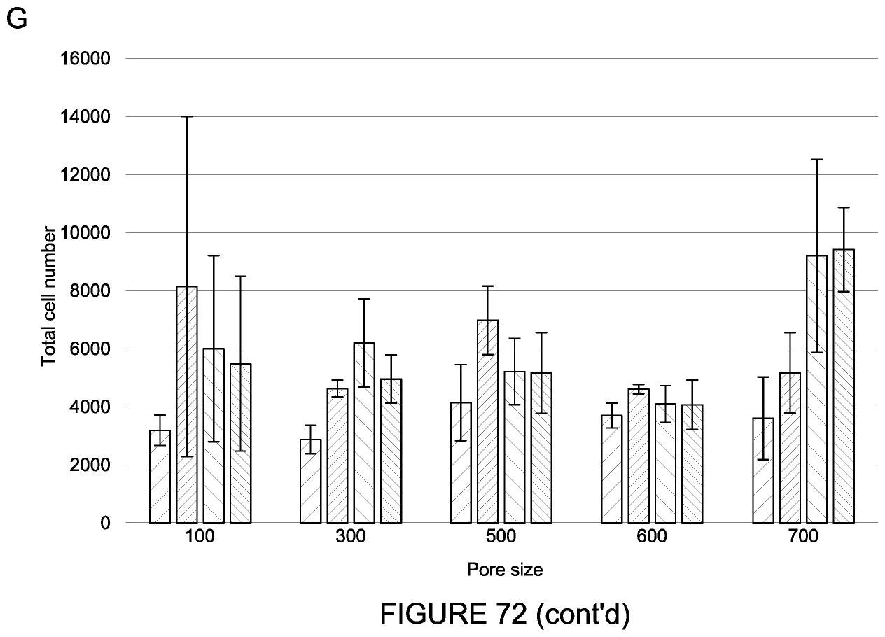

We also visualized cells to further examine effects of different pore sizes on neuronal behavior (monocultures). We measured neuronal viability with a Live/Dead assay and calcein (live, green) cells can be seen in FIG. 71, panels Ai-Bv. Quantification of neuronal viability was measured by comparing quantity of dead cells to the total cell number (FIG. 71, panel C; striatal neuron viability). We also evaluated effects of porosity on astrocyte viability using live/dead analysis at days 7 (FIG. 72, panel A) and 14 (FIG. 72, panel B). Calcein-labeled (green) cells were quantified and expressed as a percentage of total cells to determine viability (FIG. 72, panel C) of astrocytes in sponges with different porosity.

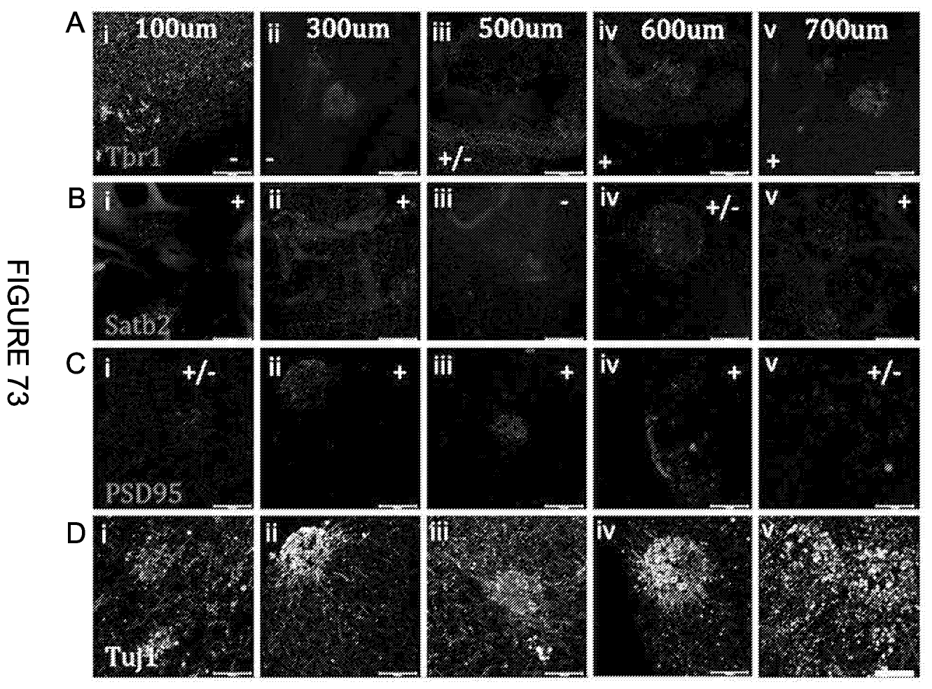

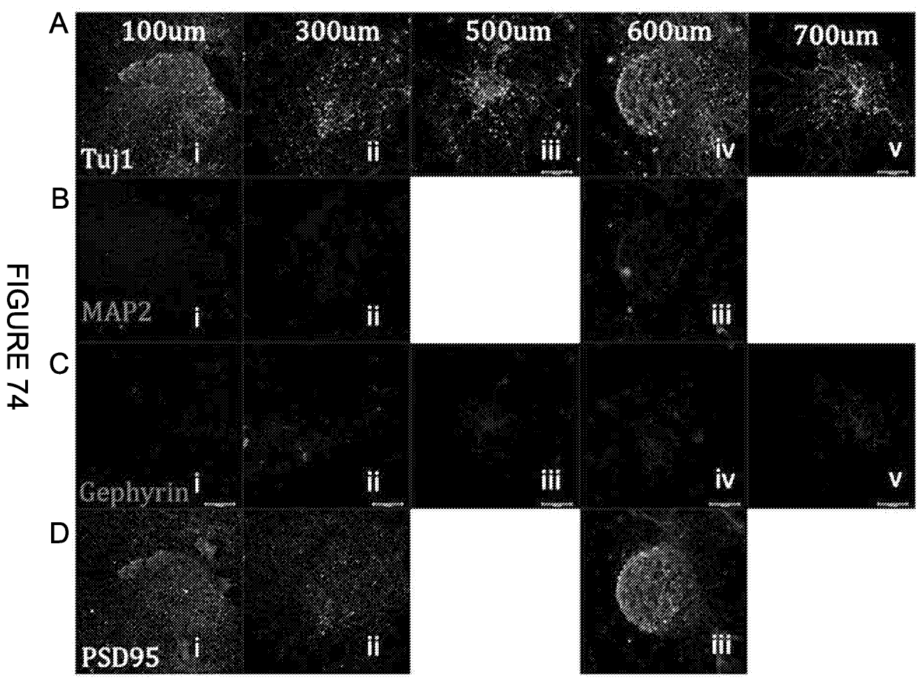

We investigated whether certain subtypes of neurons (such as neurons of specific cortical layers) appeared to prefer different porosities. We stained for markers of layer V/VI neurons (Tbr1, FIG. 73, panels Ai-Av), layer II/IV neurons (Satb2, FIG. 73, panels Bi-Bv), as well as excitatory synaptic markers (PSD95, FIG. 73, panels Ci-Cv). We also confirmed that cells displayed the pan-neuronal marker, betaIII tubulin (clone tuj1, FIG. 73, panels Di-Dv). FIG. 74, panels Ai-Diii show immunocytochemical analyses for different subtypes of striatal neurons. FIG. 74, panels Ai-Av show cells stained for the pan-neuronal marker, beta-III tubulin (clone tuj1, green). FIG. 74, panels Bi-Biii show neurons stained for the neuronal dendritic marker, MAP2 (red). FIG. 74, panels Ci-Ciii show neurons stained for an inhibitory marker, Gephyrin (red). FIG. 74, panels Di-Diii show neurons stained for the excitatory synapse marker, PSD95 (green). We found that striatal neurons of different subtypes (as identified by various cell markers) preferred different porosities. For example, inhibitory neurons (identified by gephyrin expression) showed better growth in sponges with higher porosity, whereas neurons positive for the excitatory marker PSD95 appeared to grow better in sponges with smaller porosity.

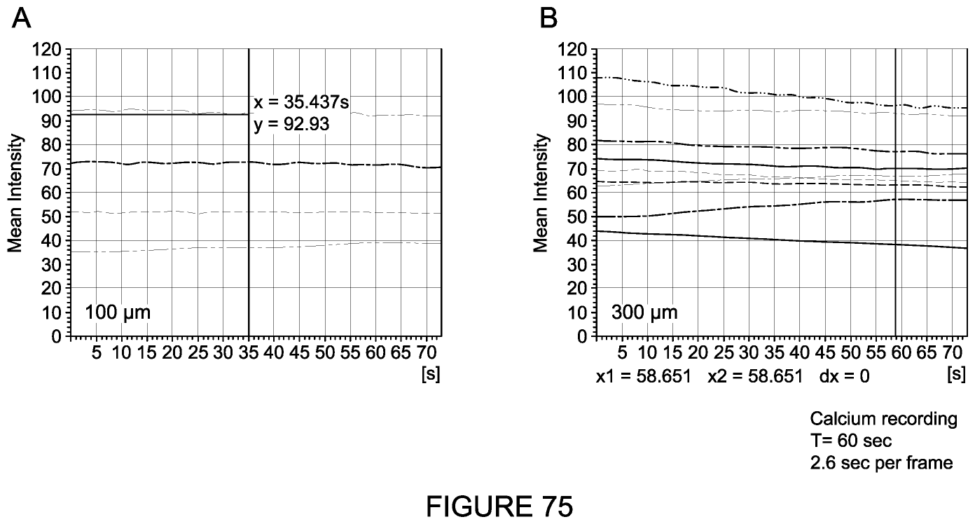

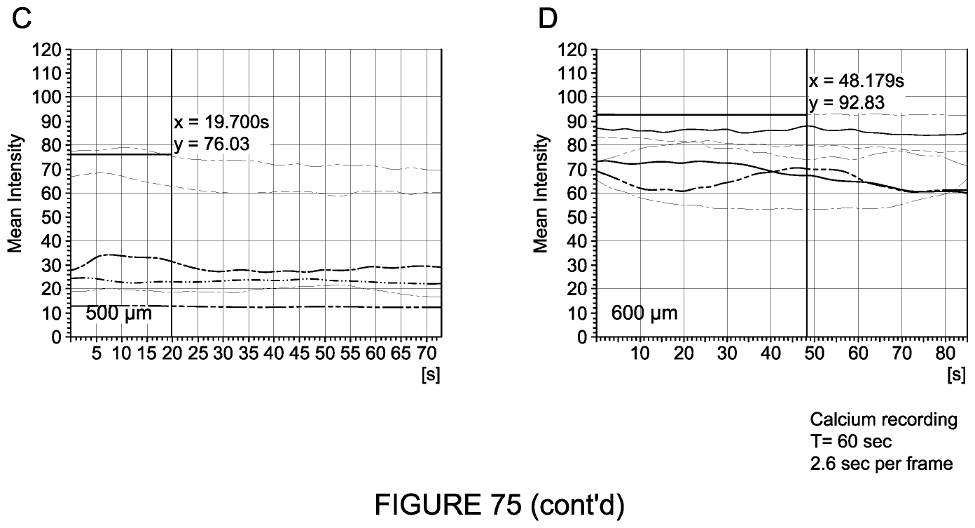

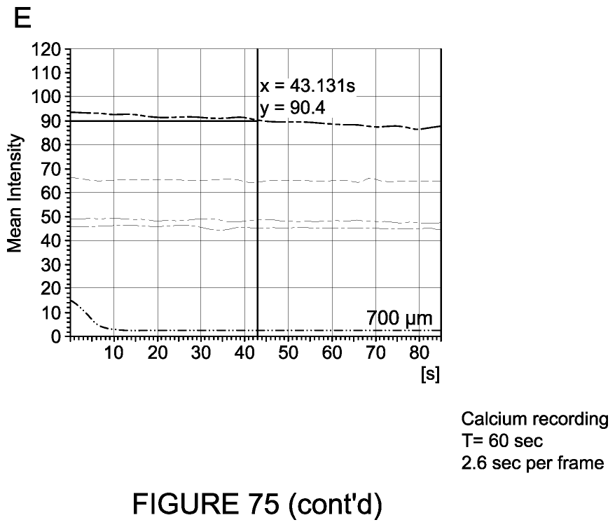

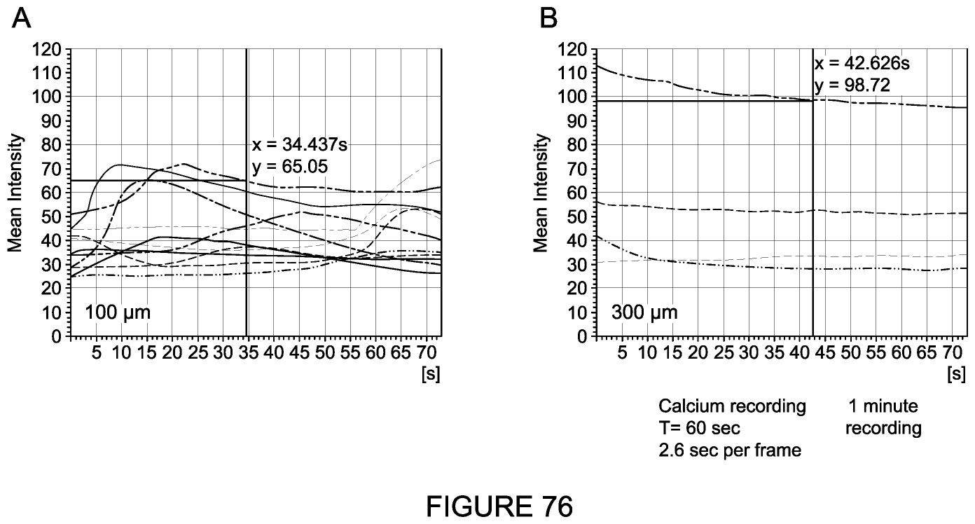

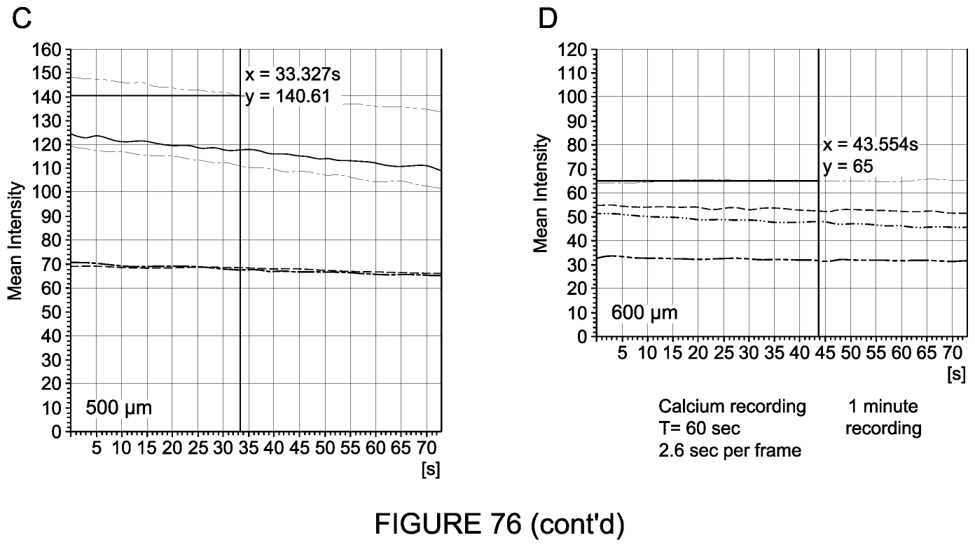

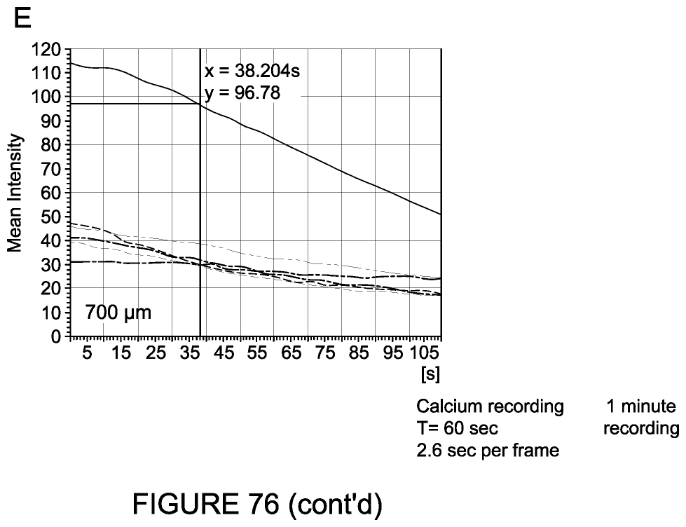

We also investigated calcium fluctuations in cortical (FIG. 75, panels A-E) and striatal (FIG. 76, panels A-E) neurons after seven days in culture. Cells exhibited differences in calcium fluctuations in sponges of different porosities.

### Example 16: 3D Intracerebral Hemorrhage Model

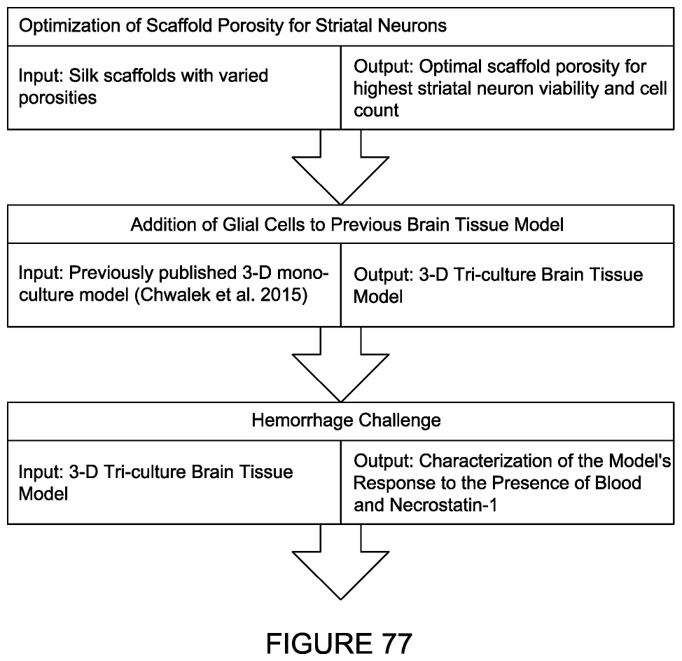

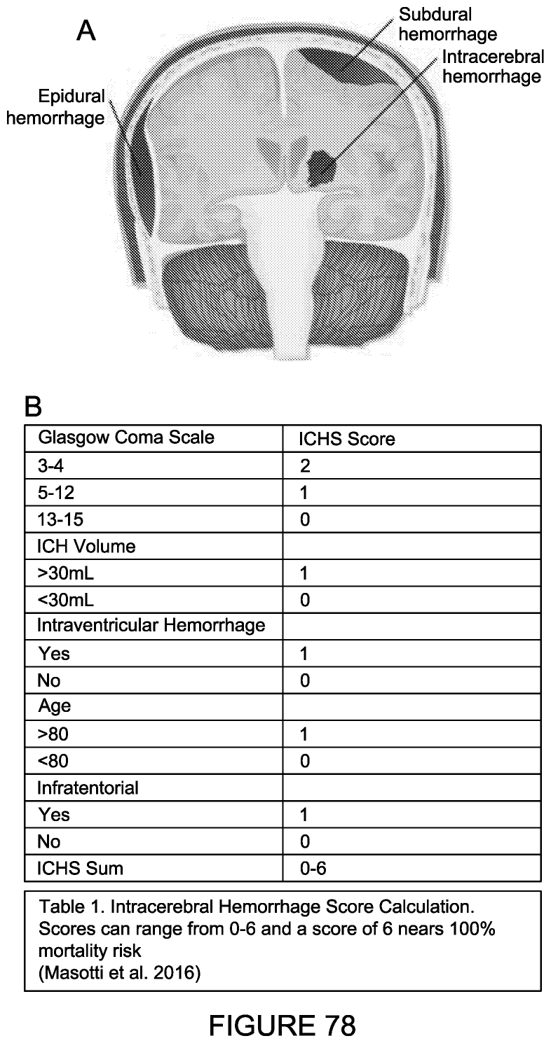

The purpose of this Example was to develop a new, physiologically relevant model of intracerebral hemorrhage (“ICH”), which includes three-dimensions, astrocytes, and microglia. This will help in gaining a better understanding of ICH and facilitate development of new treatments. A key aspect of this model is use of primary striatal neurons, rather than cortical neurons, to more closely model the striatum, which is where intracerebral hemorrhage may occur (FIG. 77). The addition of glial cells is important for hemorrhage modeling because inflammation is one of the major symptoms post-injury and astrocytes and microglia both play roles in regulating inflammation (Anttila et al. 2016, Colombo and Farina 2016). In addition, opening of the blood-brain barrier is often observed after ICH and astrocytes are key regulators of blood-brain barrier integrity and function (King et al. 2014).

The goal of this example was to develop a 3D tri-culture intracerebral hemorrhage model using primary murine striatal neurons, astrocytes, and microglia grown on a silk scaffold, representing a significant improvement over current 2D models or 3D monocultures. We developed techniques to optimize porosity of the silk scaffolds to ensure striatal neuron viability. The finished models were then tested with blood injections (to model intracerebral hemorrhage) and several different assays were performed to characterize the model. Additionally, a necrostatin-1 treatment, normally considered neuroprotective, was used to detect whether the presence of glial cells altered the protective effect of the treatment.

When developing our model, porosity and cell phenotypes were verified by a scanning electron microscopy and immunocytochemistry, respectively. Scaffold porosity was then adjusted to optimize striatal neuron growth. Models were then challenged with a blood injection to simulate a burst blood vessel, as occurs in ICH. We also used an inhibitor of necroptosis, necrostatin-1 (“nec-1”), as a surrogate for necroptosis in our samples. In brief, nec-1 inhibits RIP1/RIP3 pathways of necroptosis (which are regulated by microglia [Su et al., 2015]); and a reduction in cell death or release of reactive (e.g., oxygen, nitrogen, etc.) species in nec-1 treated samples represents inhibition of necroptosis. Assays were performed to measure cell viability, cell numbers, changes in cellular metabolism and proliferation, and release of reactive species (e.g., oxygen, nitrogen, etc.).

### Materials and Methods

Elements of Engineering Design

The objective of this study was to create a model which imitates physiological cell responses of neurons and glia within the brain and, similar to documented in vivo behavior, that shows increased inflammatory response after a blood injection challenge (mimicking intracerebral hemorrhage. We designed a three-dimensional tissue culture model of intracerebral hemorrhage utilizing primary murine neurons and glial cells to more closely recapitulate the in vivo environment and test whether the presence of glial cells would increase the inflammatory response upon hemorrhage. The results will allow improved models and treatments for intracerebral hemorrhage.

Overall Characterization

The model was characterized using immunochemistry and various cellular assays. This 3D intracerebral hemorrhage model was evaluated by quantitatively measuring the inflammatory response of samples 4 and 24 hours after hemorrhage injections. ATP, MTS, ROS-RNS, LDH, and Live/Dead assays were used to measure concentration of inflammatory markers, viability of samples, and metabolic response of cells within the scaffolds.

Silk Sponge Preparation

Silk fibroin solution was isolated according to the protocol provided in Nazarov, Jin, and Kaplan (2004). A thirty-minute boil was used, and silk was dissolved in 9.3M lithium bromide for 6 hours before undergoing dialysis and centrifugation to obtaina silk fibroin solution for use in making sponges. The fibroin solution was diluted to 6% w/v with diH2O if necessary. Next, the fibroin solution was added to a petri-dish to a height of approximately 2-3 mm. Sodium chloride was added to the petri-dish at a ratio of 2 g NaCl:1 mL fibroin solution, and spread evenly throughout the dish. The petri-dish was covered and left in a chemical hood at room temperature for 48 hours. After 48 hours, the dish was moved to a 60° C. oven and incubated for an additional hour to induce cross-linking of the fibroin fibers. The sponge was then removed from the petri-dish and washed in diH2O by hand for 20 minutes, then placed into a 5 L beaker with a magnetic stir rod and diH2O and allowed to wash for 2 more days, with the diH2O being replaced approximately every 6 hours. The sponges were cut with 6 mm and then 2 mm biopsy punches to create a doughnut-like shape, and then cut with scissors to achieve a height of 1.5-2 mm if necessary. These sponges were then autoclaved in a bottle of diH2O and stored at 4° C. until use.

SEM Porosity Verification

To characterize porosity of the sponges after crosslinking with various sized porogens, a scanning electron microscope was used to take images of the sponges. These images were then analyzed with ImageJ software to measure the diameter of the pores.

Striatal Neuron Isolation

Striatal neurons were isolated from E16 C57BL mouse embryo brains (according to Tufts University IACUC protocol, M2015-28). To begin, silk sponges were coated with 0.1 mg/mL PDL the night before the dissection. The next day, sponges were washed 2-3 times with sterile PBS and then incubated with 75:25 media (see Table 2) for at least one hour before seeding neurons. Neurons were isolated by euthanizing a pregnant mouse at E16 days gestation, in CO2 followed by cervical dislocation. Embryos were removed by opening the abdomen of the pregnant mouse and carefully removing the uterine horns. Individual mouse embryos still in their amniotic sacs were carefully separated from one another and placed into petri dishes filled with sterile Hank's Balanced Salt Solution (HBSS). Embryos were subsequently dissected from sacs, placed into a new petri dish with HBSS, decapitated with sharp scissors, and heads moved to a new dish with HBSS on ice. Striatal tissue was isolated by using a scalpel to make an incision through the skin and the skull, starting at the top of the head along the medial plane, being careful not to damage any brain tissue below. Tweezers were used to pull back the skull from the brain, and the brain was then gently scooped out of the head. Tweezers were then used to separate the midbrain and release the cortex and striatum. Tissue was moved to a new petri dish filled with HBSS. Meninges were carefully and completely dissected away from the tissue (as any meningeal blood vessels can lead to neuronal clumping and reduce the final yield of cells).

TABLE 2
Table 2. Components of the media used. A mixture of Neurobasal and N9
media was used for the tri- culture mode, with Neurobasal + B27 +
Glutamax + Anti- Anti composing 75% of the media, and DMEM +
FBS + Anti-Anti (or N9 media) composing 25% of the media.
Medium composition (500 mL)

| Product | Code | Quantity | |
|---|---|---|---|
| Neurobasal Media | 21103-049 | 360 | mL |
| DMEM | REF11995-065 | 111.25 | mL |
| B27 Supplement | 17504-044 | 7.5 | mL |
| FBS | 10082-139 | 12.5 | mL |
| Glutamax | 35050-061 | 3.75 | mL |
| Antibiotic/Antimycotic | | 5 | mL |

The cortex was separated from the striatum with tweezers and each was placed into a different 50 mL conical tubes with HBSS, on ice. Once all brain tissue was collected, HBSS was aspirated from the brain tissue and 2 mL of 0.25% trypsin and 0.3 mg/mL DNase was added to the tubes. The tissue and trypsin/DNase mixture was pipetted into a small petri dish and incubated at 37° C. for 20 minutes. 2 mL of N9 media supplemented with FBS was added, in order to stop enzymatic activity. The mixture was then pipetted into a 15 mL conical tube and dissociated by gently pipetting up and down ten times. A micropipette was used to remove any clumps remaining after initial dissociation. Cells were centrifuged at 1,100 rpm for 5 minutes, media was aspirated and the pellet was resuspended and counted. Cells were then resuspended 75:25 media. Cells were then seeded according to the protocol described below.

Astrocyte Isolation

To isolate astrocytes, cell culture flasks (T75) were coated with 0.01 mg/mL PDL the night before the dissection. The day of the dissection, flasks were washed 2-3 time with sterile PBS and incubate with N9 media for at least 1 hour before seeding, astrocytes are isolated from postnatal day 1 (P1) C57BL mouse pups. Brains were removed by rapid decapitation and heads placed in HBSS on ice. Brain tissue is then isolated following the dissection techniques used in the striatal neuron isolation protocol described above. Cells are cultured for 2-3 weeks before seeding onto 3D cultures.

Cell Seeding onto Scaffolds (Astrocytes and Neurons)

Silk fibroin sponges were pre-coated with poly-D-lysine (PDL) by applying 0.1 mg/ml PDL solution to the scaffolds in preparation for cell attachment. The sponges were incubated at 37° C. in a CO₂ incubator for at least 1 hour. PDL solution was then aspirated and sponges were thoroughly rinsed at least twice with sterile DPBS to remove any traces of PDL (unbound PDL is neurotoxic and can lead to a lack of cell attachment to the scaffolds). DPBS was then aspirated from the wells and 200 μL of culture medium was added into each well. Scaffolds were placed in a 37° C. incubator to equilibrate for at least 1 hour before cell seeding. Next, astrocytes were collected by adding a trypsin solution to each flask and detaching cells. Cells were collected, spun down and resuspended to obtain a solution with an astrocyte concentration of 500,000 cells/50 μL; cells from the neuron isolation procedure were resuspended at a concentration of 1,000,000 cells/50 μL. 50 μL of each solution (astrocyte and neuron) were added to each sponge at several spots around the circumference of the sponge for even cell distribution. Sponges with cells were incubated for 30 minutes at 37° C., followed by addition of 100 μL of media. The next day, sponges were transferred to a new 96 well plate, 100 μL of 3 mg/mL collagen in PBS was added and plates were incubated for 30 minutes. 100 μL of media was added and plates were incubated overnight. Sponges were transferred to a 24-well plate the next day 1 mL of media was added to each well. Media was changed every 2-3 days (as in Chwalek et al. 2015).

Cell Culture and Media

Sponges with astrocytes require media to be changed 2-3 times per week and sponges with neurons need 1-2 media changes per week. The media used is the same for both samples: a 75% Neurobasal and 25% N9 media mixture with several supplements (see Table 2).

Hemorrhage Injury and Necrostatin-1 Treatment

Hemorrhage injuries were performed at the Neuroscience Center at Massachusetts General Hospital. Samples were transported to the Center, incubated for one hour at 37° C., and then injected with 10 μL of mouse blood and incubated for another hour before being returned to the Tufts University Biomedical Engineering Department. Samples were evaluated at 4 and 24 hours post-injury. For necrostatin-1 treatment, samples were incubated with 250 μmol/mL necrostatin-1 for 15 minutes prior to performing a hemorrhage injury.

Experimental Groups

For all studies, groups included: untreated controls, sham, sham plus nec-1 treatment, hemorrhage (ICH) or ICH plus nec-1 treatment samples.

Cell Viability Assay

Cell viability was assessed with a Live/Dead assay, which uses a mixture of calcein and propidium iodide stains (1.2 μL of calcein (reconstituted in DMSO) and 10 μL of propidium iodide (“PI”) per 1 mL of final stain solution). Samples were washed with PBS 2-3 times and any remaining PBS was aspirated. Sponges were transferred to a 96 well plate, 100 μL of calcein/PI stain solution was added to each sample, and they were incubated at 37° C., covered in aluminum foil for 30 minutes. The stain solution was then aspirated, samples were washed with PBS 2-3 times, and then imaged using a confocal microscope with lasers of wavelengths of 488 μm (to detect calcein staining) and 552 μm (to detect PI staining).

WST1 Cell Proliferation Assay

A WST1 assay was used for analyzing the proliferation rates of cells in a culture. Cells were incubated in a well-plate for three hours in 1 mL of a media-stain mixture consisting of 90% media and 10% WST1 stain. A blank well was filled with the media-stain mixture to serve as a control/detect any background signal. After three hours, a small amount of media from each well was transferred to a clear plastic well plate and the absorbance of the media at 440 nm was measured using a plate reader.

Immunocytochemistry

Immunocytochemistry combined with morphological observation was used to determine cell phenotype. Astrocytes were identified by antibodies against S-100 and GFAP. Striatal neurons were identified by beta-III tubulin (clone Tuj1), which marks neurons, and DARPP-32, which is dopaminergic neuron-specific marker (Matamales et al. 2009). Microglia were identified using antibodies against iba-1. Prior to staining, samples were fixed with paraformaldehyde (“PFA”) at two stages. 4% PFA was first added to the cell culture media in a 1:1 ratio for a 5-10 minute incubation. After all liquid was removed, fresh 4% PFA was added for 10 min. 100 μL of blocking buffer (1% BSA, 0.1% TritonX in PBS) was then added to the wells and incubated for 1 hour. Primary antibodies were applied overnight at 4° C. or for 1 hour at room temperature. Samples were washed with PBS three times for 5-10 minutes per wash. Secondary antibodies were applied for 1 hour at room temperature and followed by washing with PBS another three times. Lastly, DAPI solution was applied for 5 min. Cells were washed twice with PBS. 100 μL of PBS was added to the samples for imaging.

DNA Quantification

DNA was quantified using a PicoGreen assay. To prepare samples for DNA quantification, they were first washed once with PBS, followed by addition of 100 μL of 0.05% Triton-X to cover the sponge. Samples were stored at −20° C. until use. For measurements, samples were thawed at room temperature and homogenized with scissors. Supernatant was collected and placed in a tube, on ice. Enough 1×TE Buffer needed for a dilution of 100 μL 1×TE buffer and 200 μL supernatant for each sample was prepared, and 4 mL of 1×TE buffer was prepared for the standards. 20×TE buffer was diluted 1:20 in diH2O to get 1×TE buffer. 1×TE buffer was then mixed with sample supernatants in separate 1.5 mL tubes. 100 μL of each sample was added to a 96-well plate, in triplicate. Standards were prepared by mixing the DNA working solution and the TE buffer to create 400 μL solutions with the following DNA concentrations: 2000 ng/mL, 1000 ng/mL, 500 ng/mL, 200 ng/mL, 100 ng/mL, 50 ng/mL, 20 ng/mL, and 0 ng/mL. Once samples were ready for analysis and the fluorescence plate reader was warmed and ready, PicoGreen stain was added to each sample. PicoGreen stain was prepared by making a 200-fold dilution of the stock PicoGreen stain in 1×TE Buffer for a final volume of 100 μL per sample plus 30%. The PicoGreen solution was protected from light and 100 μL of the diluted PicoGreen solution was added to each sample, then incubated at room temperature for 2-3 minutes. Fluorescence of samples was read using a plate-reader with 485 nm excitation and 538 nm emission. Fluorescence readings were converted into cell counts by using standard calculations to determine DNA content of each sample, which was then divided by 5.5 pg, which is the average mass of DNA in neurons (Moroz and Kohn 2013).

MTS Assay

MTS assays were conducted using a commercial kit. Briefly, the One Solution Reagent was thawed and 150 μL was added to each well with a sample, then incubated for 2 hours at 37° C. Absorbance at 490 nm was measured with a plate reader with the samples and reagent, and then again after removing samples from reagent.

ATP Assay

For ATP assays, 50 μL of detergent was added in 100 μL of media and added to each of the samples. The plate was then shaken for 5 minutes on an orbital shaker at 700 rpm to lyse the cells and release the ATP. 50 μL of reconstituted substrate solution was added to each well and then the plate was again shaken for 5 minutes at 700 rpm. The plate was placed into the dark for 10 minutes, and luminescence was measured using a plate reader.

LDH Assay

LDH assays were run on all samples and standards in duplicate, using a commercially available kit. An NADH standard was prepared using the stock NADH standard solution and the LDH assay buffer to achieve 50 μL each of the following concentrations: 0, 2.5, 5, 7.5, 10, and 12.5 nmole per 50 μL. 500 μL of LDH assay buffer was added to each sample for every 100 mg of tissue and samples were then homogenized with scissors, on ice, prior to centrifuging for 15 min at 10,000×g and 4° C. 50 μL of the supernatant was added into wells in a 96 well plate (in duplicate) and LDH buffer was added to a total volume of 50 μL per well. For each reaction (sample and standards), 2 μL of LDH substrate mix was added to 48 μL of LDH assay buffer to create a Master Reaction Mix. 50 μL of reaction mix was added to each well and contents were mixed by pipetting up and down. Plates were covered with aluminum foil and incubated for 2-3 minutes in the dark. A plate reader was used to measure absorbance at 450 nm. Multiple readings were taken for each sample. For the initial measurement, we ensured that all samples had initial readings between those blank and the highest standard. The plate was then incubated and absorbance measured every five minutes until the reading from the most active sample was higher than the value of the highest standard.

Reactive Oxygen Species-Reactive Nitrogen Species Assay

Reactive species were measured using a commercially available kit. Each sample was assayed in duplicate or triplicate. Briefly, catalyst was prepared by diluting a stock solution 1:250 in PBS and vortexing thoroughly. DCFH solution was prepared by first diluting DiOxyQ 1:5 in Priming Reagent and vortexing the mixture. DCFH solution was incubated for 30 minutes at room temperature and diluted 1:40 in stabilization solution, then vortexed and protected from light. Samples were prepared by first homogenizing the tissue on ice with scissors and then centrifuging at 10,000×g for 5 minutes. The supernatant was then used for the assay. To prepare the DCF standards, DCF standard was mixed with PBS according to Table 3. 200 μL of each DCF standard was transferred to a 96 well plate fluorescence read at 480 nm excitation and 530 nm emission. Stock H2O2 standard solution was diluted 1:4400 in diH2O for a final concentration of 2 mM. Diluted samples were used to prepare the standard according to Table 4. 50 μL of each sample or H2O2 standard was added to each plate. 50 μL of catalyst solution was then added to each well and pipetted to mix. Plates were incubated for 5 minutes at room temperature, followed by addition of 100 μL of DCFH solution to each well. Plates were then covered for 15-45 minutes at room temperature and fluorescence was read using a plate reader at 480 nm excitation/530 nm emission.

Data Analysis for Plate-Based Cellular Assays

In each plate-based assay measured using a plate-reader, blank readings were subtracted from sample readings in. Standards were used to create linear conversion equations to extrapolate concentrations or measurements of samples from plate reader output. ANOVAs were calculated per-sample for every assay followed by 2-way t-test with a=0.05 using GraphPad Prism.

### Results

Verification of Porosity

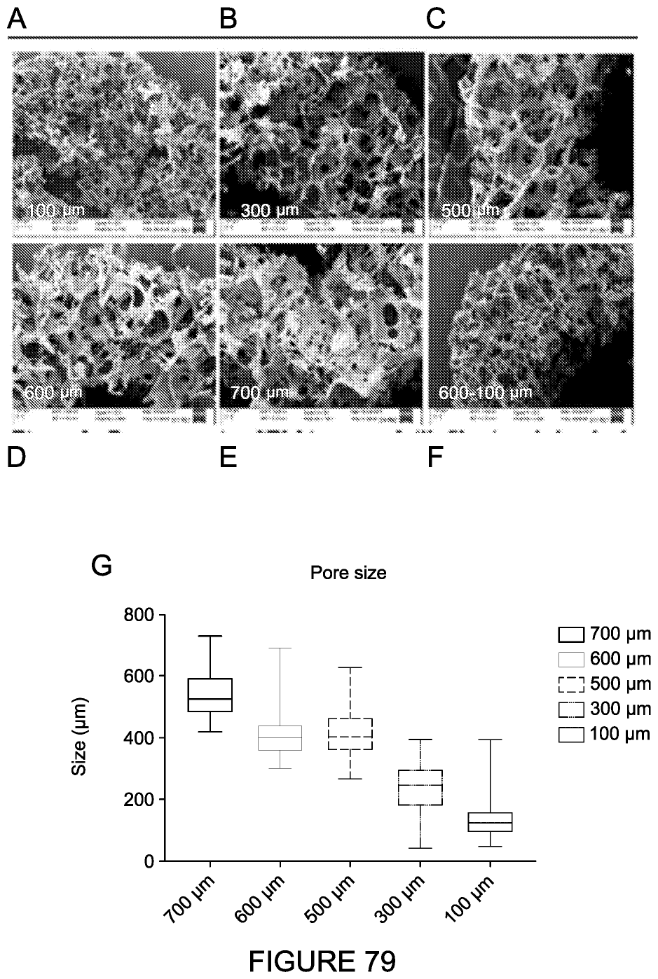

Porosity of the sponges was measured using SEM and ImageJ for post-imaging analyses. FIG. 79, panels A-F shows representative images of pores of different sizes and FIG. 79, panel G shows quantification of pore sizes.

Verification of Cell Phenotypes

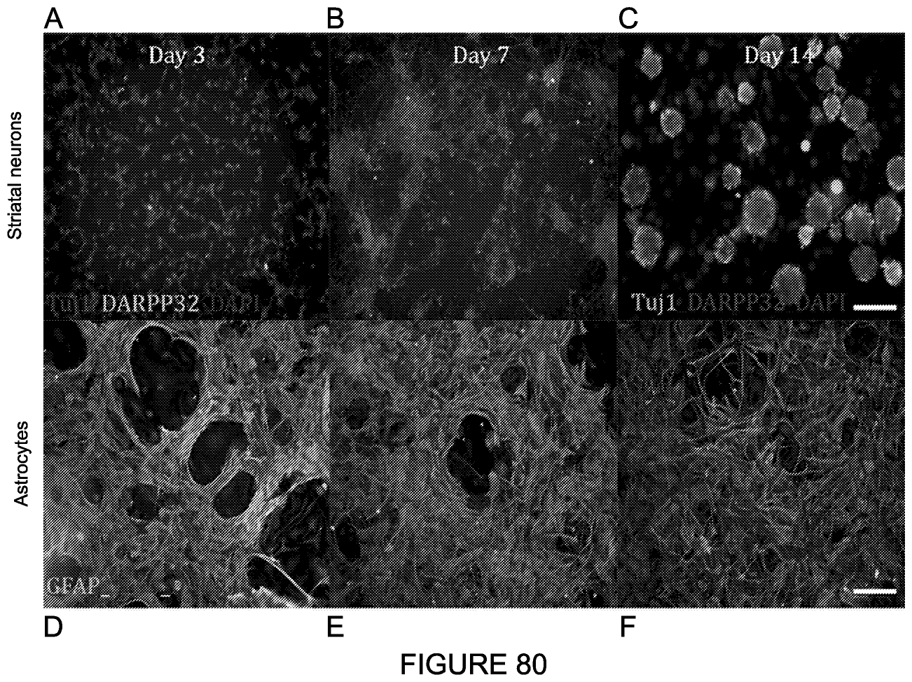

Using the dopaminergic neuron-specific (DARPP-32; FIG. 80, panels A-C, red) and astrocyte-specific (GFAP (FIG. 80, panels D-F green) and S-100 (FIG. 80, panels D-F, green) antibodies, cell types were identified We found that the overwhelming majority of cells were either dopaminergic neurons or astrocytes, according to the phenotypic markers that they expressed (FIG. 80, panels A-F).

Porosity Optimization

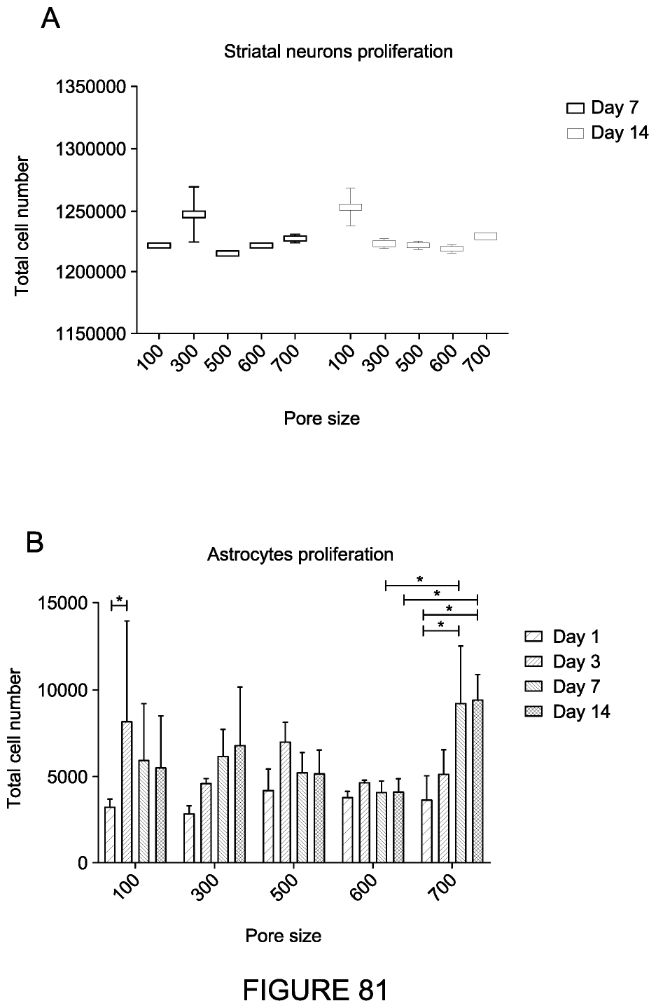

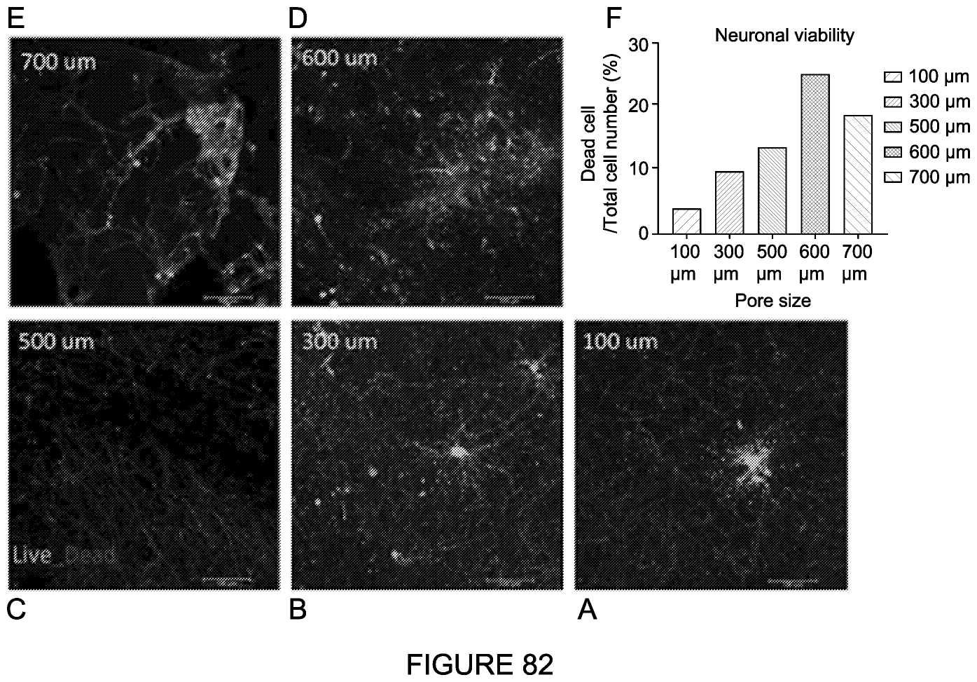

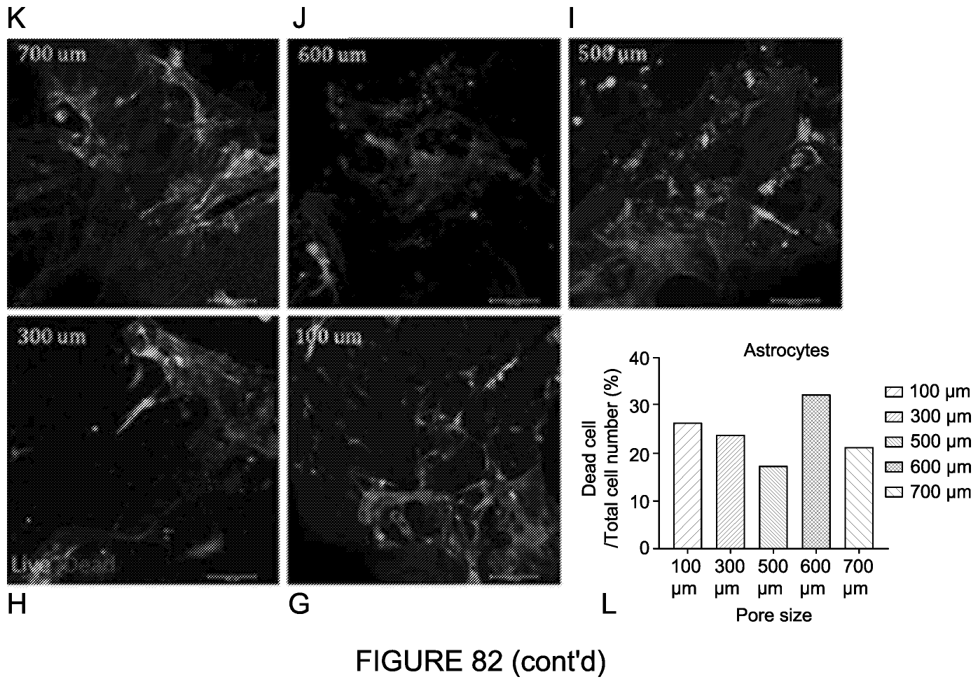

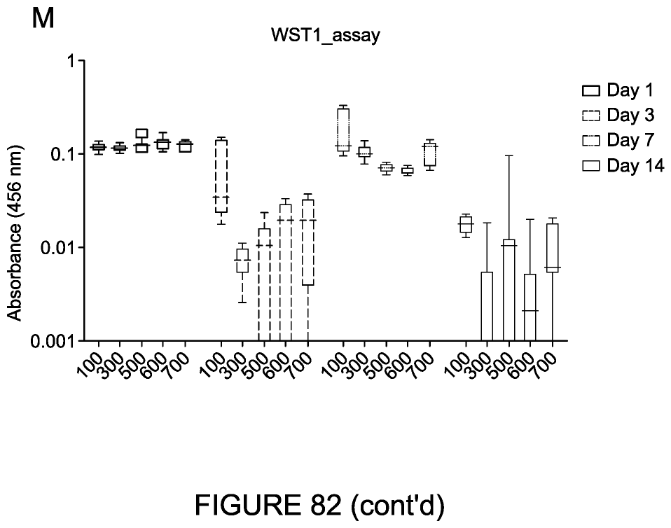

Number of striatal neurons was significantly higher (p<0.05) in scaffolds with 300 μm pores as compared to those with 600 μm pores (FIG. 81, panels A-B and 82, panels A-M), in contrast to previously published data that established 600 μm as a standard scaffold porosity for the brain model. Viability measurements from our studies showed a much higher dead cell count in the 600 μm scaffold, with the number of dead cells decreasing with decreasing porosity size (FIG. 82, panels E and K). Interestingly, astrocytes showed increased cell numbers in scaffolds with 700 um pores as compared to those with 600 μm pores after both 7 and 14 days in culture, as determined by cell number (via DNA quantification) (FIG. 82, panels K). Representative images from Live/Dead assays show that there were no statistically significant differences in numbers of dead cells in scaffolds of different porosities (FIG. 82, panels F and L). A WST1 assay was also performed in order to assess proliferation of astrocytes in scaffolds with different porosities (FIG. 82, panel M). In general, astrocytes appeared to proliferate more in scaffolds with 100 μm or 700 μm though there were no statistically significant differences between the groups at any time point.

Response of Co-Culture Models to Hemorrhage Challenge

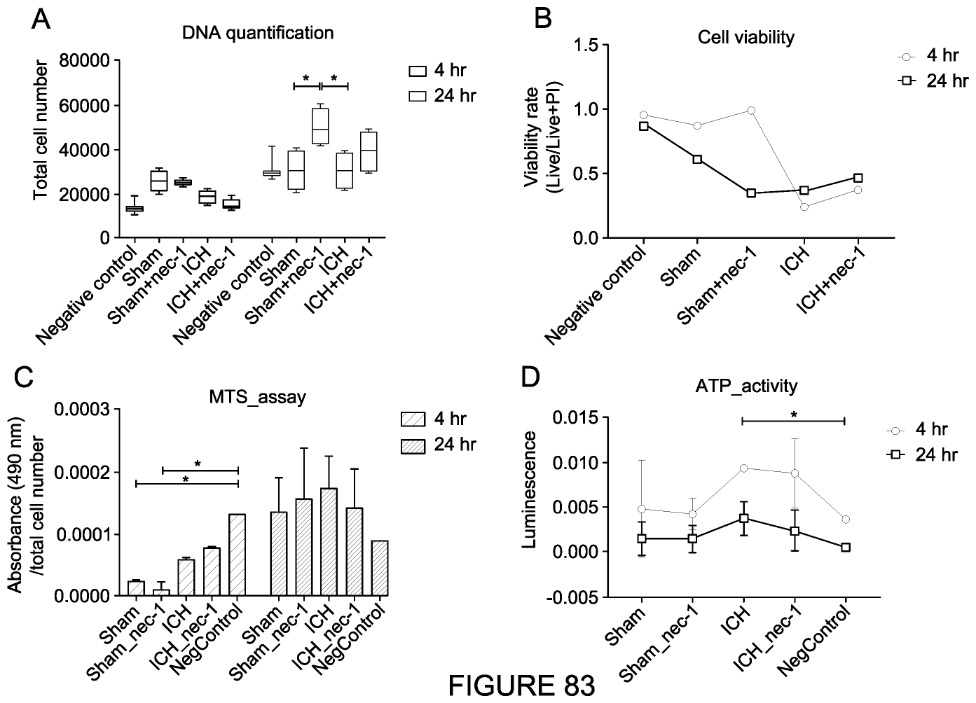

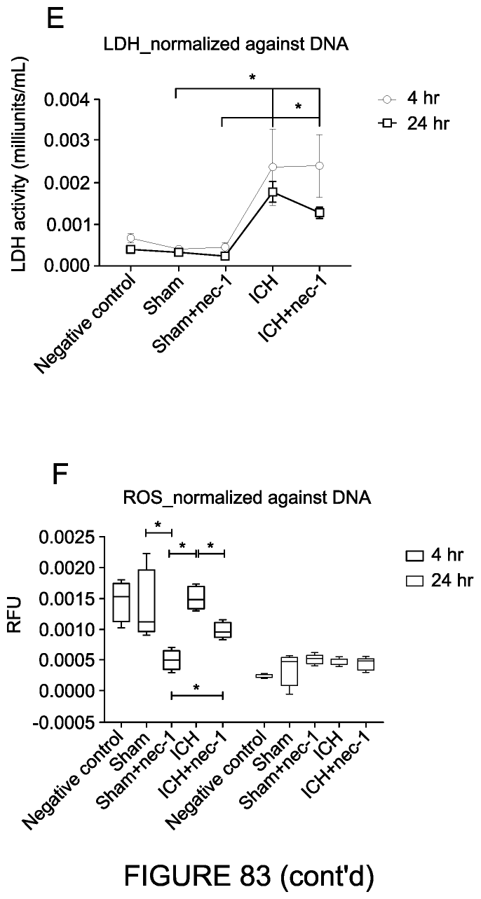

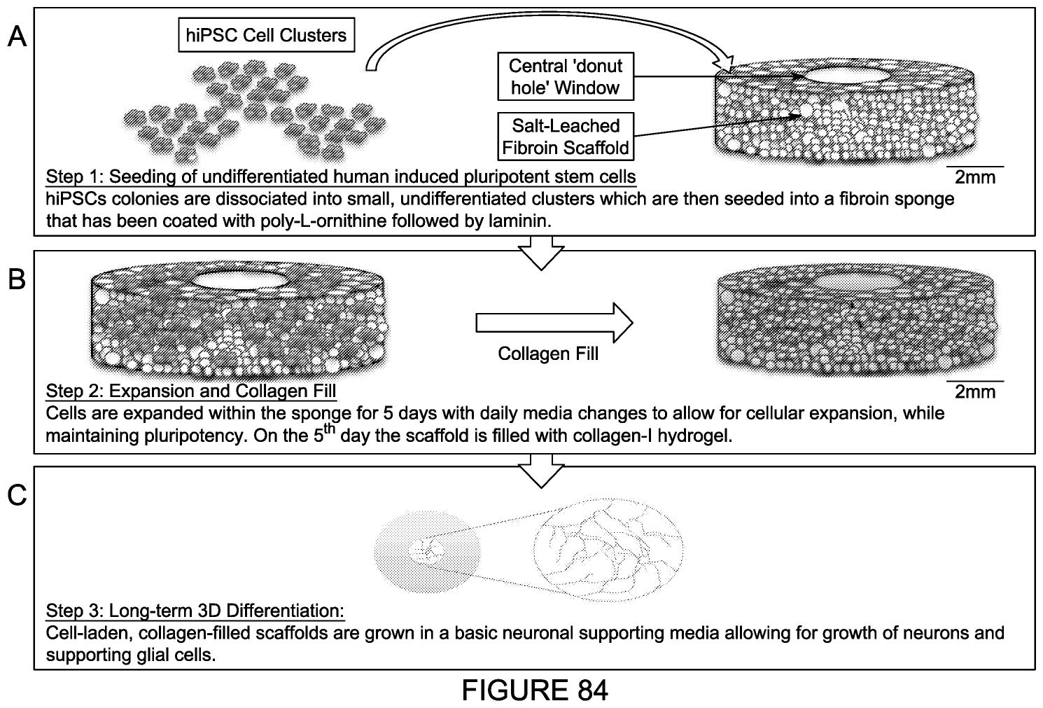

Live/Dead assays were performed on cells following hemorrhage challenges. No statistically different changes in numbers of live or dead cells was observed following hemorrhage challenge between groups, likely due to large variations between individual sham samples (FIG. 83, panels A-F). In response to the hemorrhage challenge, calcein/propidium iodide (FIG. 83, panel B). All groups displayed increased MTS metabolism between 4 and 24 hours, except for the negative control which showed a slight decrease in MTS metabolism (FIG. 83, panel C). ATP assays showed a higher level of activity in the ICH group than in the negative control, suggesting increased activity of astrocytes and microglia in response to the hemorrhage challenge. The ATP data (FIG. 83, panel D) are consistent with the MTS data which each show higher metabolic activity in the ICH groups, though these differences are not statistically significant, likely due to high variability between samples. In LDH assays, both the ICH and ICH+nec-1 groups had statistically higher levels of LDH activity than the two sham groups (FIG. 83, panel E). ROS-RNS assays showed a significantly lower level of reactive species in the Sham+nec-1 group as compared to sham alone. Additionally, the ICH group had a higher level of reactive species than treated sham and ICH groups (FIG. 83, panel F). No significant differences were observed between groups 24 hours post-injury.

### Discussion

We successfully developed an in vitro 3D model of intracerebral hemorrhage using neurons and glial cells (astrocytes and microglia).

The model showed trends in viability and metabolic response to the hemorrhage challenge. A direct mono-culture model comparison was not performed in the present study, so the effect of the glial cells alone cannot be determined in this Example; however, when compared with previous mono-culture hemorrhage data, the stabilization of the several of the metabolic assays after 24 hours suggests a brief period of astrocyte activation in response to the hemorrhage. A statistically significant increase in inflammatory markers in the tri-culture is considered a positive result and supportive of use of a tri-culture model rather than a mono-culture model. Additionally, a significant decrease in inflammatory markers after necrostatin-1 treatment supports that astrocytes and microglia are involved in the protective effect of necrostatin-1 treatment. A realistic constraint on the model is the low and variable yield of striatal neurons in a dissection from the brain of a single mouse. The number of brains required ranges from 5-10 and the exact number is not known until the dissection has already started. Additionally, yields may on the low side. At present, 60 million neurons is the highest yield obtained from a single brain, and 1 million neurons are needed for a single sample/assay, which limits the number of possible assays and sample numbers per assay. We will aim to improve cell collection in future studies, and have also considered alternative solutions such as using previously published scaffold porosity as used with silk fibroin scaffolds, as well as using glial cell lines instead of primary glial cells. Glial cell lines were not ultimately used because there was no difficulty with either altering the porosity of the sponge or seeding the glial cells onto the sponges with the neurons.

Overall, in these studies, scaffold pore sizes were smaller than expected (based on manufacturing method) across all scaffolds and intended porosities. This can likely be explained by noting that natural breaks in silk polymer of approximately 100 μm are common, and these were likely counted in the quantification of pore sizes. This would bring the averages down across all samples except the 100 μm samples, which is what we observe. We successfully verified the presence of dopaminergic neurons, astrocytes and microglia in the scaffolds using cell-specific markers: DARPP-32 (striatal neurons; FIG. 80, panels A-C, red) GFAP and S-100 (astrocytes, FIG. 80, panels D and F, green), and iba-1 (microglia, data not shown).

The porosity optimization test with the Live/Dead assay showed that striatal neurons had a higher viability in 300 μm sponges 7 and 14 days after seeding than the 600 μm control (FIG. 81). Astrocytes, on the other hand, had a higher cell count in 700 μm sponges than 600 μm sponges, 7 days after seeding. Due to potential changes in the mechanical properties of sponges with different porosities, mechanical properties of the different porosities may differ. To determine this, we will measure mechanical properties, such as, e.g., with compression tests, to determine if the neurons are responding to different properties of the sponges, such as, e.g., the Young's modulus or surface area. Viability tests after the hemorrhage challenge showed cell death in both ICH groups, with high variability in samples preventing any statistical significance in the Live/Dead assays. In the DNA quantification assay, the sham+nec-1 sample had the highest cell count and viability after 24 hours. An interesting trend in the DNA quantification data is higher cell counts observed at 24 hours as compared to 4 hours. Because neurons do not proliferate in the samples, the higher cell count must be the result of proliferation of the glial cells in response to the transportation to MGH and/or the hemorrhage challenge.

In metabolic assays, we observed a general trend towards higher activity in ICH groups as compared to that of shams or negative controls, though no results showed statistically significant differences. Importantly, this runs counter to data from previous hemorrhage challenges that use monocultures and show lower metabolic activity in ICH groups due to cell death (data not shown). This suggests that the glial cells are activated by the challenge and are producing more ATP and increasing their metabolism in response to the presence of blood. In the toxicity assays, LDH was more active in ICH groups than sham groups, likely due to the response of the cells to the presence of blood. The ROS-RNS assay showed that necrostatin-1 treatment resulted in a statistically significant decrease in the amount of reactive species present in the supernatant at 4 hours post-injury, and that the presence of blood led to a higher level of reactive species at 4 hours. After 24 hours, there are no significant differences between the groups, which suggests a transient response to the transportation and blood injections followed by stabilization. This response is indicative of activation of the astrocytes and microglia in response to the challenge. More replications with larger sample sizes are necessary to reduce the variability of the samples, but the reduction of reactive species and higher DNA content of samples treated with necrostatin-1 suggests that the treatment, which inhibits necroptosis, is affecting the samples. Since microglia are known to be activated by the presence of blood and secrete TNF-α, which then induces necroptosis in both astrocytes and neurons (Fan et al. 2016). Because of this, the model appears to be showing the response of microglia to the necrostatin-1 treatment and their activation in the ICH groups.

### CONCLUSIONS

Having a more accurate model helps patients by creating a more robust model for testing new potential drug candidates, leading to fewer false positive and false negatives. The use of our model increases the testing possibilities per mouse, as the effects of tens of different drugs can be tested for efficacy in parallel using only one pregnant mouse, rather than one adult mouse per drug or control condition/group. Because of the successful response of the model to necroptosis, one of the primary methods of neuronal damage after ICH (Su et al. 2015), this novel 3D brain tissue model is an effective tool for studying the effects of drugs on the striatum during ICH.

TABLE 3
DCF Standardization Concentrations forROS-RNS Assay

| Standard | DCF Standard | PBS | DCF |
|---|---|---|---|
| Tubes | (μL) | (μL) | (nM) |
| 1 | 10 | 990 | 10,000 |
| 2 | 100 of tube 1 | 900 | 1000 |
| 3 | 100 of tube 2 | 900 | 100 |
| 4 | 100 of tube 3 | 900 | 10 |
| 5 | 100 of tube 4 | 900 | 1 |
| 6 | 0 | 1000 | 0 |

TABLE 4
H₂O₂ Standardization Concentrations for ROS-RNS assay.

| | 2 mM H2O2 | | |
|---|---|---|---|
| Standard | Standard | PBS | H2O2 |
| Tubes | (μL) | (μL) | (μM) |
| 1 | 10 | 990 | 20 |
| 2 | 500 of tube 1 | 500 | 10 |
| 3 | 500 of tube 2 | 500 | 5 |
| 4 | 500 of tube 3 | 500 | 2.5 |
| 5 | 500 of tube 4 | 500 | 1.25 |
| 6 | 500 of tube 5 | 500 | 0.625 |
| 7 | 500 of tube 6 | 500 | 0.313 |
| 8 | 500 of tube 7 | 500 | 0.156 |
| 9 | 500 of tube 8 | 500 | 0.078 |
| 10 | 500 of tube 9 | 500 | 0.039 |
| 11 | 0 | 1000 | 0 |

### Example 17: Differentiation of Human Stem Cells in 3D Systems to Generate Functional Neural Network Tissue Models

The goal of this study was to demonstrate the viability of a novel 3D silk sponge-based tissue model for use in differentiation of human induced pluripotent stem cells (hiPSCs) to generate functional neural networks. While it does not display certain aspects of developmental organization that may be observed in other approaches, the direct differentiation of human stem cells to neurons and supporting glial cells within the this model provides a unique advantage, which is able to generate a stable and functional long-term culture model.

### Materials and Methods

Cells

The hiPSC lines ND41866*C and GM24666*A were obtained from the Coriell Biorepository (Camden, N.J., USA), the YZ1 hiPSC line was obtained from Dr. Giusepena Tesco courtesy of the University of Connecticut-Wesleyan Stem Cell Core (UCSCC, Farmington, Conn., USA).

hiPSC Maintenance

hiPSCs were grown in feeder-free conditions as previously described. In brief, plates were coated with Matrigel hESC-qualified Matrix (Corning, Corning, N.Y., USA) coated plates and maintained, with daily media changes using mTeSR1 (Stem Cell Technologies, Vancouver, BC, Canada), and were passaged 1:5 every 5-7 days using ReLeSR (Stem Cell Technologies, Vancouver, BC, Canada)

Scaffolds for 3D Model and Seeding

Silk protein was processed from Bombyx mori cocoons as described previously. The porous scaffolds were generated via salt leaching as explained previously. Scaffolds were then coated with poly-L-ornithine (15 ug/mL in PBS) (Sigma-Aldrich, St. Louis, Mo., USA) for at least 2 hours at room temperature, and then washed 2× with PBS, 1× with DMEM/F12, followed by a 2-hour laminin coating (10 ug/mL in DMEM/F12) (Roche, Basel, Switzerland). Undifferentiated hiPSCs were treated with ReLeSR (Stem Cell Technologies, Vancouver, BC, Canada) to specifically remove undifferentiated portions of the colonies. These cell clumps were then added to the scaffold at approximately 5.0×10\hat{ }6 cells/mL and incubated for 24 hours. Cells were allowed to attach to the scaffold overnight. The media was changed daily for 5 days following seeding, moving the scaffolds to fresh wells with each change. After 5 days of cellular expansion, the scaffolds were then moved to a fresh well and filled with 100 uL of a cold collagen-type I rat-tail solution (3.0-4.0 mg/mL) mixed with 10×PBS and 1N NaOH (88:10:2). These were then incubated at 37° C. until the collagen gelled (˜30 minutes). The collagen-filled scaffolds were then transferred to a 24 well plate and flooded with 1 mL of final neuro media (FNM) (Neurobasal media, Anti-Anti, Glutamax, with B-27 supplement) with media changed every 3-4 days.

For neural rosette formation the undifferentiated colonies were treated with Gentle Cell Dissociation Reagent and seeded at 3.0×10⁶ cells per well in an Aggrewell 800 plate (Stem Cell Technologies, Vancouver, BC, Canada) in StemDIFF Neural Induction Media (Stem Cell Technologies, Vancouver, BC, Canada). Half media changes were done daily. On day 5, spheres were collected from the Aggrewell 800 then plated onto a PLO/L-coated plate, remaining in StemDIFF Neural Induction Media with neural rosettes appearing as early as 1 day post-plating. On day 11, the rosettes were specifically selected for by using Neural Rosette Selection Reagent (Stem Cell Technologies, Vancouver, BC, Canada). These neural rosettes were then seeded into the scaffolds under the same conditions as previously mentioned. Cells were grown in scaffolds under these conditions for 3 months prior to analysis.

Nucleic Acid and Protein Isolation

At the time of desired analysis scaffolds were snap frozen using liquid nitrogen and can be stored at −80° C. These frozen scaffolds were then homogenized using a pre-chilled Spectrum Bessman Tissue Pulverizer (Fisher Scientific, Hampton, N.H., USA). DNA, RNA and protein were then collected independently using the AllPrep DNA/RNA/Protein Mini Kit (Qiagen, Hilden, Germany), according to the manufacterer's instructions. DNA was quantified using Quant-iT PicoGreen dsDNA Assay kit (Thermo-Fisher, Cambridge, Mass., USA), according to the manufacterer's instructions. Following extraction. RNA was quantified using a NanoDrop 2000 Spectrophotometer (Thermo-Fisher, Cambridge, Mass., USA)

RT-PCR

cDNA was generated using High-Capacity cDNA Reverse Transcription Kit (Thermo Fisher, Cambridge, Mass., USA), according to the manufacterer's instructions. This cDNA was used for qPCR using the Taqman system (Fast Advanced Mastermix supplemental figure for full list of probes) (Thermo Fisher, Cambridge, Mass., USA). For the 12-week time course qPCR, a Biomark HD (Fluidigm, South San Francisco, Calif., USA) was used in order to limit variability observed with the large number of samples and genes being tested, following their protocol for “Fast gene expression analysis using Taqman gene expression assays on the Biomark HD system.” In brief the cDNA was preamplified using fluidigm preamp mastermix, and Taqman assay primer/probes. The preamplified cDNA and taqman primer/probes (Table 5) are then used in combination with a 96.96 Dynamic Array™ IFC (Fluidigm, South San Francisco, Calif., USA), allowing for direct comparison of samples.

Immunocytochemistry

Cultures were fixed in 4% PFA (Santa Cruz, Dallas, Tex., USA) for 10-20 minutes, washed with 1×PBS, and then blocked with blocking solution (Goat Serum (15 mL), TritonX-100 (Sigma Aldrich, Natick, Mass., USA), sodium azide (5 mL), in 1×PBS (fill to 250 mL)) for 1 hour. Primary antibodies (beta-III-tubulin and glial fibrillary acidic protein), were diluted in the blocking solution at 1:500, and kept at 4° C. overnight. Cultures were washed 3 times with 1×PBS followed by incubation with secondary antibodies, diluted 1:250 in the blocking solution and allowed to incubate overnight at 4° C. The cultures were then washed 3 times with 1×PBS and stained with DAPI for 20 minutes.

Confocal Imaging and Analysis

A Leica SP8 Confocal microscope (Leica, Wetzlar, GER) was used to collect source image stacks. ImageJ was used to measure beta-III-tubulin density and to perform intensity measurements for calcium imaging.

Functional Experiments

Extracellular Field Potential Recordings (“EFPs”):

Scaffolds were recorded in a bath consisting of NaCl 140 mM, KCl 2.8 mM, CaCl₂ 2 mM, MgCl₂ 2 mM, HEPES 10 mM, and D-glucose 10 mM, with pH adjusted to 7.4 with NaOH. Sharp glass microelectrodes were filled with extracellular solution and had a resistance of 60-80 Mohms. The recording electrode was placed near the edge of the central window. Fast field potential changes (spikes) were recorded with an NPI amplifier at a bandwidth of 0.3-10 kHz and were further amplified with an A-M Systems Differential AC amplifier (Model 1700) to a combined total gain of 10,000×. The signals were digitized at 10 KHz by a Molecular Devices digitizer (Digidata 1550) using a Dell Optiplex GX620 computer with pClamp 10 software (Molecular Devices). Electrical stimulations (40 V, 1 ms pulses) of the cultured tissues were generated via a Grass S44 Stimulator, passed through a Grass Stimulus Isolation Unit (SIU5), and delivered through a platinum parallel bipolar electrode (FHC PBSB0875) with a distance of 800 μm between two tips positioned near the recording electrode. Activity inhibitors were used to confirm neuronal activity by use of glutamate receptor blockers (6-cyano-7-nitroquinoxaline-2,3-dione (CNQX) 50 μM, (2R)-amino-5-phosphonopentanoate (AP5) 250 μM) as well as the neuronal sodium channel blocker tetrodotoxin (TTX, 5 uM). After electrical stimulation (40V, 1 ms, at 0.1 Hz) and TTX addition, the cultures were stimulated again and the responses subtracted from the pre-TTX recordings to identify neural component of the stimulation response.

Calcium Imaging

Fluo-4 (Invitrogen, Carlsbad, Calif., USA) was used as described in the manufacturer's manual. In brief Fluo-4 was dissolved in Pluronic-F127 (Life Technologies, Carlsbad, Calif., USA) at a volume of 1 uL/ug. The resuspended Fluo-4 was then diluted in artificial cerebrospinal fluid (NaCl: 140 nM, KCl: 2.8 nM, CaCl₂: 2 nM, MgCl₂: 2 nM, HEPES: 10 mM, Glucose: 10 mM, pH7.4) 1:1000. Cells were incubated in the Fluo-4 solution for 60 mins at 37° C. After the incubation the cells were washed once with PBS and supplied with fresh artificial cerebrospinal fluid and imaged for 3 minutes in a climate-controlled chamber at 5% CO₂ and 37° C. on a Keyence BZ-X700 microscope (Itasca, Ill., USA).

Statistics:

Prism 5 by GraphPad Software was used to perform statistical analyses To assess changes in DNA content in cultures a two-way ANOVA (initial seeding density, time point) was performed with a Bonferroni post-hoc test on group averages. Prism was also used to generate the box-and-whisker plot for percent of beta-III tubulin coverage of max projections.

### Results

Attachment

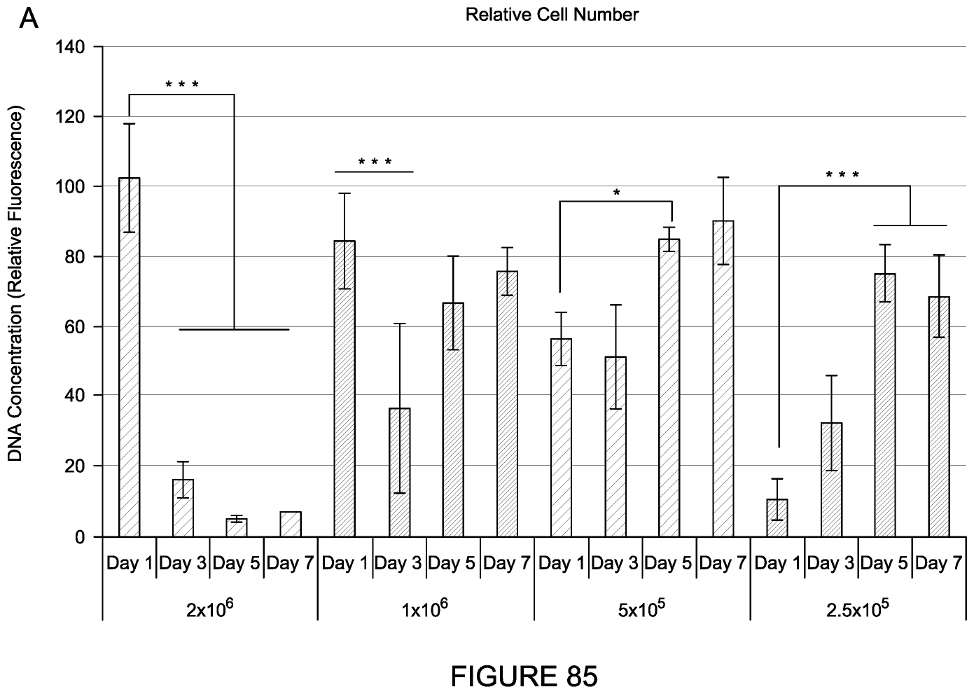

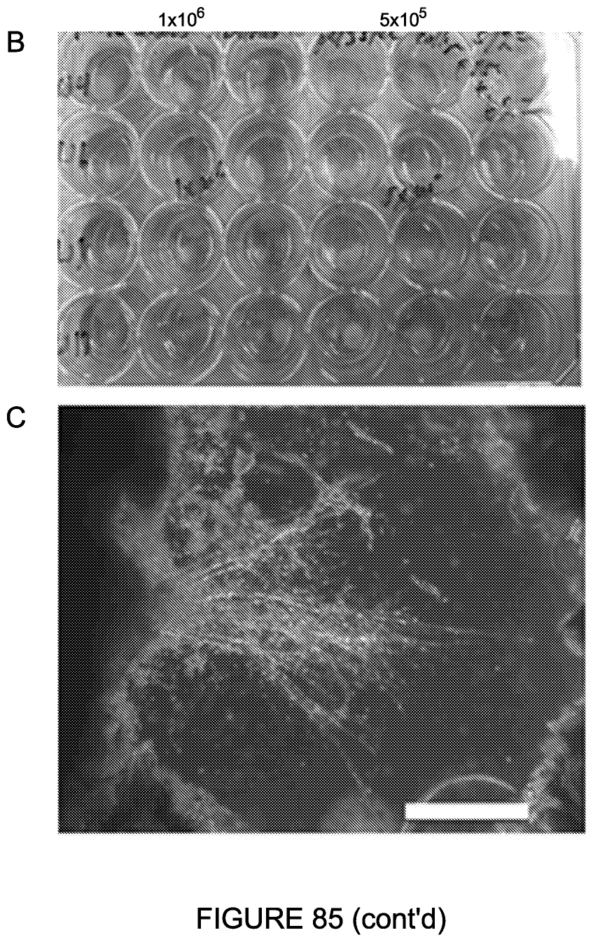

We initially set out to determine optimal conditions for cellular attachment to promote neural growth and support cell expansion within the novel scaffolds (FIG. 85, panels A-C). The porous donut-shaped scaffolds were coated with poly-ornithine (PLO), followed by laminin (PLO/L) to support cell attachment. The coated scaffolds were then seeded with clusters of hiPSCs derived from gentle colony dissociation using ReLeSR. Scaffolds were sized to fit within the wells of a 96 well plate and media volume was kept minimal to maximize capillary action in the sponge scaffolds. The cell population within the scaffolds, as determined by DNA content, was observed to have the highest density in the largest concentration condition (2×10⁶ cells/mL) at the initial time point, day 1 (FIG. 85). Initial DNA levels in the more concentrated starting conditions (2×10⁷ and 1×10⁷ cells/mL) experienced a significant loss by the third day of culture. To maximize cell content without significant loss, scaffolds were seeded with 5×10⁵ cells, and attachment and expansion was allowed for 5 days with daily media changes. On the 5^{th} day, the media was aspirated from the scaffolds, which were then filled with collagen-I hydrogel, and transferred to a 24 well plate and flooded with neural-supporting growth media.

Differentiation

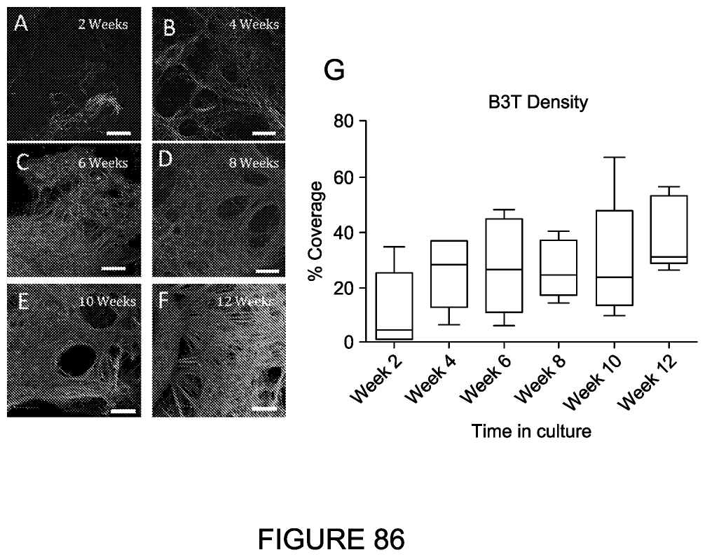

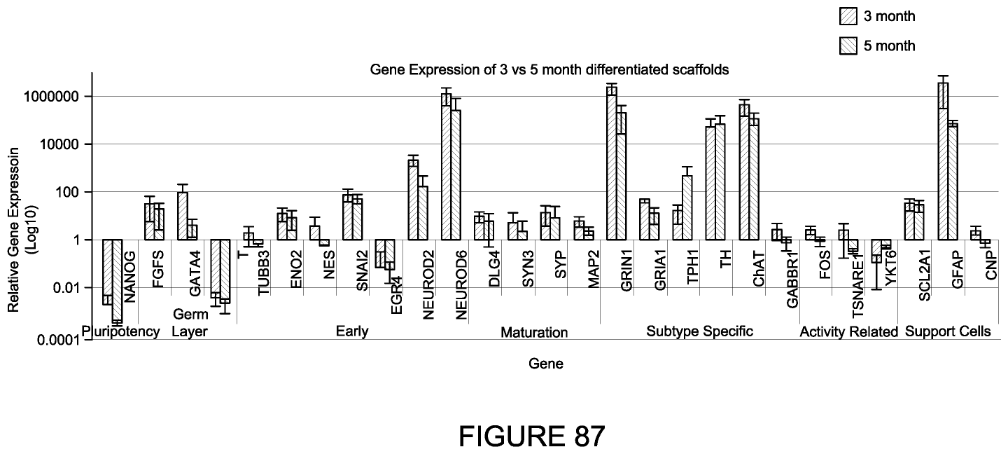

After scaffolds were transferred to the neural-supporting media, neuronal cells were observed as early as two weeks based on beta-III-tubulin staining (B3T+; FIG. 86, panels A-G) and gene expression (FIG. 87). Over time in culture the presence of B3T+ cells increased from a mean of 11.32% B3T coverage (range: 0.937%-34.59%) at week 2 (FIG. 86, panel A) to a mean of 38.65% coverage (range: 25.91%-56.21%) by week 12 (FIG. 86, panel F). The increase in B3T density demonstrated the continued growth of neurons within the tissue model over the 12-week period. In addition to specific neuronal subtypes, astrocytes were also observed. qPCR was performed to determine relative gene expression of the differentiated cultures (FIG. 87). cDNA derived from 3 and 5 month differentiated scaffolds was compared to that from the maintained hiPSCs. As differentiation progresses the expression of pluripotent markers (NANOG) is down regulated, while neural markers show various levels of up regulation. Notably the up regulation of multiple genes signifying various subtypes of neurons (GRIN1 and GRIA1—glutamatergic, TPH1—serotonergic, TH—dopaminergic, ChAT—cholinergic) indicates a diverse population within the 3D model in addition to the synaptic markers (DLG4, SYN3, and SYP) signifying the generation of network connections. We also observe high levels of expression of GFAP, a common marker for astrocytes. With increasing culture duration, expression of the pluripotent gene marker (NANOG) decreased, and expression of neural specific markers increased (ENO2, SNAI2, NEUROD2, NEUROD6). We also tested for germ-layer markers and observed an increase in both the endo- and ectodermal gene expression (GATA4 and FGF5, respectively) but saw very low expression of the mesodermal marker brachyury (T). The GATA4 expression, while undesirable, is not unexpected as the differentiation procedure is relatively undirected. As the culture length was extended the expression of the endodermal marker decreased. The upregulation of neural specific markers such as MAP2, ENO2, NEUROD2, NEUROD6, as well as synaptic gene expression (DLG4, SYP3 and SYN) compared to the control hiPSCs suggested the presence of a diverse population of neurons forming a network within the model. We also tested genetic markers that suggested the production of specific neurotransmitters (TH—dopamine, ChAT—acetylcholine, and TPH1—serotonin), which were all significantly upregulated, as were markers for both NMDA (GRIN1) and AMPA (GRIA1) glutamatergic receptors. The GABA receptor (GABBR1), does not appear to be increased which suggests little to no presence of inhibitory neurons. We also looked for markers of non-neuronal supporting cells (GFAP, SCL2A1 and CNP) to determine the existence of astrocytes, and glucose transporters found in the blood-brain-barrier. The low levels of CNP suggested that there were no oligodendrocytes within the tissue model as these are the cells responsible for the myelination of neurons.

Function

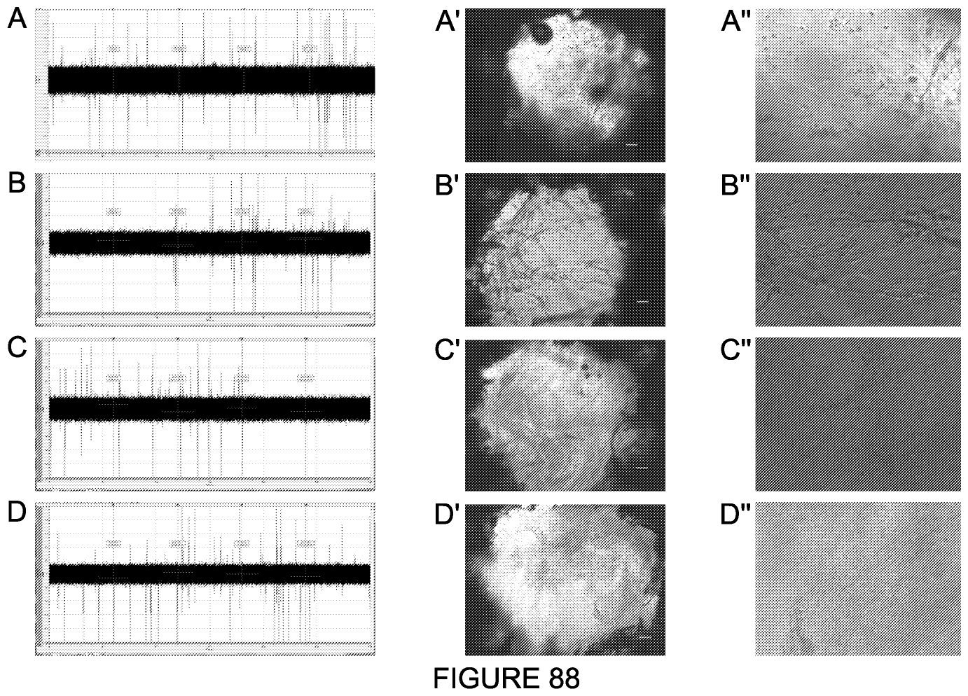

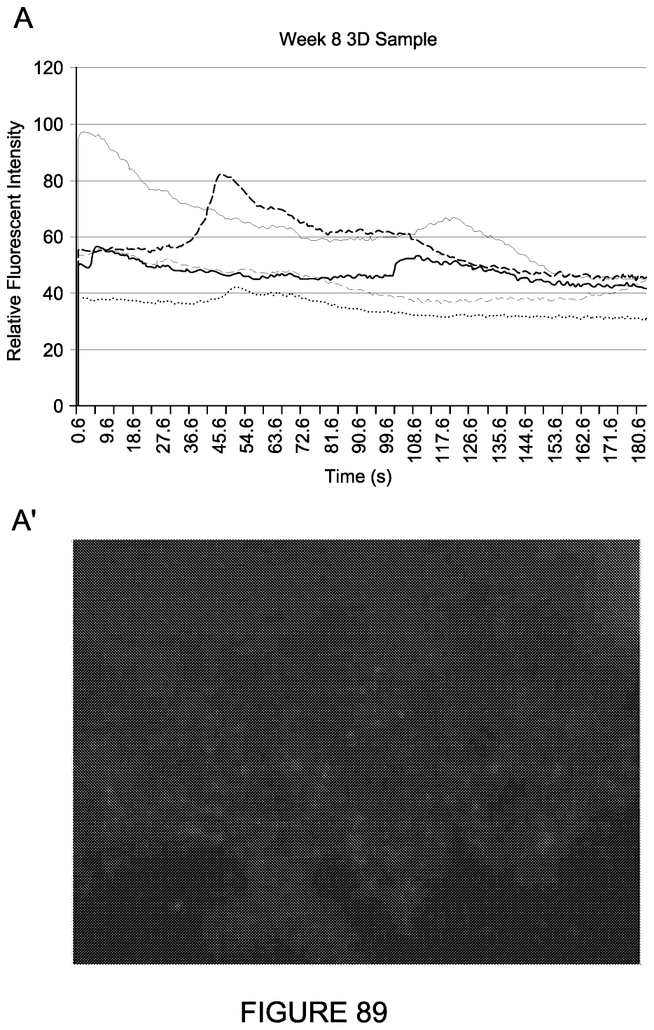

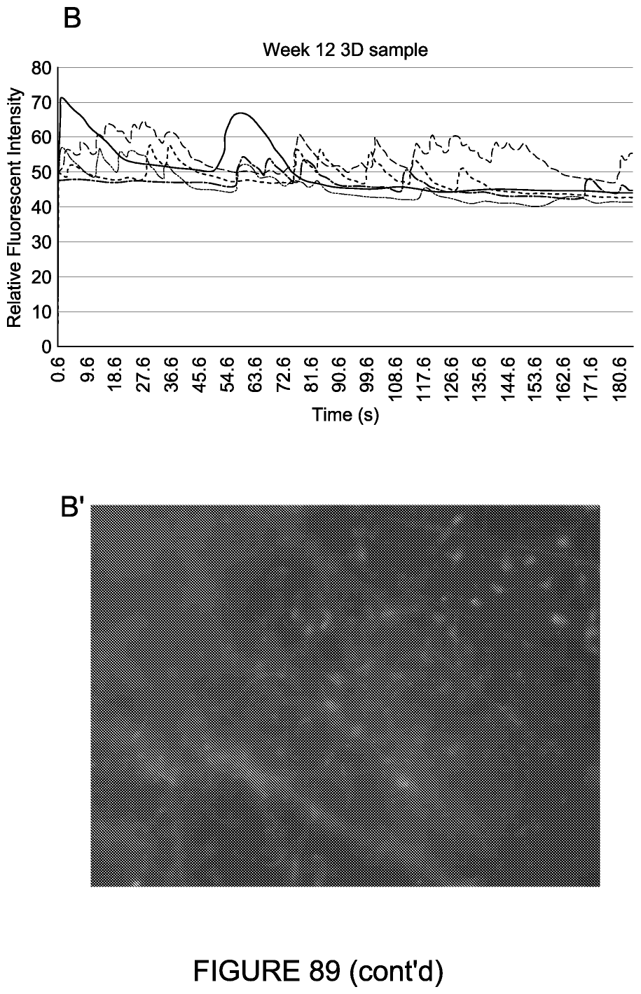

In traditional 2D cultures, electrophysiology is conducted through various patch-clamping methods, gathering functional information from single cells. When patch-clamping was attempted in our three-dimensional cultures, the recording electrodes often became damaged or clogged and as our engineered environment is critical for this culture approach, testing on 2D hiPSC-derived neuronal cultures could not and did not provide sufficient functional information. In order to determine activity within the scaffolds, local field potential (LFP) was utilized (FIG. 88, panels A-D″). The presence of both spontaneous and inducible activity within the scaffold by 10 weeks of growth indicates the presence of healthy, functioning neurons in our 3D network model. This function was able to be partially blocked using CNQX and AP-5 (AMPA/kainate receptor and NMDA receptor antagonists, respectively) indicating the presence of glutamatergic neurons within our model. Activity was also blocked using the sodium channel blocker tetrodotoxin (TTX). Calcium imaging using Fluo-4 showed results that were similar to and consistent with the LFP data, with activity seen in scaffolds as early as 8 weeks (FIG. 89), increasing through 12 weeks (FIG. 89) and observed at 7 months (data not shown).

Sporadic Alzheimer's Disease hiPSCs—

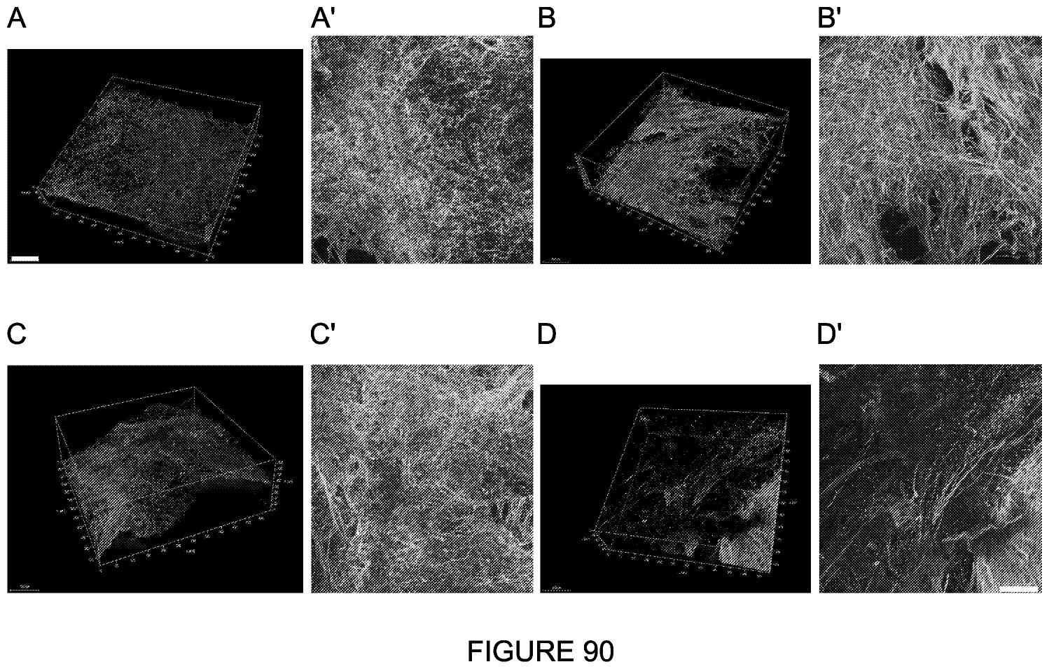

To assess the potential of this novel 3D tissue model for disease studies, three-dimensional differentiation of hiPSCs to neurons was confirmed with hiPSC cultures derived from patients diagnosed with neurodegenerative diseases such as Alzheimer's disease (AD). hiPSC cultures were derived from a patient diagnosed with Sporadic AD. A neural network was generated within the 3D tissue model with the AD derived hiPSCs (FIG. 90, panels A-D). FIG. 90, panels A-D′ show growth of neurons and astrocytes differentiated from hiPSCs for 8 months in cells from a patient diagnosed with Alzheimer's disease (Panels A and C) or a healthy control (panels B and D). Cells were stained with GFAP (red, astrocytes) and B3T (green, neurons). Panels A′, B′, C′, and D′ show the max projection of the confocal image in panels A, B, C, and D, respectively.

### Discussion

The described model improves and expands upon previous work published by our lab, which utilized rat primary cortical neurons to demonstrate the feasibility of this scaffold to support neuronal growth over long culture periods. By adapting this model for use with human iPS-derived neurons, we have generated a novel system that can be used in conjunction with patient-derived hiPSCs to model disease progression and possibly allow for testing of therapeutics and their respective effects on the disease state and impact on the neural network. Our lab has also previously demonstrated beneficial effects on growth of rat neurons by inclusion of brain-derived extracellular matrix components, which in combination with the human iPS-derived neurons described in this Example, could provide an avenue for matrix-related effects on disease or injury states within the neural model. The ability of this model to grow primary neurons as well as induced neural stem cells, and neurons derived directly from human induced pluripotent stem cells demonstrates the versatility of this scaffold for neural growth and the potential for numerous experimental applications.

There have been multiple models developed that mimic brain development and neurological disease states in engineered systems utilizing hiPSCs. Many of these approaches rely on the progression of neuronal differentiation from pluripotent cells to embryoid bodies and utilize the natural architecture developed in these spheroids with minimal external direction to develop organoids that exhibit multiple characteristics of the human brain. Due to the dense nature of these models, and lack of active perfusion through the models, the size of these models are limited. The model described in this Example shows the direct differentiation of hiPSCs to neurons, bypassing spheroid and neural rosette stages, all while within a 3D scaffold supporting the formation of a functional three-dimensional neural network. While this model also lacks an active perfusion system the nature of the porous scaffolding allowing increased levels of media diffusion.

When working with induced pluripotent stem cells, consistency of differentiation across multiple lines is necessary for model utility. Here, growth was not limited to a single hiPSC line, but was also demonstrated possible from sporadic AD patient-derived hiPSCs. This demonstration of neural network development across different patient disease states indicated the possibility of using these cultures to study early progression of chronic diseases in controlled 3D tissue cultures. One issue that could be optimized was the differing growth kinetics across hiPSC lines, which may necessitate variations in seeding and expansion prior to the collagen filling and cell differentiation process. This tissue model could also provide insight into early markers for diagnosis and possible therapeutics. The long-term in vitro stability of this model could also present an option for investigating later stages of neurodegenerative diseases that require long culture periods in vitro prior to observable deficits.

This model could also be useful to study the development and plasticity of neural networks under various stimulation conditions or after injury. In conclusion, this model provides an accessible option for studying neural growth from stem cells in three dimensions, and its durable versatility supports use in a variety of applications.

### CONCLUSIONS

We have developed a novel 3D human stem cell-derived neural tissue model that allows for the direct integration of undifferentiated stem cells into three-dimensional cultures and differentiation with consistency to a functioning network consisting of interconnected neurons and astrocytes. This tissue model supported the growth and neuronal differentiation of both healthy and disease-derived hiPSCs, while eliminating the preliminary differentiation steps typically needed with other stem cell-derived neural tissue models. This streamlined model limits the reagents and growth factors required for successful differentiation, supporting utility in terms of higher throughput for various screening applications. In addition, the sustainable cultivation in vitro provides a suitable 3D tissue system for the study of neurodegenerative disease states.

TABLE 5

| | | | Harvard |
|---|---|---|---|
| | | | Primer Bank |
| Marker | Gene (direction) | Sequence | ID |
| House Keeping | GIP Forward | CAAGGACCGCTTCAACCACTT | 296080692c1 |
| House Keeping | GIP Reverse | CCAGGATGGGTGTGTTTGACC | 296080692c1 |
| House Keeping | 18s rRNA Forward | GTAACCCGTTGAACCCCATT | — |
| House Keeping | 18s rRNA Reverse | CCATCCAATCGGTAGTAGCG | — |
| Pluripotency | KLF4 Forward | CCCACATGAAGCGACTTCCC | 194248076c1 |
| Pluripotency | KLF4 Reverse | CAGGTCCAGGAGATCGTTGAA | 194248076c1 |
| Pluripotency | NANOG Forward | TTTGTGGGCCTGAAGAAAACT | 153945815c1 |
| Pluripotency | NANOG Reverse | AGGGCTGTCCTGAATAAGCAG | 153945815c1 |
| Pluripotency | POU5F1 Forward | GTGTTCAGCCAAAAGACCATCT | 227430409c1 |
| Pluripotency | POU5F1 Reverse | GGCCTGCATGAGGGTTTCT | 227430409c1 |
| Endoderm | GATA4 Forward | CGACACCCCAATCTCGATATG | 172072611c1 |
| Endoderm | GATA4 Reverse | GTTGCACAGATAGTGACCCGT | 172072611c1 |
| Endoderm | SOX17 Forward | GTGGACCGCACGGAATTTG | 145275218c1 |
| Endoderm | SOX17 Reverse | GGAGATTCACACCGGAGTCA | 145275218c1 |
| Mesoderm | PAX3 Forward | AGCTCGGCGGTGTTTTTATCA | 300116151c1 |
| Mesoderm | PAX3 Reverse | CTGCACAGGATCTTGGAGACG | 300116151c1 |
| Mesoderm | T Forward | TATGAGCCTCGAATCCACATAGT | 19743811c1 |
| Mesoderm | T Reverse | CCTCGTTCTGATAAGCAGTCAC | 19743811c1 |
| Ectoderm | SNAI2 Forward | CGAACTGGACACACATACAGTG | 324072669c1 |
| Ectoderm | SNAI2 Reverse | CTGAGGATCTCTGGTTGTGGT | 324072669c1 |
| Ectoderm | TP63 Forward | CCACCTGGACGTATTCCACTG | 169234656c2 |
| Ectoderm | TP63 Reverse | TCGAATCAAATGACTAGGAGGGG | 169234656c2 |
| Early Neuronal | NES Forward | CTGCTACCCTTGAGACACCTG | 38176299c1 |
| Early Neuronal | NES Reverse | GGGCTCTGATCTCTGCATCTAC | 38176299c1 |
| Early Neuronal | OTX2 Forward | CAAAGTGAGACCTGCCAAAAAGA | 27436932c1 |
| Early Neuronal | OTX2 Reverse | TGGACAAGGGATCTGACAGTG | 27436932c1 |

### EQUIVALENTS AND SCOPE

While the present invention has been described herein in conjunction with various embodiments and examples, it is not intended that the scope be limited to such embodiments or examples. On the contrary, the present invention encompasses various alternatives, modifications, and equivalents, as will be appreciated by those of skill in the art. Accordingly, the descriptions, methods and diagrams of should not be read as limited to the described order of elements unless stated to that effect.

1. A composition comprising
a plurality of human nerve cells; a plurality of glial cells; and
silk fibroin.

2. The composition of claim 1, wherein the silk fibroin comprises a three dimensional matrix.

3. The composition of claim 2, wherein the silk fibroin is selected from the group consisting of silkworm silk fibroin, spider silk fibroin, silk tropoelastin blends, one or more genetically engineered variants of silk fibroin, and combinations thereof.

4. The composition of claim 2, wherein the matrix is a scaffold.

5. The composition of claim 2, wherein the three dimensional matrix comprises a plurality of pores.

6. The composition of claim 5, wherein the pores are interconnected.

7. The composition of claim 5, wherein the plurality of pores is substantially random.

8. The composition of claim 5, wherein the plurality of pores is substantially aligned.

9. The composition of claim 5, wherein the pores are about 50 um to about 1000 um in size.

10. The composition of claim 1, wherein the plurality of glial cells is human glial cells.

11. The composition of claim 1, wherein the plurality of glial cells is selected from the group consisting of glial progenitor cells, ependymal cells, NG2 cells, astrocytes, microglia, oligodendrocytes, Schwann cells, tanycytes, and combinations thereof.

12. The composition of claim 1, further comprising a plurality of endothelial cells.

13. The composition of claim 12, wherein the endothelial cells are human endothelial cells.

14. The composition of claim 12, wherein the endothelial cells comprise a monolayer.

15. The composition of claim 14, wherein the monolayer is characterized in that it is able to impede the flow of at least one fluid from an area substantially devoid of the plurality of human nerve cells and the plurality of glial cells into an area comprising the substantial majority of the plurality of human nerve cells and the plurality of glial cells.

16. The composition of claim 15, wherein the at least one fluid is a liquid or a gas.

17. The composition of claim 1, further comprising a plurality of brain microvascular endothelial cells.

18. The composition of claim 1, wherein the plurality of human nerve cells is selected from the group consisting of neurons, neural stem cells, induced pluripotent stem cells, and combinations thereof.

19. The composition of claim 18, wherein the plurality of neurons comprise at least one of motor neurons and sensory neurons.

20. A composition comprising a porous, three dimensional silk fibroin matrix comprising a plurality of pores, a conduit within the matrix sufficient to support flow of at least one fluid, a plurality of brain microvascular endothelial cells, and at least one extracellular matrix (ECM) agent.

21-37. (canceled)
